SEQUENCE LISTING (189 sequences)

SEQ ID NO: 1 - protein, 384 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 2 - protein, 386 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 3 - protein, 461 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 4 - protein, 462 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 5 - protein, 435 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 6 - protein, 435 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 7 - protein, 589 aa, synthetic construct
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 8 - protein, 593 aa, synthetic construct
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 9 - protein, 513 aa, synthetic construct
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 10 - protein, 435 aa, synthetic construct
MKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 11 - protein, 456 aa, synthetic construct
MALPVTALLLPLALLLHAARPHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 12 - protein, 432 aa, synthetic construct
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 13 - protein, 166 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 14 - protein, 503 aa, synthetic construct
QKSPQPLTRRATMGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSSEGLAGGPTAGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEPIIGGNTPLVFGKGTRLSVIANIQNPDPAVYQLRDSKSSD

SEQ ID NO: 15 - protein, 195 aa, synthetic construct
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 16 - protein, 242 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 17 - protein, 215 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHR

SEQ ID NO: 18 - protein, 373 aa, synthetic construct
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 19 - protein, 215 aa, synthetic construct
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 20 - protein, 209 aa, synthetic construct
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 21 - protein, 23 aa, synthetic construct
VTGISLLPPLGVAISVIIIFYCY

SEQ ID NO: 22 - protein, 25 aa, synthetic construct
PILLTISILSFFSVALLVILACVLW

SEQ ID NO: 23 - protein, 21 aa, synthetic construct
LLPSWAITLISVNGIFVICCL

SEQ ID NO: 24 - protein, 25 aa, synthetic construct
YALIPIPLAVITTCIVLYMNGILKC

SEQ ID NO: 25 - protein, 21 aa, synthetic construct
IVVGVVCTLLVALLMAALYLV

SEQ ID NO: 26 - protein, 24 aa, synthetic construct
IFMYLLTVFLITQMIGSALFAVYL

SEQ ID NO: 27 - protein, 20 aa, synthetic construct
MALPVTALLLPLALLLHAAR

SEQ ID NO: 28 - protein, 28 aa, synthetic construct
PILLTCPTISILSFFSVALLVILACVLW

SEQ ID NO: 29 - protein, 13 aa, synthetic construct
ILLPPCLTISILS

SEQ ID NO: 30 - protein, 11 aa, synthetic construct
ILLTISKCHLS

SEQ ID NO: 31 - protein, 13 aa, synthetic construct
IFSCGPLTISILS

SEQ ID NO: 32 - protein, 12 aa, synthetic construct
ILLTCHLISILS

SEQ ID NO: 33 - protein, 16 aa, synthetic construct
ILLTPPVCSVTISILS

SEQ ID NO: 34 - protein, 14 aa, synthetic construct
ILLFCRKDTISILS

SEQ ID NO: 35 - protein, 11 aa, synthetic construct
ILLRCTISILS

SEQ ID NO: 36 - protein, 11 aa, synthetic construct
ILPCPLISILS

SEQ ID NO: 37 - protein, 12 aa, synthetic construct
ILRCPSTISILS

SEQ ID NO: 38 - protein, 11 aa, synthetic construct
ILLTHRGCILS

SEQ ID NO: 39 - protein, 12 aa, synthetic construct
ILPLCSAISILS

SEQ ID NO: 40 - protein, 14 aa, synthetic construct
ILPIYRCVLISILS

SEQ ID NO: 41 - protein, 11 aa, synthetic construct
ILLECTISILS

SEQ ID NO: 42 - protein, 11 aa, synthetic construct
IFTCPSISILS

SEQ ID NO: 43 - protein, 12 aa, synthetic construct
ILCPSPTISILS

SEQ ID NO: 44 - protein, 12 aa, synthetic construct
ILLTSHQPCILS

SEQ ID NO: 45 - protein, 22 aa, synthetic construct
ILLTISILSCSTISILSFFSVA

SEQ ID NO: 46 - protein, 12 aa, synthetic construct
ILLTISICQSVA

SEQ ID NO: 47 - protein, 12 aa, synthetic construct
ICGIRETISILS

SEQ ID NO: 48 - protein, 11 aa, synthetic construct
ILLGCTISILS

SEQ ID NO: 49 - protein, 11 aa, synthetic construct
ILLSCTISILS

SEQ ID NO: 50 - protein, 11 aa, synthetic construct
ILLGCNISILS

SEQ ID NO: 51 - protein, 13 aa, synthetic construct
ILLTMRPCGSILS

SEQ ID NO: 52 - protein, 13 aa, synthetic construct
ILLTKKCTNSILS

SEQ ID NO: 53 - protein, 11 aa, synthetic construct
SFFSGALLVIL

SEQ ID NO: 54 - protein, 12 aa, synthetic construct
SFFSVEKVLVIL

SEQ ID NO: 55 - protein, 12 aa, synthetic construct
SFFSVEKALVIL

SEQ ID NO: 56 - protein, 12 aa, synthetic construct
SFFSVEAVLVIL

SEQ ID NO: 57 - protein, 16 aa, synthetic construct
ILLTMPEQDCPITILS

SEQ ID NO: 58 - protein, 16 aa, synthetic construct
NNSSGASWCLTISILS

SEQ ID NO: 59 - protein, 13 aa, synthetic construct
EMDPICPPISILS

SEQ ID NO: 60 - protein, 9 aa, synthetic construct
NLSCTKLTL

SEQ ID NO: 61 - protein, 29 aa, synthetic construct
PILLPPCLTISILSFFSVALLVILACVLW

SEQ ID NO: 62 - protein, 9 aa, synthetic construct
NLSCTKLTL

SEQ ID NO: 63 - protein, 27 aa, synthetic construct
PILLTISKCHLSFFSVALLVILACVLW

SEQ ID NO: 64 - protein, 29 aa, synthetic construct
PIFSCGPLTISILSFFSVALLVILACVLW

SEQ ID NO: 65 - protein, 28 aa, synthetic construct
PILLTCHLISILSFFSVALLVILACVLW

SEQ ID NO: 66 - protein, 32 aa, synthetic construct
PILLTPPVCSVTISILSFFSVALLVILACVLW

SEQ ID NO: 67 - protein, 30 aa, synthetic construct
PILLFCRKDTISILSFFSVALLVILACVLW

SEQ ID NO: 68 - protein, 27 aa, synthetic construct
PILLRCTISILSFFSVALLVILACVLW

SEQ ID NO: 69 - protein, 27 aa, synthetic construct
PILPCPLISILSFFSVALLVILACVLW

SEQ ID NO: 70 - protein, 32 aa, synthetic construct
PILLTMPEQDCPITILSFFSVALLVILACVLW

SEQ ID NO: 71 - protein, 31 aa, synthetic construct
NNSSGASWCLTISILSFFSVALLVILACVLW

SEQ ID NO: 72 - protein, 28 aa, synthetic construct
EMDPICPPISILSFFSVALLVILACVLW

SEQ ID NO: 73 - protein, 28 aa, synthetic construct
PILRCPSTISILSFFSVALLVILACVLW

SEQ ID NO: 74 - protein, 27 aa, synthetic construct
PILLTHRGCILSFFSVALLVILACVLW

SEQ ID NO: 75 - protein, 28 aa, synthetic construct
PILPLCSAISILSFFSVALLVILACVLW

SEQ ID NO: 76 - protein, 30 aa, synthetic construct
PILPIYRCVLISILSFFSVALLVILACVLW

SEQ ID NO: 77 - protein, 27 aa, synthetic construct
PILLECTISILSFFSVALLVILACVLW

SEQ ID NO: 78 - protein, 27 aa, synthetic construct
PIFTCPSISILSFFSVALLVILACVLW

SEQ ID NO: 79 - protein, 28 aa, synthetic construct
PILCPSPTISILSFFSVALLVILACVLW

SEQ ID NO: 80 - protein, 28 aa, synthetic construct
PILLTSHQPCILSFFSVALLVILACVLW

SEQ ID NO: 81 - protein, 33 aa, synthetic construct
PILLTISILSCSTISILSFFSVALLVILACVLW

SEQ ID NO: 82 - protein, 23 aa, synthetic construct
PILLTISICQSVALLVILACVLW

SEQ ID NO: 83 - protein, 28 aa, synthetic construct
PICGIRETISILSFFSVALLVILACVLW

SEQ ID NO: 84 - protein, 27 aa, synthetic construct
PILLGCTISILSFFSVALLVILACVLW

SEQ ID NO: 85 - protein, 27 aa, synthetic construct
PILLSCTISILSFFSVALLVILACVLW

SEQ ID NO: 86 - protein, 30 aa, synthetic construct
EMDPCRPHLTISILSFFSVALLVILACVLW

SEQ ID NO: 87 - protein, 27 aa, synthetic construct
PILLGCNISILSFFSVALLVILACVLW

SEQ ID NO: 88 - protein, 29 aa, synthetic construct
PILLTMRPCGSILSFFSVALLVILACVLW

SEQ ID NO: 89 - protein, 29 aa, synthetic construct
PILLTKKCTNSILSFFSVALLVILACVLW

SEQ ID NO: 90 - protein, 25 aa, synthetic construct
PILLTISILSFFSGALLVILACVLW

SEQ ID NO: 91 - protein, 26 aa, synthetic construct
PILLTISILSFFSVEKVLVILACVLW

SEQ ID NO: 92 - protein, 26 aa, synthetic construct
PILLTISILSFFSVEKALVILACVLW

SEQ ID NO: 93 - protein, 26 aa, synthetic construct
PILLTISILSFFSVEAVLVILACVLW

SEQ ID NO: 94 - protein, 30 aa, synthetic construct
SGEMDPTCLTISILSFFSVALLVILACVLW

SEQ ID NO: 95 - protein, 36 aa, synthetic construct
SGEMDPITLYCKTLLTISILSFFSVALLVILACVLW

SEQ ID NO: 96 - protein, 27 aa, synthetic construct
PILLGCTISILSFFSVALLVILACVLW

SEQ ID NO: 97 - protein, 27 aa, synthetic construct
PISPCITISILSFFSVALLVILACVLW

SEQ ID NO: 98 - protein, 28 aa, synthetic construct
PILLTISILSFFSGFSVALLVILACVLW

SEQ ID NO: 99 - protein, 33 aa, synthetic construct
PIDTRVYNSICLTISILSFFSVALLVILACVLW

SEQ ID NO: 100 - protein, 37 aa, synthetic construct
PILLTISILSFFSVSLILIVPCACELALLVILACVLW

SEQ ID NO: 101 - protein, 29 aa, synthetic construct
PILLSRCLTISILSFFSVALLVILACVLW

SEQ ID NO: 102 - protein, 31 aa, synthetic construct
PIWAALLNCETISILSFFSVALLVILACVLW

SEQ ID NO: 103 - protein, 28 aa, synthetic construct
PILLTNDCSSILSFFSVALLVILACVLW

SEQ ID NO: 104 - protein, 25 aa, synthetic construct
PILLTISILSPLGEALLVILACVLW

SEQ ID NO: 105 - protein, 28 aa, synthetic construct
PILNPCLTISILSFFSVALLVILACVLW

SEQ ID NO: 106 - protein, 28 aa, synthetic construct
PILLTCPTISILSFFSVALLVILACVLW

SEQ ID NO: 107 - protein, 30 aa, synthetic construct
EMDPSANCGAISILSFFSVALLVILACVLW

SEQ ID NO: 108 - protein, 29 aa, synthetic construct
PILLVSCPTISILSFFSVALLVILACVLW

SEQ ID NO: 109 - protein, 27 aa, synthetic construct
PILLIISIQWLSFFSVALLVILACVLW

SEQ ID NO: 110 - protein, 30 aa, synthetic construct
EMDQSPSCLTISILSFFSVALLVILACVLW

SEQ ID NO: 111 - protein, 30 aa, synthetic construct
EMDPCLEGLTISILSFFSVALLVILACVLW

SEQ ID NO: 112 - protein, 24 aa, synthetic construct
PILLTISILSFFWNLLVILACVLW

SEQ ID NO: 113 - protein, 28 aa, synthetic construct
EMDRFCPHISILSFFSVALLVILACVLW

SEQ ID NO: 114 - protein, 24 aa, synthetic construct
EMDLKCILSFFSVALLVILACVLW

SEQ ID NO: 115 - protein, 30 aa, synthetic construct
PIFHPFNCGPISILSFFSVALLVILACVLW

SEQ ID NO: 116 - protein, 28 aa, synthetic construct
PILLMCPTISILSFFSVALLVILACVLW

SEQ ID NO: 117 - protein, 28 aa, synthetic construct
PILLTISILSFFSGPSLALLVILACVLW

SEQ ID NO: 118 - protein, 29 aa, synthetic construct
PILRLECVTISILSFFSVALLVILACVLW

SEQ ID NO: 119 - protein, 25 aa, synthetic construct
PIPQGGCILSFFSVALLVILACVLW

SEQ ID NO: 120 - protein, 25 aa, synthetic construct
EMDIQSCILSFFSVALLVILACVLW

SEQ ID NO: 121 - protein, 29 aa, synthetic construct
PIFPHQHCTISILSFFSVALLVILACVLW

SEQ ID NO: 122 - protein, 16 aa, synthetic construct
PITLYCKTLLTISILS

SEQ ID NO: 123 - protein, 11 aa, synthetic construct
ISPCITISILS

SEQ ID NO: 124 - protein, 8 aa, unidentified
VWPSLPDH

SEQ ID NO: 125 - protein, 7 aa, unidentified
KKRIKPI

SEQ ID NO: 126 - protein, 13 aa, unidentified
KKTLEHLCKKPRK

SEQ ID NO: 127 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPQQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 128 - DNA, 1565 bp, Homo sapiens
  source 1..1565: mol_type = genomic DNA
agtctcacttcagttccttttgcatgaagagctcagaatcaaaagaggaaaccaacccctaagatgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtccagagaaaggtctggacatctatctcatcattggcatatgtggaggaggcagcctcttgatggtctttgtggcactgctcgttttctatatcaccaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaaaaaagatagaaactgtctttttcaataaaaagcactgtggatttctgccctcctgatgtgcatatccgtacttccatgaggtgttttctgtgtgcagaacattgtcacctcctgaggctgtgggccacagccacctctgcatcttcgaactcagccatgtggtcaacatctggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgcttaagagactctggagtttcttatgtgccctggtggacacttgcccaccatcctgtgagtaaaagtgaaataaaagctttgactaga

SEQ ID NO: 129 - DNA, 1643 bp, Homo sapiens
  source 1..1643: mol_type = genomic DNA
agtctcacttcagttccttttgcatgaagagctcagaatcaaaagaggaaaccaacccctaagatgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtccaggaggcagcatccttggccagagtaatgggctctctgcctggacccctcccagccatcccacttctcttccttttgcagagaaaggtctggacatctatctcatcattggcatatgtggaggaggcagcctcttgatggtctttgtggcactgctcgttttctatatcaccaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaaaaaagatagaaactgtctttttcaataaaaagcactgtggatttctgccctcctgatgtgcatatccgtacttccatgaggtgttttctgtgtgcagaacattgtcacctcctgaggctgtgggccacagccacctctgcatcttcgaactcagccatgtggtcaacatctggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgcttaagagactctggagtttcttatgtgccctggtggacacttgcccaccatcctgtgagtaaaagtgaaataaaagctttgactaga

SEQ ID NO: 130 - protein, 385 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 131 - DNA, 2406 bp, Homo sapiens
  source 1..2406: mol_type = genomic DNA
cgggaaggatgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctgggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtgactcggctaggtggggcaaggctgggcagtgtccgagagagcacccctctctgcatctgaccacgtgctacccccatgctggaggtgacatctcttacgcccaacccttccccactgcacacacctcagaggctgttcttggggccctacaccttgaggaggggcaggtaaactcctgtcctttacacattcggctccctggagccagactctggtcttctttgggtaaacgtgtgacgggggaaagccaaggtctggagaagctcccaggaacaatcgatggccttgcagcactcacacaggacccccttcccctaccccctcctctctgccgcaatacaggaacccccaggggaaagatgagcttttctaggctacaattttctcccaggaagctttgatttttaccgtttcttccctgtattttctttctctactttgaggaaaccaaagtaaccttttgcacctgctctcttgtaatgatatagccagaaaaacgtgttgccttgaaccacttccctcatctctcctccaagacactgtggacttggtcaccagctcctcccttgttctctaagttccactgagctccatgtgccccctctaccatttgcagagtcctgcacagttttctggctggagcctagaacaggcctcccaagttttaggacaaacagctcagttctagtctctctggggccacacagaaactctttttgggctcctttttctccctctggatcaaagtaggcaggaccatgggaccaggtcttggagctgagcctctcacctgtactcttccgaaaaatcctcttcctctgaggctggatcctagccttatcctctgatctccatggcttcctcctccctcctgccgactcctgggttgagctgttgcctcagtcccccaacagatgcttttctgtctctgcctccctcaccctgagccccttccttgctctgcacccccatatggtcatagcccagatcagctcctaacccttatcaccagctgcctcttctgtgggtgacccaggtccttgtttgctgttgatttctttccagaggggttgaacagggatcctggtttcaatgacggttggaaatagaaatttccagagaagagagtattgggtagatattttttctgaatacaaagtgatgtgtttaaatactgcaattaaagtgatactgaaacacaaaaaaaaaaaaaaaaaaaaaaaaaaaaaaaaaaaaaaaaa

SEQ ID NO: 132 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 133 - DNA, 2824 bp, Homo sapiens
  source 1..2824: mol_type = genomic DNA
aagtaacagaagttagaaggggaaatgtcgcctctctgaagattacccaaagaaaaagtgatttgtcattgctttatagactgtaagaagagaacatctcagaagtggagtcttaccctgaaatcaaaggatttaaagaaaaagtggaatttttcttcagcaagctgtgaaactaaatccacaacctttggagacccaggaacaccctccaatctctgtgtgttttgtaaacatcactggagggtcttctacgtgagcaattggattgtcatcagccctgcctgttttgcacctgggaagtgccctggtcttacttgggtccaaattgttggctttcacttttgaccctaagcatctgaagccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaagatctgaaggtctcacctccatttgcaattgacctcttctgggaacttcctcagatggacaagattaccccaccttgccctttacgtatctgctcttaggtgcttcttcacttcagttgctttgcaggaagtgtctagaggaatatggtgggcacagaagtagctctggtgaccttgatcaaggggttttgaaatgcagaattcttgagttctggaagggactttagagaataccagtgttattaatgacaaaggcactgaggcccagggaggtgacccgaattataaaggccagcgccagaacccagatttcctaactctggtgctctttccctttatcagtttgactgtggcctgttaactggtatatacatatatatgtcaggcaaagtgctgctggaagtagaatttgtccaataacaggtcaacttcagagactatctgatttcctaatgtcagagtagaagattttatgctgctgtttacaaaagcccaatgtaatgcataggaagtatggcatgaacatctttaggagactaatggaaatattattggtgtttacccagtattccatttttttcattgtgttctctattgctgctctctcactcccccatgaggtacagcagaaaggagaactatccaaaactaatttcctctgacatgtaagacgaatgatttaggtacgtcaaagcagtagtcaaggaggaaagggatagtccaaagacttaactggttcatattggactgataatctctttaaatggctttatgctagtttgacctcatttgtaaaatatttatgagaaagttctcatttaaaatgagatcgttgtttacagtgtatgtactaagcagtaagctatcttcaaatgtctaaggtagtaactttccatagggcctccttagatccctaagatggctttttctccttggtatttctgggtctttctgacatcagcagagaactggaaagacatagccaactgctgttcatgttactcatgactcctttctctaaaactgccttccacaattcactagaccagaagtggacgcaacttaagctgggataatcacattatcatctgaaaatctggagttgaacagcaaaagaagacaacatttctcaaatgcacatctcatggcagctaagccacatggctgggatttaaagcctttagagccagcccatggctttagctacctcactatgctgcttcacaaaccttgctcctgtgtaaaactatattctcagtgtagggcagagaggtctaacaccaacataaggtactagcagtgtttcccgtattgacaggaatacttaactcaataattcttttcttttccatttagtaacagttgtgatgactatgtttctattctaagtaattcctgtattctacagcagatactttgtcagcaatactaagggaagaaacaaagttgaaccgtttctttaataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 134 - protein, 250 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 135 - DNA, 753 bp, Homo sapiens
  source 1..753: mol_type = genomic DNA
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaattag

SEQ ID NO: 136 - protein, 503 aa, Homo sapiens
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 137 - DNA, 4157 bp, Homo sapiens
  source 1..4157: mol_type = genomic DNA
ctctctggccgcccctggctttatttctcgcgcgcttggggtctctcccagtctccgtctctccatttctcctggggggcggggagggggggtctccaaaaaccgcggcggcggcggcggccgctccaggcgcccgttccggagtcggggggaggcccagccgggaggggggaagggggggagccttagtcatttccccgctccagcctgctcccgcccgagcgcgcactcacggccgctctccctcctcgctccgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtaacatcaccccagcagatgccggcacctactactgtgtgaagttccggaaagggagccctgacacggagtttaagtctggagcaggcactgagctgtctgtgcgtgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccagagggctggggcggtgcaggctctgggacccagggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactgttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggaccccttccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaacgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattattggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatgaaaaaaaaaaaaaaaaaaaaaaaaaaaaaaaaagaaaaaaaaaaaaa

SEQ ID NO: 138 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 139 - DNA, 5024 bp, Homo sapiens
  source 1..5024: mol_type = genomic DNA
agcggagtgggtcctgcctgtgacgcgcggcggcggtcggtcctgcctgtaacggcggcggcggctgctgctccagacacctgcggcggcggcggcgaccacgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttttttttttgactaatttcacatgctctaaaaaccttcaaaggtgattatttttctcctggaaactccaggtccattctgtttaaatccctaagaatgtcagaattaaaataacagggctatcgcgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctaaaaaaaaaaaaacaaaaaaaaaaaaaaaa

SEQ ID NO: 140 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 141 - DNA, 870 bp, Homo sapiens
  source 1..870: mol_type = genomic DNA
tctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcat

SEQ ID NO: 142 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 143 - DNA, 12464 bp, Homo sapiens
  source 1..12464: mol_type = genomic DNA
ttaataaatgcctgtctccaggtgctgggtgggaggtgggatggaatggaatgaggtgaggacgcatggatgcatggatgaatggatgggaagttgagacgacgcgcccacacgagggaatttcctttgaaagagagcgaaattctgagttgggaaactcttccttgaaacgcctccccataccccagctgtggccttcccgttttctgcgtggtggtgtggggggaacttcctcaggcctctccgcagtggagcctctttcggttctgccaggatacctagaggcagcggagagcggggcagggaggggaaaaccgtgagggtccctgtggcaggccccagcacccatgggatctctctccggtcgcaggaagcaggctagctcctagcccgcctcggcttggcctttgtgggacctgggggcaaagaagaagagctgtctctgggaccatgcctcctcccgtacacagcaagatgcgtccctaaactcccgggggaattagacttgtgggaatgttctggggaaactcctgcgcggtgaattgctgggggctccgccccccccgataggtggaccgcgattggtctttgaagaccccgcccctttcctgggcggggccaaggctggggcaggggagtcagcagaggcctcgctcgggcgcccagtggtcctgccgcctggtctcacctcgccatggttcgtctgcctctgcagtgcgtcctctggggctgcttgctgaccgctgtgagttgtttttgccccgaccagacgggagttgggagtggggaatgagaaggaaagggaaggaagacttcggggaagaggccttcctggctgatttttgtgggggcaggagggtgggtgggagctgggcaaggtgcccccgctcctggctgaatggggtgggctgcctctctcttctcccgggctggggtcccgggagcggcctacaggggccgctcagggaaggcactggctgcccaagcgtgcctagacggcctggacgggtttagggagcctcagaggctggccacacagagactggtagggggttcagagggcgggaagtgaggcggaccaagggaaggggcgggtctggcccgtttcctgtccccttcttattgtggacagatgccagcctctgtaagtagttatcatctccttgccagctggggctgccttcttccagggcatcttgtgggaacaagagatgggtgcagaggcccaggtacttttgtgagaaggcaaggagcttttaacatcgccttccaccccgaaccgtatcttgggtgttccaacctaggaggaatccccagggctttgcctttttctcctgaatttaagatgacataggagacccctggggagatgaacagtttatgggacacaataaagggttaggagaccagagttctggttggctctgacagggctggtgatcagagggctggagaaaccaggggtttctccaggcaccagaggggctcagagccaaccaagcatatctccgggattttcagaagcctacacttgactcactttttgtttaaatgtatttttgtagttcctcattctggaggctgggaatcccccaagtacctggctccttcatcccagcccctctggcctccccctactttagagggctgtagattcctgcctgaagcctgggcaggaatgacccatggtatcaaggaaagcaagggaagcagcaagggaagagagggagtggggaggctgctttggtcccacagctttcactttcacctgaagcaatggctcttagggaacagggaggcagggggagggcggagctggaaagaggtaaaggggggcccttgtggtaggagtggagaaagagccagaggaggtggggtgaagggtgtgatccaggcttctcaagagcagagtttgccctcataactcccaactttggctccaggtagaggctgggctgtgacaacaatgtcagaagctatctattgagggcttcttgtgtgtcaggctctgagccaaacactgcctgttttctttgtctgatttctcacaactcccccattatacagatgggcaaattgaggctcagaaagggggattgtcttgccaaaggtctcatagctagctaatggaagaacctggttgtgaatctacatctgcatgattcccgagcctgcctctcagatagtgagagtctccaagctctggtcctgagctgttttgtggcagaaggaccagaactatggggagtgagaactggagattgacagacttttaggggagcgttttatttctcatgtgtttgaagatggtatcaaggactttcctatctttgggagtgtgggagctccacgttcacaggatggtgtcttgcaatgagctggtggggggcagtagccttttctacttcctttcccattttgggtaagacacatttctgtaagtaatttgctgagatacccaggttgaatgagagccaccagttaggtaggattctggacagccagccaggtagccgggctgcttgccatatatcatgcaagcagaaacaaatgaatgatgattaaaattgccatttaatgagcacctactatgttcctgacactgtgctaggccatatacatgtattctttcttatcttcgtaatccaacctgcagggcaggcattattactcccattttagagatagagaaactgaggctaagagaagcaaaataactagtaagtgttacaaagtcaggactggagtctaaagctgtctgactctcaaacttgtgttcttttcactggctgttcccaaactgtgggacagttttaaggagcacatggacatagaattaaacatacacttactttacagttcttttaaaaatccttctcattttttcaaagaggaagtctctggagctagaatagagttaatgcctctcaaaggcttgctaatccttcttttaaaacaaaaatcaagagcaggcctgggagggccttcaacaagcaaacaaccagctgggttttaataaccttgttttgtttccccagaatttatttttagggttaccttttatttatgagaagtgatactggttcttgtctcttggcaatgatgtgaggtttacatttaaagtaaatgtaccggccaggcacggtggcttgtgcctgtaatcccagcactttgggaggccaaggcagtcagatcacttgaggtcaggagttttagatcagcctggccaacatggtgaaaccctgtctctactaaaaatacataaattagccgggcatagtggtacacacctgtaatcccagctactcaggaggctgaggctggagaattgcttgaacccaggagatagaggttgcagtgggctgagatgatgccactgcactccagcctgggcgatggagcgagactctgtctcaaaaaataaaataaaagtattgaaattaacaataagtaattaatagcatgggtggtacctggatgtagtaaaatggtgaagatgaaacacaagttgatggagagaggagcattgagacctgagttctcatttggactctgtcactgtgagactctgggcaagtgaccctcctctttggtgctcagtctcaactatctgtaaaatgaaagtgtgagtttacccttccagctttacattctagcattttatgagggaagggctggatgaacagatgatgaggagttggaggaagaaaacatgatgggctttggaaaggagcaggaagggaagcagaagaataggaggaagaggccaagtgctaaacatagccccaaacagcactgggaccagctgaagtcagccagcttcaggactccaggggagctgctggagtccccatatcctatgggatctttgggaagaggaatgactcaggcatcaagccccaaggaattctgttctgttcagagaatattgtgagtttacagtaccattgctttgtaaaaataccagaatgattctctgggtgcgattataatcagctcagttgacaatttacttgaaaacaaacatgccaaatatcatgcaggttccactttctgttttgacttgcacttcagtttgcagcctctgtcctggatgacttttacctttctgctgaagaagttgcaacggagatttcaagatcccttcaaattgcacaattctgtttttaggtccatccagaaccacccactgcatgcagagaaaaacagtacctaataaacagtcagtgctgttctttgtgccagccaggtgagatgccaaccctctagccccatcatggagtccccctttgctttggtggcagacgcagaccccatatgttaactgtaaactcaaatctgaaacgacccatttcccagccctgcttcactgtcagaatgttctggttccctctctaccaggtaaaactctgtctaccctgaactagggatcccagcttctccatcttcctcgcctgattatgaaggatccaagactttcatctttgaatcccctaccctaaagcctggcctgatcattgtgtggttagtgtctgactcatggagttggccagagccctccctcatttcctgatgttttccaggacagaaactggtgagtgactgcacagagttcactgaaacggaatgccttccttgcggtgaaagcgaattcctagacacctggaacagagagacacactgccaccagcacaaatactgcgaccccagtgcgtgcgctgttgggaaagggacgcttgggaaccgggctgatattcccgacaatgcagccattctaattttatgtagccagggtctgctctgattggttggagtccgggctgtactgatcattaaatgatttgattgccatctctacttggaagagggtctgaggaagaaagagcaggcaatgtggggagtgaggctcagagcatggcccagcagggggttcccatccttcctgcccttctcttctcagacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattggtaagtggctcatctgggaatcagttttggagggggacagaggagcttagggcccaaggtgaggggctgggcagtgggcacttagccccagaggcagaggaagcagaggctccaacctatgtcggtatccccactggagtgagctgcagacgggaccttgttcattctgccttctgccatggggatctgcctttgaagggcaatgggagaagtcctcctggggactgcagctgtcgggggcagtaccacatcgggggaagagtgctcaaggcaggagctcttcccgtcctgcctggccactggctgccttgtgagccggacaggtggtccactgtgatggttaatgtccccctccccacccactcccagctacaggggtttctgataccatctgcgagccctgcccagtcggcttcttctccaatgtgtcatctgctttcgaaaaatgtcacccttggacaaggtataagcactcatcccttgtgtttcctgctctaagagtggcatggagctgcctccattctctccagccacctgtcctgtccctgctcccagaggtccacacacactcatgtacttgtgaagcatctgcagagtggcctcatggccaaccagacaggcacatttccacattttttttgcctgctgtctctttgaggtaatagacactgttgatctctcgcttcatgagagcctcctatcttgggggtattgggacacttattttagctttccttctgcccctcctgcttctcctcagttttcctcgtcttgctttcaccttacctggctttctagggctttctgggctctgggtgctcaccctgagggcctccctctcttacctccaactccaaacccacaccaggtcctgccactggctgtctacgtgttttgggaacttactgtctccactgttgtcactttagtttgggcctcatcactgtggtctgggtgatgccttttctgcctcctggcctccctgcctctgtctctcccctcctgctggttctgtctccatcctcttgccaacatgagcgttcgacagtttctttcaaatcatgacactctcctatttgagatgcttcctgtctctctgttggaactaagactccttagcatggcacccaaccttcctgttgcatttcctgctctctttcctgcatcgcatagcttcatgctacttgcaatcctctgaacacactgttcattctcttccatcaaactcatctgcctggaataccttaaacatgggccccaggccaggcgcggtggctcttgcctgtaatctcagcactttggatgccaaggcgggtggatcacttgaggtcaggagttcaagaccagccagcacaacatggtaaaaacccatctctactaaaaataccaaaaaattagctgggtgtggtggtgggcgcctgtaatcccagctcctcgggaggctgaggcaggagaatcacttgaacccggaaggtggagtttgcagtgagccaagatagcgccactgcactccagcctgggcaacagagcgacattctgtctcaaaaaacaaacacctgccccattaactttttgcatttgatttttaaaaatgggcaagataggcacatgggacagaaggcacaaaagagccaaagtgatgtctttctcccatccctgccccttaggctcccagttctttctggagggagccattgttccttgcatatccttccagagattctacatataaacaaaccaacacacacacacacacacacaaacacacacaaaatttccctccttttacttttgcacaaataggagtatacattttatttgttaactgtctgcctttccctaatagattgaaaattccttaaatgtagaaacttggccttttttttttcttccattgatacatcccctatacctggaacagtacctgacgcatggtaggtgcttaaatttttactgataaatgttgactgataactggaggcaccactggtatagtttttttttttttttttttttttttttttttttgagacagagtctcactctgtcgcccaggctggagtgcagtggcgcaatctcggctcactgcaagctctgcctcccaggttcacgccattctcctgcctcagcctcctgagtagctgggactataggcgcccgccaccacacccggctaatttttttgtatttttagtagagacggcgtttcaccgtgttagccaggatggtcttgatctcctgacctcgtgatccgtctgccttggcctcccaaagtgctgggattacaggcgtgagccaccgtgcccggccaccagtggtatagtattaatggaatcagtgcattggcttacgtatctgattacagctcagtaagtgtgtgaccctcactgagcctcagtctcctcatctgaaaaatgggaatgaccttcatttcacaaggcttgagctaaaaacatgtaaagtgtattgtaaattcctgaatgctctactcatgtaagactaaagtaggccgggcgtggtggctcacacctgtaattgcagcactttgggaggccgaggagggcagatcatgaggtcaagagatcgagaccatcctggctaatatggtaaaaccctgtctctactaaaaatacaaaaattagctgggcgtggtggcgcacatctgtagtcccagctactcaggaggcggaggcaggagaattgcttgaacctgggaggtggaggttgcagtgagctgagatcgcgccactgcattccagccagtctggcgaaagagcaagactctgtctcaaaaaaaaaaaaaaaaaaaaaaaaagactaaagtacatggtttcttcaaagcttctctctctttctcccaccttagatgatttttcctttgcaatgtcctgtgtccattccgccccactcctcctggggccacctggaccaggtcttcatcatctcatatctatatgtttgctgtgtctcctggctggccactcttctgtaatttctcctcctctgagctctctgggcagctgaatcttctcactagtgaagtcgcctggttggatgctgatgagactgaccagctgaatccagttgaaaacttcacacttggcagtgatctggttctaaagacacaattttccatagtttcctaacaccatcctgcatgccacctgccttatttccccacatcacatcgtcccacttagcgggactgcactgctgatccaaattttacatcctttagggcccactcaggtcatatgtcctcagggaagtctttctggaagaaccttaaaccagaggttctcaacagggggcagttttgctccctgtggaacgtttgccaatgtctggacacatttcattcgtcacaaacggagagggggatgctacagggatctggcggatagaggccagggatgctgctgaacatctgcaatgcataggacagcccacccccacccccacacccccagtaaataatgatccagcccaagtgtcactggtgctgacgttgagtaaccctatcttaagctgaactcatcatctctccattccagccttggtggattctgtctcctctgaaccattcccatctcactttagcctacctagatcacaaagcttggcactcattatagactcccctatttattactccttcaagatgtgcaagaatcttttctctgcacttttaagttctgtaagaagagtctgtgtcgttcctataataaccagcataggacgttgcacgtgttgtgtgctcagtgaacctggatttgttgattgttgactgactcactctagagttggaaatcttatgcttggggaaacttaatatctctttctttctctgtgtgtgtgcatttgtgcacgtgtctgtgcatagctgtgagaccaaagacctggttgtgcaacaggcaggcacaaacaagactgatgttgtctgtggtgagtcctggacaatgggccctggagaaagcctaggaaggtgggaactgaagggggagatgaggcacacaggaacactggatgggaaaaaggggaggggaggcagtttgggggtgtggtatcacagctctgccacttatcttgggagtctgggcaaatcacttcccctctcttagcctcagtttcttcatctgtaaaatgggatgataacagcacttccttagtaggttttgattttagagtgagaaggttggcctacagtaaagatcagataatgtaaatcagtgaaaaaggtcaggggtaagaaaattacattctctttacctaacgctaaatgaccagttaatgggtgcagcacaccaacatggtacatgtatacatatgtaacaaacctgcacattatgcacatgtaccctaaagcttaaagtataataataataaaatttaaaaaaacgaaaaatacattctctttgctttttctcaaaatgtactttcctctttgtagggctgggactagaatgaggtgagcaaggcacttgccctcgggcgcaatatttaagaaggtgccataaaagtgtagtaatcaaggtaaattcattttgatgcaatatttttaaaaataaaaattaatgcaaagaaatccatgatgagcaagatagcaacattttaaataaagaacaggatccgaccctgtgtttgcatgaccctgcctcactcacctcaccctaatcctggccctggttccagtaaaaggaataggcagccagcctgcaggccgtagtttgctgacttggtgtccgcctgatgattttcaaaatatggcattaaaagaatgtttaccttgatgactgagtgttttggacatccttttcaattttgtcctgaaacaatttcatcccttgcctcacgctagtctccgccctgccttttggtctttcttttattttcccactttgaaaaaaaaattcggcatgagaaatactttacctttcccctccactcttctataccaaaagcaacatgcagacatgaatcatgctagacctcggcattgggcagagagcagggagtggcggggagcatggtgagcaggtggtgacagccactgccaccactcgcttctagatggttcccaggtggggaggctgccaactggaacccagtcttcccagtttgtaagagaaatcagatgtctaggtttgaatatgtgatctcccagtttaaaaatgtcggcaaatatttccaaacgttaagaaaatgttctggctcctttaaagacatctgccagccacatttccccaaggaccgcggtttgaaccttctgatgtagatgagctctgacattggaagattctggagtctgacaagtcacagcaggttgagggtagggagaaactgcaggtgaggggtgcatgctgaagtcctgatttctccaggtccccaggatcggctgagagccctggtggtgatccccatcatcttcgggatcctgtttgccatcctcttggtgctggtctttatcagtgagtcctcaggtggggaggtgttgggggagggaggggagaccacctgtttcttatctggcctctccaactccccatcctttttttttttttttttttttttagaaaaggtggccaagaagccaaccaataaggtaggtcacccctgagaacccgggacagagttttgacaaactgggaagatggcctcacggttgcctatggggcagtaaaactgattcagagtctgtctctgcagccagtggggtggcagcagaattggggactgtcatccccacccaccatgctccttccatccagagctcaatcccccacagaactgcccctggcaccactggcagagcctaacactggctgttcttcactcctttcctggcattcaacgcgtggggagctgcatctttgggccttggggctgggtcaaatgggtgggagcaaatgtggcagccccttaagcccactggctcccactctggaagctcttcgtcgcccttggtgtggccagcagggggcaggaggcacccgaggaatcagcactgacccgccgtctgggaaaggggggagggcttggggaagggatccgcttcccagggaggggctcctcagaggcacagctgcccctgctgctgggggtgacctcacaccttgcctctccaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctgggggagggttggttaaagggagatttggctttcccataatgcttcatcatttttcccaaaaggagagtgaattcacataatgcttatgtaattaaaaaatcatcaaacatgtaaaaagaaaaacgggggtgaacatgctgggtgacatgagctatttaacctgctgtcaggctcacggaatgagggcattttctgtagataaataagaatgtccccaggctgctgcccctccagggtggtttccatgtgtgctcacatgtggtattgagattgcaaagtgctcttcccatttgattcatgttcacaaaaatagccttccccagcagggtgggtctgcatccctccctcttttacagaggtggaaatgaggtc

SEQ ID NO: 144 - DNA, 1514 bp, synthetic construct
  source 1..1514: mol_type = other DNA
ctcaataaaagagcccacaacccctcactcggcgcgccaccatgggcacaagacttctctgctgggtggtgcttggatttctgggcacagatcatacaggagctggagttagccagtctcctaggtacaaagtggccaagagaggacaggatgtggctctgagatgtgaccctattagcggacatgtgagcctgttttggtaccagcaagctctgggacaaggacccgagtttctgacctacttccagaatgaagcccagctggataaatctggactgcctagcgaccggttcttcgccgaaagacctgaaggatctgttagcaccctgaagattcagagaacacagcaggaggactctgccgtgtacctgtgtgcctcttcttctgaaggactggctggaggacctacagctggagaactgttttttggagagggctctaggctgacagttttggaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatgaagacctttgccggattctccttcctgttcctgtggctgcagctggattgtatgagcagaggcgaagatgtggaacagagcctgtttctgagcgtgagagagggagatagcagcgtgatcaattgcacctacaccgattctagcagcacctacctgtactggtacaagcaggaacctggagccggattacaactgctgacctacatcttcagcaacatggacatgaagcaggaccagagactgaccgtgctgctgaacaagaaggacaagcacctgagcctgagaattgccgatacacagacaggagatagcgccatctacttctgtgccgagcctatcattggcggcaatacacctctggtgtttggaaagggcacaaggctgtctgtgattgccaacatccagaatcccgaccctgctgtgtaccagctgcgggacagcaagagcagcgac

SEQ ID NO: 145 - protein, 6 aa, synthetic construct
LSVIGF

SEQ ID NO: 146 - protein, 6 aa, synthetic construct
LLVIVL

SEQ ID NO: 147 - protein, 28 aa, unidentified
KKRIKPIVWPSLPDHKKTLEHLCKKPRK

SEQ ID NO: 148 - protein, 22 aa, unidentified
NCRNTGPWLKKVLKCNTPDPSK

SEQ ID NO: 149 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 150 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 151 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 152 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 153 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 154 - protein, 459 aa, Homo sapiens [sequence omitted]

SEQ ID NO: 155 - DNA, 1503 bp, Homo sapiens
  source 1..1503: mol_type = genomic DNA
cctccctcccttcctcttactctcattcatttcatacacactggctcacacatctactctctctctctatctctctcagaatgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcatctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaaactgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtgaagtgtaagaaacccagactgaacttaccgtgagcgacaaagat

SEQ ID NO: 156 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 157 - DNA, 1746 bp, Homo sapiens
  source 1..1746: mol_type = genomic DNA
cccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggaaaaaaaaaaaaaaaaaaaaaaaaaaaaaaaaaaaaaaaaaaaaaaaaaaaaaaaaaaaaaaaaaaa

SEQ ID NO: 158 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 159 - DNA, 5804 bp, Homo sapiens
  source 1..5804: mol_type = genomic DNA
cctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcgggtgagtggtccccagcccgggctcggcggggcgccgggggtcttcctggggtccccgcctctccgctgcgcttgacagtcgggcccggcaacccggcccccgggcggaaacgaggaaagtttcccccgcgacactcacgcagcccgactcccgtagctgcagggattgtgagtttttcttgaaaaagagaaggaaagttcagttgcaaggggcgcggggcacgtttggtccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagcaaatggctactcaaccaccccaacccctcaccacccctcaccacggtacaatggattttattacaaattgcatacaccagataattctaatctgatgtgaaggaattattttgcctttcttcagattcattctcatgacatcaagttcatttgaaattgcataacatcttcaggaattcattggcaggctgcctggcagttggataatcatttaatatatctttctctctcctcagttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggtaagcaagccttttaagaagttattctttcttttcccctttttacataatgtattctcatagtacacacagtcagtgtatctctgtctcctaaatgtaaacacctgttccatttccctttcctttagaccatctctctttcgattattaaatgtagtttctaggggtgttctctgcatgtatgtgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgcgaatgctggagaacaggaaccagctgccgttgttaggaacaacttcatgaaggaaaagtattccagattgcctttctgtctggaggccatattattcatttattctctttctctctctccctctcccctcgcttccaatgaatctcttcactctaggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaaggtgagttttcttctcttaagggtgtgggacctgagatctgtgccaattttttgtatccttggtctgcagtgtcatagagcacattcctcctgtggtggattgcatacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagctgaagtttgaaggaagagcaggggatgacgaacagatggccagaggcagggaaggctgaacgagcatgcacttgcatccctgaaataaaaattaacaatatcgtatctacaaaaactatgcagatgctaaaatctatagatgctcaggcatgaacccacttcctgacagtacttacctaccacatccaactccttctctccttgttttgtttccccatcagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtaagttagagctagtgctagatcccctttaccttgagcctggcctcaccctacctcttgatccatatctcctggctcttatctcaaacagccctgtactctggacactggtctagggaatctagccaaagtatggagtctgccttgagcatactctgctctgtcctgcctgagcatttttgctaatggacagcatttctcctcctatcttcaaatccttcccagttcagcacattttttccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctatctgtacctttctgtgcatttctcattctttaaaacagcactttgattttttaaaaaaatcctctgcacgtgtcaggggccaccatcagctatattgtgaaaataaaaaggcagctggaattaaatgatgggcctcactgtctgtttttgctataggtgggaactgcaagatacatggctccagaagtcctagaatccaggatgaatttggagaatgttgagtccttcaagcagaccgatgtctactccatggctctggtgctctgggaaatgacatctcgctgtaatgcagtgggaggtaggtgtggaccagcatcattgtgtagtggtaaacttgtcttcaaaataagatcatgtgttgcttcgagcattattccaggggttacaaagcagttattagagctagttgagatctgatattatacaaccatcccagaactatttggggttatgctaaacagcctagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttacttagtgcttcatgctccccagccaggcatctcaccatgctcatttcctttggctgcacatgccattctcagtgaccctgtgtttgctggctttcttcacagaagtaaaagattatgagcctccatttggttccaaggtgcgggagcacccctgtgtcgaaagcatgaaggacaacgtgttgagagatcgagggcgaccagaaattcccagcttctggctcaaccaccaggtaaggagtgagtgtttacaaaggtcagtaagattcaaccaagttgcctcttaggtggcagagaattctggaatattgagcttaaatctgagggaaggtcccattgtgttctatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgttgggagtgttagtgtaccccagaagcccagcagaggatcattgtgctgtcaggacctattgtgatcagatagcaccagagccaaaaagtatggtaggttttgagcacagctgccaccagcacaaaccccccaccaccctttccacatggaactggctggcctgcagcagcaggcactcagtcagcacatgttaaatgcacaggcacttttggaccctgcttgcactcactatagcaacaaggtcagcaggccaccttgccttccgcggagcccaccaactcatggtgccctttggatctctttcccgctacagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcaca

SEQ ID NO: 160 - protein, 4 aa, synthetic construct
PPCL

SEQ ID NO: 161 - protein, 5 aa, synthetic construct
FSCGP

SEQ ID NO: 162 - protein, 7 aa, synthetic construct
PPVCSVT

SEQ ID NO: 163 - protein, 5 aa, synthetic construct
FCRKD

SEQ ID NO: 164 - protein, 4 aa, synthetic construct
PCPL

SEQ ID NO: 165 - protein, 4 aa, synthetic construct
RCPS

SEQ ID NO: 166 - protein, 4 aa, synthetic construct
HRGC

SEQ ID NO: 167 - protein, 5 aa, synthetic construct
PLCSA

SEQ ID NO: 168 - protein, 7 aa, synthetic construct
PIYRCVL

SEQ ID NO: 169 - protein, 5 aa, synthetic construct
FTCPS

SEQ ID NO: 170 - protein, 4 aa, synthetic construct
CPSP

SEQ ID NO: 171 - protein, 5 aa, synthetic construct
SHQPC

SEQ ID NO: 172 - protein, 8 aa, synthetic construct
CSTISILS

SEQ ID NO: 173 - protein, 6 aa, synthetic construct
CGIREI

SEQ ID NO: 174 - protein, 4 aa, synthetic construct
RPCG

SEQ ID NO: 175 - protein, 5 aa, synthetic construct
KKCTN

SEQ ID NO: 176 - protein, 4 aa, synthetic construct
ASWC

SEQ ID NO: 177 - protein, 4 aa, synthetic construct
CRPH

SEQ ID NO: 178 - protein, 7 aa, synthetic construct
ITLYCKT

SEQ ID NO: 179 - protein, 4 aa, synthetic construct
SPCI

SEQ ID NO: 180 - protein, 4 aa, synthetic construct
GFSV

SEQ ID NO: 181 - protein, 9 aa, synthetic construct
DTRVYNSIC

SEQ ID NO: 182 - protein, 12 aa, synthetic construct
SLILIVPCACEL

SEQ ID NO: 183 - protein, 4 aa, synthetic construct
LSRC

SEQ ID NO: 184 - protein, 8 aa, synthetic construct
WAALLNCE

SEQ ID NO: 185 - protein, 4 aa, synthetic construct
NDCS

SEQ ID NO: 186 - protein, 4 aa, synthetic construct
FFSV

SEQ ID NO: 187 - protein, 4 aa, synthetic construct
PLGE

SEQ ID NO: 188 - protein, 119 aa, synthetic construct
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASSSEGLAGGPTAGELFFGEGSRLTVL

SEQ ID NO: 189 - protein, 113 aa, synthetic construct
GEDVEQSLFLSVREGDSSVINCTYTDSSSTYLYWYKQEPGAGLQLLTYIFSNMDMKQDQRLTVLLNKKDKHLSLRIADTQTGDSAIYFCAEPIIGGNTPLVFGKGTRLSVIAN

(19) United States
(12) Patent Application Publication
(10) Pub. No.: US 2025/0281614 A1
(43) Pub. Date: Sep. 11, 2025

(54) CHIMERIC RECEPTORS COMPRISING INTERLEUKIN 7 RECEPTOR (IL7R) DOMAINS

(71) Applicant: Affini-T Therapeutics, Inc., Watertown, MA (US)

(72) Inventors: Gary Shapiro, Watertown, MA (US); Xingyue He, Watertown, MA (US)

(73) Assignee: Affini-T Therapeutics, Inc., Watertown, MA (US)

(21) Appl. No.: 19/090,095

(22) Filed: Mar. 25, 2025

Related U.S. Application Data
(63) Continuation of application No. PCT/US2023/075271, filed on Sep. 27, 2023.
(60) Provisional application No. 63/377,230, filed on Sep. 27, 2022.

(51) Int. Cl. A61K 40/42; A61K 38/17; A61K 40/11; A61K 40/32; C07K 14/715; C07K 14/725; C07K 16/40

(52) U.S. Cl. CPC A61K 40/4253; A61K 38/1793; A61K 40/11; A61K 40/32; C07K 14/7051; C07K 14/7155; C07K 16/40

(57) ABSTRACT

The present disclosure relates to fusion proteins and methods of using them. The fusion protein may include an intracellular domain comprising an intracellular domain of an interleukin receptor polypeptide, or variant thereof that contributes to an interleukin signal in a host cell. The present disclosure also relates to uses of cells expressing such fusion proteins to treat certain diseases, such as cancer.

### CROSS-REFERENCE TO RELATED APPLICATIONS

This application is a continuation under 35 U.S.C. § 111(a) of PCT International Patent Application No. PCT/US2023/075271, filed Sep. 27, 2023, designating the United States and published in English, which claims priority to and the benefit of U.S. Provisional Application No. 63/377,230, filed Sep. 27, 2022, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Oct. 12, 2023, is named “218378-040003US_SL.xml” and is 221,252 bytes in size.

### BACKGROUND

Cell therapy with chimeric T cells has shown promise in preclinical models and early phase clinical trials; however, such cell therapy lacks efficacy due to several factors, including limited in vivo T cell expansion post-infusion or administration. Several challenges remain to be addressed for successful adoptive T cell immunotherapy for treating cancers. Among the challenges are achieving substantial expansion and persistence of adoptively transferred Tcells for durable anti-tumor efficacy. While providing recombinant or transgenic cytokines may achieve benefits, this tactic is associated with significant toxicities that may limit clinical use. In addition, successful T cell immunotherapy is further limited by insufficient T cell reactivity, inability to overcome inhibitory effects of the immunosuppressive tumor microenvironment (TME), inability to activate endogenous T cells or NK cells, and insufficient survival in the periphery. In view of the current limitations for useful and effective T cell therapies to treat individuals with cancers or tumors, alternative approaches for producing tumor-directed T cells are urgently needed to provide tumor-directed T cells that exhibit expansion, persistence, and cytotoxicity within tumors following administration. The present disclosure satisfies this need.

### SUMMARY

Described and provided herein are fusion proteins, compositions, cells that include the fusion proteins, and methods of their use.

In an aspect, the present disclosure provides a fusion protein. The fusion protein includes an extracellular domain including one of the following: (i) an extracellular domain of a Cluster of Differentiation 80 (CD80) polypeptide; (ii) an extracellular domain of a Cluster of Differentiation 58 (CD58) polypeptide; (iii) an extracellular domain of a Signal Regulatory Protein Alpha (SIRPα) polypeptide; (iv) an extracellular domain of Cluster of Differentiation 40L (CD40L) polypeptide; (v) an extracellular domain of a Cluster of Differentiation (CD2) receptor; or (vi) an extracellular domain of Transforming Growth Factor beta receptor 2 (TGFβR2). The fusion protein also includes an intracellular domain of an interleukin receptor polypeptide that is capable of interleukin signaling. The fusion protein also includes a transmembrane domain disposed between the extracellular domain and the intracellular domain.

In another aspect, the present disclosure provides a fusion protein. The fusion protein includes an extracellular domain including an extracellular domain of a transforming growth factor beta receptor II (TGFBR2) polypeptide; or a transforming growth factor beta receptor I (TGFβR1) polypeptide, where the extracellular domain is capable of binding a TGFβ1, TGFβ2, or TGFβ3 polypeptide. The fusion protein also includes an intracellular domain including an intracellular domain of an IL7RA polypeptide capable of IL7R alpha signaling. The fusion protein also includes a transmembrane domain disposed between the extracellular domain and the intracellular domain, where the transmembrane domain includes a mutation that confers constitutive IL-7 signaling on the IL7R alpha intracellular domain.

In another aspect, the present disclosure provides a fusion protein. The fusion protein includes an extracellular domain including a CD58 extracellular domain. The fusion protein also includes an IL7RA transmembrane domain including an amino acid sequence having at least 85% amino acid sequence identity to any one of amino acid sequences IFTCPSISILS (SEQ ID NO: 42), ILLTSHQPCILS (SEQ ID NO: 44), PITLYCKTLLTISILS (SEQ ID NO: 122), or ISPCITISILS (SEQ ID NO: 123). The fusion protein also includes an intracellular domain including an intracellular domain of an IL7RA polypeptide capable of IL7R alpha signaling.

In another aspect, the present disclosure provides a fusion protein. The fusion protein includes an extracellular domain including a CD80 extracellular domain. The fusion protein also includes an IL7RA transmembrane including an amino acid sequence having at least 85% amino acid sequence identity to any one of amino acid sequences IFTCPSISILS (SEQ ID NO: 42), ILLTSHQPCILS (SEQ ID NO: 44), PITLYCKTLLTISILS (SEQ ID NO: 122), or ISPCITISILS (SEQ ID NO: 123). The fusion protein also includes an intracellular domain including an intracellular domain of an IL7RA polypeptide capable of IL7R alpha signaling.

In another aspect, the present disclosure provides a fusion protein. The fusion protein includes an extracellular domain including a CD58 or a CD80 extracellular domain. The fusion protein also includes an intracellular domain including an intracellular domain of an IL7RA polypeptide capable of IL7R alpha signaling. The fusion protein also includes a transmembrane domain disposed between the extracellular domain and the intracellular domain, where the transmembrane domain includes a mutation that confers constitutive IL-7 signaling on the IL7R alpha intracellular domain.

In another aspect, the present disclosure provides a nucleic acid molecule encoding the fusion protein of any one of the above aspects, or embodiments thereof.

In another aspect, the present disclosure provides a vector including the nucleic acid of any one of the above aspects, or embodiments thereof.

In another aspect, the present disclosure provides a host cell. The host cell includes the fusion protein of any one of the above aspects, or embodiments thereof and a nucleic acid molecule encoding a transgenic T cell receptor (TCR) receptor or chimeric antigen receptor (CAR), or an antigen binding fragment thereof.

In another aspect, the present disclosure provides a CD4+ or CD8+ T cell derived from a subject having a neoplasia. The T-cell includes a fusion protein of any one of the above aspects, or embodiments thereof. The T-cell also includes a nucleic acid molecule encoding a transgenic T cell receptor (TCR) receptor or chimeric antigen receptor (CAR), or an antigen binding fragment thereof, where the TCR or the antigen binding fragment thereof specifically binds an HLA:peptide complex including a G12-mutant KRAS polypeptide or fragment thereof. The T-cell also includes one or more genetic alterations that reduce expression of a TRAC polypeptide, TRBC polypeptide, a T cell receptor polypeptide, and/or an MHC polypeptide.

In another aspect, the present disclosure provides a CD4+ or CD8+ T cell derived from a subject having a cancer. The T-cell includes a fusion protein including a CD34 extracellular domain, an IL7 receptor alpha transmembrane domain including the following amino acid sequence: PILLTCPTISILSFFSVALLVILACVLW (SEQ ID NO: 28), and an IL7 receptor alpha intracellular domain capable of IL7 receptor alpha signaling in the absence of ligand binding to the extracellular domain. The T cell also includes a nucleic acid molecule encoding a transgenic T cell receptor (TCR) receptor or chimeric antigen receptor (CAR), or an antigen binding fragment thereof, where the TCR or the antigen binding fragment thereof specifically binds an HLA:peptide complex including a G12-mutant KRAS polypeptide or fragment thereof. The T cell also includes one or more genetic alterations that reduce expression of a TRAC polypeptide, TRBC polypeptide, a T cell receptor polypeptide, and/or an MHC polypeptide.

In another aspect, the present disclosure provides a CD4+ or CD8+ T cell derived from a subject having cancer. The T-cell includes a fusion protein including a CD58 extracellular domain, an IL7 receptor alpha transmembrane domain comprising the following amino acid sequence: PILLTCPTISILSFFSVALLVILACVLW (SEQ ID NO: 28), and an IL7 receptor alpha intracellular domain capable of IL7 receptor alpha signaling in the absence of ligand binding to the extracellular domain. The T cell also includes a nucleic acid molecule encoding a transgenic T cell receptor (TCR) receptor or chimeric antigen receptor (CAR), or an antigen binding fragment thereof, where the TCR or the antigen binding fragment thereof specifically binds an HLA:peptide complex including a G12-mutant KRAS polypeptide or fragment thereof. The T cell also includes one or more genetic alterations that reduce expression of a TRAC polypeptide, TRBC polypeptide, a T cell receptor polypeptide, and/or an MHC polypeptide.

In another aspect, the present disclosure provides a CD4+ or CD8+ T cell derived from a subject having a cancer. The T-cell includes a fusion protein including a CD80 extracellular domain, an IL7 receptor alpha transmembrane domain including the following amino acid sequence: PILLTCPTISILSFFSVALLVILACVLW (SEQ ID NO: 28), and an IL7 receptor alpha intracellular domain capable of IL7 receptor alpha signaling in the absence of ligand binding to the extracellular domain. The T cell also includes a nucleic acid molecule encoding a transgenic T cell receptor (TCR) receptor or chimeric antigen receptor (CAR), or an antigen binding fragment thereof, where the TCR or the antigen binding fragment thereof specifically binds an HLA:peptide complex including a G12-mutant KRAS polypeptide or fragment thereof. The T cell also includes one or more genetic alterations that reduce expression of a TRAC polypeptide, TRBC polypeptide, a T cell receptor polypeptide, and/or an MHC polypeptide.

In another aspect, the present disclosure provides a pharmaceutical composition including an effective amount of a nucleic acid molecule or a vector of any one of the above aspects, or embodiments thereof.

In another aspect, the present disclosure provides a pharmaceutical composition including an effective amount of the cell of any one of the above aspects, or embodiments thereof.

In another aspect, the present disclosure provides a method of treating cancer in a subject, the method involving administering to the subject an effective amount of the cell of any one of the above aspects, or embodiments thereof, or a pharmaceutical composition including said cell.

In another aspect, the present disclosure provides a method of activating a bystander T cell in a subject having a cancer, the method involving administering to the subject an effective amount of the cell of any one of the above aspects, or embodiments thereof, or a pharmaceutical composition including said cell.

In another aspect, the present disclosure provides a kit for treating cancer in a subject, the kit including the pharmaceutical composition of any one of the above aspects, or embodiments thereof, and directions for treating the subject.

In any of the above aspects, or embodiments thereof, the fusion protein, when expressed by a host cell and contacted with a cognate ligand of the extracellular domain initiates interleukin signaling in said host cell.

In any of the above aspects, or embodiments thereof, the fusion protein, when expressed by a host cell initiates interleukin signaling in said host cell in the absence of a ligand.

In any of the above aspects, or embodiments thereof, the fusion protein, when expressed by a host cell forms a homodimer.

In any of the above aspects, or embodiments thereof, the transmembrane domain includes a transmembrane domain of one of the following polypeptides: TGRβR2, interleukin-7 receptor alpha subunit, CD80, CD58, Signal Regulatory Protein Alpha (SIRPα), CD40 Ligand (CD40L), or CD2.

In any of the above aspects, or embodiments thereof, the intracellular domain of the interleukin receptor polypeptide includes an IL7R alpha subunit intracellular domain.

In any of the above aspects, or embodiments thereof, the interleukin signaling initiates an increase in any one or more of Janus kinase 1 (JAK1) activity, Janus kinase 3 (JAK3) activity, phosphoinositide 3-kinase (PI3K) activity, and phosphorylation of STAT5.

In any of the above aspects, or embodiments thereof, the intracellular domain includes a sequence having at least 85% amino acid sequence identity to the following amino acid sequence:

(SEQ ID NO: 15)
KKRIKPIVWPSLPDHKKTLEHLCKKPRKNLNVSFNPESFLDCQIHRV
DDIQARDEVEGFLQDTFPQQLEESEKQRLGGDVQSPNCPSEDVVITP
ESFGRDSSLTCLAGNVSACDAPILSSSRSLDCRESGKNGPHVYQDLL
LSLGTTNSTLPPPFSLQSGILTLNPVAQGQPILTSLGSNQEEAYVTM
SSFYQNQ.

In any of the above aspects, or embodiments thereof, the intracellular domain includes: (a) a BOX1 motif comprising VWPSLPDH (SEQ ID NO: 124); and/or (b) a tyrosine (Y) at amino acid position 185 of the sequence shown in SEQ ID NO: 15, where amino acid position 185 is indicated in bold and with underlining.

In any of the above aspects, or embodiments thereof, the intracellular domain includes a FERM domain comprising amino acids KKRIKPI (SEQ ID NO: 125). In any of the above aspects, or embodiments thereof, the FERM domain is at amino acid positions 1-6 of SEQ ID NO: 15, or at a corresponding position in the intracellular domain of any of the above aspects, or embodiments thereof. In any of the above aspects, or embodiments thereof, the intracellular domain includes a FERM domain which includes amino acids KKTLEHLCKKPRK (SEQ ID NO: 126). In any of the above aspects, or embodiments thereof, the FERM domain is at amino acid positions 16-28 of SEQ ID NO:15, or at a corresponding position in the intracellular domain of any of the above aspects, or embodiments thereof.

In any of the above aspects, or embodiments thereof, the transmembrane domain includes a Cysteine, Proline, Threonine (CPT) insert.

In any of the above aspects, or embodiments thereof, the transmembrane domain includes a sequence having at least 85% amino acid sequence identity to a transmembrane domain of Transforming Growth Factor β receptor 2 (TGRBR2), interleukin-7 receptor alpha subunit, CD80, CD58, Signal Regulatory Protein Alpha (SIRPα), CD40 Ligand (CD40L), CD2, or a transmembrane domain listed in Table 1.

In any of the above aspects, or embodiments thereof, the extracellular domain has at least 85% amino acid sequence identity to an extracellular domain of CD80, CD58, SIRPα, CD40L, CD2 or an extracellular domain of Table 1.

In any of the above aspects, or embodiments thereof, the fusion protein includes a Interleukin-7 receptor alpha juxtamembrane sequence comprising at least about 8 amino acids of the following sequence: NLSCTKLTL (SEQ ID NO: 60).

In any of the above aspects, or embodiments thereof, the IL-7 signaling involves any one or more of: (i) phosphorylation of STAT5; (ii) phosphorylation of the intracellular domain of the fusion protein by JAK1 or JAK3; or (iii) STAT5-mediated transcription.

In any of the above aspects, or embodiments thereof, the extracellular domain of Cluster of Differentiation 80 (CD80) polypeptide binds a CD28 or CTLA-4 polypeptide.

In any of the above aspects, or embodiments thereof, the extracellular domain of CD80 polypeptide includes a sequence having at least 80% sequence identity to the following:

(SEQ ID NO: 16)
MGHTRRQGTSPSKCPYLNFFQLLVLAGLSHFCSGVIHVTKEVKEVAT
LSCGHNVSVEELAQTRIYWQKEKKMVLTMMSGDMNIWPEYKNRTIFD
ITNNLSIVILALRPSDEGTYECVVLKYEKDAFKREHLAEVTLSVKAD
FPTPSISDFEIPTSNIRRIICSTSGGFPEPHLSWLENGEELNAINTT
VSQDPETELYAVSSKLDFNMTTNHSFMCLIKYGHLRVNQTFNWNTTK
QEHFPDN.

In any of the above aspects, or embodiments thereof, the sequence of the extracellular domain of the CD80 polypeptide includes one or more of the following amino acids Leu25, Asn63, Arg29, Asp60, Lys86, Gln-33, Tyr31, Met38, Val39, Met47, Ile49, Trp50, Tyr53, Ile67, Phe108, Pro111, Ile113, Gln157, Asp158, Glu162, or Leu163.

In any of the above aspects, or embodiments thereof, extracellular domain includes a Cluster of Differentiation 58 (CD58) extracellular domain.

In any of the above aspects, or embodiments thereof, the extracellular domain of the CD58 polypeptide includes a sequence having at least 85% amino acid sequence identity to the following sequence:

(SEQ ID NO: 17)
MVAGSDAGRALGVLSVVCLLHCFGFISCFSQQIYGVVYGNVTFHVPS
NVPLKEVLWKKQKDKVAELENSEFRAFSSFKNRVYLDTVSGSLTIYN
LTSSDEDEYEMESPNITDTMKFFLYVLESLPSPTLTCALTNGSIEVQ
CMIPEHYNSHRGLIMYSWDCPMEQCKRNSTSIYFKMENDLPQKIQCT
LSNPLFNTTSSIILTTCIPSSGHSRHR.

In any of the above aspects, or embodiments thereof, the sequence of the extracellular domain of CD58 includes one or more residues corresponding to Glu25, Lys29, Lys32, Asp33, Lys34, Glu37, Glu39, Glu42, Arg44, Ser47, Glu78, or Asp84.

In any of the above aspects, or embodiments thereof, the extracellular domain includes the extracellular domain of the SIRPα polypeptide.

In any of the above aspects, or embodiments thereof, the extracellular domain of the SIRPα polypeptide, includes a sequence having at least 85% amino acid sequence identity to

(SEQ ID NO: 18)
MEPAGPAPGRLGPLLCLLLAASCAWSGVAGEEELQVIQPDKSVLVAA
GETATLRCTATSLIPVGPIQWFRGAGPGRELIYNQKEGHFPRVTTVS
DLTKRNNMDFSIRIGNITPADAGTYYCVKFRKGSPDDVEFKSGAGTE
LSVRAKPSAPVVSGPAARATPQHTVSFTCESHGFSPRDITLKWFKNG
NELSDFQTNVDPVGESVSYSIHSTAKVVLTREDVHSQVICEVAHVTL
QGDPLRGTANLSETIRVPPTLEVTQQPVRAENQVNVTCQVRKFYPQR
LQLTWLENGNVSRTETASTVTENKDGTYNWMSWLLVNVSAHRDDVKL
TCQVEHDGQPAVSKSHDLKVSAHPKEQGSNTAAENTGSNERNIY.

In any of the above aspects, or embodiments thereof, the sequence of the extracellular domain of the SIRPα polypeptide includes one or more residues corresponding to L30, G34, Q52, S66, T67, R69, K93, G97, S98, K53, E54, K96, D100, S29, 136, Q37, 131, V33, P35, 136, K68, or F74.

In any of the above aspects, or embodiments thereof, the extracellular component includes the extracellular domain of said CD40L.

In any of the above aspects, or embodiments thereof, the extracellular domain of CD40L includes a sequence having at least 85% amino acid sequence identity to

(SEQ ID NO: 19)
HRRLDKIEDERNLHEDFVFMKTIQRCNTGERSLSLLNCEEIKSQFEG
FVKDIMLNKEETKKENSFEMQKGDQNPQIAAHVISEASSKTTSVLQW
AEKGYYTMSNNLVTLENGKQLTVKRQGLYYIYAQVTFCSNREASSQA
PFIASLCLKSPGRFERILLRAANTHSSAKPCGQQSIHLGGVFELQPG
ASVFVNVTDPSQVSHGTGFTSFGLLKL.

In any of the above aspects, or embodiments thereof, the sequence of the extracellular domain of CD40L, includes one or more amino acid residues corresponding to A130, P217, V247, F253, 1190, E129, K143, G144, Y145, Y146, C178, C218, Q220, S245, Q246, S248, G250, T251, G252, G199, R200, R203, Q232, K133, E142, H249, S132, T134, R207, 1127, S128, S185, Q186, A187, F201, H249, Y170, H224, Q121, H125, T147, Y172, Q174, L195, L205, L206, R207, A208, A209, N210, T211, A215, G219, Q221, S222, L225, G226, G227, V228, F229, E230, T251, G252, L259, or L261.

In any of the above aspects, or embodiments thereof, the extracellular component includes the extracellular domain of the CD2 receptor.

In any of the above aspects, or embodiments thereof, the extracellular domain of the CD2 receptor includes a sequence having at least 85% amino acid sequence identity to the following sequence:

(SEQ ID NO: 20)
MSFPCKFVASFLLIFNVSSKGAVSKEITNALETWGALGQDINLDIPS
FQMSDDIDDIKWEKTSDKKKIAQFRKEKETFKEKDTYKLFKNGTLKI
KHLKTDDQDIYKVSIYDTKGKNVLEKIFDLKIQERVSKPKISWTCIN
TTLTCEVMNGTDPELNLYQDGKHLKLSQRVITHKWTTSLSAKFKCTA
GNKVSKESSVEPVSCPEKGLD.

In any of the above aspects, or embodiments thereof, the sequence of the extracellular domain of the CD2 receptor includes one or more residues corresponding to Lys43, Tyr86, Asn92, Glu95, Asn92, Asp32, Gly90, Arg48, Lys51, Asp31, Lys89, Lys34, Lys41.

In any of the above aspects, or embodiments thereof, the fusion protein, when expressed by a host cell and bound to a TGFβ1, TGFβ2, or TGFβ3 polypeptide induces IL-7 signaling in the host cell.

In any of the above aspects, or embodiments thereof, the transmembrane domain includes a transmembrane domain of a TGFβR2, an IL7RA, a CD80, a CD58, a SIRPα, a CD40L, or a CD2 polypeptide, or a variant thereof.

In any of the above aspects, or embodiments thereof, the extracellular domain of the TGFβR2 receptor, or the portion or variant thereof includes a sequence having at least 85% amino acid sequence identity to the following sequence

(SEQ ID NO: 13)
MGRGLLRGLWPLHIVLWTRIASTIPPHVQKSVNNDMIVTDNNGAVKF
PQLCKFCDVRFSTCDNQKSCMSNCSITSICEKPQEVCVAVWRKNDEN
ITLETVCHDPKLPYHDFILEDAASPKCIMKEKKKPGETFFMCSCSSD
ECNDNIIFSEEYNTSNPDLLLVIFQ.

In any of the above aspects, or embodiments thereof, the sequence of the extracellular domain of the TGFβR2 receptor includes one or more residues corresponding to Glu119, Asp32, Glu75, Tyr85, Ser49-Cys54, Leu27, Phe30, Ile50, Thr51, or Ile53.

In any of the above aspects, or embodiments thereof, the transmembrane domain has at least 85% amino acid sequence identity to a constitutively active IL-7 receptor alpha transmembrane domain including the following amino acid sequence: PILLTCPTISILSFFSVALLVILACVLW (SEQ ID NO: 28), where the sequence comprises the CPT bold amino acid sequences.

In any of the above aspects, or embodiments thereof, the transmembrane domain has at least 85% amino acid sequence identity to a constitutively active IL-7 receptor alpha transmembrane domain including the following amino acid sequence:

(SEQ ID NO: 28)
PILLTCPTISILSFFSVALLVILACVLW.

In any of the above aspects, or embodiments thereof, the nucleic acid molecule includes DNA, RNA, or a hybrid of DNA and RNA.

In any of the above aspects, or embodiments thereof, the nucleic acid molecule includes one or more modified nucleobases.

In any of the above aspects, or embodiments thereof, the nucleic acid molecule is operably linked to a promoter.

In any of the above aspects, or embodiments thereof, the promoter is a murine stem cell virus (MSCV) promoter.

In any of the above aspects, or embodiments thereof, the vector is capable of delivering the nucleic acid molecule to a hematopoietic progenitor cell or a human immune system cell.

In any of the above aspects, or embodiments thereof, the vector is a viral vector.

In any of the above aspects, or embodiments thereof, the viral vector is a lentiviral vector, an adenovirus-associated vector (AAV), or a γ-retroviral vector.

In any of the above aspects, or embodiments thereof, the viral vector is a lentiviral vector.

In any of the above aspects, or embodiments thereof, the nucleic acid molecule encodes a transgenic TCR.

In any of the above aspects, or embodiments thereof, the TCR or the antigen binding fragment thereof specifically binds an HLA:peptide complex including a G12-mutant KRAS polypeptide.

In any of the above aspects, or embodiments thereof, the HLA:peptide complex includes an HLA-A11 polypeptide.

In any of the above aspects, or embodiments thereof, the HLA-A11 polypeptide includes an HLA-A11:01 polypeptide.

In any of the above aspects, or embodiments thereof, the host cell is an immune cell.

In any of the above aspects, or embodiments thereof, the host cell is a T cell, a CD4+ T cell, a CD8+ T cell, a CD4− CD8− double negative T cell, or a γδ T cell, a natural killer cell, a natural killer T cell, a dendritic cell, or any combination thereof.

In any of the above aspects, or embodiments thereof, the host cell is a T cell.

In any of the above aspects, or embodiments thereof, the T cell is a naïve T cell, a central memory T cell, a stem cell memory T cell, or an effector memory T cell.

In any of the above aspects, or embodiments thereof, the host cell includes one or more genetic alterations that reduce expression of a TRBC polypeptide, a T cell receptor polypeptide, and/or an MHC polypeptide.

In any of the above aspects, or embodiments thereof, the genetic alteration is a chromosomal gene knockout, mutation, or indel.

In any of the above aspects, or embodiments thereof, the chromosomal gene knockout, mutation, or indel is in one or more of a TRBC gene locus, a T cell receptor gene locus, a MHC gene locus.

In any of the above aspects, or embodiments thereof, the composition further includes a pharmaceutically acceptable excipient.

In any of the above aspects, or embodiments thereof, the composition further includes (i) a composition comprising at least about 30% CD4+ T cells, combined with (ii) a composition comprising at least about 30% CD8+ T cells, in about a 1:1 ratio.

In any of the above aspects, or embodiments thereof, the cancer is a hematologic cancer or solid tumor.

In any of the above aspects, or embodiments thereof, the cancer is selected from the group consisting of a cancer of the head or neck, melanoma, pancreatic cancer, cholangiocarcinoma, hepatocellular cancer, breast cancer, gastric cancer, lung cancer, prostate cancer, esophageal cancer, mesothelioma, colorectal cancer, and glioblastoma.

In any of the above aspects, or embodiments thereof, the pancreatic cancer is pancreatic ductal adenocarcinoma (PDAC).

In any of the above aspects, or embodiments thereof, the breast cancer is triple-negative breast cancer (TNBC).

In any of the above aspects, or embodiments thereof, the lung cancer is small-cell lung cancer or non-small-cell lung cancer.

In any of the above aspects, or embodiments thereof, the cancer is colorectal cancer.

In any of the above aspects, or embodiments thereof, the cell is derived from the subject or from a healthy donor.

Compositions and articles defined by the invention were isolated or otherwise manufactured in connection with the examples provided below. Other features and advantages of the invention will be apparent from the detailed description, and from the claims.

### Definitions

Prior to setting forth this disclosure in more detail, it may be helpful to an understanding thereof to provide definitions of certain terms to be used herein. Additional definitions are set forth throughout this disclosure.

As used in the specification and claims, the singular forms “a,” “an,” and “the” include plural references unless the context clearly dictates otherwise. For example, the term “a transmembrane receptor” can include a plurality of transmembrane receptors.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated.

The term “about” or “approximately” means within an acceptable error range for the particular value, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, such as within 5-fold, or within 2-fold, of a value. Where particular values are described in the application and claims, unless otherwise stated, the term “about” means within an acceptable error range for the particular value may be assumed.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

As used herein, the term “adoptive immune therapy” or “adoptive immunotherapy” refers to administration of naturally occurring or genetically engineered, disease- or antigen-specific immune cells. In one embodiment, the immune cells are T cells. Adoptive cellular immunotherapy may be autologous (immune cells are from the recipient), allogeneic (immune cells are from a donor of the same species) or syngeneic (immune cells are from a donor genetically identical to the recipient).

By “agent” is meant a polypeptide or nucleic acid molecule, or a cell expressing a polypeptide or nucleic acid molecule described herein. In some embodiments, an agent is a fusion protein of the present disclosure. In some embodiments, an agent is a polynucleotide comprising a sequence encoding a fusion protein of the present disclosure. In some embodiments, an agent is a host cell comprising and/or expressing a fusion protein of the present disclosure and/or a polynucleotide comprising a sequence encoding a fusion protein of the present disclosure.

By “alteration” is meant a change in the structure, expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

An “altered domain” or “altered protein” refers to a motif, region, domain, peptide, polypeptide, or protein with a non-identical sequence identity to a wild-type motif, region, domain, peptide, polypeptide, or protein (e.g., a wild type TCRα chain, TCRβ chain, TCRα constant domain, TCRβ constant domain) of at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9%).

Altered domains or altered proteins or derivatives can include those based on all possible codon choices for the same amino acid and codon choices based on conservative amino acid substitutions. For example, the following six groups each contain amino acids that are conservative substitutions for one another: 1) alanine (ala; A), serine (ser; S), threonine (thr; T); 2) aspartic acid (asp; D), glutamic acid (glu; E); 3) asparagine (asn; N), glutamine (gln; Q); 4) arginine (arg; R), lysine (lys; K); 5) Isoleucine (ile; I), leucine (L), methionine (met; M), valine (val; V); and 6) phenylalanine (phe; F), tyrosine (tyr; Y), tryptophan (trp; W). (See also WO97/09433 at page 10, Lehninger, Biochemistry, 2nd Edition, Worth Publishers, Inc., NY, NY, pp. 71-77, 1975; Lewin Genes IV, Oxford University Press, NY and Cell Press, Cambridge, MA, p. 8, 1990; Creighton, Proteins, W.H. Freeman and Company 1984). In addition, individual substitutions, deletions or additions that alter, add or delete, a single amino acid or a small percentage of amino acids in an encoded sequence are also “conservative substitutions.

“By “ameliorate” is meant decrease, suppress, attenuate, diminish, arrest, or stabilize the development or progression of a disease.

“Antigen” or “Ag” as used herein refers to a molecule that provokes an immune response. This immune response may involve antibody production, activation of specific immunologically competent cells (e.g., T cells), or both. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Example biological samples that can contain one or more antigens include tissue samples, tumor samples, cells, biological fluids, or combinations thereof. Antigens can be produced by cells that have been modified or genetically engineered to express an antigen, or that endogenously (e.g., without modification or genetic engineering by human intervention) express a mutation or polymorphism that is immunogenic.

By “BOX1 motif” is meant a domain comprising amino acids VWPSLPDH (SEQ ID NO: 124) that functions in a Janus kinases (JAK) interaction.

By “Cluster of Differentiation 2 (CD2) polypeptide” is meant a polypeptide or fragment thereof having at least about 85% amino acid identity to GENBANK Accession No. AAH33583.1, and having CD58 binding activity. An exemplary polypeptide sequence is provided below:

>AAH33583.1 CD2 molecule [Homo sapiens]
(SEQ ID NO: 127)
MSFPCKFVASFLLIFNVSSKGAVSKEITNALETWGALGQDINLDIPS
FQMSDDIDDIKWEKTSDKKKIAQFRKEKETFKEKDTYKLFKNGTLKI
KHLKTDDQDIYKVSIYDTKGKNVLEKIFDLKIQERVSKPKISWTCIN
TTLTCEVMNGTDPELNLYQDGKHLKLSQRVITHKWTTSLSAKFKCTA
GNKVSKESSVEPVSCPEKGLDIYLIIGICGGGSLLMVFVALLVFYIT
KRKKQRSRRNDEELETRAHRVATEERGRKPQQIPASTPQNPATSQHP
PPPPGHRSQAPSHRPPPPGHRVQHQPQKRPPAPSGTQVHQQKGPPLP
RPRVQPKPPHGAAENSLSPSSN

In the present disclosure, it will be understood that Cluster of Differentiation 2 can also be referred-to as CD2, LFA-2, CD2 Antigen (P50), Sheep Red Blood Cell Receptor, SRBC, T-Cell Surface Antigen T11/Leu-5, or T11.

By “CD2 polynucleotide” is meant a nucleic acid molecule encoding a CD2 polypeptide. Exemplary sequences of CD2 polynucleotides are provided below:

>NM_001767.5 Homo sapiens CD2 molecule (CD2),
transcript variant 2, mRNA
(SEQ ID NO: 128)
AGTCTCACTTCAGTTCCTTTTGCATGAAGAGCTCAGAATCAAAAGAG
GAAACCAACCCCTAAGATGAGCTTTCCATGTAAATTTGTAGCCAGCT
TCCTTCTGATTTTCAATGTTTCTTCCAAAGGTGCAGTCTCCAAAGAG
ATTACGAATGCCTTGGAAACCTGGGGTGCCTTGGGTCAGGACATCAA
CTTGGACATTCCTAGTTTTCAAATGAGTGATGATATTGACGATATAA
AATGGGAAAAAACTTCAGACAAGAAAAAGATTGCACAATTCAGAAAA
GAGAAAGAGACTTTCAAGGAAAAAGATACATATAAGCTATTTAAAAA
TGGAACTCTGAAAATTAAGCATCTGAAGACCGATGATCAGGATATCT
ACAAGGTATCAATATATGATACAAAAGGAAAAAATGTGTTGGAAAAA
ATATTTGATTTGAAGATTCAAGAGAGGGTCTCAAAACCAAAGATCTC
CTGGACTTGTATCAACACAACCCTGACCTGTGAGGTAATGAATGGAA
CTGACCCCGAATTAAACCTGTATCAAGATGGGAAACATCTAAAACTT
TCTCAGAGGGTCATCACACACAAGTGGACCACCAGCCTGAGTGCAAA
ATTCAAGTGCACAGCAGGGAACAAAGTCAGCAAGGAATCCAGTGTCG
AGCCTGTCAGCTGTCCAGAGAAAGGTCTGGACATCTATCTCATCATT
GGCATATGTGGAGGAGGCAGCCTCTTGATGGTCTTTGTGGCACTGCT
CGTTTTCTATATCACCAAAAGGAAAAAACAGAGGAGTCGGAGAAATG
ATGAGGAGCTGGAGACAAGAGCCCACAGAGTAGCTACTGAAGAAAGG
GGCCGGAAGCCCCACCAAATTCCAGCTTCAACCCCTCAGAATCCAGC
AACTTCCCAACATCCTCCTCCACCACCTGGTCATCGTTCCCAGGCAC
CTAGTCATCGTCCCCCGCCTCCTGGACACCGTGTTCAGCACCAGCCT
CAGAAGAGGCCTCCTGCTCCGTCGGGCACACAAGTTCACCAGCAGAA
AGGCCCGCCCCTCCCCAGACCTCGAGTTCAGCCAAAACCTCCCCATG
GGGCAGCAGAAAACTCATTGTCCCCTTCCTCTAATTAAAAAAGATAG
AAACTGTCTTTTTCAATAAAAAGCACTGTGGATTTCTGCCCTCCTGA
TGTGCATATCCGTACTTCCATGAGGTGTTTTCTGTGTGCAGAACATT
GTCACCTCCTGAGGCTGTGGGCCACAGCCACCTCTGCATCTTCGAAC
TCAGCCATGTGGTCAACATCTGGAGTTTTTGGTCTCCTCAGAGAGCT
CCATCACACCAGTAAGGAGAAGCAATATAAGTGTGATTGCAAGAATG
GTAGAGGACCGAGCACAGAAATCTTAGAGATTTCTTGTCCCCTCTCA
GGTCATGTGTAGATGCGATAAATCAAGTGATTGGTGTGCCTGGGTCT
CACTACAAGCAGCCTATCTGCTTAAGAGACTCTGGAGTTTCTTATGT
GCCCTGGTGGACACTTGCCCACCATCCTGTGAGTAAAAGTGAAATAA
AAGCTTTGACTAGA
>NM_001328609.2 Homo sapiens CD2 molecule (CD2),
transcript variant 1, mRNA
(SEQ ID NO: 129)
AGTCTCACTTCAGTTCCTTTTGCATGAAGAGCTCAGAATCAAAAGAG
GAAACCAACCCCTAAGATGAGCTTTCCATGTAAATTTGTAGCCAGCT
TCCTTCTGATTTTCAATGTTTCTTCCAAAGGTGCAGTCTCCAAAGAG
ATTACGAATGCCTTGGAAACCTGGGGTGCCTTGGGTCAGGACATCAA
CTTGGACATTCCTAGTTTTCAAATGAGTGATGATATTGACGATATAA
AATGGGAAAAAACTTCAGACAAGAAAAAGATTGCACAATTCAGAAAA
GAGAAAGAGACTTTCAAGGAAAAAGATACATATAAGCTATTTAAAAA
TGGAACTCTGAAAATTAAGCATCTGAAGACCGATGATCAGGATATCT
ACAAGGTATCAATATATGATACAAAAGGAAAAAATGTGTTGGAAAAA
ATATTTGATTTGAAGATTCAAGAGAGGGTCTCAAAACCAAAGATCTC
CTGGACTTGTATCAACACAACCCTGACCTGTGAGGTAATGAATGGAA
CTGACCCCGAATTAAACCTGTATCAAGATGGGAAACATCTAAAACTT
TCTCAGAGGGTCATCACACACAAGTGGACCACCAGCCTGAGTGCAAA
ATTCAAGTGCACAGCAGGGAACAAAGTCAGCAAGGAATCCAGTGTCG
AGCCTGTCAGCTGTCCAGGAGGCAGCATCCTTGGCCAGAGTAATGGG
CTCTCTGCCTGGACCCCTCCCAGCCATCCCACTTCTCTTCCTTTTGC
AGAGAAAGGTCTGGACATCTATCTCATCATTGGCATATGTGGAGGAG
GCAGCCTCTTGATGGTCTTTGTGGCACTGCTCGTTTTCTATATCACC
AAAAGGAAAAAACAGAGGAGTCGGAGAAATGATGAGGAGCTGGAGAC
AAGAGCCCACAGAGTAGCTACTGAAGAAAGGGGCCGGAAGCCCCACC
AAATTCCAGCTTCAACCCCTCAGAATCCAGCAACTTCCCAACATCCT
CCTCCACCACCTGGTCATCGTTCCCAGGCACCTAGTCATCGTCCCCC
GCCTCCTGGACACCGTGTTCAGCACCAGCCTCAGAAGAGGCCTCCTG
CTCCGTCGGGCACACAAGTTCACCAGCAGAAAGGCCCGCCCCTCCCC
AGACCTCGAGTTCAGCCAAAACCTCCCCATGGGGCAGCAGAAAACTC
ATTGTCCCCTTCCTCTAATTAAAAAAGATAGAAACTGTCTTTTTCAA
TAAAAAGCACTGTGGATTTCTGCCCTCCTGATGTGCATATCCGTACT
TCCATGAGGTGTTTTCTGTGTGCAGAACATTGTCACCTCCTGAGGCT
GTGGGCCACAGCCACCTCTGCATCTTCGAACTCAGCCATGTGGTCAA
CATCTGGAGTTTTTGGTCTCCTCAGAGAGCTCCATCACACCAGTAAG
GAGAAGCAATATAAGTGTGATTGCAAGAATGGTAGAGGACCGAGCAC
AGAAATCTTAGAGATTTCTTGTCCCCTCTCAGGTCATGTGTAGATGC
GATAAATCAAGTGATTGGTGTGCCTGGGTCTCACTACAAGCAGCCTA
TCTGCTTAAGAGACTCTGGAGTTTCTTATGTGCCCTGGTGGACACTT
GCCCACCATCCTGTGAGTAAAAGTGAAATAAAAGCTTTGACTAGA

By “Cluster of Differentiation 34 polypeptide” (CD34) is meant a protein, or fragment thereof, having at least about 85% amino acid identity to GENBANK Accession No. AAH39146.2 that specifically binds an anti-CD34 antibody. An exemplary polypeptide sequence is provided below:

>AAH39146.2 CD34 molecule [Homo sapiens]
(SEQ ID NO: 130)
MLVRRGARAGPRMPRGWTALCLLSLLPSGFMSLDNNGTATPELPTQGTFS
NVSTNVSYQETTTPSTLGSTSLHPVSQHGNEATTNITETTVKFTSTSVIT
SVYGNTNSSVQSQTSVISTVFTTPANVSTPETTLKPSLSPGNVSDLSTTS
TSLATSPTKPYTSSSPILSDIKAEIKCSGIREVKLTQGICLEQNKTSSCA
EFKKDRGEGLARVLCGEEQADADAGAQVCSLLLAQSEVRPQCLLLVLANR
TEISSKLQLMKKHQSDLKKLGILDFTEQDVASHQSYSQKTLIALVTSGAL
LAVLGITGYFLMNRRSWSPTGERLGEDPYYTENGGGQGYSSGPGTSPEAQ
GKASVNRGAQENGTGQATSRNGHSARQHVVADTEL

By “CD34 polynucleotide” is meant a polynucleotide encoding a CD34 polypeptide. An exemplary sequence of a CD34 polynucleotide is provided below:

>BC039146.1 Homo sapiens CD34 molecule, mRNA (cDNA
clone MGC: 21247 IMAGE: 4746591), complete cds
(SEQ ID NO: 131)
CGGGAAGGATGCTGGTCCGCAGGGGCGCGCGCGCAGGGCCCAGGATGCCG
CGGGGCTGGACCGCGCTTTGCTTGCTGAGTTTGCTGCCTTCTGGGTTCAT
GAGTCTTGACAACAACGGTACTGCTACCCCAGAGTTACCTACCCAGGGAA
CATTTTCAAATGTTTCTACAAATGTATCCTACCAAGAAACTACAACACCT
AGTACCCTTGGAAGTACCAGCCTGCACCCTGTGTCTCAACATGGCAATGA
GGCCACAACAAACATCACAGAAACGACAGTCAAATTCACATCTACCTCTG
TGATAACCTCAGTTTATGGAAACACAAACTCTTCTGTCCAGTCACAGACC
TCTGTAATCAGCACAGTGTTCACCACCCCAGCCAACGTTTCAACTCCAGA
GACAACCTTGAAGCCTAGCCTGTCACCTGGAAATGTTTCAGACCTTTCAA
CCACTAGCACTAGCCTTGCAACATCTCCCACTAAACCCTATACATCATCT
TCTCCTATCCTAAGTGACATCAAGGCAGAAATCAAATGTTCAGGCATCAG
AGAAGTGAAATTGACTCAGGGCATCTGCCTGGAGCAAAATAAGACCTCCA
GCTGTGCGGAGTTTAAGAAGGACAGGGGAGAGGGCCTGGCCCGAGTGCTG
TGTGGGGAGGAGCAGGCTGATGCTGATGCTGGGGCCCAGGTATGCTCCCT
GCTCCTTGCCCAGTCTGAGGTGAGGCCTCAGTGTCTACTGCTGGTCTTGG
CCAACAGAACAGAAATTTCCAGCAAACTCCAACTTATGAAAAAGCACCAA
TCTGACCTGAAAAAGCTGGGGATCCTAGATTTCACTGAGCAAGATGTTGC
AAGCCACCAGAGCTATTCCCAAAAGACCCTGATTGCACTGGTCACCTCGG
GAGCCCTGCTGGCTGTCTTGGGCATCACTGGCTATTTCCTGATGAATCGC
CGCAGCTGGAGCCCCACAGGAGAAAGGCTGGGCGAAGACCCTTATTACAC
GGAAAACGGTGGAGGCCAGGGCTATAGCTCAGGACCTGGGACCTCCCCTG
AGGCTCAGGGAAAGGCCAGTGTGAACCGAGGGGCTCAGGAAAACGGGACC
GGCCAGGCCACCTCCAGAAACGGCCATTCAGCAAGACAACACGTGGTGGC
TGATACCGAATTGTGACTCGGCTAGGTGGGGCAAGGCTGGGCAGTGTCCG
AGAGAGCACCCCTCTCTGCATCTGACCACGTGCTACCCCCATGCTGGAGG
TGACATCTCTTACGCCCAACCCTTCCCCACTGCACACACCTCAGAGGCTG
TTCTTGGGGCCCTACACCTTGAGGAGGGGCAGGTAAACTCCTGTCCTTTA
CACATTCGGCTCCCTGGAGCCAGACTCTGGTCTTCTTTGGGTAAACGTGT
GACGGGGGAAAGCCAAGGTCTGGAGAAGCTCCCAGGAACAATCGATGGCC
TTGCAGCACTCACACAGGACCCCCTTCCCCTACCCCCTCCTCTCTGCCGC
AATACAGGAACCCCCAGGGGAAAGATGAGCTTTTCTAGGCTACAATTTTC
TCCCAGGAAGCTTTGATTTTTACCGTTTCTTCCCTGTATTTTCTTTCTCT
ACTTTGAGGAAACCAAAGTAACCTTTTGCACCTGCTCTCTTGTAATGATA
TAGCCAGAAAAACGTGTTGCCTTGAACCACTTCCCTCATCTCTCCTCCAA
GACACTGTGGACTTGGTCACCAGCTCCTCCCTTGTTCTCTAAGTTCCACT
GAGCTCCATGTGCCCCCTCTACCATTTGCAGAGTCCTGCACAGTTTTCTG
GCTGGAGCCTAGAACAGGCCTCCCAAGTTTTAGGACAAACAGCTCAGTTC
TAGTCTCTCTGGGGCCACACAGAAACTCTTTTTGGGCTCCTTTTTCTCCC
TCTGGATCAAAGTAGGCAGGACCATGGGACCAGGTCTTGGAGCTGAGCCT
CTCACCTGTACTCTTCCGAAAAATCCTCTTCCTCTGAGGCTGGATCCTAG
CCTTATCCTCTGATCTCCATGGCTTCCTCCTCCCTCCTGCCGACTCCTGG
GTTGAGCTGTTGCCTCAGTCCCCCAACAGATGCTTTTCTGTCTCTGCCTC
CCTCACCCTGAGCCCCTTCCTTGCTCTGCACCCCCATATGGTCATAGCCC
AGATCAGCTCCTAACCCTTATCACCAGCTGCCTCTTCTGTGGGTGACCCA
GGTCCTTGTTTGCTGTTGATTTCTTTCCAGAGGGGTTGAACAGGGATCCT
GGTTTCAATGACGGTTGGAAATAGAAATTTCCAGAGAAGAGAGTATTGGG
TAGATATTTTTTCTGAATACAAAGTGATGTGTTTAAATACTGCAATTAAA
GTGATACTGAAACACAAAAAAAAAAAAAAAAAAAAAAAAAAAAAAAAAAA
AAAAAA

By “Cluster of Differentiation 80 polypeptide” (CD80) is meant a protein or fragment thereof having at least about 85% amino acid identity to GENBANK Accession No. AAH42665.1, and having CD28 and/or CTLA-4 binding activity. An exemplary polypeptide sequence is provided below:

>AAH42665.1 CD80 molecule [Homo sapiens]
(SEQ ID NO: 132)
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDNLLPSWAIT
LISVNGIFVICCLTYCFAPRCRERRRNERLRRESVRPV

In the present disclosure, it will be understood that Cluster of Differentiation 80 (CD80) can also be referred to asB7-1, B7, B7.1, BB1, CD28LG, CD28LG1, LAB7, or CD80 molecule.

By “CD80 polynucleotide” is meant a nucleic acid molecule encoding a CD80 polypeptide. An exemplary sequence of a CD80 polynucleotide is provided below:

>BC042665.1 Homo sapiens CD80 molecule, mRNA (cDNA
clone MGC: 34467 IMAGE: 5181343), complete cds
(SEQ ID NO: 133)
AAGTAACAGAAGTTAGAAGGGGAAATGTCGCCTCTCTGAAGATTACCCAA
AGAAAAAGTGATTTGTCATTGCTTTATAGACTGTAAGAAGAGAACATCTC
AGAAGTGGAGTCTTACCCTGAAATCAAAGGATTTAAAGAAAAAGTGGAAT
TTTTCTTCAGCAAGCTGTGAAACTAAATCCACAACCTTTGGAGACCCAGG
AACACCCTCCAATCTCTGTGTGTTTTGTAAACATCACTGGAGGGTCTTCT
ACGTGAGCAATTGGATTGTCATCAGCCCTGCCTGTTTTGCACCTGGGAAG
TGCCCTGGTCTTACTTGGGTCCAAATTGTTGGCTTTCACTTTTGACCCTA
AGCATCTGAAGCCATGGGCCACACACGGAGGCAGGGAACATCACCATCCA
AGTGTCCATACCTCAATTTCTTTCAGCTCTTGGTGCTGGCTGGTCTTTCT
CACTTCTGTTCAGGTGTTATCCACGTGACCAAGGAAGTGAAAGAAGTGGC
AACGCTGTCCTGTGGTCACAATGTTTCTGTTGAAGAGCTGGCACAAACTC
GCATCTACTGGCAAAAGGAGAAGAAAATGGTGCTGACTATGATGTCTGGG
GACATGAATATATGGCCCGAGTACAAGAACCGGACCATCTTTGATATCAC
TAATAACCTCTCCATTGTGATCCTGGCTCTGCGCCCATCTGACGAGGGCA
CATACGAGTGTGTTGTTCTGAAGTATGAAAAAGACGCTTTCAAGCGGGAA
CACCTGGCTGAAGTGACGTTATCAGTCAAAGCTGACTTCCCTACACCTAG
TATATCTGACTTTGAAATTCCAACTTCTAATATTAGAAGGATAATTTGCT
CAACCTCTGGAGGTTTTCCAGAGCCTCACCTCTCCTGGTTGGAAAATGGA
GAAGAATTAAATGCCATCAACACAACAGTTTCCCAAGATCCTGAAACTGA
GCTCTATGCTGTTAGCAGCAAACTGGATTTCAATATGACAACCAACCACA
GCTTCATGTGTCTCATCAAGTATGGACATTTAAGAGTGAATCAGACCTTC
AACTGGAATACAACCAAGCAAGAGCATTTTCCTGATAACCTGCTCCCATC
CTGGGCCATTACCTTAATCTCAGTAAATGGAATTTTTGTGATATGCTGCC
TGACCTACTGCTTTGCCCCAAGATGCAGAGAGAGAAGGAGGAATGAGAGA
TTGAGAAGGGAAAGTGTACGCCCTGTATAACAGTGTCCGCAGAAGCAAGG
GGCTGAAAAGATCTGAAGGTCTCACCTCCATTTGCAATTGACCTCTTCTG
GGAACTTCCTCAGATGGACAAGATTACCCCACCTTGCCCTTTACGTATCT
GCTCTTAGGTGCTTCTTCACTTCAGTTGCTTTGCAGGAAGTGTCTAGAGG
AATATGGTGGGCACAGAAGTAGCTCTGGTGACCTTGATCAAGGGGTTTTG
AAATGCAGAATTCTTGAGTTCTGGAAGGGACTTTAGAGAATACCAGTGTT
ATTAATGACAAAGGCACTGAGGCCCAGGGAGGTGACCCGAATTATAAAGG
CCAGCGCCAGAACCCAGATTTCCTAACTCTGGTGCTCTTTCCCTTTATCA
GTTTGACTGTGGCCTGTTAACTGGTATATACATATATATGTCAGGCAAAG
TGCTGCTGGAAGTAGAATTTGTCCAATAACAGGTCAACTTCAGAGACTAT
CTGATTTCCTAATGTCAGAGTAGAAGATTTTATGCTGCTGTTTACAAAAG
CCCAATGTAATGCATAGGAAGTATGGCATGAACATCTTTAGGAGACTAAT
GGAAATATTATTGGTGTTTACCCAGTATTCCATTTTTTTCATTGTGTTCT
CTATTGCTGCTCTCTCACTCCCCCATGAGGTACAGCAGAAAGGAGAACTA
TCCAAAACTAATTTCCTCTGACATGTAAGACGAATGATTTAGGTACGTCA
AAGCAGTAGTCAAGGAGGAAAGGGATAGTCCAAAGACTTAACTGGTTCAT
ATTGGACTGATAATCTCTTTAAATGGCTTTATGCTAGTTTGACCTCATTT
GTAAAATATTTATGAGAAAGTTCTCATTTAAAATGAGATCGTTGTTTACA
GTGTATGTACTAAGCAGTAAGCTATCTTCAAATGTCTAAGGTAGTAACTT
TCCATAGGGCCTCCTTAGATCCCTAAGATGGCTTTTTCTCCTTGGTATTT
CTGGGTCTTTCTGACATCAGCAGAGAACTGGAAAGACATAGCCAACTGCT
GTTCATGTTACTCATGACTCCTTTCTCTAAAACTGCCTTCCACAATTCAC
TAGACCAGAAGTGGACGCAACTTAAGCTGGGATAATCACATTATCATCTG
AAAATCTGGAGTTGAACAGCAAAAGAAGACAACATTTCTCAAATGCACAT
CTCATGGCAGCTAAGCCACATGGCTGGGATTTAAAGCCTTTAGAGCCAGC
CCATGGCTTTAGCTACCTCACTATGCTGCTTCACAAACCTTGCTCCTGTG
TAAAACTATATTCTCAGTGTAGGGCAGAGAGGTCTAACACCAACATAAGG
TACTAGCAGTGTTTCCCGTATTGACAGGAATACTTAACTCAATAATTCTT
TTCTTTTCCATTTAGTAACAGTTGTGATGACTATGTTTCTATTCTAAGTA
ATTCCTGTATTCTACAGCAGATACTTTGTCAGCAATACTAAGGGAAGAAA
CAAAGTTGAACCGTTTCTTTAATAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAA

By “Cluster of Differentiation 58 polypeptide” (CD58) is meant a protein or fragment thereof having at least about 85% amino acid identity to GENBANK Accession No. AAV38620.1, and having CD2 binding activity. An exemplary polypeptide sequence is provided below:

>AAV38620.1 CD58 antigen, (lymphocyte function-
associated antigen 3) [Homo sapiens]
(SEQ ID NO: 134)
MVAGSDAGRALGVLSVVCLLHCFGFISCFSQQIYGVVYGNVTFHVPSNVP
LKEVLWKKQKDKVAELENSEFRAFSSFKNRVYLDTVSGSLTIYNLTSSDE
DEYEMESPNITDTMKFFLYVLESLPSPTLTCALINGSIEVQCMIPEHYNS
HRGLIMYSWDCPMEQCKRNSTSIYFKMENDLPQKIQCTLSNPLFNTTSSI
ILTTCIPSSGHSRHRYALIPIPLAVITTCIVLYMNGILKCDRKPDRTNSN

In the present disclosure, it will be understood that Cluster of Differentiation 58 can also be referred to as CD58 or Lymphocyte Function-Associated Antigen 3.

“By CD58 polynucleotide” is meant a nucleic acid molecule encoding a CD58 polypeptide. An exemplary sequence of a CD58 polynucleotide is provided below:

>BT019817.1 Homo sapiens CD58 antigen, (lymphocyte
function-associated antigen 3) mRNA, complete cds
(SEQ ID NO: 135)
ATGGTTGCTGGGAGCGACGCGGGGGGGGCCCTGGGGGTCCTCAGCGTGGT
CTGCCTGCTGCACTGCTTTGGTTTCATCAGCTGTTTTTCCCAACAAATAT
ATGGTGTTGTGTATGGGAATGTAACTTTCCATGTACCAAGCAATGTGCCT
TTAAAAGAGGTCCTATGGAAAAAACAAAAGGATAAAGTTGCAGAACTGGA
AAATTCTGAATTCAGAGCTTTCTCATCTTTTAAAAATAGGGTTTATTTAG
ACACTGTGTCAGGTAGCCTCACTATCTACAACTTAACATCATCAGATGAA
GATGAGTATGAAATGGAATCGCCAAATATTACTGATACCATGAAGTTCTT
TCTTTATGTGCTTGAGTCTCTTCCATCTCCCACACTAACTTGTGCATTGA
CTAATGGAAGCATTGAAGTCCAATGCATGATACCAGAGCATTACAACAGC
CATCGAGGACTTATAATGTACTCATGGGATTGTCCTATGGAGCAATGTAA
ACGTAACTCAACCAGTATATATTTTAAGATGGAAAATGATCTTCCACAAA
AAATACAGTGTACTCTTAGCAATCCATTATTTAATACAACATCATCAATC
ATTTTGACAACCTGTATCCCAAGCAGCGGTCATTCAAGACACAGATATGC
ACTTATACCCATACCATTAGCAGTAATTACAACATGTATTGTGCTGTATA
TGAATGGTATTCTGAAATGTGACAGAAAACCAGACAGAACCAACTCCAAT
TAG

By “Signal Regulatory Protein Alpha (SIRPα) polypeptide” is meant a polypeptide or fragment thereof having at least about 85% amino acid identity to GENBANK Accession No. AAH38510.1, and having CD47 binding activity. An exemplary polypeptide sequence is provided below:

>AAH38510.1 Signal-regulatory protein alpha [Homo
sapiens]
(SEQ ID NO: 136)
MEPAGPAPGRLGPLLCLLLAASCAWSGVAGEEELQVIQPDKSVSVAAGES
AILHCTVTSLIPVGPIQWFRGAGPARELIYNQKEGHFPRVTTVSESTKRE
NMDFSISISNITPADAGTYYCVKFRKGSPDTEFKSGAGTELSVRAKPSAP
VVSGPAARATPQHTVSFTCESHGFSPRDITLKWFKNGNELSDFQTNVDPV
GESVSYSIHSTAKVVLTREDVHSQVICEVAHVTLQGDPLRGTANLSETIR
VPPTLEVTQQPVRAENQVNVTCQVRKFYPQRLQLTWLENGNVSRTETAST
VTENKDGTYNWMSWLLVNVSAHRDDVKLTCQVEHDGQPAVSKSHDLKVSA
HPKEQGSNTAAENTGSNERNIYIVVGVVCTLLVALLMAALYLVRIRQKKA
QGSTSSTRLHEPEKNAREITQDTNDITYADLNLPKGKKPAPQAAEPNNHT
EYASIQTSPQPASEDTLTYADLDMVHLNRTPKQPAPKPEPSFSEYASVQV
PRK

In the present disclosure, it will be understood that Signal Regulatory Protein Alpha can also be referred to as signal regulatory protein u, SIRPα, or SIRPA.

By “SIRPα polynucleotide” is meant a nucleic acid molecule encoding a SIRPα polypeptide. An exemplary sequence of a SIRPα polynucleotide is provided below:

>BC038510.2 Homo sapiens signal-regulatory protein
alpha, mRNA (cDNA clone MGC: 43149 IMAGE:
5261789), complete cds
(SEQ ID NO: 137)
CTCTCTGGCCGCCCCTGGCTTTATTTCTCGCGCGCTTGGGGTCTCTCCCA
GTCTCCGTCTCTCCATTTCTCCTGGGGGGCGGGGAGGGGGGGTCTCCAAA
AACCGCGGCGGCGGCGGCGGCCGCTCCAGGCGCCCGTTCCGGAGTCGGGG
GGAGGCCCAGCCGGGAGGGGGGAAGGGGGGGAGCCTTAGTCATTTCCCCG
CTCCAGCCTGCTCCCGCCCGAGCGCGCACTCACGGCCGCTCTCCCTCCTC
GCTCCGCAGCCGCGGCCCATGGAGCCCGCCGGCCCGGCCCCCGGCCGCCT
CGGGCCGCTGCTCTGCCTGCTGCTCGCCGCGTCCTGCGCCTGGTCAGGAG
TGGCGGGTGAGGAGGAGCTGCAGGTGATTCAGCCTGACAAGTCCGTATCA
GTTGCAGCTGGAGAGTCGGCCATTCTGCACTGCACTGTGACCTCCCTGAT
CCCTGTGGGGCCCATCCAGTGGTTCAGAGGAGCTGGACCAGCCCGGGAAT
TAATCTACAATCAAAAAGAAGGCCACTTCCCCCGGGTAACAACTGTTTCA
GAGTCCACAAAGAGAGAAAACATGGACTTTTCCATCAGCATCAGTAACAT
CACCCCAGCAGATGCCGGCACCTACTACTGTGTGAAGTTCCGGAAAGGGA
GCCCTGACACGGAGTTTAAGTCTGGAGCAGGCACTGAGCTGTCTGTGCGT
GCCAAACCCTCTGCCCCCGTGGTATCGGGCCCTGCGGCGAGGGCCACACC
TCAGCACACAGTGAGCTTCACCTGCGAGTCCCACGGCTTCTCACCCAGAG
ACATCACCCTGAAATGGTTCAAAAATGGGAATGAGCTCTCAGACTTCCAG
ACCAACGTGGACCCCGTAGGAGAGAGCGTGTCCTACAGCATCCACAGCAC
AGCCAAGGTGGTGCTGACCCGCGAGGACGTTCACTCTCAAGTCATCTGCG
AGGTGGCCCACGTCACCTTGCAGGGGGACCCTCTTCGTGGGACTGCCAAC
TTGTCTGAGACCATCCGAGTTCCACCCACCTTGGAGGTTACTCAACAGCC
CGTGAGGGCAGAGAACCAGGTGAATGTCACCTGCCAGGTGAGGAAGTTCT
ACCCCCAGAGACTACAGCTGACCTGGTTGGAGAATGGAAACGTGTCCCGG
ACAGAAACGGCCTCAACCGTTACAGAGAACAAGGATGGTACCTACAACTG
GATGAGCTGGCTCCTGGTGAATGTATCTGCCCACAGGGATGATGTGAAGC
TCACCTGCCAGGTGGAGCATGACGGGCAGCCAGCGGTCAGCAAAAGCCAT
GACCTGAAGGTCTCAGCCCACCCGAAGGAGCAGGGCTCAAATACCGCCGC
TGAGAACACTGGATCTAATGAACGGAACATCTATATTGTGGTGGGTGTGG
TGTGCACCTTGCTGGTGGCCCTACTGATGGCGGCCCTCTACCTCGTCCGA
ATCAGACAGAAGAAAGCCCAGGGCTCCACTTCTTCTACAAGGTTGCATGA
GCCCGAGAAGAATGCCAGAGAAATAACACAGGACACAAATGATATCACAT
ATGCAGACCTGAACCTGCCCAAGGGGAAGAAGCCTGCTCCCCAGGCTGCG
GAGCCCAACAACCACACGGAGTATGCCAGCATTCAGACCAGCCCGCAGCC
CGCGTCGGAGGACACCCTCACCTATGCTGACCTGGACATGGTCCACCTCA
ACCGGACCCCCAAGCAGCCGGCCCCCAAGCCTGAGCCGTCCTTCTCAGAG
TACGCCAGCGTCCAGGTCCCGAGGAAGTGAATGGGACCGTGGTTTGCTCT
AGCACCCATCTCTACGCGCTTTCTTGTCCCACAGGGAGCCGCCGTGATGA
GCACAGCCAACCCAGTTCCCAGAGGGCTGGGGCGGTGCAGGCTCTGGGAC
CCAGGGCCAGGGTGGCTCTTCTCTCCCCACCCCTCCTTGGCTCTCCAGCA
CTTCCTGGGCAGCCACGGCCCCCTCCCCCCACATTGCCACATACCTGGAG
GCTGACGTTGCCAAACCAGCCAGGGAACCAACCTGGGAAGTGGCCAGAAC
TGCCTGGGGTCCAAGAACTGTTGTGCCTCCGTCCATCACCATGTGGGTTT
TGAAGACCCTCGACTGCCTCCCCGATGCTCCGAAGCCTGATCTTCCAGGG
TGGGGAGGAGAAAATCCCACCTCCCCTGACCTCCACCACCTCCACCACCA
CCACCACCACCACCACCACCACCACCACTACCACCACCACCCAACTGGGG
CTAGAGTGGGGAAGATTTCCCCTTTAGATCAAACTGCCCCTTCCATGGAA
AAGCTGGAAAAAAACTCTGGAACCCATATCCAGGCTTGGTGAGGTTGCTG
CCAACAGTCCTGGCCTCCCCCATCCCTAGGCTAAAGAGCCATGAGTCCTG
GAGGAGGAGAGGACCCCTTCCAAAGGACTGGAGACAAAACCCTCTGCTTC
CTTGGGTCCCTCCAAGACTCCCTGGGGCCCAACTGTGTTGCTCCACCCGG
ACCCATCTCTCCCTTCTAGACCTGAGCTTGCCCCTCCAGCTAGCACTAAG
CAACATCTCGCTGTGGACGCCTGTAAATTACTGAGAAATGTGAAACGTGC
AATCTTGAAACTGAGGTGTTAGAAAACTTGATCTGTGGTGTTTTGTTTTG
TTTTTTTTCTTAAAACAACAGCAACGTGATCTTGGCTGTCTGTCATGTGT
TGAAGTCCATGGTTGGGTCTTGTGAAGTCTGAGGTTTAACAGTTTGTTGT
CCTGGAGGGATTTTCTTACAGCGAAGACTTGAGTTCCTCCAAGTCCCAGA
ACCCCAAGAATGGGCAAGAAGGATCAGGTCAGCCACTCCCTGGAGACACA
GCCTTCTGGCTGGGACTGACTTGGCCATGTTCTCAGCTGAGCCACGCGGC
TGGTAGTGCAGCCTTCTGTGACCCCGCTGTGGTAAGTCCAGCCTGCCCAG
GGCTGCTGAGGGCTGCCTCTTGACAGTGCAGTCTTATCGAGACCCAACGC
CTCAGTCTGCTCATCCGTAAAGTGGGGATAGTGAAGATGACACCCCTCCC
CACCACCTCTCATAAGCACTTTAGGAACACACAGAGGGTAGGGATAGTGG
CCCTGGCCGTCTATCCTACCCCTTTAGTGACCGCCCCCATCCCGGCTTTC
TGAGCTGATCCTTGAAGAAGAAATCTTCCATTTCTGCTCTCAAACCCTAC
TGGGATCAAACTGGAATAAATTGAAGACAGCCAGGGGGATGGTGCAGCTG
TGAAGCTCGGGCTGATTCCCCCTCTGTCCCAGAAGGTTGGCCAGAGGGTG
TGACCCAGTTACCCTTTAACCCCCACCCTTCCAGTCGGGTGTGAGGGCCT
GACCGGGCCCAGGGCAAGCAGATGTCGCAAGCCCTATTTATTCAGTCTTC
ACTATAACTCTTAGAGTTGAGACGCTAATGTTCATGACTCCTGGCCTTGG
GATGCCCAAGGGATTTCTGGCTCAGGCTGTAAAAGTAGCTGAGCCATCCT
GCCCATTCCTGGAGGTCCTACAGGTGAAACTGCAGGAGCTCAGCATAGAC
CCAGCTCTCTGGGGGATGGTCACCTGGTGATTTCAATGATGGCATCCAGG
AATTAGCTGAGCCAACAGACCATGTGGACAGCTTTGGCCAGAGCTCCCGT
GTGGCATCTGGGAGCCACAGTGACCCAGCCACCTGGCTCAGGCTAGTTCC
AAATTCCAAAAGATTGGCTTGTAAACCTTCGTCTCCCTCTCTTTTACCCA
GAGACAGCACATACGTGTGCACACGCATGCACACACACATTCAGTATTTT
AAAAGAATGTTTTCTTGGTGCCATTTTCATTTTATTTTATTTTTTAATTA
TTGGAGGGGGAAATAAGGGAATAAGGCCAAGGAAGATGTATAGCTTTAGC
TTTAGCCTGGCAACCTGGAGAATCCACATACCTTGTGTATTGAACCCCAG
GAAAAGGAAGAGGTCGAACCAACCCTGCGGAAGGAGCATGGTTTCAGGAG
TTTATTTTAAGACTGCTGGGAAGGAAACAGGCCCCATTTTGTATATAGTT
GCAACTTAAACTTTTTGGCTTGCAAAATATTTTTGTAATAAAGATTTCTG
GGTAATAATGAAAAAAAAAAAAAAAAAAAAAAAAAAAAAAAAAGAAAAAA
AAAAAAA

By “Cluster of Differentiation 47 (CD47) polypeptide” is meant a protein or fragment thereof having at least about 85% amino acid identity to GENBANK Accession No. AAH37306.1, and having SIRPα binding activity. An exemplary polypeptide sequence is provided below:

>AAH37306.1 CD47 protein [Homo sapiens]
(SEQ ID NO: 138)
MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNOKTIQ
PPRNN

By “CD47 polynucleotide” is meant a nucleic acid molecule encoding a CD47 polypeptide. An exemplary sequence of a CD47 polynucleotide is provided below:

>BC037306.1 Homo sapiens CD47 molecule, mRNA (cDNA clone MGC: 33903
IMAGE: 5260986) , complete cds
(SEQ ID NO: 139)
AGCGGAGTGGGTCCTGCCTGTGACGCGCGGCGGCGGTCGGTCCTGCCTGTAACGGCGGCGGCGGCTGCTG
CTCCAGACACCTGCGGCGGCGGCGGCGACCACGCGGCGGGCGCGGAGATGTGGCCCCTGGTAGCGGCGCT
GTTGCTGGGCTCGGCGTGCTGCGGATCAGCTCAGCTACTATTTAATAAAACAAAATCTGTAGAATTCACG
TTTTGTAATGACACTGTCGTCATTCCATGCTTTGTTACTAATATGGAGGCACAAAACACTACTGAAGTAT
ACGTAAAGTGGAAATTTAAAGGAAGAGATATTTACACCTTTGATGGAGCTCTAAACAAGTCCACTGTCCC
CACTGACTTTAGTAGTGCAAAAATTGAAGTCTCACAATTACTAAAAGGAGATGCCTCTTTGAAGATGGAT
AAGAGTGATGCTGTCTCACACACAGGAAACTACACTTGTGAAGTAACAGAATTAACCAGAGAAGGTGAAA
CGATCATCGAGCTAAAATATCGTGTTGTTTCATGGTTTTCTCCAAATGAAAATATTCTTATTGTTATTTT
CCCAATTTTTGCTATACTCCTGTTCTGGGGACAGTTTGGTATTAAAACACTTAAATATAGATCCGGTGGT
ATGGATGAGAAAACAATTGCTTTACTTGTTGCTGGACTAGTGATCACTGTCATTGTCATTGTTGGAGCCA
TTCTTTTCGTCCCAGGTGAATATTCATTAAAGAATGCTACTGGCCTTGGTTTAATTGTGACTTCTACAGG
GATATTAATATTACTTCACTACTATGTGTTTAGTACAGCGATTGGATTAACCTCCTTCGTCATTGCCATA
TTGGTTATTCAGGTGATAGCCTATATCCTCGCTGTGGTTGGACTGAGTCTCTGTATTGCGGCGTGTATAC
CAATGCATGGCCCTCTTCTGATTTCAGGTTTGAGTATCTTAGCTCTAGCACAATTACTTGGACTAGTTTA
TATGAAATTTGTGGCTTCCAATCAGAAGACTATACAACCTCCTAGGAATAACTGAAGTGAAGTGATGGAC
TCCGATTTGGAGAGTAGTAAGACGTGAAAGGAATACACTTGTGTTTAAGCACCATGGCCTTGATGATTCA
CTGTTGGGGAGAAGAAACAAGAAAAGTAACTGGTTGTCACCTATGAGACCCTTACGTGATTGTTAGTTAA
GTTTTTATTCAAAGCAGCTGTAATTTAGTTAATAAAATAATTATGATCTATGTTGTTTGCCCAATTGAGA
TCCAGTTTTTTGTTGTTATTTTTAATCAATTAGGGGCAATAGTAGAATGGACAATTTCCAAGAATGATGC
CTTTCAGGTCCTAGGGCCTCTGGCCTCTAGGTAACCAGTTTAAATTGGTTCAGGGTGATAACTACTTAGC
ACTGCCCTGGTGATTACCCAGAGATATCTATGAAAACCAGTGGCTTCCATCAAACCTTTGCCAACTCAGG
TTCACAGCAGCTTTGGGCAGTTATGGCAGTATGGCATTAGCTGAGAGGTGTCTGCCACTTCTGGGTCAAT
GGAATAATAAATTAAGTACAGGCAGGAATTTGGTTGGGAGCATCTTGTATGATCTCCGTATGATGTGATA
TTGATGGAGATAGTGGTCCTCATTCTTGGGGGTTGCCATTCCCACATTCCCCCTTCAACAAACAGTGTAA
CAGGTCCTTCCCAGATTTAGGGTACTTTTATTGATGGATATGTTTTCCTTTTATTCACATAACCCCTTGA
AACCCTGTCTTGTCCTCCTGTTACTTGCTTCTGCTGTACAAGATGTAGCACCTTTTCTCCTCTTTGAACA
TGGTCTAGTGACACGGTAGCACCAGTTGCAGGAAGGAGCCAGACTTGTTCTCAGAGCACTGTGTTCACAC
TTTTCAGCAAAAATAGCTATGGTTGTAACATATGTATTCCCTTCCTCTGATTTGAAGGCAAAAATCTACA
GTGTTTCTTCACTTCTTTTCTGATCTGGGGCATGAAAAAAGCAAGATTGAAATTTGAACTATGAGTCTCC
TGCATGGCAACAAAATGTGTGTCACCATCAGGCCAACAGGCCAGCCCTTGAATGGGGATTTATTACTGTT
GTATCTATGTTGCATGATAAACATTCATCACCTTCCTCCTGTAGTCCTGCCTCGTACTCCCCTTCCCCTA
TGATTGAAAAGTAAACAAAACCCACATTTCCTATCCTGGTTAGAAGAAAATTAATGTTCTGACAGTTGTG
ATCGCCTGGAGTACTTTTAGACTTTTAGCATTCGTTTTTTACCTGTTTGTGGATGTGTGTTTGTATGTGC
ATACGTATGAGATAGGCACATGCATCTTCTGTATGGACAAAGGTGGGGTACCTACAGGAGAGCAAAGGTT
AATTTTGTGCTTTTAGTAAAAACATTTAAATACAAAGTTCTTTATTGGGTGGAATTATATTTGATGCAAA
TATTTGATCACTTAAAACTTTTAAAACTTCTAGGTAATTTGCCACGCTTTTTGACTGCTCACCAATACCC
TGTAAAAATACGTAATTCTTCCTGTTTGTGTAATAAGATATTCATATTTGTAGTTGCATTAATAATAGTT
ATTTCTTAGTCCATCAGATGTTCCCGTGTGCCTCTTTTATGCCAAATTGATTGTCATATTTCATGTTGGG
ACCAAGTAGTTTGCCCATGGCAAACCTAAATTTATGACCTGCTGAGGCCTCTCAGAAAACTGAGCATACT
AGCAAGACAGCTCTTCTTGAAAAAAAAAATATGTATACACAAATATATACGTATATCTATATATACGTAT
GTATATACACACATGTATATTCTTCCTTGATTGTGTAGCTGTCCAAAATAATAACATATATAGAGGGAGC
TGTATTCCTTTATACAAATCTGATGGCTCCTGCAGCACTTTTTCCTTCTGAAAATATTTACATTTTGCTA
ACCTAGTTTGTTACTTTAAAAATCAGTTTTGATGAAAGGAGGGAAAAGCAGATGGACTTGAAAAAGATCC
AAGCTCCTATTAGAAAAGGTATGAAAATCTTTATAGTAAAATTTTTTATAAACTAAAGTTGTACCTTTTA
ATATGTAGTAAACTCTCATTTATTTGGGGTTCGCTCTTGGATCTCATCCATCCATTGTGTTCTCTTTAAT
GCTGCCTGCCTTTTGAGGCATTCACTGCCCTAGACAATGCCACCAGAGATAGTGGGGGAAATGCCAGATG
AAACCAACTCTTGCTCTCACTAGTTGTCAGCTTCTCTGGATAAGTGACCACAGAAGCAGGAGTCCTCCTG
CTTGGGCATCATTGGGCCAGTTCCTTCTCTTTAAATCAGATTTGTAATGGCTCCCAAATTCCATCACATC
ACATTTAAATTGCAGACAGTGTTTTGCACATCATGTATCTGTTTTGTCCCATAATATGCTTTTTACTCCC
TGATCCCAGTTTCTGCTGTTGACTCTTCCATTCAGTTTTATTTATTGTGTGTTCTCACAGTGACACCATT
TGTCCTTTTCTGCAACAACCTTTCCAGCTACTTTTGCCAAATTCTATTTGTCTTCTCCTTCAAAACATTC
TCCTTTGCAGTTCCTCTTCATCTGTGTAGCTGCTCTTTTGTCTCTTAACTTACCATTCCTATAGTACTTT
ATGCATCTCTGCTTAGTTCTATTAGTTTTTTGGCCTTGCTCTTCTCCTTGATTTTAAAATTCCTTCTATA
GCTAGAGCTTTTCTTTCTTTCATTCTCTCTTCCTGCAGTGTTTTGCATACATCAGAAGCTAGGTACATAA
GTTAAATGATTGAGAGTTGGCTGTATTTAGATTTATCACTTTTTAATAGGGTGAGCTTGAGAGTTTTCTT
TCTTTCTGTTTTTTTTTTTTTTTTTTTTGACTAATTTCACATGCTCTAAAAACCTTCAAAGGTGATTATT
TTTCTCCTGGAAACTCCAGGTCCATTCTGTTTAAATCCCTAAGAATGTCAGAATTAAAATAACAGGGCTA
TCGCGTAATTGGAAATATTTCTTTTTTCAGGATGCTATAGTCAATTTAGTAAGTGACCACCAAATTGTTA
TTTGCACTAACAAAGCTCAAAACACGATAAGTTTACTCCTCCATCTCAGTAATAAAAATTAAGCTGTAAT
CAACCTTCTAGGTTTCTCTTGTCTTAAAATGGGTATTCAAAAATGGGGATCTGTGGTGTATGTATGGAAA
CACATACTCCTTAATTTACCTGTTGTTGGAAACTGGAGAAATGATTGTCGGGCAACCGTTTATTTTTTAT
TGTATTTTATTTGGTTGAGGGATTTTTTTATAAACAGTTTTACTTGTGTCATATTTTAAAATTACTAACT
GCCATCACCTGCTGGGGTCCTTTGTTAGGTCATTTTCAGTGACTAATAGGGATAATCCAGGTAACTTTGA
AGAGATGAGCAGTGAGTGACCAGGCAGTTTTTCTGCCTTTAGCTTTGACAGTTCTTAATTAAGATCATTG
AAGACCAGCTTTCTCATAAATTTCTCTTTTTGAAAAAAAGAAAGCATTTGTACTAAGCTCCTCTGTAAGA
CAACATCTTAAATCTTAAAAGTGTTGTTATCATGACTGGTGAGAGAAGAAAACATTTTGTTTTTATTAAA
TGGAGCATTATTTACAAAAAGCCATTGTTGAGAATTAGATCCCACATCGTATAAATATCTATTAACCATT
CTAAATAAAGAGAACTCCAGTGTTGCTATGTGCAAGATCCTCTCTTGGAGCTTTTTTGCATAGCAATTAA
AGGTGTGCTATTTGTCAGTAGCCATTTTTTTGCAGTGATTTGAAGACCAAAGTTGTTTTACAGCTGTGTT
ACCGTTAAAGGTTTTTTTTTTTATATGTATTAAATCAATTTATCACTGTTTAAAGCTTTGAATATCTGCA
ATCTTTGCCAAGGTACTTTTTTATTTAAAAAAAAACATAACTTTGTAAATATTACCCTGTAATATTATAT
ATACTTAATAAAACATTTTAAGCTAAAAAAAAAAAAACAAAAAAAAAAAAAAAA

By “Cluster of Differentiation 40 Ligand polypeptide” (CD40L) is meant a protein or fragment thereof having at least about 85% amino acid identity to GENBANK Accession No. AAH74950.1, and having CD40 binding activity. An exemplary polypeptide sequence is provided below:

>AAH74950.1 CD40 ligand [Homo sapiens]
(SEQ ID NO: 140)
MIETYNQTSPRSAATGLPISMKIFMYLLTVFLITQMIGSALFAVYLHRRL
DKIEDERNLHEDFVFMKTIQRCNTGERSLSLLNCEEIKSQFEGFVKDIML
NKEETKKENSFEMQKGDQNPQIAAHVISEASSKTTSVLQWAEKGYYTMSN
NLVTLENGKQLTVKRQGLYYIYAQVTFCSNREASSQAPFIASLCLKSPGR
FERILLRAANTHSSAKPCGQQSIHLGGVFELQPGASVFVNVTDPSQVSHG
TGFTSFGLLKL

In the present disclosure, it will be understood that Cluster of Differentiation 40 Ligand can also be referred to as CD40 ligand, CD40LG, or CD154.

By “CD40L polynucleotide” is meant a nucleic acid molecule encoding a CD40L polypeptide. An exemplary sequence of a CD40L polynucleotide is provided below:

>BC074950.2 Homo sapiens CD40 ligand, mRNA (cDNA
clone MGC: 103892 IMAGE: 30915276), complete cds
(SEQ ID NO: 141)
TCTGCCAGAAGATACCATTTCAACTTTAACACAGCATGATCGAAACATAC
AACCAAACTTCTCCCCGATCTGCGGCCACTGGACTGCCCATCAGCATGAA
AATTTTTATGTATTTACTTACTGTTTTTCTTATCACCCAGATGATTGGGT
CAGCACTTTTTGCTGTGTATCTTCATAGAAGGTTGGACAAGATAGAAGAT
GAAAGGAATCTTCATGAAGATTTTGTATTCATGAAAACGATACAGAGATG
CAACACAGGAGAAAGATCCTTATCCTTACTGAACTGTGAGGAGATTAAAA
GCCAGTTTGAAGGCTTTGTGAAGGATATAATGTTAAACAAAGAGGAGACG
AAGAAAGAAAACAGCTTTGAAATGCAAAAAGGTGATCAGAATCCTCAAAT
TGCGGCACATGTCATAAGTGAGGCCAGCAGTAAAACAACATCTGTGTTAC
AGTGGGCTGAAAAAGGATACTACACCATGAGCAACAACTTGGTAACCCTG
GAAAATGGGAAACAGCTGACCGTTAAAAGACAAGGACTCTATTATATCTA
TGCCCAAGTCACCTTCTGTTCCAATCGGGAAGCTTCGAGTCAAGCTCCAT
TTATAGCCAGCCTCTGCCTAAAGTCCCCCGGTAGATTCGAGAGAATCTTA
CTCAGAGCTGCAAATACCCACAGTTCCGCCAAACCTTGCGGGCAACAATC
CATTCACTTGGGAGGAGTATTTGAATTGCAACCAGGTGCTTCGGTGTTTG
TCAATGTGACTGATCCAAGCCAAGTGAGCCATGGCACTGGCTTCACGTCC
TTTGGCTTACTCAAACTCTGAACAGTGTCACCTTGCAGGCTGTGGTGGAG
CTGACGCTGGGAGTCTTCAT

By “Cluster of Differentiation 40 polypeptide” (CD40) is meant a protein or fragment thereof having at least about 85% amino acid identity to GENBANK Accession No. ABK41937.1, and having CD40L binding activity. An exemplary polypeptide sequence is provided below:

>ABK41937.1 CD40 molecule, TNF receptor super-
family member 5 [Homo sapiens]
(SEQ ID NO: 142)
MVRLPLQCVLWGCLLTAVHPEPPTACREKQYLINSQCCSLCQPGQKLVSD
CTEFTETECLPCGESEFLDTWNRETHCHQHKYCDPNLGLRVQQKGTSETD
TICTCEEGWHCTSEACESCVLHRSCSPGFGVKQIATGVSDTICEPCPVGF
FSNVSSAFEKCHPWTSCETKDLVVQQAGTNKTDVVCGPQDRLRALVVIPI
IFGILFAILLVLVFIKKVAKKPTNKAPHPKQEPQEINFPDDLPGSNTAAP
VQETLHGCQPVTQEDGKESRISVQERQ

By “CD40 polynucleotide” is meant a nucleic acid molecule encoding a CD40 polypeptide. An exemplary sequence of a CD40 polynucleotide is provided below:

>EF064754.1 Homo sapiens CD40 molecule, TNF receptor superfamily member 5
(CD40) gene, complete cds
(SEQ ID NO: 143)
TTAATAAATGCCTGTCTCCAGGTGCTGGGTGGGAGGTGGGATGGAATGGAATGAGGTGAGGACGCATGGA
TGCATGGATGAATGGATGGGAAGTTGAGACGACGCGCCCACACGAGGGAATTTCCTTTGAAAGAGAGCGA
AATTCTGAGTTGGGAAACTCTTCCTTGAAACGCCTCCCCATACCCCAGCTGTGGCCTTCCCGTTTTCTGC
GTGGTGGTGTGGGGGGAACTTCCTCAGGCCTCTCCGCAGTGGAGCCTCTTTCGGTTCTGCCAGGATACCT
AGAGGCAGCGGAGAGCGGGGCAGGGAGGGGAAAACCGTGAGGGTCCCTGTGGCAGGCCCCAGCACCCATG
GGATCTCTCTCCGGTCGCAGGAAGCAGGCTAGCTCCTAGCCCGCCTCGGCTTGGCCTTTGTGGGACCTGG
GGGCAAAGAAGAAGAGCTGTCTCTGGGACCATGCCTCCTCCCGTACACAGCAAGATGCGTCCCTAAACTC
CCGGGGGAATTAGACTTGTGGGAATGTTCTGGGGAAACTCCTGCGCGGTGAATTGCTGGGGGCTCCGCCC
CCCCCGATAGGTGGACCGCGATTGGTCTTTGAAGACCCCGCCCCTTTCCTGGGGGGGGCCAAGGCTGGGG
CAGGGGAGTCAGCAGAGGCCTCGCTCGGGCGCCCAGTGGTCCTGCCGCCTGGTCTCACCTCGCCATGGTT
CGTCTGCCTCTGCAGTGCGTCCTCTGGGGCTGCTTGCTGACCGCTGTGAGTTGTTTTTGCCCCGACCAGA
CGGGAGTTGGGAGTGGGGAATGAGAAGGAAAGGGAAGGAAGACTTCGGGGAAGAGGCCTTCCTGGCTGAT
TTTTGTGGGGGCAGGAGGGTGGGTGGGAGCTGGGCAAGGTGCCCCCGCTCCTGGCTGAATGGGGTGGGCT
GCCTCTCTCTTCTCCCGGGCTGGGGTCCCGGGAGCGGCCTACAGGGGCCGCTCAGGGAAGGCACTGGCTG
CCCAAGCGTGCCTAGACGGCCTGGACGGGTTTAGGGAGCCTCAGAGGCTGGCCACACAGAGACTGGTAGG
GGGTTCAGAGGGCGGGAAGTGAGGCGGACCAAGGGAAGGGGCGGGTCTGGCCCGTTTCCTGTCCCCTTCT
TATTGTGGACAGATGCCAGCCTCTGTAAGTAGTTATCATCTCCTTGCCAGCTGGGGCTGCCTTCTTCCAG
GGCATCTTGTGGGAACAAGAGATGGGTGCAGAGGCCCAGGTACTTTTGTGAGAAGGCAAGGAGCTTTTAA
CATCGCCTTCCACCCCGAACCGTATCTTGGGTGTTCCAACCTAGGAGGAATCCCCAGGGCTTTGCCTTTT
TCTCCTGAATTTAAGATGACATAGGAGACCCCTGGGGAGATGAACAGTTTATGGGACACAATAAAGGGTT
AGGAGACCAGAGTTCTGGTTGGCTCTGACAGGGCTGGTGATCAGAGGGCTGGAGAAACCAGGGGTTTCTC
CAGGCACCAGAGGGGCTCAGAGCCAACCAAGCATATCTCCGGGATTTTCAGAAGCCTACACTTGACTCAC
TTTTTGTTTAAATGTATTTTTGTAGTTCCTCATTCTGGAGGCTGGGAATCCCCCAAGTACCTGGCTCCTT
CATCCCAGCCCCTCTGGCCTCCCCCTACTTTAGAGGGCTGTAGATTCCTGCCTGAAGCCTGGGCAGGAAT
GACCCATGGTATCAAGGAAAGCAAGGGAAGCAGCAAGGGAAGAGAGGGAGTGGGGAGGCTGCTTTGGTCC
CACAGCTTTCACTTTCACCTGAAGCAATGGCTCTTAGGGAACAGGGAGGCAGGGGGAGGGCGGAGCTGGA
AAGAGGTAAAGGGGGGCCCTTGTGGTAGGAGTGGAGAAAGAGCCAGAGGAGGTGGGGTGAAGGGTGTGAT
CCAGGCTTCTCAAGAGCAGAGTTTGCCCTCATAACTCCCAACTTTGGCTCCAGGTAGAGGCTGGGCTGTG
ACAACAATGTCAGAAGCTATCTATTGAGGGCTTCTTGTGTGTCAGGCTCTGAGCCAAACACTGCCTGTTT
TCTTTGTCTGATTTCTCACAACTCCCCCATTATACAGATGGGCAAATTGAGGCTCAGAAAGGGGGATTGT
CTTGCCAAAGGTCTCATAGCTAGCTAATGGAAGAACCTGGTTGTGAATCTACATCTGCATGATTCCCGAG
CCTGCCTCTCAGATAGTGAGAGTCTCCAAGCTCTGGTCCTGAGCTGTTTTGTGGCAGAAGGACCAGAACT
ATGGGGAGTGAGAACTGGAGATTGACAGACTTTTAGGGGAGCGTTTTATTTCTCATGTGTTTGAAGATGG
TATCAAGGACTTTCCTATCTTTGGGAGTGTGGGAGCTCCACGTTCACAGGATGGTGTCTTGCAATGAGCT
GGTGGGGGGCAGTAGCCTTTTCTACTTCCTTTCCCATTTTGGGTAAGACACATTTCTGTAAGTAATTTGC
TGAGATACCCAGGTTGAATGAGAGCCACCAGTTAGGTAGGATTCTGGACAGCCAGCCAGGTAGCCGGGCT
GCTTGCCATATATCATGCAAGCAGAAACAAATGAATGATGATTAAAATTGCCATTTAATGAGCACCTACT
ATGTTCCTGACACTGTGCTAGGCCATATACATGTATTCTTTCTTATCTTCGTAATCCAACCTGCAGGGCA
GGCATTATTACTCCCATTTTAGAGATAGAGAAACTGAGGCTAAGAGAAGCAAAATAACTAGTAAGTGTTA
CAAAGTCAGGACTGGAGTCTAAAGCTGTCTGACTCTCAAACTTGTGTTCTTTTCACTGGCTGTTCCCAAA
CTGTGGGACAGTTTTAAGGAGCACATGGACATAGAATTAAACATACACTTACTTTACAGTTCTTTTAAAA
ATCCTTCTCATTTTTTCAAAGAGGAAGTCTCTGGAGCTAGAATAGAGTTAATGCCTCTCAAAGGCTTGCT
AATCCTTCTTTTAAAACAAAAATCAAGAGCAGGCCTGGGAGGGCCTTCAACAAGCAAACAACCAGCTGGG
TTTTAATAACCTTGTTTTGTTTCCCCAGAATTTATTTTTAGGGTTACCTTTTATTTATGAGAAGTGATAC
TGGTTCTTGTCTCTTGGCAATGATGTGAGGTTTACATTTAAAGTAAATGTACCGGCCAGGCACGGTGGCT
TGTGCCTGTAATCCCAGCACTTTGGGAGGCCAAGGCAGTCAGATCACTTGAGGTCAGGAGTTTTAGATCA
GCCTGGCCAACATGGTGAAACCCTGTCTCTACTAAAAATACATAAATTAGCCGGGCATAGTGGTACACAC
CTGTAATCCCAGCTACTCAGGAGGCTGAGGCTGGAGAATTGCTTGAACCCAGGAGATAGAGGTTGCAGTG
GGCTGAGATGATGCCACTGCACTCCAGCCTGGGCGATGGAGCGAGACTCTGTCTCAAAAAATAAAATAAA
AGTATTGAAATTAACAATAAGTAATTAATAGCATGGGTGGTACCTGGATGTAGTAAAATGGTGAAGATGA
AACACAAGTTGATGGAGAGAGGAGCATTGAGACCTGAGTTCTCATTTGGACTCTGTCACTGTGAGACTCT
GGGCAAGTGACCCTCCTCTTTGGTGCTCAGTCTCAACTATCTGTAAAATGAAAGTGTGAGTTTACCCTTC
CAGCTTTACATTCTAGCATTTTATGAGGGAAGGGCTGGATGAACAGATGATGAGGAGTTGGAGGAAGAAA
ACATGATGGGCTTTGGAAAGGAGCAGGAAGGGAAGCAGAAGAATAGGAGGAAGAGGCCAAGTGCTAAACA
TAGCCCCAAACAGCACTGGGACCAGCTGAAGTCAGCCAGCTTCAGGACTCCAGGGGAGCTGCTGGAGTCC
CCATATCCTATGGGATCTTTGGGAAGAGGAATGACTCAGGCATCAAGCCCCAAGGAATTCTGTTCTGTTC
AGAGAATATTGTGAGTTTACAGTACCATTGCTTTGTAAAAATACCAGAATGATTCTCTGGGTGCGATTAT
AATCAGCTCAGTTGACAATTTACTTGAAAACAAACATGCCAAATATCATGCAGGTTCCACTTTCTGTTTT
GACTTGCACTTCAGTTTGCAGCCTCTGTCCTGGATGACTTTTACCTTTCTGCTGAAGAAGTTGCAACGGA
GATTTCAAGATCCCTTCAAATTGCACAATTCTGTTTTTAGGTCCATCCAGAACCACCCACTGCATGCAGA
GAAAAACAGTACCTAATAAACAGTCAGTGCTGTTCTTTGTGCCAGCCAGGTGAGATGCCAACCCTCTAGC
CCCATCATGGAGTCCCCCTTTGCTTTGGTGGCAGACGCAGACCCCATATGTTAACTGTAAACTCAAATCT
GAAACGACCCATTTCCCAGCCCTGCTTCACTGTCAGAATGTTCTGGTTCCCTCTCTACCAGGTAAAACTC
TGTCTACCCTGAACTAGGGATCCCAGCTTCTCCATCTTCCTCGCCTGATTATGAAGGATCCAAGACTTTC
ATCTTTGAATCCCCTACCCTAAAGCCTGGCCTGATCATTGTGTGGTTAGTGTCTGACTCATGGAGTTGGC
CAGAGCCCTCCCTCATTTCCTGATGTTTTCCAGGACAGAAACTGGTGAGTGACTGCACAGAGTTCACTGA
AACGGAATGCCTTCCTTGCGGTGAAAGCGAATTCCTAGACACCTGGAACAGAGAGACACACTGCCACCAG
CACAAATACTGCGACCCCAGTGCGTGCGCTGTTGGGAAAGGGACGCTTGGGAACCGGGCTGATATTCCCG
ACAATGCAGCCATTCTAATTTTATGTAGCCAGGGTCTGCTCTGATTGGTTGGAGTCCGGGCTGTACTGAT
CATTAAATGATTTGATTGCCATCTCTACTTGGAAGAGGGTCTGAGGAAGAAAGAGCAGGCAATGTGGGGA
GTGAGGCTCAGAGCATGGCCCAGCAGGGGGTTCCCATCCTTCCTGCCCTTCTCTTCTCAGACCTAGGGCT
TCGGGTCCAGCAGAAGGGCACCTCAGAAACAGACACCATCTGCACCTGTGAAGAAGGCTGGCACTGTACG
AGTGAGGCCTGTGAGAGCTGTGTCCTGCACCGCTCATGCTCGCCCGGCTTTGGGGTCAAGCAGATTGGTA
AGTGGCTCATCTGGGAATCAGTTTTGGAGGGGGACAGAGGAGCTTAGGGCCCAAGGTGAGGGGCTGGGCA
GTGGGCACTTAGCCCCAGAGGCAGAGGAAGCAGAGGCTCCAACCTATGTCGGTATCCCCACTGGAGTGAG
CTGCAGACGGGACCTTGTTCATTCTGCCTTCTGCCATGGGGATCTGCCTTTGAAGGGCAATGGGAGAAGT
CCTCCTGGGGACTGCAGCTGTCGGGGGCAGTACCACATCGGGGGAAGAGTGCTCAAGGCAGGAGCTCTTC
CCGTCCTGCCTGGCCACTGGCTGCCTTGTGAGCCGGACAGGTGGTCCACTGTGATGGTTAATGTCCCCCT
CCCCACCCACTCCCAGCTACAGGGGTTTCTGATACCATCTGCGAGCCCTGCCCAGTCGGCTTCTTCTCCA
ATGTGTCATCTGCTTTCGAAAAATGTCACCCTTGGACAAGGTATAAGCACTCATCCCTTGTGTTTCCTGC
TCTAAGAGTGGCATGGAGCTGCCTCCATTCTCTCCAGCCACCTGTCCTGTCCCTGCTCCCAGAGGTCCAC
ACACACTCATGTACTTGTGAAGCATCTGCAGAGTGGCCTCATGGCCAACCAGACAGGCACATTTCCACAT
TTTTTTTGCCTGCTGTCTCTTTGAGGTAATAGACACTGTTGATCTCTCGCTTCATGAGAGCCTCCTATCT
TGGGGGTATTGGGACACTTATTTTAGCTTTCCTTCTGCCCCTCCTGCTTCTCCTCAGTTTTCCTCGTCTT
GCTTTCACCTTACCTGGCTTTCTAGGGCTTTCTGGGCTCTGGGTGCTCACCCTGAGGGCCTCCCTCTCTT
ACCTCCAACTCCAAACCCACACCAGGTCCTGCCACTGGCTGTCTACGTGTTTTGGGAACTTACTGTCTCC
ACTGTTGTCACTTTAGTTTGGGCCTCATCACTGTGGTCTGGGTGATGCCTTTTCTGCCTCCTGGCCTCCC
TGCCTCTGTCTCTCCCCTCCTGCTGGTTCTGTCTCCATCCTCTTGCCAACATGAGCGTTCGACAGTTTCT
TTCAAATCATGACACTCTCCTATTTGAGATGCTTCCTGTCTCTCTGTTGGAACTAAGACTCCTTAGCATG
GCACCCAACCTTCCTGTTGCATTTCCTGCTCTCTTTCCTGCATCGCATAGCTTCATGCTACTTGCAATCC
TCTGAACACACTGTTCATTCTCTTCCATCAAACTCATCTGCCTGGAATACCTTAAACATGGGCCCCAGGC
CAGGCGCGGTGGCTCTTGCCTGTAATCTCAGCACTTTGGATGCCAAGGCGGGTGGATCACTTGAGGTCAG
GAGTTCAAGACCAGCCAGCACAACATGGTAAAAACCCATCTCTACTAAAAATACCAAAAAATTAGCTGGG
TGTGGTGGTGGGCGCCTGTAATCCCAGCTCCTCGGGAGGCTGAGGCAGGAGAATCACTTGAACCCGGAAG
GTGGAGTTTGCAGTGAGCCAAGATAGCGCCACTGCACTCCAGCCTGGGCAACAGAGCGACATTCTGTCTC
AAAAAACAAACACCTGCCCCATTAACTTTTTGCATTTGATTTTTAAAAATGGGCAAGATAGGCACATGGG
ACAGAAGGCACAAAAGAGCCAAAGTGATGTCTTTCTCCCATCCCTGCCCCTTAGGCTCCCAGTTCTTTCT
GGAGGGAGCCATTGTTCCTTGCATATCCTTCCAGAGATTCTACATATAAACAAACCAACACACACACACA
CACACACAAACACACACAAAATTTCCCTCCTTTTACTTTTGCACAAATAGGAGTATACATTTTATTTGTT
AACTGTCTGCCTTTCCCTAATAGATTGAAAATTCCTTAAATGTAGAAACTTGGCCTTTTTTTTTTCTTCC
ATTGATACATCCCCTATACCTGGAACAGTACCTGACGCATGGTAGGTGCTTAAATTTTTACTGATAAATG
TTGACTGATAACTGGAGGCACCACTGGTATAGTTTTTTTTTTTTTTTTTTTTTTTTTTTTTTTTTGAGAC
AGAGTCTCACTCTGTCGCCCAGGCTGGAGTGCAGTGGCGCAATCTCGGCTCACTGCAAGCTCTGCCTCCC
AGGTTCACGCCATTCTCCTGCCTCAGCCTCCTGAGTAGCTGGGACTATAGGCGCCCGCCACCACACCCGG
CTAATTTTTTTGTATTTTTAGTAGAGACGGCGTTTCACCGTGTTAGCCAGGATGGTCTTGATCTCCTGAC
CTCGTGATCCGTCTGCCTTGGCCTCCCAAAGTGCTGGGATTACAGGCGTGAGCCACCGTGCCCGGCCACC
AGTGGTATAGTATTAATGGAATCAGTGCATTGGCTTACGTATCTGATTACAGCTCAGTAAGTGTGTGACC
CTCACTGAGCCTCAGTCTCCTCATCTGAAAAATGGGAATGACCTTCATTTCACAAGGCTTGAGCTAAAAA
CATGTAAAGTGTATTGTAAATTCCTGAATGCTCTACTCATGTAAGACTAAAGTAGGCCGGGCGTGGTGGC
TCACACCTGTAATTGCAGCACTTTGGGAGGCCGAGGAGGGCAGATCATGAGGTCAAGAGATCGAGACCAT
CCTGGCTAATATGGTAAAACCCTGTCTCTACTAAAAATACAAAAATTAGCTGGGCGTGGTGGCGCACATC
TGTAGTCCCAGCTACTCAGGAGGCGGAGGCAGGAGAATTGCTTGAACCTGGGAGGTGGAGGTTGCAGTGA
GCTGAGATCGCGCCACTGCATTCCAGCCAGTCTGGCGAAAGAGCAAGACTCTGTCTCAAAAAAAAAAAAA
AAAAAAAAAAAAGACTAAAGTACATGGTTTCTTCAAAGCTTCTCTCTCTTTCTCCCACCTTAGATGATTT
TTCCTTTGCAATGTCCTGTGTCCATTCCGCCCCACTCCTCCTGGGGCCACCTGGACCAGGTCTTCATCAT
CTCATATCTATATGTTTGCTGTGTCTCCTGGCTGGCCACTCTTCTGTAATTTCTCCTCCTCTGAGCTCTC
TGGGCAGCTGAATCTTCTCACTAGTGAAGTCGCCTGGTTGGATGCTGATGAGACTGACCAGCTGAATCCA
GTTGAAAACTTCACACTTGGCAGTGATCTGGTTCTAAAGACACAATTTTCCATAGTTTCCTAACACCATC
CTGCATGCCACCTGCCTTATTTCCCCACATCACATCGTCCCACTTAGCGGGACTGCACTGCTGATCCAAA
TTTTACATCCTTTAGGGCCCACTCAGGTCATATGTCCTCAGGGAAGTCTTTCTGGAAGAACCTTAAACCA
GAGGTTCTCAACAGGGGGCAGTTTTGCTCCCTGTGGAACGTTTGCCAATGTCTGGACACATTTCATTCGT
CACAAACGGAGAGGGGGATGCTACAGGGATCTGGCGGATAGAGGCCAGGGATGCTGCTGAACATCTGCAA
TGCATAGGACAGCCCACCCCCACCCCCACACCCCCAGTAAATAATGATCCAGCCCAAGTGTCACTGGTGC
TGACGTTGAGTAACCCTATCTTAAGCTGAACTCATCATCTCTCCATTCCAGCCTTGGTGGATTCTGTCTC
CTCTGAACCATTCCCATCTCACTTTAGCCTACCTAGATCACAAAGCTTGGCACTCATTATAGACTCCCCT
ATTTATTACTCCTTCAAGATGTGCAAGAATCTTTTCTCTGCACTTTTAAGTTCTGTAAGAAGAGTCTGTG
TCGTTCCTATAATAACCAGCATAGGACGTTGCACGTGTTGTGTGCTCAGTGAACCTGGATTTGTTGATTG
TTGACTGACTCACTCTAGAGTTGGAAATCTTATGCTTGGGGAAACTTAATATCTCTTTCTTTCTCTGTGT
GTGTGCATTTGTGCACGTGTCTGTGCATAGCTGTGAGACCAAAGACCTGGTTGTGCAACAGGCAGGCACA
AACAAGACTGATGTTGTCTGTGGTGAGTCCTGGACAATGGGCCCTGGAGAAAGCCTAGGAAGGTGGGAAC
TGAAGGGGGAGATGAGGCACACAGGAACACTGGATGGGAAAAAGGGGAGGGGAGGCAGTTTGGGGGTGTG
GTATCACAGCTCTGCCACTTATCTTGGGAGTCTGGGCAAATCACTTCCCCTCTCTTAGCCTCAGTTTCTT
CATCTGTAAAATGGGATGATAACAGCACTTCCTTAGTAGGTTTTGATTTTAGAGTGAGAAGGTTGGCCTA
CAGTAAAGATCAGATAATGTAAATCAGTGAAAAAGGTCAGGGGTAAGAAAATTACATTCTCTTTACCTAA
CGCTAAATGACCAGTTAATGGGTGCAGCACACCAACATGGTACATGTATACATATGTAACAAACCTGCAC
ATTATGCACATGTACCCTAAAGCTTAAAGTATAATAATAATAAAATTTAAAAAAACGAAAAATACATTCT
CTTTGCTTTTTCTCAAAATGTACTTTCCTCTTTGTAGGGCTGGGACTAGAATGAGGTGAGCAAGGCACTT
GCCCTCGGGCGCAATATTTAAGAAGGTGCCATAAAAGTGTAGTAATCAAGGTAAATTCATTTTGATGCAA
TATTTTTAAAAATAAAAATTAATGCAAAGAAATCCATGATGAGCAAGATAGCAACATTTTAAATAAAGAA
CAGGATCCGACCCTGTGTTTGCATGACCCTGCCTCACTCACCTCACCCTAATCCTGGCCCTGGTTCCAGT
AAAAGGAATAGGCAGCCAGCCTGCAGGCCGTAGTTTGCTGACTTGGTGTCCGCCTGATGATTTTCAAAAT
ATGGCATTAAAAGAATGTTTACCTTGATGACTGAGTGTTTTGGACATCCTTTTCAATTTTGTCCTGAAAC
AATTTCATCCCTTGCCTCACGCTAGTCTCCGCCCTGCCTTTTGGTCTTTCTTTTATTTTCCCACTTTGAA
AAAAAAATTCGGCATGAGAAATACTTTACCTTTCCCCTCCACTCTTCTATACCAAAAGCAACATGCAGAC
ATGAATCATGCTAGACCTCGGCATTGGGCAGAGAGCAGGGAGTGGCGGGGAGCATGGTGAGCAGGTGGTG
ACAGCCACTGCCACCACTCGCTTCTAGATGGTTCCCAGGTGGGGAGGCTGCCAACTGGAACCCAGTCTTC
CCAGTTTGTAAGAGAAATCAGATGTCTAGGTTTGAATATGTGATCTCCCAGTTTAAAAATGTCGGCAAAT
ATTTCCAAACGTTAAGAAAATGTTCTGGCTCCTTTAAAGACATCTGCCAGCCACATTTCCCCAAGGACCG
CGGTTTGAACCTTCTGATGTAGATGAGCTCTGACATTGGAAGATTCTGGAGTCTGACAAGTCACAGCAGG
TTGAGGGTAGGGAGAAACTGCAGGTGAGGGGTGCATGCTGAAGTCCTGATTTCTCCAGGTCCCCAGGATC
GGCTGAGAGCCCTGGTGGTGATCCCCATCATCTTCGGGATCCTGTTTGCCATCCTCTTGGTGCTGGTCTT
TATCAGTGAGTCCTCAGGTGGGGAGGTGTTGGGGGAGGGAGGGGAGACCACCTGTTTCTTATCTGGCCTC
TCCAACTCCCCATCCTTTTTTTTTTTTTTTTTTTTTTTAGAAAAGGTGGCCAAGAAGCCAACCAATAAGG
TAGGTCACCCCTGAGAACCCGGGACAGAGTTTTGACAAACTGGGAAGATGGCCTCACGGTTGCCTATGGG
GCAGTAAAACTGATTCAGAGTCTGTCTCTGCAGCCAGTGGGGTGGCAGCAGAATTGGGGACTGTCATCCC
CACCCACCATGCTCCTTCCATCCAGAGCTCAATCCCCCACAGAACTGCCCCTGGCACCACTGGCAGAGCC
TAACACTGGCTGTTCTTCACTCCTTTCCTGGCATTCAACGCGTGGGGAGCTGCATCTTTGGGCCTTGGGG
CTGGGTCAAATGGGTGGGAGCAAATGTGGCAGCCCCTTAAGCCCACTGGCTCCCACTCTGGAAGCTCTTC
GTCGCCCTTGGTGTGGCCAGCAGGGGGCAGGAGGCACCCGAGGAATCAGCACTGACCCGCCGTCTGGGAA
AGGGGGGAGGGCTTGGGGAAGGGATCCGCTTCCCAGGGAGGGGCTCCTCAGAGGCACAGCTGCCCCTGCT
GCTGGGGGTGACCTCACACCTTGCCTCTCCAGGCCCCCCACCCCAAGCAGGAACCCCAGGAGATCAATTT
TCCCGACGATCTTCCTGGCTCCAACACTGCTGCTCCAGTGCAGGAGACTTTACATGGATGCCAACCGGTC
ACCCAGGAGGATGGCAAAGAGAGTCGCATCTCAGTGCAGGAGAGACAGTGAGGCTGCACCCACCCAGGAG
TGTGGCCACGTGGGCAAACAGGCAGTTGGCCAGAGAGCCTGGTGCTGCTGCTGCTGTGGCGTGAGGGTGA
GGGGCTGGCACTGACTGGGCATAGCTCCCCGCTTCTGCCTGCACCCCTGCAGTTTGAGACAGGAGACCTG
GCACTGGATGCAGAAACAGTTCACCTTGAAGAACCTCTCACTTCACCCTGGAGCCCATCCAGTCTCCCAA
CTTGTATTAAAGACAGAGGCAGAAGTTTGGTGGTGGTGGTGTTGGGGTATGGTTTAGTAATATCCACCAG
ACCTTCCGATCCAGCAGTTTGGTGCCCAGAGAGGCATCATGGTGGCTTCCCTGCGCCCAGGAAGCCATAT
ACACAGATGCCCATTGCAGCATTGTTTGTGATAGTGAACAACTGGAAGCTGCTTAACTGTCCATCAGCAG
GAGACTGGCTAAATAAAATTAGAATATATTTATACAACAGAATCTCAAAAACACTGTTGAGTAAGGAAAA
AAAGGCATGCTGCTGAATGATGGGTATGGAACTTTTTAAAAAAGTACATGCTTTTATGTATGTATATTGC
CTATGGATATATGTATAAATACAATATGCATCATATATTGATATAACAAGGGTTCTGGAAGGGTACACAG
AAAACCCACAGCTCGAAGAGTGGTGACGTCTGGGGTGGGGAAGAAGGGTCTGGGGGGGGTTGGTTAAAG
GGAGATTTGGCTTTCCCATAATGCTTCATCATTTTTCCCAAAAGGAGAGTGAATTCACATAATGCTTATG
TAATTAAAAAATCATCAAACATGTAAAAAGAAAAACGGGGGTGAACATGCTGGGTGACATGAGCTATTTA
ACCTGCTGTCAGGCTCACGGAATGAGGGCATTTTCTGTAGATAAATAAGAATGTCCCCAGGCTGCTGCCC
CTCCAGGGTGGTTTCCATGTGTGCTCACATGTGGTATTGAGATTGCAAAGTGCTCTTCCCATTTGATTCA
TGTTCACAAAAATAGCCTTCCCCAGCAGGGTGGGTCTGCATCCCTCCCTCTTTTACAGAGGTGGAAATGA
GGTC

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The terms “complementarity determining region (CDR)” and “CDR,” are synonymous with “hypervariable region” or “HVR,” are known in the art to refer to sequences of amino acids within immunoglobulin (e.g., TCR) variable regions. CDRs confer antigen specificity and binding affinity and are separated from one another in primary amino acid sequence by framework regions. In general, there are three CDRs in each TCR α-chain variable region (αCDR1, αCDR2, αCDR3) and three CDRs in each TCR β-chain variable region (βCDR1, βCDR2, βCDR3). In TCRs, CDR3 is thought to be the main CDR responsible for recognizing processed antigen. In general, CDR1 and CDR2 interact mainly or exclusively with the MHC.

CDR1 and CDR2 are encoded within the variable gene segment of a TCR variable region-coding sequence, whereas CDR3 is encoded by the region spanning the variable and joining segments for Vα, or the region spanning variable, diversity, and joining segments for Vβ. Thus, if the identity of the variable gene segment of a Vα or Vβ is known, the sequences of their corresponding CDR1 and CDR2 can be deduced; e.g., according to a numbering scheme as described herein. Compared with CDR1 and CDR2, CDR3, and in particular CDR30, is typically significantly more diverse due to the addition and loss of nucleotides during the recombination process.

TCR variable domain sequences can be aligned to a numbering scheme (e.g., Kabat, Chothia, EU, IMGT, Enhanced Chothia, and Aho), allowing equivalent residue positions to be annotated and for different molecules to be compared using, for example, ANARCI software tool (2016, Bioinformatics 15:298-300). Kabat numbering is described, for example, by Wu and Kabat, J Exp Med. (1970) 132:211-50. 10.1084. A numbering scheme provides a standardized delineation of framework regions and CDRs in the TCR variable domains. In certain embodiments, a CDR of the present disclosure is identified according to the IMGT numbering scheme (Lefranc et al., Dev. Comp. Immunol. 27:55, 2003; imgt.org/IMGTindex/V-QUEST.php). In some embodiments, a CDR (e.g., CDR3) is identified or defined in accordance with the IMGT junction definition. In some embodiments, a CDR (e.g., CDR3) is identified or defined in accordance with the IMGT definition. In some embodiments, a CDR of the present disclosure is identified or defined according to the Kabat numbering scheme or method. In some embodiments, a CDR of the present disclosure is identified or defined according to the Chothia numbering scheme or method. In some embodiments, a CDR of the present disclosure is identified or defined according to the EU numbering scheme or method. In some embodiments, a CDR of the present disclosure is identified or defined according to the enhanced Chothia numbering scheme or method. In some embodiments, a CDR or defined of the present disclosure is identified according to the Aho numbering scheme or method.

The source of a TCR as used in the present disclosure may be from any of a variety of animal species, such as a human, mouse, rat, rabbit, or other mammal. TCR constant domain sequences may be from, for example, human, mouse, marsupial (e.g., opossum, bandicoot, wallaby), shark, or non-human primate. In certain embodiments, TCR constant domain sequences are human or comprise engineered variants of human sequences. TCR constant domains may be engineered to improve pairing, expression, stability, or any combination of these. See, e.g., Cohen et al., Cancer Res, 2007; Kuball et al., Blood 2007; and Haga-Freidman et al., Journal of Immunology 2009. Examples of engineering in TCR Cα and Cβ include mutation of a native amino acid to a cysteine so that a disulfide bond forms between the introduced cysteine of one TCR constant domain and a native cysteine of the other TCR constant domain. Such mutations can include T48C in Cα, T57C in Cβ, or both. Mutations to improve stability can include a mutation in the Cα transmembrane domain from the sequence LSVIGF (SEQ ID NO: 145) to the sequence LLVIVL (SEQ ID NO: 146) (“L-V-L” mutation; see Haga-Friedman et al., J Immunol 188:5538-5546 (2012), the TCR mutations and mutant TCR constant domain sequences of which are incorporated herein by reference).

As used herein, the term “Cluster of Differentiation 8 (CD8) co-receptor” or “CD8” means a transmembrane glycoprotein that serves as a co-receptor for the T-cell receptor. In some embodiments, CD8, functions either as an alpha-alpha homodimer or an alpha-beta heterodimer. The CD8 co-receptor assists in the function of cytotoxic T cells (CD8+) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). There are five (5) human CD8 beta chain isoforms (see UniProtKB identifier P10966) and a single human CD8 alpha chain isoform (see UniProtKB identifier P01732).

“Cluster of Differentiation 4 (CD4)” is a glycoprotein that assists the TCR in communicating with antigen-presenting cells (see, Campbell & Reece, Biology 909 (Benjamin Cummings, Sixth Ed., 2002)). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHCII molecule (CD4 binds MHCII β2, while the TCR complex binds MHCII α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce or recruit various types of immune system cells, including T helper cells, and immune responses.

In certain embodiments, a TCR is found on the surface of T cells (or T lymphocytes) and associates with a CD3 complex. “CD3” is a multi-protein complex of six chains (see, Abbas and Lichtman, 2003; Janeway et al., p. 172 and 178, 1999) that is associated with antigen signaling in T cells. In mammals, the complex comprises a CD3γ chain, a CD3δ chain, two CD3ε chains, and a homodimer of CD3ζ chains. The CD3γ, CD3β, and CD3ε chains are related cell surface proteins of the immunoglobulin superfamily containing a single immunoglobulin domain. The transmembrane regions of the CD3γ, CD3β, and CD3ε chains are negatively charged, which is believed to allow these chains to associate with positively charged regions of T cell receptor chains. The intracellular tails of the CD3γ, CD3β, and CD3ε chains each contain a single conserved motif known as an immunoreceptor tyrosine-based activation motif or ITAM, whereas each CD3ζ chain has three. Without wishing to be bound by theory, it is believed that the ITAMs are important for the signaling capacity of a TCR complex. CD3 as used in the present disclosure may be from various animal species, including human, mouse, rat, or other mammals.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Chimeric antigen receptor” (CAR) refers to a fusion protein that is engineered to contain two or more naturally occurring amino acid sequences, domains, or motifs, linked together in a way that does not occur naturally or does not occur naturally in a host cell, which fusion protein can function as a receptor when present on a surface of a cell. CARs can include an extracellular portion comprising an antigen-binding domain (e.g., obtained or derived from an immunoglobulin or immunoglobulin-like molecule, such as a TCR binding domain derived or obtained from a TCR specific for a cancer antigen, a scFv derived or obtained from an antibody, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016), Stone et al., Cancer Immunol. Immunother., 63(11):1163 (2014), and Walseng et al., Scientific Reports 7:10713 (2017), which CAR constructs and methods of making the same are incorporated by reference herein). CARs of the present disclosure that specifically bind to a Ras antigen (e.g., in the context of a peptide:HLA complex) comprise a TCR Vα domain and a Vβ domain.

The term “chimeric antigen receptor” or alternatively a “CAR” may be used herein to refer to a recombinant polypeptide construct comprising at least an extracellular antigen binding domain (e.g., any of the antigen-binding domains described herein or disclosed in the art), a transmembrane domain (e.g., any of the transmembrane domains described herein or disclosed in the art), and a cytoplasmic signaling domain (also referred to herein as “an intracellular or intrinsic signaling domain”; e.g., any of the costimulatory domains described herein or disclosed in the art) comprising a functional signaling domain derived from a stimulatory molecule. Non-limiting aspects of chimeric antigen receptors are described in, e.g., Kershaw et al., Nature Reviews Immunol. 5(12):928-940, 2005; Eshhar et al., Proc. Natl. Acad. Sci. U.S.A. 90(2):720-724, 1993; Sadelain et al., Curr. Opin. Immunol. 21(2): 215-223, 2009; WO 2015/142675; WO 2015/150526; and WO 2014/134165, the disclosures of each of which are incorporated herein by reference in their entirety. In some embodiments, a CAR comprises an antigen-specific TCR binding domain (see, e.g., Walseng et al., Scientific Reports 7:10713, 2017; the TCR CAR constructs and methods of which are hereby incorporated by reference in their entirety).

By “constitutively activated IL-7 receptor” is meant a polypeptide having at least about 85% amino acid sequence identity to an Interleukin-7 receptor (IL7R), an IL7R subunit, or a fragment thereof, that mediates downstream IL7R signaling in the absence of ligand binding. For example, IL-7R signaling is carried out via Janus kinase 1 (JAK1), JAK3, and/or phosphoinositide 3-kinase (PI3K). IL-7R is a heterodimeric complex consisting of the α-chain (CD127) and the common cytokine receptor γ-chain. Constitutively active IL-7 receptors have been reported to transmit IL-7 signaling without the need for ligand or the common receptor gamma chain (γc), as a result of IL-7Rα homodimerization due to cysteine and/or proline insertions in the transmembrane domain. Constitutively active IL-7 receptors are known in the art and described herein. See, for example, Zhang et al. “The genetic basis of early T-cell precursor acute lymphoblastic leukaemia.” Nature 2012; 481:157-63; Shochat et al., “Gain-of-function mutations in interleukin-7 receptor-α (IL7R) in childhood acute lymphoblastic leukemias.” J Exp Med. 2011; 208:901-8; Shochat et al. “Novel activating mutations lacking cysteine in type I cytokine receptors in acute lymphoblastic leukemia,” Blood. 2014; 124:106-10; and Shum et al., “Constitutive signaling from an engineered IL-7 receptor promotes durable tumor elimination by tumor redirected T-cells,” Cancer Discov. 2017 November; 7(11): 1238-1247, each of which is incorporated herein by reference in its entirety. In embodiments, a constitutively active IL-7 receptor signal leads to the activation and phosphorylation of signal transducer and activator of transcription 5 (STAT5). In an embodiment, the IL-7R subunit is the alpha chain of IL7R. In some embodiments, a constitutively active IL-7 receptor comprises an IL7R alpha (IL7RA) transmembrane domain having a Cysteine, Proline, Threonine (CPT) insert and an intracellular IL7RA domain.

By “decreases” is meant a reduction by at least about 5% relative to a reference level. A decrease may be by 5%, 10%, 15%, 20%, 25% or 50%, or even by as much as 75%, 85%, 95% or more and any intervening percentages.

“Detect” refers to identifying the presence, absence or amount of the analyte to be detected.

By “disease” is meant any condition or disorder that damages or interferes with the normal function of a cell, tissue, or organ. Examples of diseases include a cancer or an infection, such as a bacterial, viral, or fungal infection. Examples of cancers include solid tumors, ascites tumors, blood or lymph or other hematological malignancies; connective tissue malignancies; metastatic disease; minimal residual disease following transplantation of organs or stem cells; multi-drug resistant cancers, primary or secondary malignancies, angiogenesis related to malignancy, or other forms of cancer. A cancer may also include a carcinoma, a sarcoma, a glioma, a lymphoma, a leukemia, a myeloma, or any combination thereof. In some embodiments, cancer comprises a cancer of the head or neck, melanoma, pancreatic cancer including but not limited to pancreatic ductal adenocarcinoma (PDAC), cholangiocarcinoma, hepatocellular cancer, breast cancer including but not limited to triple-negative breast cancer (TNBC), gastric cancer, lung cancer including but not limited to small-cell lung cancer and non-small-cell lung cancer, prostate cancer, esophageal cancer, mesothelioma, colorectal cancer, glioblastoma, or any combination thereof. In certain embodiments, the cancer comprises a solid tumor. In some embodiments, the solid tumor is a sarcoma or a carcinoma.

The term “effective amount” or “therapeutically effective amount” refers to the quantity of a composition, for example a composition comprising immune cells such as lymphocytes (e.g., T lymphocytes or NK cells) comprising a fusion protein of the present disclosure, that is sufficient to result in a predetermined activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

By “effector immune cell” is meant a cell capable of creating an immune response within a subject. Effector immune cells can be categorized as belonging to either the “innate” or “adaptive” immune compartments. The innate immune compartment is constituted by effector cells (and their molecular products) that lack antigen specificity (such as neutrophils, monocytes, macrophages, complement, and acute phase proteins) and generally provide protection against exposure to acute pathogenic factors. Conversely, cellular effectors of the adaptive immune compartment (such as T and B lymphocytes) demonstrate high antigen specificity and promote the establishment of immunological memory to various pathogens by coordinating the actions of both the adaptive and the innate compartments.

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “FERM” domain is meant a plasma membrane localization signal widely found amongst members of the ezrin, moesin, and radixin (ERM) protein family. Exemplary amino acid sequences of FERM domains are provided below:

(SEQ ID NO: 147)
KKRIKPIVWPSLPDHKKTLEHLCKKPRK
(SEQ ID NO: 148)
NCRNTGPWLKKVLKCNTPDPSK

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, a “fusion protein” refers to a polypeptide that has at least two distinct domains that are not naturally found together in a protein. A nucleic acid molecule encoding a fusion protein may be recombinantly engineered, and such fusion proteins can be made using methods of protein synthesis known in the art. A fusion protein may further comprise other components (e.g., a via covalent bond), such as a tag (e.g., a histidine tag) or a bioactive molecule. In certain embodiments, a fusion protein expressed or produced by a host cell (e.g., T cell) locates to the cell surface, where the fusion protein comprises a transmembrane domain located between an extracellular domain comprising a binding domain and an intracellular domain comprising a signaling domain.

A “host cell” or “cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

As used herein, an “intracellular domain” refers to a fragment of a polypeptide that is located in the cytoplasm of a host cell. In some embodiments, an intracellular domain is capable of transmitting signals via an intracellular signaling domain, for example, or by interacting with a signaling molecule or with another intracellular domain.

As used herein, an “intracellular signaling domain” refers to an intracellular portion of a molecule capable of propagating a signal. In some embodiments, the intracellular signaling domain may directly or indirectly propagate a response, such as a co-stimulatory, positive, activating biological or physiological response in a cell when receiving the appropriate signal, or promote downstream immune cell signaling (e.g., T cell receptor signaling) or immune cell activation (e.g., T cell activation). Non-limiting examples of intracellular signaling domains are described herein. Additional examples of intracellular signaling domains are known in the art.

See, e.g., Chen et al., Nature Reviews Immunol. 13:227-242, 2013 By “Interleukin 2” (IL-2) is meant a polypeptide, or fragment thereof, having at least about 85% amino acid identity to GENBANK Accession No. AAH70338.1 that binds an IL2 receptor. An exemplary polypeptide sequence is provided below:

>AAH70338.1 Interleukin 2 [Homo sapiens]
(SEQ ID NO: 149)
MYRMQLLSCIALSLALVTNSAPTSSSTKKTQLQLEHLLLDLQMIL
NGINNYKNPKLTRMLTFKFYMPKKATELKHLQCLEEELKPLEEVL
NLAQSKNFHLRPRDLISNINVIVLELKGSETTFMCEYADETATIV
EFLNRWITFCQSIISTLT

By “Interleukin-2 receptor Subunit Alpha” (IL2RA) is meant a polypeptide, or fragment thereof, having at least about 85% amino acid identity to GENBANK Accession No. AAS55572.1 and having IL-2 receptor biological activity. An exemplary polypeptide sequence is provided below:

>AAS55572.1 interleukin 2 receptor,
alpha [Homo sapiens]
(SEQ ID NO: 150)
MDSYLLMWGLLTFIMVPGCQAELCDDDPPEIPHATFKAMAYKEGT
MLNCECKRGFRRIKSGSLYMLCTGNSSHSSWDNQCQCTSSATRNT
TKQVTPQPEEQKERKTTEMQSPMQPVDQASLPGHCREPPPWENEA
TERIYHFVVGQMVYYQCVQGYRALHRGPAESVCKMTHGKTRWTQP
QLICTGEMETSQFPGEEKPQASPEGRPESETSCLVTTTDFQIQTE
MAATMETSIFTTEYQVAVAGCVFLLISVLLLSGLTWQRRQRKSRR
TI

By “Interleukin-2 receptor Subunit Beta” (IL2RB) is meant a polypeptide or fragment thereof having at least about 85% amino acid identity to GENBANK Accession No. AAM54040.1. An exemplary polypeptide sequence is provided below:

>AAM54040.1 interleukin 2 receptor,
beta [Homo sapiens]
(SEQ ID NO: 151)
MAAPALSWRLPLLILLLPLATSWASAAVNGTSQFTCFYNSRANIS
CVWSQDGALQDTSCQVHAWPDRRRWNQTCELLPVSQASWACNLIL
GAPDSQKLTTVDIVTLRVLCREGVRWRVMAIQDFKPFENLRLMAP
ISLQVVHVETHRCNISWEISQASHYFERHLEFEARTLSPGHTWEE
APLLTLKQKQEWICLETLTPDTQYEFQVRVKPLQGEFTTWSPWSQ
PLAFRTKPAALGKDTIPWLGHLLVGLSGAFGFIILVYLLINCRNT
GPWLKKVLKCNTPDPSKFFSQLSSEHGGDVQKWLSSPFPSSSFSP
GGLAPEISPLEVLERDKVTQLLLQQDKVPEPASLSSNHSLTSCFT
NQGYFFFHLPDALEIEACQVYFTYDPYSEEDPDEGVAGAPTGSSP
QPLQPLSGEDDAYCTFPSRDDLLLFSPSLLGGPSPPSTAPGGSGA
GEERMPPSLQERVPRDWDPQPLGPPTPGVPDLVDFQPPPELVLRE
AGEEVPDAGPREGVSFPWSRPPGQGEFRALNARLPLNTDAYLSLQ
ELQGQDPTHLV

By “Interleukin-2 receptor Subunit Gamma” (IL2Rβ) is meant a polypeptide or fragment thereof having at least about 85% amino acid identity to GENBANK Accession No. AAC37524.1. An exemplary polypeptide sequence is provided below:

>AAC37524.1 interleukin-2 receptor gamma
subunit [Homo sapiens]
(SEQ ID NO: 152)
MLKPSLPFTSLLFLQLPLLGVGLNTTILTPNGNEDTTADFFLTTM
PTDSLSVSTLPLPEVQCFVFNVEYMNCTWNSSSEPQPTNLTLHYW
YKNSDNDKVQKCSHYLFSEEITSGCQLQKKEIHLYQTFVVQLQDP
REPRRQATQMLKLQNLVIPWAPENLTLHKLSESQLELNWNNRFLN
HCLEHLVQYRTDWDHSWTEQSVDYRHKFSLPSVDGQKRYTFRVRS
RFNPLCGSAQHWSEWSHPIHWGSNTSKENPFLFALEAVVISVGSM
GLIISLLCVYFWLERTMPRIPTLKNLEDLVTEYHGNFSAWSGVSK
GLAESLQPDYSERLCLVSEIPPKGGALGEGPGASPCNQHSPYWAP
PCYTLKPET

IL2R a heterotrimeric protein expressed on the surface of certain immune cells, such as lymphocytes, that binds and responds to a cytokine called Interleukin-2 (IL-2). IL-2 is a 15.5-16 kDa protein that regulates the activities of white blood cells (leukocytes, or lymphocytes) that are responsible for immunity. IL-2 is part of the body's natural response to microbial infection, and in discriminating between foreign (“non-self”) and “self”. IL-2 mediates its effects by binding to IL2R, which is expressed by lymphocytes.

IL2R has three forms, generated by different combinations of three different proteins, often referred to as “chains”: α (alpha) (also called IL2RA, IL2Rα, CD25, or Tac antigen), β (beta) (also called IL2RB, IL2Rβ, or CD122), and γ (gamma) (also called IL2RG, IL2Rγ, γc, common gamma chain, or CD132); these subunits are also parts of receptors for other cytokines. The β and γ chains of the IL2R are members of the type I cytokine receptor family.

In the present disclosure, it will be understood that Interleukin-2 receptor can also be referred-to as IL2R, as IL2RA, or as IL-2R.

By “Interleukin 7” (IL7) is meant a polypeptide or fragment thereof having at least about 85% amino acid identity to GENBANK Accession No. AAH47698.1 that binds an IL7 receptor. An exemplary polypeptide sequence is provided below:

>AAH47698.1 Interleukin 7 [Homo sapiens]
(SEQ ID NO: 153)
MFHVSFRYIFGLPPLILVLLPVASSDCDIEGKDGKQYESVLMVSI
DQLLDSMKEIGSNCLNNEFNFFKRHICDANKEGMFLFRAARKLRQ
FLKMNSTGDFDLHLLKVSEGTTILLNCTGQVKGRKPAALGEAQPT
KSLEENKSLKEQKKLNDLCFLKRLLQEIKTCWNKILMGTKEH

By “Interleukin-7 receptor polypeptide” (IL7R) is meant a polypeptide comprising at least an Interleukin-7 receptor (IL7R) alpha subunit, or a fragment thereof, that binds IL-7 and/or that mediates IL-7R signaling. In some embodiments, an IL7R is a heterodimer comprising an IL7R alpha subunit and a common gamma chain/IL-2 receptor (IL2R gamma). In some embodiments, an IL7R is a homodimer comprising two constitutively active IL7R alpha subunits, or fragments thereof

By “Interleukin-7 receptor alpha subunit” (IL7RA) is meant a polypeptide, or fragment thereof, having at least about 85% amino acid identity to GENBANK Accession No. AAH69999.1 and having IL7R signaling activity. An exemplary polypeptide sequence is provided below:

>AAH69999.1 Interleukin 7 receptor [Homo sapiens]
(SEQ ID NO: 154)
MTILGTTFGMVFSLLQVVSGESGYAQNGDLEDAELDDYSFSCYSQ
LEVNGSQHSLTCAFEDPDVNITNLEFEICGALVEVKCLNFRKLQE
IYFIETKKFLLIGKSNICVKVGEKSLTCKKIDLTTIVKPEAPFDL
SVIYREGANDFVVTFNTSHLQKKYVKVLMHDVAYRQEKDENKWTH
VNLSSTKLTLLQRKLQPAAMYEIKVRSIPDHYFKGFWSEWSPSYY
FRTPEINNSSGEMDPILLTISILSFFSVALLVILACVLWKKRIKP
IVWPSLPDHKKTLEHLCKKPRKNLNVSFNPESFLDCQIHRVDDIQ
ARDEVEGFLQDTFPQQLEESEKQRLGGDVQSPNCPSEDVVITPES
FGRDSSLTCLAGNVSACDAPILSSSRSLDCRESGKNGPHVYQDLL
LSLGTTNSTLPPPFSLQSGILTLNPVAQGQPILTSLGSNQEEAYV
TMSSFYQNQ

In the present disclosure, it will be understood that Interleukin-7 receptor-α can also be referred-to as IL7RA, IL7R-α, IL-7R-alpha, ILRA, Interleukin-7 receptor subunit alpha, interleukin 7 receptor, Cluster of Differentiation 127, CD127, or CDW127. In embodiments, IL7RA is a constitutively active IL7RA.

By “IL7 receptor signaling activity” is meant one or more of activation of JAK1, JAK3, PI3K, phosphorylation of STAT5, or any other biological activity initiated in response to IL-7 bind to the IL7 receptor.

By “IL7 receptor polynucleotide” is meant a nucleotide sequence encoding an IL7 receptor or subunit or fragment thereof. In an embodiment, an IL7 receptor polynucleotide encodes an IL7R alpha subunit. In one embodiment, an IL7 receptor polynucleotide is provided GenBank: BC069999.1

| (SEQ ID NO: 155) | |
|---|---|
| 1 | cctccctccc ttcctcttac tctcattcat ttcatacaca ctggctcaca catctactct |
| 61 | ctctctctat ctctctcaga atgacaattc taggtacaac ttttggcatg gttttttctt |
| 121 | tacttcaagt cgtttctgga gaaagtggct atgctcaaaa tggagacttg gaagatgcag |
| 181 | aactggatga ctactcattc tcatgctata gccagttgga agtgaatgga tcgcagcact |
| 241 | cactgacctg tgcttttgag gacccagatg tcaacatcac caatctggaa tttgaaatat |
| 301 | gtggggccct cgtggaggta aagtgcctga atttcaggaa actacaagag atatatttca |
| 361 | tcgagacaaa gaaattctta ctgattggaa agagcaatat atgtgtgaag gttggagaaa |
| 421 | agagtctaac ctgcaaaaaa atagacctaa ccactatagt taaacctgag gctccttttg |
| 481 | acctgagtgt catctatcgg gaaggagcca atgactttgt ggtgacattt aatacatcac |
| 541 | acttgcaaaa gaagtatgta aaagttttaa tgcacgatgt agcttaccgc caggaaaagg |
| 601 | atgaaaacaa atggacgcat gtgaatttat ccagcacaaa actgacactc ctgcagagaa |
| 661 | agctccaacc ggcagcaatg tatgagatta aagttcgatc catccctgat cactatttta |
| 721 | aaggcttctg gagtgaatgg agtccaagtt attacttcag aactccagag atcaataata |
| 781 | gctcagggga gatggatcct atcttactaa ccatcagcat tttgagtttt ttctctgtcg |
| 841 | ctctgttggt catcttggcc tgtgtgttat ggaaaaaaag gattaagcct atcgtatggc |
| 901 | ccagtctccc cgatcataag aagactctgg aacatctttg taagaaacca agaaaaaatt |
| 961 | taaatgtgag tttcaatcct gaaagtttcc tggactgcca gattcatagg gtggatgaca |
| 1021 | ttcaagctag agatgaagtg gaaggttttc tgcaagatac gtttcctcag caactagaag |
| 1081 | aatctgagaa gcagaggctt ggaggggatg tgcagagccc caactgccca tctgaggatg |
| 1141 | tagtcatcac tccagaaagc tttggaagag attcatccct cacatgcctg gctgggaatg |
| 1201 | tcagtgcatg tgacgcccct attctctect cttccaggtc cctagactgc agggagagtg |
| 1261 | gcaagaatgg gcctcatgtg taccaggacc tcctgcttag ccttgggact acaaacagca |
| 1321 | cgctgccccc tccattttct ctccaatctg gaatcctgac attgaaccca gttgctcagg |
| 1381 | gtcagcccat tcttacttcc ctgggatcaa atcaagaaga agcatatgtc accatgtcca |
| 1441 | gcttctacca aaaccagtga agtgtaagaa acccagactg aacttaccgt gagcgacaaa |
| 1501 | gat |

As used herein, an “immune system cell” means any cell involved in the generation of an immune response. In one embodiment, an immune cell originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4+ T cell, a CD8+ T cell, a CD4− CD8− double negative T cell, a γδ T cell, a regulatory T cell, a natural killer cell, a natural killer T cell, and a dendritic cell. Macrophages and dendritic cells can be referred to as “antigen presenting cells” or “APCs,” which are specialized cells that can activate T cells when a major histocompatibility complex (MHC) receptor on the surface of the APC complexed with a peptide interacts with a TCR on the surface of a T cell. In some embodiments, the immune cell expresses a transgenic or exogenous T cell receptor (“TCR-T cell”) or a chimeric antigen receptor (“CAR-T cell”).

By “immunomodulatory” or “modulating an immune response” is meant effecting an increase or a decrease in an immune response. In some embodiments, immunomodulatory fusion proteins of the present disclosure are effective to enhance an immune response in a subject in response to a disease (e.g., cancer).

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “juxtamembrane” is meant adjacent to a membrane. In one embodiment, a juxtamembrane domain comprises a fragment (e.g., 5, 10, 15, 20, 30, 40 amino acids) of a transmembrane polypeptide domain that is proximal to the plasma membrane when expressed by a cell.

As used herein, “TCR complex” refers to a complex formed by the association of CD3 with TCR. For example, a TCR complex can be composed of a CD3γ chain, a CD3β chain, two CD3ε chains, a homodimer of CD3ζ chains, a TCRα chain, and a TCRβ chain. Alternatively, a TCR complex can be composed of a CD3γ chain, a CD3β chain, two CD3ε chains, a homodimer of CD3ζ chains, a TCRγ chain, and a TCRβ chain.

A “component of a TCR complex”, as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers. In some embodiments, a “peptide” (e.g., a peptide antigen) refers to a polymer of about 8-15 amino acid residues in length.

Any polypeptide of this disclosure can, as encoded by a polynucleotide sequence, comprise a “signal peptide” (also known as a leader sequence, leader peptide, or transit peptide). Signal peptides target newly synthesized polypeptides to their appropriate location inside or outside the cell. In some contexts, signal peptides are from about 15 to about 22 amino acids in length. A signal peptide may be removed from the polypeptide during, or once localization (e.g., membrane insertion) or secretion is completed. Polypeptides that have a signal peptide are referred to herein as a “pre-protein” and polypeptides having their signal peptide removed are referred to herein as “mature” proteins or polypeptides. In any of the herein disclosed embodiments, a binding protein or fusion protein comprises, or is, a mature protein, or is or comprises a pre-protein.

A “linker” refers to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In certain embodiments, a linker is comprised of about two to about 35 amino acids, for instance, or about four to about 20 amino acids or about eight to about 15 amino acids or about 15 to about 25 amino acids. Example linkers include glycine-serine linkers.

By “marker” is meant any protein or polynucleotide having an alteration in expression level or activity that is associated with a disease or disorder.

A “neoantigen,” as used herein, refers to a host cellular product containing a structural change, alteration, or mutation that creates a new antigen or antigenic epitope that has not previously been observed in the subject's genome (i.e., in a sample of healthy tissue from the subject) or been “seen” or recognized by the host's immune system, which: (a) is processed by the cell's antigen-processing and transport mechanisms and presented on the cell surface in association with an MHC (e.g., HLA) molecule; and (b) elicits an immune response (e.g., a cellular (T cell) response). Neoantigens may originate, for example, from coding polynucleotides having alterations (substitution, addition, deletion) that result in an altered or mutated product, or from the insertion of an exogenous nucleic acid molecule or protein into a cell, or from exposure to environmental factors (e.g., chemical, radiological) resulting in a genetic change. Neoantigens may arise separately from a tumor antigen or may arise from or be associated with a tumor antigen. “Tumor neoantigen” (or “tumor specific neoantigen”) refers to a protein comprising a neoantigenic determinant associated with, arising from, or arising within a tumor cell or plurality of cells within a tumor. Tumor neoantigenic determinants are found on, for example, antigenic tumor proteins or peptides that contain one or more somatic mutations or chromosomal rearrangements encoded by the DNA of tumor cells (e.g., pancreas cancer, lung cancer, colorectal cancers), as well as proteins or peptides from viral open reading frames associated with virus-associated tumors (e.g., cervical cancers, some head and neck cancers). The terms “antigen” and “neoantigen” are used interchangeably herein when referring to a Ras antigen comprising a mutation as disclosed herein.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three-dimensional structural characteristics, as well as specific charge characteristics. By “ameliorate” is meant decrease, suppress, attenuate, diminish, arrest, or stabilize the development or progression of a disease.

“Major histocompatibility complex” (MHC) refers to glycoproteins that deliver peptide antigens to a cell surface of all nucleated cells. MHC class I molecules are heterodimers having a membrane spanning a chain (with three a domains) and a non-covalently associated β2 microglobulin. MHC class II molecules are composed of two transmembrane glycoproteins, α and β, both of which span the membrane. Each chain comprises two domains. MHC class I molecules deliver peptides originating in the cytosol to the cell surface, where a peptide:MHC complex is recognized by CD8+ T cells. MHC class II molecules deliver peptides originating in the vesicular system to the cell surface, where they are recognized by CD4+ T cells. Human MHC is referred to as human leukocyte antigen (HLA). HLAs corresponding to “class I” MHC present peptides from inside the cell and include, for example, HLA-A, HLA-B, and HLA-C. Alleles include, for example, HLA A*11, such as HLA-A*11:01. HLAs corresponding to “class II” MHC present peptides from outside the cell and include, for example, HLA-DP, HLA-DM, HLA-DOA, HLA-DOB, HLA-DQ, and HLA-DR.

Principles of antigen processing by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MHC gene that is relevant for antigen presentation) APC and T cells, are well-established (see, e.g., Murphy, Janeway's Immunobiology (8th Ed.) 2011 Garland Science, NY; chapters 6, 9 and 16). For example, processed antigen peptides originating in the cytosol (e.g., tumor antigen, intracellular pathogen) are generally from about 7 amino acids to about 11 amino acids in length and will associate with class I MHC (HLA) molecules, whereas peptides processed in the vesicular system (e.g., bacterial, viral) will vary in length from about 10 amino acids to about 25 amino acids and associate with class II MHC (HLA) molecules.

As used herein, “nucleic acid” or “nucleic acid molecule” or “polynucleotide” refers to any of deoxyribonucleic acid (DNA), ribonucleic acid (RNA), oligonucleotides, polynucleotides, fragments thereof generated, for example, by the polymerase chain reaction (PCR) or by in vitro translation, and also to fragments generated by any of ligation, scission, endonuclease action, or exonuclease action. In certain embodiments, the nucleic acids of the present disclosure are produced by PCR. Nucleic acids can be composed of monomers that are naturally occurring nucleotides (such as deoxyribonucleotides and ribonucleotides), analogs of naturally occurring nucleotides (e.g., α-enantiomeric forms of naturally occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single-stranded or double-stranded.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such a nucleic acid can be part of a vector and/or such nucleic acid or polypeptide can be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

As used herein, the terms “recombinant”, “engineered”, and “modified” refer to a cell, microorganism, nucleic acid molecule, polypeptide, protein, plasmid, or vector that has been modified by introduction of an exogenous nucleic acid molecule, or refers to a cell or microorganism that has been genetically engineered by human intervention—that is, modified by introduction of a heterologous nucleic acid molecule, or refers to a cell or microorganism that has been altered such that expression of an endogenous nucleic acid molecule or gene is controlled, deregulated or constitutive, where such alterations or modifications can be introduced by genetic engineering. Human-generated genetic alterations can include, for example, modifications introducing nucleic acid molecules (which may include an expression control element, such as a promoter) encoding one or more proteins or enzymes, or other nucleic acid molecule additions, deletions, substitutions, or other functional disruption of or addition to a cell's genetic material. Example modifications include those in coding regions or functional fragments thereof of heterologous or homologous polypeptides from a reference or parent molecule.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s). In certain embodiments, a mutation is a substitution of one or three codons or amino acids, a deletion of one to about 5 codons or amino acids, or a combination thereof. A “conservative substitution” is recognized in the art as a substitution of one amino acid for another amino acid that has similar properties. Example conservative substitutions are well known in the art (see, e.g., WO 97/09433 at page 10; Lehninger, Biochemistry, 2nd Edition; Worth Publishers, Inc. NY, NY, pp. 71-77, 1975; Lewin, Genes IV, Oxford University Press, NY and Cell Press, Cambridge, MA, p. 8, 1990).

The term “construct” refers to any polynucleotide that contains a recombinant nucleic acid molecule. A “transgene” or “transgene construct” refers to a construct that contains two or more genes operably linked in an arrangement that is not found in nature. The term “operably-linked” (or “operably linked” herein) refers to the association of two or more nucleic acid molecules on a single nucleic acid fragment so that the function of one is affected by the other. For example, a promoter is operably-linked with a coding sequence when it can affect the expression of that coding sequence (i.e., the coding sequence is under the transcriptional control of the promoter). “Unlinked” means that the associated genetic elements are not closely associated with one another and the function of one does not affect the other. In some embodiments, the genes present in a transgene are operably linked to an expression control sequence (e.g., a promoter).

A construct (e.g., a transgene) can be present in a vector (e.g., a bacterial vector, a viral vector) or can be integrated into a genome. A “vector” is a nucleic acid molecule that is capable of transporting another nucleic acid molecule. Vectors can be, for example, plasmids, cosmids, viruses, a RNA vector or a linear or circular DNA or RNA molecule that can include chromosomal, non-chromosomal, semi-synthetic or synthetic nucleic acid molecules. Example vectors are those capable of autonomous replication (episomal vector) or expression of nucleic acid molecules to which they are linked (expression vectors). Vectors useful in the compositions and methods of this disclosure are described further herein.

The term “expression,” as used herein, refers to the process by which a polypeptide is produced based on the encoding sequence of a nucleic acid molecule, such as a gene. The process can include transcription, post-transcriptional control, post-transcriptional modification, translation, post-translational control, post translational modification, or any combination thereof.

The term “introduced” in the context of inserting a nucleic acid molecule into a cell, means “transfection,” or “transformation,” or “transduction” and includes reference to the incorporation of a nucleic acid molecule into a eukaryotic or prokaryotic cell wherein the nucleic acid molecule can be incorporated into the genome of a cell (e.g., a chromosome, a plasmid, a plastid, or a mitochondrial DNA), converted into an autonomous replicon, or transiently expressed (e.g., transfected mRNA).

As used herein, “heterologous” or “exogenous” nucleic acid molecule, construct, or sequence refers to a nucleic acid molecule or portion of a nucleic acid molecule that is not native to a host cell but can be homologous to a nucleic acid molecule or portion of a nucleic acid molecule from the host cell. The source of the heterologous or exogenous nucleic acid molecule, construct or sequence can be from a different genus or species. In certain embodiments, a heterologous or exogenous nucleic acid molecule is added (i.e., not endogenous or native) to a host cell or host genome by, for example, conjugation, transformation, transfection, transduction, electroporation, or the like, wherein the added molecule can integrate into the host genome or exist as extra-chromosomal genetic material (e.g., as a plasmid or other form of self-replicating vector), and can be present in multiple copies. In addition, “heterologous” refers to a non-native enzyme, protein or other activity encoded by an exogenous nucleic acid molecule introduced into the host cell, even if the host cell encodes a homologous protein or activity. Moreover, a cell comprising a “modification” or a “heterologous” polynucleotide or binding protein includes progeny of that cell, regardless of whether the progeny were themselves transduced, transfected, or otherwise manipulated or changed. By “reduces” is meant a negative alteration of at least 10%, 25%, 50%, 75%, or 100%.

By “operably linked” refers to a functional linkage between a regulatory sequence and a coding sequence, where a first polynucleotide is positioned adjacent to a second polynucleotide that directs transcription of the first polynucleotide when appropriate molecules (e.g., transcriptional activator proteins) are bound to the second polynucleotide. The described components are therefore in a relationship permitting them to function in their intended manner. For example, placing a coding sequence under regulatory control of a promoter means positioning the coding sequence such that the expression of the coding sequence is controlled by the promoter.

By “positioned for expression” is meant that the polynucleotide of the disclosure (e.g., a DNA molecule) is positioned adjacent to a DNA sequence that directs transcription and translation of the sequence (i.e., facilitates the production of, for example, a recombinant microRNA molecule described herein).

The term “promoter” as used herein refers to a sequence of DNA that directs the expression (transcription) of a gene. A promoter may direct the transcription of a prokaryotic or eukaryotic gene. A promoter may be “inducible”, initiating transcription in response to an inducing agent or, in contrast, a promoter may be “constitutive”, whereby an inducing agent does not regulate the rate of transcription. A promoter may be regulated in a tissue-specific or tissue-preferred manner, such that it is only active in transcribing the operable linked coding region in a specific tissue type or types. In some embodiments, any promoter suitable for use in a given host cell may be used for expression of the fusion proteins of the present disclosure. For example, when the host is an animal cell, an SR.alpha. promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used.

By “reduces” is meant a negative alteration of at least 10%, 25%, 50%, 75%, or 100%.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, at least about 100 nucleotides or at least about 300 nucleotides or any integer thereabout or therebetween.

In some contexts, the term “variant” as used herein, refers to at least one fragment of the full-length sequence referred to, more specifically one or more amino acid or nucleic acid sequence which is, relative to the full-length sequence, truncated at one or both termini by one or more amino acids. Such a fragment includes or encodes for a peptide having at least 6, 7, 8, 10, 12, 15, 20, 25, 50, 75, 100, 150, or 200 successive amino acids of the original sequence or a variant thereof. The total length of the variant may be at least 6, 7, 8, 9, 10, 11, 12, 20, 25, 30, 40, 50, 60, 70, 80, 90, 100, or more amino acids.

In some embodiments, the term “variant” relates not only to at least one fragment, but also to a polypeptide or a fragment thereof including amino acid sequences that are at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to the reference amino acid sequence referred to or the fragment thereof, wherein amino acids other than those essential for the biological activity or the fold or structure of the polypeptide are deleted or substituted, one or more such essential amino acids are replaced in a conservative manner, and/or amino acids are added such that the biological activity of the polypeptide is preserved. The state of the art includes various methods that may be used to align two given nucleic acid or amino acid sequences and to calculate the degree of identity (see, e.g., Arthur Lesk (2008), Introduction to bioinformatics, Oxford University Press, 2008, 3rd edition). In some embodiments, the Clustal W software can be used using default settings (Larkin, M. A., et al. (2007). Clustal W and Clustal X version 2.0. Bioinformatics, 23, 2947-2948).

In certain embodiments, variants may, in addition, include chemical modifications, for example, isotopic labels or covalent modifications such as glycosylation, phosphorylation, acetylation, decarboxylation, citrullination, hydroxylation and the like. Methods for modifying polypeptides are known and in general will be employed so as not to abolish or substantially diminish a desired activity of the polypeptide.

In an embodiment, the term “variant” of a nucleic acid molecule includes nucleic acids the complementary strand of which hybridizes, for example, under stringent conditions, to the reference or wild type nucleic acid. Stringency of hybridization reactions is readily determinable by one of ordinary skill in the art, and in general is an empirical calculation dependent on probe length, washing temperature, and salt concentration. In general, longer probes require higher temperatures for proper annealing, while shorter probes less so. Hybridization generally depends on the ability of denatured DNA to reanneal to complementary strands present in an environment below their melting temperature: the higher the degree of desired homology between the probe and hybridizable sequence, the higher the relative temperature which may be used. As a result, higher relative temperatures can make the reaction conditions more stringent, while lower temperatures less so. For additional details and explanation of stringency of hybridization reactions, see Ausubel, F. M. (1995), Current Protocols in Molecular Biology. John Wiley & Sons, Inc. Moreover, the person skilled in the art may follow the instructions given in the manual Boehringer Mannheim GmbH (1993) The DIG System Users Guide for Filter Hybridization, Boehringer Mannheim GmbH, Mannheim, Germany and in Liebl, W., Ehrmann, M., Ludwig, W., and Schleifer, K. H. (1991) International Journal of Systematic Bacteriology 41: 255-260 on how to identify DNA sequences by means of hybridization. In an embodiment, stringent conditions are applied for any hybridization, i.e., hybridization occurs only if the probe is 70% or more identical to the target sequence. Probes having a lower degree of identity with respect to the target sequence may hybridize, but such hybrids are unstable and will be removed in a washing step under stringent conditions, for example, lowering the concentration of salt to 2×SSC or, optionally and subsequently, to 0.5×SSC, while the temperature is, for example, about 50° C.-68° C., about 52° C.-68° C., about 54° C.-68° C., about 56° C.-68° C., about 58° C.-68° C., about 60° C.-68° C., about 62° C.-68° C., about 64° C.-68° C., or about 66° C.-68° C. In an embodiment, the temperature is about 64° C.-68° C. or about 66° C.-68° C. It is possible to adjust the concentration of salt to 0.2×SSC or even 0.1×SSC. Nucleic acid sequences having a degree of identity with respect to the reference or wild type sequence of at least 70%, at least 80%, at least 85%, at least 90%, at least 91%, at least 92%, at least 93%, at least 94%, at least 95%, at least 96%, at least 97%, at least 98%, at least 99%, or at least 99.5% may be isolated. In an embodiment, the term variant of a nucleic acid sequence, as used herein, refers to any nucleic acid sequence that encodes the same amino acid sequence and variants thereof as the reference nucleic acid sequence, in line with the degeneracy of the genetic code.

A “functional variant” refers to a polypeptide or polynucleotide that is structurally similar or substantially structurally similar to a parent or reference compound of this disclosure, but differs, in some contexts slightly, in composition (e.g., one base, atom or functional group is different, added, or removed; or one or more amino acids are mutated, inserted, or deleted), such that the polypeptide or encoded polypeptide is capable of performing at least one function of the encoded parent polypeptide with at least 50% efficiency, or at least 55%, at least 60%, at least 70%, at least 75%, at least 80%, at least 85%, at least 90%, at least 95%, at least 96%, at least 97%, at least 98%, at least 99%, at least 99.9%, or at least 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (KD) constant), avidity, or activation of a host cell. As used herein, a “functional portion” or “functional fragment” refers to a polypeptide or polynucleotide that comprises only a domain, motif, portion or fragment of a parent or reference compound, and the polypeptide or encoded polypeptide retains at least 50% activity associated with the domain, portion or fragment of the parent or reference compound, or at least 55 at least 60%, at least 70%, at least 75%, at least 80%, at least 85%, at least 90%, at least 95%, at least 96%, at least 97%, at least 98%, at least 99%, at least 99.9%, or at least 100% level of activity of the parent polypeptide, or provides a biological benefit (e.g., effector function).

A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (alternatively or additionally, no more than 20% or 10%, or no more than a log difference as compared to the parent or reference with regard to affinity), such as an assay for measuring binding affinity or measuring effector function (e.g., cytokine release). Functional variants of specifically disclosed binding proteins and polynucleotides are contemplated.

An “altered domain” or “altered protein” refers to a motif, region, domain, peptide, polypeptide, or protein with a non-identical sequence identity to a wild type motif, region, domain, peptide, polypeptide, or protein (e.g., a wild type TCRα chain, TCRβ chain, TCRα constant domain, or TCRβ constant domain) of at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9%).

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event.

By “reference” is meant a standard or control condition. In an embodiment, a reference subject is a healthy control subject. In an embodiment, a reference subject is an untreated subject having a disease of the present disclosure. In an embodiment, a reference subject is an unmodified cell, such as an unmodified host cell (e.g., immune cell, such as a T-cell) of the present disclosure. In an embodiment, a reference standard is an immune response in an unmodified immune cell. Immune response is characterized in some embodiments, for example, by levels of STAT5 phosphorylation, or Janus Kinase (JAK) activity, or STAT5 mediated transcription.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 nM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

As used herein, the term “optimally aligned” in the context of two or more nucleic acids or polypeptide sequences, refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “specifically binds” is meant a compound or antibody that recognizes and binds a polypeptide of the invention, but which does not substantially recognize and bind other molecules in a sample, for example, a biological sample, which naturally includes a polypeptide of the invention.

The terms “subject,” “individual,” and “patient” are used interchangeably herein to refer to a vertebrate, such as a mammal, such as a human. Mammals include, but are not limited to, murines, simians, humans, farm animals, sport animals, and pets.

A “T cell” or “T lymphocyte” is an immune system cell that matures in the thymus and produces a T cell receptor (TCR). T cells can be naïve (“TN”; not exposed to antigen; increased expression of CD62L, CCR7, CD28, CD3, CD127, and CD45RA, and decreased or no expression of CD45RO as compared to TCM (described herein)), memory T cells (TM) (antigen experienced and long-lived), including stem cell memory T cells, and effector cells (antigen-experienced, cytotoxic). TM can be further divided into subsets of central memory T cells (TCM, expresses CD62L, CCR7, CD28, CD95, CD45RO, and CD127) and effector memory T cells (TEM, express CD45RO, decreased expression of CD62L, CCR7, CD28, and CD45RA). Effector T cells (TE) refers to antigen-experienced CD8+ cytotoxic T lymphocytes that express CD45RA, have decreased expression of CD62L, CCR7, and CD28 as compared to TCM, and are positive for granzyme and perform. Helper T cells (TH) are CD4+ cells that influence the activity of other immune cells by releasing cytokines. CD4+ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other example T cells include regulatory T cells, such as CD4+ CD25+ (Foxp3+) regulatory T cells and Treg17 cells, as well as Tr1, Th3, CD8+CD28−, and Qa-1 restricted T cells.

“T cell receptor” (TCR) refers to an immunoglobulin superfamily member having a variable binding domain, a constant domain, a transmembrane region, and a short cytoplasmic tail; see, e. g., Janeway et al., Immunobiology: The Immune System in Health and Disease, 3rd Ed., Current Biology Publications, p. 433, 1997) capable of specifically binding to an antigen peptide bound to a MHC receptor. A TCR can be found on the surface of a cell or in soluble form and generally is comprised of a heterodimer having a and R chains (also known as TCR α and TCRβ, respectively), or γ and δ chains (also known as TCRγ and TCRδ, respectively). In certain embodiments, a polynucleotide encoding a binding protein of this disclosure, e.g., a TCR, can be codon optimized to enhance expression in a particular host cell, such, for example, as a cell of the immune system, a hematopoietic stem cell, a T cell, a primary T cell, a T cell line, a NK cell, or a natural killer T cell (Scholten et al., Clin. Immunol. 119:135, 2006). Exemplary T cells that can express binding proteins and TCRs of this disclosure include CD4+ T cells, CD8+ T cells, and related subpopulations thereof (e.g., naïve, central memory, stem cell memory, effector memory).

Like other immunoglobulins (e.g., antibodies), the extracellular portion of TCR chains (e.g., α-chain, β-chain) contain two immunoglobulin domains, a variable domain (e.g., α-chain variable domain or Vα, β-chain variable domain or Vβ; typically amino acids 1 to 116 based on Kabat numbering (Kabat et al., “Sequences of Proteins of Immunological Interest, US Dept. Health and Human Services, Public Health Service National Institutes of Health, 1991, 5th ed.)) at the N-terminus, and one constant domain (e.g., α-chain constant domain or Cα, typically 5 amino acids 117 to 259 based on Kabat, β-chain constant domain or Cβ, typically amino acids 117 to 295 based on Kabat) adjacent the cell membrane. Also, like immunoglobulins, the variable domains contain complementary determining regions (CDRs) separated by framework regions (FRs) (see, e.g., Jores et al., Proc. Nat'l Acad. Sci. USA 87:9138, 1990; Chothia et al., EMBO J. 7:3745, 1988; see also Lefranc et al., Dev. Comp. Immunol. 27:55, 2003). The source of a TCR as used in the present disclosure may be from various animal species, such as a human, mouse, rat, rabbit, or other mammal.

The term “transmembrane domain” also referred to as “TMD,” is a membrane-spanning protein domain. Transmembrane domains may perform a variety of functions including but not limited to anchoring transmembrane proteins to the membrane, facilitating molecular transport of molecules such as ions and proteins across biological membranes, signal transduction across the membrane, assisting in vesicle fusion, and mediating transport and sorting of transmembrane proteins. Non-limiting examples of transmembrane domains are described herein. Additional examples of transmembrane domains are known in the art.

By “Transforming Growth Factor Beta polypeptide” (TGFβ) is meant a protein or fragment thereof having at least about 85% amino acid identity to GENBANK Accession No. AAH22242.1, and having TGFβR2 binding activity. An exemplary polypeptide sequence is provided below:

>AAH22242.1 Transforming growth factor,
beta 1 [Homo sapiens]
(SEQ ID NO: 156)
MPPSGLRLLLLLLPLLWLLVLTPGRPAAGLSTCKT
IDMELVKRKRIEAIRGQILSKLRLASPPSQGEVPP
GPLPEAVLALYNSTRDRVAGESAEPEPEPEADYYA
KEVTRVLMVETHNEIYDKFKQSTHSIYMFFNTSEL
REAVPEPVLLSRAELRLLRLKLKVEQHVELYQKYS
NNSWRYLSNRLLAPSDSPEWLSFDVTGVVRQWLSR
GGEIEGFRLSAHCSCDSRDNTLQVDINGFTTGRRG
DLATIHGMNRPFLLLMATPLERAQHLQSSRHRRAL
DTNYCFSSTEKNCCVRQLYIDFRKDLGWKWIHEPK
GYHANFCLGPCPYIWSLDTQYSKVLALYNQHNPGA
SAAPCCVPQALEPLPIVYYVGRKPKVEQLSNMIVR
SCKCS

In the present disclosure, it will be understood that Transforming Growth Factor Beta can also be referred-to as TGFβ, TGFB, Transforming Growth Factor Beta 1, Latency-Associated Peptide, or LAP.

By “TGFR beta polynucleotide” is meant a nucleic acid molecule encoding a TGFR beta polypeptide. An exemplary sequence of a TGFR beta polynucleotide is provided below:

>BC022242.1 Homo sapiens transforming growth
factor, beta 1, mRNA (cDNA clone
MGC: 22008 IMAGE: 4399762), complete cds
(SEQ ID NO: 157)
CCCAGACCTCGGGCGCACCCCCTGCACGCCGCCTTCATCCCCGGCCTGTCTCCTGAGCCCCCGCGCATCC
TAGACCCTTTCTCCTCCAGGAGACGGATCTCTCTCCGACCTGCCACAGATCCCCTATTCAAGACCACCCA
CCTTCTGGTACCAGATCGCGCCCATCTAGGTTATTTCCGTGGGATACTGAGACACCCCCGGTCCAAGCCT
CCCCTCCACCACTGCGCCCTTCTCCCTGAGGACCTCAGCTTTCCCTCGAGGCCCTCCTACCTTTTGCCGG
GAGACCCCCAGCCCCTGCAGGGGGGGGGCCTCCCCACCACACCAGCCCTGTTCGCGCTCTCGGCAGTGCC
GGGGGGCGCCGCCTCCCCCATGCCGCCCTCCGGGCTGCGGCTGCTGCTGCTGCTGCTACCGCTGCTGTGG
CTACTGGTGCTGACGCCTGGCCGGCCGGCCGCGGGACTATCCACCTGCAAGACTATCGACATGGAGCTGG
TGAAGCGGAAGCGCATCGAGGCCATCCGCGGCCAGATCCTGTCCAAGCTGCGGCTCGCCAGCCCCCCGAG
CCAGGGGGAGGTGCCGCCCGGCCCGCTGCCCGAGGCCGTGCTCGCCCTGTACAACAGCACCCGCGACCGG
GTGGCCGGGGAGAGTGCAGAACCGGAGCCCGAGCCTGAGGCCGACTACTACGCCAAGGAGGTCACCCGCG
TGCTAATGGTGGAAACCCACAACGAAATCTATGACAAGTTCAAGCAGAGTACACACAGCATATATATGTT
CTTCAACACATCAGAGCTCCGAGAAGCGGTACCTGAACCCGTGTTGCTCTCCCGGGCAGAGCTGCGTCTG
CTGAGGCTCAAGTTAAAAGTGGAGCAGCACGTGGAGCTGTACCAGAAATACAGCAACAATTCCTGGCGAT
ACCTCAGCAACCGGCTGCTGGCACCCAGCGACTCGCCAGAGTGGTTATCTTTTGATGTCACCGGAGTTGT
GCGGCAGTGGTTGAGCCGTGGAGGGGAAATTGAGGGCTTTCGCCTTAGCGCCCACTGCTCCTGTGACAGC
AGGGATAACACACTGCAAGTGGACATCAACGGGTTCACTACCGGCCGCCGAGGTGACCTGGCCACCATTC
ATGGCATGAACCGGCCTTTCCTGCTTCTCATGGCCACCCCGCTGGAGAGGGCCCAGCATCTGCAAAGCTC
CCGGCACCGCCGAGCCCTGGACACCAACTATTGCTTCAGCTCCACGGAGAAGAACTGCTGCGTGCGGCAG
CTGTACATTGACTTCCGCAAGGACCTCGGCTGGAAGTGGATCCACGAGCCCAAGGGCTACCATGCCAACT
TCTGCCTCGGGCCCTGCCCCTACATTTGGAGCCTGGACACGCAGTACAGCAAGGTCCTGGCCCTGTACAA
CCAGCATAACCCGGGCGCCTCGGCGGCGCCGTGCTGCGTGCCGCAGGCGCTGGAGCCGCTGCCCATCGTG
TACTACGTGGGCCGCAAGCCCAAGGTGGAGCAGCTGTCCAACATGATCGTGCGCTCCTGCAAGTGCAGCT
GAGGTCCCGCCCCGCCCCGCCCCGCCCCGGCAGGCCCGGCCCCACCCCGCCCCGCCCCCGCTGCCTTGCC
CATGGGGGCTGTATTTAAGGACACCCGTGCCCCAAGCCCACCTGGGGCCCCATTAAAGATGGAGAGAGGA
AAAAAAAAAAAAAAAAAAAAAAAAAAAAAAAAAAAAAAAAAAAAAAAAAAAAAAAAAAAAAAAAAA

By “Transforming Growth Factor Beta Receptor 2 polypeptide” (TGFβR2) is meant a protein or fragment thereof having at least about 85% amino acid identity to GENBANK Accession No. ABG65632.1, and having TGFβ binding activity. An exemplary polypeptide sequence is provided below:

>ABG65632.1 transforming growth factor beta
receptor II [Homo sapiens]
(SEQ ID NO: 158)
MGRGLLRGLWPLHIVLWTRIASTIPPHVQKSVNND
MIVTDNNGAVKFPQLCKFCDVRFSTCDNQKSCMSN
CSITSICEKPQEVCVAVWRKNDENITLETVCHDPK
LPYHDFILEDAASPKCIMKEKKKPGETFFMCSCSS
DECNDNIIFSEEYNTSNPDLLLVIFQVTGISLLPP
LGVAISVIIIFYCYRVNRQQKLSSTWETGKTRKLM
EFSEHCAIILEDDRSDISSTCANNINHNTELLPIE
LDTLVGKGRFAEVYKAKLKQNTSEQFETVAVKIFP
YEEYASWKTEKDIFSDINLKHENILQFLTAEERKT
ELGKQYWLITAFHAKGNLQEYLTRHVISWEDLRKL
GSSLARGIAHLHSDHTPCGRPKMPIVHRDLKSSNI
LVKNDLTCCLCDFGLSLRLDPTLSVDDLANSGQVG
TARYMAPEVLESRMNLENVESFKQTDVYSMALVLW
EMTSRCNAVGEVKDYEPPFGSKVREHPCVESMKDN
VLRDRGRPEIPSFWLNHQGIQMVCETLTECWDHDP
EARLTAQCVAERFSELEHLDRLSGRSCSEEKIPED
GSLNTTK

In the present disclosure, it will be understood that Transforming Growth Factor Beta Receptor 2 can also be referred-to as TGFβR2 or TGFBR2.

By “Transforming Growth Factor Beta Receptor 2 polynucleotide” is meant a nucleic acid molecule encoding a TGFBR2 polypeptide. An exemplary sequence of a Transforming Growth Factor Beta Receptor 2 polynucleotide is provided below:

>AH015833.2 Homo sapiens chromosome 3 transforming growth factor
beta receptor II (TGFBR2) gene, complete cds
(SEQ ID NO: 159)
CCTCCTGGCTGGCGAGCGGGCGCCACATCTGGCCCGCACATCTGCGCTGCCGGCCCGGCGCGGGGTCCGG
AGAGGGCGCGGCGCGGAGGCGCAGCCAGGGGTCCGGGAAGGCGCCGTCCGCTGCGCTGGGGGCTCGGTCT
ATGACGAGCAGCGGGGTCTGCCATGGGTCGGGGGCTGCTCAGGGGCCTGTGGCCGCTGCACATCGTCCTG
TGGACGCGTATCGCCAGCACGATCCCACCGCACGTTCAGAAGTCGGGTGAGTGGTCCCCAGCCCGGGCTC
GGCGGGGCGCCGGGGGTCTTCCTGGGGTCCCCGCCTCTCCGCTGCGCTTGACAGTCGGGCCCGGCAACCC
GGCCCCCGGGCGGAAACGAGGAAAGTTTCCCCCGCGACACTCACGCAGCCCGACTCCCGTAGCTGCAGGG
ATTGTGAGTTTTTCTTGAAAAAGAGAAGGAAAGTTCAGTTGCAAGGGGCGCGGGGCACGTTTGGTCCNNN
NNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNAAGCAAATGGCTACTCAACCACCCCAACCCCTCACCACCCCTC
ACCACGGTACAATGGATTTTATTACAAATTGCATACACCAGATAATTCTAATCTGATGTGAAGGAATTAT
TTTGCCTTTCTTCAGATTCATTCTCATGACATCAAGTTCATTTGAAATTGCATAACATCTTCAGGAATTC
ATTGGCAGGCTGCCTGGCAGTTGGATAATCATTTAATATATCTTTCTCTCTCCTCAGTTAATAACGACAT
GATAGTCACTGACAACAACGGTGCAGTCAAGTTTCCACAACTGTGTAAATTTTGTGATGTGAGATTTTCC
ACCTGTGACAACCAGAAATCCTGCATGAGCAACTGCAGCATCACCTCCATCTGTGAGAAGCCACAGGAAG
TCTGTGTGGCTGTATGGTAAGCAAGCCTTTTAAGAAGTTATTCTTTCTTTTCCCCTTTTTACATAATGTA
TTCTCATAGTACACACAGTCAGTGTATCTCTGTCTCCTAAATGTAAACACCTGTTCCATTTCCCTTTCCT
TTAGACCATCTCTCTTTCGATTATTAAATGTAGTTTCTAGGGGTGTTCTCTGCATGTATGTGTNNNNNNN
NNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNTGCGAATGCTGGAGAACAGGAACCAGCTGCCGTTGTTAGGAACAACT
TCATGAAGGAAAAGTATTCCAGATTGCCTTTCTGTCTGGAGGCCATATTATTCATTTATTCTCTTTCTCT
CTCTCCCTCTCCCCTCGCTTCCAATGAATCTCTTCACTCTAGGAGAAAGAATGACGAGAACATAACACTA
GAGACAGTTTGCCATGACCCCAAGCTCCCCTACCATGACTTTATTCTGGAAGATGCTGCTTCTCCAAAGT
GCATTATGAAGGAAAAAAAAAAGCCTGGTGAGACTTTCTTCATGTGTTCCTGTAGCTCTGATGAGTGCAA
TGACAACATCATCTTCTCAGAAGGTGAGTTTTCTTCTCTTAAGGGTGTGGGACCTGAGATCTGTGCCAAT
TTTTTGTATCCTTGGTCTGCAGTGTCATAGAGCACATTCCTCCTGTGGTGGATTGCATACNNNNNNNNNN
NNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNAGCTGAAGTTTGAAGGAAGAGCAGGGGATGACGAACAGATGGCCAGAGGC
AGGGAAGGCTGAACGAGCATGCACTTGCATCCCTGAAATAAAAATTAACAATATCGTATCTACAAAAACT
ATGCAGATGCTAAAATCTATAGATGCTCAGGCATGAACCCACTTCCTGACAGTACTTACCTACCACATCC
AACTCCTTCTCTCCTTGTTTTGTTTCCCCATCAGAATATAACACCAGCAATCCTGACTTGTTGCTAGTCA
TATTTCAAGTGACAGGCATCAGCCTCCTGCCACCACTGGGAGTTGCCATATCTGTCATCATCATCTTCTA
CTGCTACCGCGTTAACCGGCAGCAGAAGCTGAGTTCAACCTGGGAAACCGGCAAGACGCGGAAGCTCATG
GAGTTCAGCGAGCACTGTGCCATCATCCTGGAAGATGACCGCTCTGACATCAGCTCCACGTGTGCCAACA
ACATCAACCACAACACAGAGCTGCTGCCCATTGAGCTGGACACCCTGGTGGGGAAAGGTCGCTTTGCTGA
GGTCTATAAGGCCAAGCTGAAGCAGAACACTTCAGAGCAGTTTGAGACAGTGGCAGTCAAGATCTTTCCC
TATGAGGAGTATGCCTCTTGGAAGACAGAGAAGGACATCTTCTCAGACATCAATCTGAAGCATGAGAACA
TACTCCAGTTCCTGACGGCTGAGGAGCGGAAGACGGAGTTGGGGAAACAATACTGGCTGATCACCGCCTT
CCACGCCAAGGGCAACCTACAGGAGTACCTGACGCGGCATGTCATCAGCTGGGAGGACCTGCGCAAGCTG
GGCAGCTCCCTCGCCCGGGGGATTGCTCACCTCCACAGTGATCACACTCCATGTGGGAGGCCCAAGATGC
CCATCGTGCACAGGGACCTCAAGAGCTCCAATATCCTCGTGAAGAACGACCTAACCTGCTGCCTGTGTGA
CTTTGGGCTTTCCCTGCGTCTGGACCCTACTCTGTCTGTGGATGACCTGGCTAACAGTGGGCAGGTAAGT
TAGAGCTAGTGCTAGATCCCCTTTACCTTGAGCCTGGCCTCACCCTACCTCTTGATCCATATCTCCTGGC
TCTTATCTCAAACAGCCCTGTACTCTGGACACTGGTCTAGGGAATCTAGCCAAAGTATGGAGTCTGCCTT
GAGCATACTCTGCTCTGTCCTGCCTGAGCATTTTTGCTAATGGACAGCATTTCTCCTCCTATCTTCAAAT
CCTTCCCAGTTCAGCACATTTTTTCCNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNCTATCTGTACCTTT
CTGTGCATTTCTCATTCTTTAAAACAGCACTTTGATTTTTTAAAAAAATCCTCTGCACGTGTCAGGGGCC
ACCATCAGCTATATTGTGAAAATAAAAAGGCAGCTGGAATTAAATGATGGGCCTCACTGTCTGTTTTTGC
TATAGGTGGGAACTGCAAGATACATGGCTCCAGAAGTCCTAGAATCCAGGATGAATTTGGAGAATGTTGA
GTCCTTCAAGCAGACCGATGTCTACTCCATGGCTCTGGTGCTCTGGGAAATGACATCTCGCTGTAATGCA
GTGGGAGGTAGGTGTGGACCAGCATCATTGTGTAGTGGTAAACTTGTCTTCAAAATAAGATCATGTGTTG
CTTCGAGCATTATTCCAGGGGTTACAAAGCAGTTATTAGAGCTAGTTGAGATCTGATATTATACAACCAT
CCCAGAACTATTTGGGGTTATGCTAAACAGCCTAGNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNGTTAC
TTAGTGCTTCATGCTCCCCAGCCAGGCATCTCACCATGCTCATTTCCTTTGGCTGCACATGCCATTCTCA
GTGACCCTGTGTTTGCTGGCTTTCTTCACAGAAGTAAAAGATTATGAGCCTCCATTTGGTTCCAAGGTGC
GGGAGCACCCCTGTGTCGAAAGCATGAAGGACAACGTGTTGAGAGATCGAGGGCGACCAGAAATTCCCAG
CTTCTGGCTCAACCACCAGGTAAGGAGTGAGTGTTTACAAAGGTCAGTAAGATTCAACCAAGTTGCCTCT
TAGGTGGCAGAGAATTCTGGAATATTGAGCTTAAATCTGAGGGAAGGTCCCATTGTGTTCTATNNNNNNN
NNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNGTGTTGGGAGTGTTAGTGTACCCCAGAAGCCCAGCAGAGGATCATTG
TGCTGTCAGGACCTATTGTGATCAGATAGCACCAGAGCCAAAAAGTATGGTAGGTTTTGAGCACAGCTGC
CACCAGCACAAACCCCCCACCACCCTTTCCACATGGAACTGGCTGGCCTGCAGCAGCAGGCACTCAGTCA
GCACATGTTAAATGCACAGGCACTTTTGGACCCTGCTTGCACTCACTATAGCAACAAGGTCAGCAGGCCA
CCTTGCCTTCCGCGGAGCCCACCAACTCATGGTGCCCTTTGGATCTCTTTCCCGCTACAGGGCATCCAGA
TGGTGTGTGAGACGTTGACTGAGTGCTGGGACCACGACCCAGAGGCCCGTCTCACAGCCCAGTGTGTGGC
AGAACGCTTCAGTGAGCTGGAGCATCTGGACAGGCTCTCGGGGAGGAGCTGCTCGGAGGAGAAGATTCCT
GAAGACGGCTCCCTAAACACTACCAAATAGCTCTTCTGGGGCAGGCTGGGCCATGTCCAAAGAGGCTGCC
CCTCTCACCAAAGAACAGAGGCAGCAGGAAGCTGCCCCTGAACTGATGCTTCCTGGAAAACCAAGGGGGT
CACTCCCCTCCCTGTAAGCTGTGGGGATAAGCAGAAACAACAGCAGCAGGGAGTGGGTGACATAGAGCAT
TCTATGCCTTTGACATTGTCATAGGATAAGCTGTGTTAGCACTTCCTCAGGAAATGAGATTGATTTTTAC
AATAGCCAATAACATTTGCACTTTATTAATGCCTGTATATAAATATGAATAGCTATGTTTTATATATATA
TATATATATCTATATATGTCTATAGCTCTATATATATAGCCATACCTTGAAAAGAGACAAGGAAAAACAT
CAAATATTCCCAGGAAATTGGTTTTATTGGAGAACTCCAGAACCAAGCAGAGAAGGAAGGGACCCATGAC
AGCATTAGCATTTGACAATCACACATGCAGTGGTTCTCTGACTGTAAAACAGTGAACTTTGCATGAGGAA
AGAGGCTCCATGTCTCACAGCCAGCTATGACCACATTGCACTTGCTTTTGCAAAATAATCATTCCCTGCC
TAGCACTTCTCTTCTGGCCATGGAACTAAGTACAGTGGCACTGTTTGAGGACCAGTGTTCCCGGGGTTCC
TGTGTGCCCTTATTTCTCCTGGACTTTTCATTTAAGCTCCAAGCCCCAAATCTGGGGGGCTAGTTTAGAA
ACTCTCCCTCAACCTAGTTTAGAAACTCTACCCCATCTTTAATACCTTGAATGTTTTGAACCCCACTTTT
TACCTTCATGGGTTGCAGAAAAATCAGAACAGATGTCCCCATCCATGCGATTGCCCCACCATCTACTAAT
GAAAAATTGTTCTTTTTTTCATCTTTCCCCTGCACTTATGTTACTATTCTCTGCTCCCAGCCTTCATCCT
TTTCTAAAAAGGAGCAAATTCTCACTCTAGGCTTTATCGTGTTTACTTTTTCATTACACTTGACTTGATT
TTCTAGTTTTCTATACAAACACCAATGGGTTCCATCTTTCTGGGCTCCTGATTGCTCAAGCACA

The term “variable region” or “variable domain” refers to the domain of an immunoglobulin superfamily binding protein (e.g., a TCR α-chain or n-chain (or γ chain and δ chain for γδ TCRs)) that is involved in binding of the immunoglobulin superfamily binding protein (e.g., TCR) to antigen. The variable domains of the α chain and β chain (Vα and Vβ, respectively) of a native TCR generally have similar structures, with each domain comprising four generally conserved framework regions (FRs) and three CDRs. The Vα domain is encoded by two separate DNA segments, the variable gene segment and the joining gene segment (V-J); the Vβ domain is encoded by three separate DNA segments, the variable gene segment, the diversity gene segment, and the joining gene segment (V-D-J). A single Vα or Vβ domain may be sufficient to confer antigen-binding specificity. Furthermore, TCRs that bind a particular antigen may be isolated using a Vα or Vβ domain from a TCR that binds the antigen to screen a library of complementary Vα or Vβ domains, respectively.

The terms “treatment” and “treating,” as used herein, refer to an approach for obtaining beneficial or predetermined results including but not limited to a therapeutic benefit or a prophylactic benefit. For example, a treatment can comprise administering a system or cell population disclosed herein. “Therapeutic benefit” means any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

### BRIEF DESCRIPTION OF THE DRAWINGS

FIG. 1A provides schematics of TCR-T cells comprising a fusion protein having a CD34 extracellular domain, a IL7RA transmembrane domain with a Cysteine, Proline, Threonine (CPT) insert, and an IL7RA intracellular domain or a fusion protein comprising a Fas extracellular domain, a Fas-BB transmembrane domain, and a 4-1BB intracellular domain. The “CPT insert” is an insertion of a trimer peptide of cysteine, proline, threonine (CPT) into the transmembrane domain, which insertion results in a constitutively active IL7 receptor. Fas-BB is also referred to as Fas-41BB or Fas-4-1BB.

FIGS. 1B and 1C provide schematics of an immunomodulatory fusion protein (IFP) (i.e., a CD34-IL7RA) (FIG. 1B) and control constructs (FIG. 1C). MSCV denotes a Murine Stem Cell Virus retroviral promoter. P2A denotes a 2A peptide from porcine teschovirus-1. TCRb and TCRα denote the alpha and beta chains of a T-cell receptor.

FIG. 1D provides flow cytometric comparisons of phosphorylated STAT5 (pSTAT5) in primary CD8+ T cells (Cell A) transduced to express an anti-KRAS G12V TCR and a constitutively active CD34-IL7RA fusion protein (caIL7RA-Cell A) or a control (FasBB or CD8a). The Y axis quantifies fluorescence (counts) and the X axis quantifies phycoerythrin at 561 wavelength.

FIG. 1E is quantifies results for flow cytometric comparisons of pSTAT5 in CD8+ T cells expressing the fusion proteins described in FIG. 1D with a different anti-KRAS G12V TCR (Cell B).

FIG. 2A is a plot showing growth kinetics observed in a live tumor-visualization assay of HLA-A11+, KRAS G12V-expressing tumor cell lines cultured in the presence or absence of primary CD8+ T cells expressing an anti-KRAS G12V TCR and a constitutively active CD34-IL7RA fusion protein (caIL7RA-Cell A) or a control (FasBB or CD8a).

FIG. 2B is a plot showing growth kinetics observed in a live tumor-visualization assay of HLA-A11+, KRAS G12V-expressing tumor cell lines cultured in the presence or absence of CD8+ T cells expressing the same fusion proteins and a different anti-KRAS G12V TCR (Cell B).

FIGS. 3A-3C are plots showing growth kinetics observed in a live tumor-visualization assay of HLA-A11+, KRAS G12V-expressing tumor cell lines cultured in the presence or absence of primary CD8+ T cells transduced to express an anti-KRAS G12V TCR and a constitutively active CD34-IL7RA fusion protein (caIL7RA-Cell A) or a control (FasBB or CD8a). FIG. 3A shows growth kinetics of SW480 tumor cells cultured at a 1:1 tumor cell to effector cell ratio. FIG. 3B shows growth kinetics of SW527 tumor cells cultured at a 1:3 tumor cell to effector cell ratio. FIG. 3C shows growth kinetics of SW620 tumor cells cultured at a 1:1 tumor cell to effector cell ratio. “UTD” denotes cancer cells cul

FIG. 4 is a graph showing T cell count of T cells transduced to express an anti-KRAS G12V TCR and a constitutively active CD34-IL7RA fusion protein (caIL7RA-Cell A) or a control (FasBB or CD8a). after 6 days of co-culture with tumor cells (i.e., SW480, SW527, or SW620 cells) at a 1:1 ratio as measured by flow cytometry

FIGS. 5A-5B are graphs showing cell counts CD8+ T cells transduced to express an anti-KRAS G12V TCR and a constitutively active CD34-IL7RA fusion protein (caIL7RA-Cell A) or a control (FasBB) and untransduced (UTD) controls that were activated and expanded and then transferred to no cytokine-containing medium (FIG. 5A) or medium augmented with cytokine from day 3 to day 10 (FIG. 5B).

FIG. 6 provides schematics of conditionally active fusion proteins comprising TGFβR2, CD80, SIRPα, CD58, CD40L, and CD2 extracellular domains, IL7RA intracellular domains, and either an IL7RA transmembrane or a transmembrane derived from the same protein from which the extracellular domain was derived.

FIG. 7 are readouts of flow cytometric analysis to detect expression in T cells transduced with a polynucleotide encoding an anti-KRAS G12V TCR and an immunomodulatory fusion protein comprising a transmembrane domain derived from the same protein from which the extracellular domain was derived from or from IL7R. Expression of the transgenic TCR was used as a control. IFP denotes: immunomodulatory fusion protein.

FIGS. 8A-8F are readouts from flow cytometric analysis of phosphorylated STAT5 (pSTAT5) levels in primary CD8+ T cells transduced with polynucleotides encoding an anti-KRAS G12V TCR and a conditional (FIGS. 8A, 8B, 8C, 8D, and 8E) or a constitutive (FIG. 8F) IL7R fusion protein.

FIG. 9 provides schematics of IL7R fusion proteins comprising TGFbR2, CD80, SIRPα, CD58, CD40L, and CD2 extracellular domains.

FIG. 10 shows readouts of flow cytometric analyses to detect T cells expressing a constitutively active IL7R fusion protein.

FIG. 11 shows readouts of flow cytometric analyses to detect phosphorylated STAT5 (pSTAT5) levels in primary CD8+ T cells transduced with polynucleotides encoding an anti-KRAS G12V TCR and an IL7R fusion protein.

FIGS. 12A-12C are graphs showing growth kinetics of HLA-A11+, KRAS G12V-expressing tumor cell lines in a live tumor-visualization assay. FIG. 12A shows growth kinetics of SW527 tumor cells in the presence or absence of primary CD8+ T cells transduced with polynucleotides encoding an anti-KRAS GT2V TCR and an IL7R fusion protein at a 2:1 tumor to effector cell ratio. FIG. 12B shows growth kinetics of SW620 tumor cells in the presence or absence of primary CD8+ T cells transduced with polynucleotides encoding an anti-KRAS G12V TCR and an IL7R fusion protein at a 5:1 tumor to effector cell ratio. FIG. 12C shows growth kinetics of CFPAC1 tumor cells in the presence or absence of primary CD8+ T cells transduced with polynucleotides encoding an anti-KRAS G12V TCR and an IL7R fusion protein at a 5:1 tumor to effector cell ratio.

FIGS. 13A-13C are graphs showing T cell counts determined by flow cytometry on Day 7 of co-culture of TCR- and IL7R fusion protein-transduced T cells with tumor cells at an effector cell to tumor cell ratio of either 5:1 or 2:1. FIG. 13A shows T cell counts after co-culture with SW527 tumor cells. FIG. 13B shows T cell counts after co-culture with SW620 tumor cells. FIG. 13C shows T cell counts after co-culture with CFPAC1 tumor cells.

FIG. 14 are flow cytometric readouts showing expression of IL7R fusion protein.

FIG. 15 is a flow cytometric readout showing expression of IL7R fusion proteins having a constitutively active, inverted, deleted, or mutated IL7R intracellular domain.

FIG. 16 are graphs of a flow cytometric analysis of phosphorylated STAT5 (pSTAT5) in primary CD8-T cells transduced with polynucleotides encoding an anti-KRAS G12V TCR and an IL7R fusion protein.

FIG. 17 is a graph showing activation as measured by CD137 expression identifying specifically activated CD8+ T cells transduced with polynucleotides encoding an anti-KRAS G12V TCR and an IL7R fusion protein following peptide-specific activation.

FIGS. 18A-18D are graphs showing growth kinetics of HLA-A11+, KRAS G12V-expressing tumor cell lines in live tumor-visualization assay in the presence or absence of primary CD8+ T cells transduced with polynucleotides encoding an anti-KRAS G12V TCR and an IL7R fusion protein. FIG. 18A shows growth kinetics of SW527 tumor cells in the presence or absence of T cells transduced with polynucleotides encoding an anti-KRAS G12V TCR and an IL7R fusion protein. FIG. 18B shows growth kinetics of SW620 tumor cells in the presence or absence of T cells transduced with polynucleotides encoding an anti-KRAS G12V TCR and an IL7R fusion protein. FIG. 18C shows growth kinetics of CFPAC-1 tumor cells in the presence or absence of T cells transduced with polynucleotides encoding an anti-KRAS G12V TCR and an IL7R fusion protein. FIG. 18D shows growth kinetics of Dang tumor cells in the presence or absence of T cells transduced with polynucleotides encoding an anti-KRAS G12V TCR and an IL7R fusion protein.

FIG. 19 is a graph showing T cell count after 8 days as measured by flow cytometry of primary CD8+ T cells transduced with polynucleotides encoding an anti-KRAS G12V TCR and an IL7R fusion protein f.

FIG. 20A and FIG. 20B are graphs showing the growth kinetics of HPAF-II and PANC1+, KRAS G12D-expressing tumor cell lines, respectively, in a live tumor-visualization assay in the presence or absence of G12D TCR-T cells mixed with caIL7RA− primary CD8+ T cells transduced with polynucleotides encoding an anti-KRAS G12V TCR and an IL7R fusion protein.

FIG. 21 is a graph showing cell proliferation by counting cells for primary CD8+ T cells 5 transduced with polynucleotides encoding an anti-KRAS G12V TCR and an IL7R fusion protein and activated and expanded in medium without cytokine stimulation.

FIG. 22 are flow cytometric readouts of detection of expression of T cells transduced with polynucleotides encoding an anti-KRAS G12V TCR and an IL7R fusion protein.

FIG. 23 are flow cytometric readouts of phosphorylated STAT5 (pSTAT5) detected in primary T cells transduced with polynucleotides encoding an anti-KRAS G12V TCR and an IL7R fusion protein.

FIGS. 24A-24B are graphs showing CD137 expression as a measure of T cell activation following peptide-specific activation.

FIGS. 25A-25D show growth kinetics of HLA-A11+, KRAS G12V-expressing tumor cell lines in a live tumor-visualization assay in the presence or absence of caIL7RA-TCR_{KASG12V}-transduced primary T cells. FIG. 25A shows growth kinetics of SW527 tumor cells in the presence or absence of T cells transduced with polynucleotides encoding an anti-KRAS G12V TCR and an IL7R fusion protein. FIG. 25B shows growth kinetics of SW620 tumor cells in the presence or absence of T cells transduced with polynucleotides encoding an anti-KRAS G12V TCR and an IL7R fusion protein. FIG. 25C shows growth kinetics of CFPAC-1 tumor cells in the presence or absence of T cells transduced with polynucleotides encoding an anti-KRAS G12V TCR and an IL7R fusion protein. FIG. 25D shows growth kinetics of Dang tumor cells in the presence or absence of T cells transduced with polynucleotides encoding an anti-KRAS G12V TCR and an IL7R fusion protein.

FIGS. 26A-26B are graphs showing T cell count after 8 days as measured by flow cytometry of primary CD8+ T cells (FIG. 26A) and primary CD4+-T cells, wherein each T cell type was transduced with polynucleotides encoding an anti-KRAS G12V TCR and an IL7R fusion protein.

FIG. 27 shows growth kinetics of HLA-A11+, KRAS G12V-expressing tumor cell lines in live tumor-visualization assay in the presence or absence of caIL7RA-TCR_{KASG12V}-transduced primary T cells.

FIGS. 28A-28D are readouts showing phosphorylated STAT5 in T cells transduced with a polynucleotide encoding a CD58-IL7RA fusion protein having a mutated IL7RA transmembrane domain. FIG. 28A shows phosphorylated STAT5 in T cells transduced with a polynucleotide encoding a CD58-IL7RA fusion protein comprising a mutated IL7RA transmembrane having the amino acid sequence of SEQ ID NO: 42 (IFTCPSISILS). FIG. 28B shows phosphorylated STAT5 in T cells transduced with a polynucleotide encoding a CD58-IL7RA fusion protein comprising a mutated IL7RA transmembrane having the amino acid sequence of SEQ ID NO: 44 (ILLTSHQPCILS). FIG. 28C shows phosphorylated STAT5 in T cells transduced with a polynucleotide encoding a CD58-IL7RA fusion protein comprising a mutated IL7RA transmembrane having the amino acid sequence of SEQ ID NO: 122 (PITLYCKTLLTISILS). FIG. 28D shows phosphorylated STAT5 in T cells transduced with a polynucleotide encoding a CD58-IL7RA fusion protein comprising a mutated IL7RA transmembrane having the amino acid sequence of SEQ ID NO: 123 (ISPCITISILS). The presence of a shoulder and/or peak to the right of the main peak in each of FIGS. 28A-28D indicates that these fusion proteins were constitutively active.

FIGS. 29A and 29B show tumor cell killing activity of the T cells described in FIGS. 28A-28D. FIG. 29A is a graph showing changes in tumor volume when SW527 tumor cells are cocultured with the T cells described in FIGS. 28A-28D at a 5:1 effector-to-target cell ratio. FIG. 29B is a graph showing changes in tumor volume when SW620 tumor cells are cocultured with the T cells described in FIGS. 28A-28D at a 5:1 effector-to-target cell ratio. In FIGS. 29A-29B, the different CD58-IL7RA fusion proteins are denoted as follows: (1) CD58-IL7R-18 corresponds to the fusion protein of FIG. 28A; (2) CD58-IL7R-20 corresponds to the fusion protein of FIG. 28B; (3) CD58-IL7R-35 corresponds to the fusion protein of FIG. 28C; (4) CD58-IL7R-37 corresponds to the fusion protein of FIG. 28D.

FIGS. 30A-30D are readouts showing phosphorylated STAT5 in T cells transduced with a polynucleotide encoding a CD80-IL7RA fusion protein having a mutated IL7RA transmembrane domain. FIG. 30A shows phosphorylated STAT5 in T cells transduced with a polynucleotide encoding a CD80-IL7RA fusion protein comprising a mutated IL7RA transmembrane having the amino acid sequence of SEQ ID NO: 42 (IFTCPSISILS). FIG. 30B shows phosphorylated STAT5 in T cells transduced with a polynucleotide encoding a CD58-IL7RA fusion protein comprising a mutated IL7RA transmembrane having the amino acid sequence of SEQ ID NO: 44 (ILLTSHQPCILS). FIG. 30C shows phosphorylated STAT5 in T cells transduced with a polynucleotide encoding a CD80-IL7RA fusion protein comprising a mutated IL7RA transmembrane having the amino acid sequence of SEQ ID NO: 122 (PITLYCKTLLTISILS). FIG. 30D shows phosphorylated STAT5 in T cells transduced with a polynucleotide encoding a CD80-IL7RA fusion protein comprising a mutated IL7RA transmembrane having the amino acid sequence of SEQ ID NO: 123 (ISPCITISILS). The presence of a shoulder and/or peak to the right of the main peak in each of FIGS. 30A-30D indicates that these fusion proteins were constitutively active.

FIGS. 31A and 31B show tumor cell killing activity of the T cells described in FIGS. 30A-30D. FIG. 31A is a graph showing changes in tumor volume when SW527 tumor cells are cocultured with the T cells described in FIGS. 30A-30D at a 5:1 effector-to-target cell ratio. FIG. 31B is a graph showing changes in tumor volume when SW620 tumor cells are cocultured with the T cells described in FIGS. 30A-30D at a 5:1 effector-to-target cell ratio. In FIGS. 31A-31B, the different CD80-IL7RA fusion proteins are denoted as follows: (1) CD80-IL7R-18 corresponds to the fusion protein of FIG. 30A; (2) CD80-IL7R-20 corresponds to the fusion protein of FIG. 30B; (3) CD80-IL7R-35 corresponds to the fusion protein of FIG. 30C; (4) CD80-IL7R-37 corresponds to the fusion protein of FIG. 30D.

### DETAILED DESCRIPTION

Improved alternative chimeric T cells may be useful in immunotherapy for anti-tumor efficacy while maintaining low toxicity. Accordingly, herein are described fusion proteins, which are suitable for use in tumor-killing immunotherapy procedures. The present disclosure includes methods and compositions for increasing TCR-T cell survival and homeostatic proliferation post infusion and enhancing T cell expansion in vivo.

### Fusion Proteins

The fusion proteins of the present disclosure comprise an extracellular domain and an intracellular domain separated by a transmembrane domain. In some embodiments, the extracellular domain comprises or consists of an extracellular binding domain, and the intracellular domain comprises or consists of an intracellular signaling domain, wherein the extracellular binding domain and the intracellular signaling domain are from or are derived from different proteins (i.e., the extracellular binding domain and the intracellular signaling domain are not observed in the same polypeptide in nature). In some embodiments, the transmembrane domain is from or derived from the same protein from which the extracellular binding domain is from or derived from. In some embodiments, the transmembrane domain is from or derived from the same protein from which the intracellular binding domain is from or derived from. In some embodiments, the transmembrane domain is from or derived from a different protein than the proteins from which the extracellular binding domain and the intracellular signaling domain are from or derived from.

In some embodiments, the extracellular domain of a fusion protein provided herein comprises or is derived from the extracellular domain of a CD80, a CD58, a CD2, a SIRPα, a CD47L, or a TGFβR2 polypeptide, or a portion or variant thereof that is capable of binding to a CD28 or CTLA-4, CD2, CD47, CD40, CD58, or TGFβ, respectively. In some embodiments, the intracellular domain of a fusion protein provided herein comprises or is derived from an Interleukin 7 Receptor A (IL7RA) polypeptide, or a portion or variant thereof that is capable of contributing to an IL-7 signal in a host cell. In some embodiments, the transmembrane domain of a fusion protein provided herein comprises or is derived from a transmembrane of a TGFβR2, an IL7RA, a CD80, a CD58, a SIRPα, a CD40L, a CD2, an IL2RA, an IL2RB, an IL2RG, an IL4R, an IL9R, an IL21R, an IL15R, or a CD8 polypeptide, or a portion or variant thereof comprising at least one hydrophobic amino acid residue and capable of embedding or otherwise interacting with a cell of plasma membrane.

In some embodiments, a transmembrane domain disclosed herein comprises one or more mutations relative to the amino acid sequence of the wild type transmembrane domain. In some embodiments, the transmembrane domain is at least 50, 60, 65, 70, 75, 80, 85, 90, 91, 92, 93, 94, 95, 96, 97, 98, or 99 percent identical to the corresponding wild type transmembrane domain.

The mutation(s) may be a substitution, insertion, deletion, or combination thereof. In some embodiments, the one or more mutations comprises inclusion of at least one cysteine or at least one proline.

a. Intracellular Domains

The present disclosure provides, in part, fusion proteins comprising an intracellular domain comprising an intracellular domain of an IL7RA polypeptide or a portion or variant thereof that is capable of contributing to an IL-7 signal in a host cell. In some embodiments, an intracellular domain of an IL7RA polypeptide or a portion or variant thereof comprises an amino acid sequence having at least 80%, at least 85%, at least 90%, at least 95%, at least 99%, or greater sequence identity to SEQ ID NO: 22.

In some embodiments, an intracellular domain of an IL7RA polypeptide or a portion or variant thereof further comprises: one or more residues of a BOX1 motif corresponding to residues 8-15 (VWPSLPDH (SEQ ID NO: 124)) relative to SEQ ID NO: 15 when optimally aligned, or Y185 relative to SEQ ID NO: 15 when optimally aligned. In some embodiments, an intracellular domain of an IL7RA polypeptide or a portion or variant thereof further comprises one or more residues of a FERM domain corresponding to residues 1-6 (KKRIKPI (SEQ ID NO: 125)) or residues 16-28 (KKTLEHLCKKPRK (SEQ ID NO: 126)) relative to SEQ ID NO:15 when optimally aligned.

b. Transmembrane Domains

In some embodiments, the fusion proteins provided herein comprise a transmembrane domain from or derived from an IL7RA polypeptide. In some embodiments, the transmembrane domain is a wild type IL7RA transmembrane domain. In some embodiments, an IL7RA transmembrane domain comprises the amino acid sequence PILLTISILSFFSVALLVILACVLW (SEQ ID NO: 22). In some embodiments, an IL7RA transmembrane domain comprises an amino acid sequence having one or more mutations relative to SEQ ID NO: 22. In some embodiments, an IL7RA transmembrane domain comprises an amino acid sequence having at least 80%, 85%, 90%, 95%, or 99% or greater sequence identity to SEQ ID NO: 22. In some embodiments, the mutation enables or facilitates homodimerization of the receptor. In some embodiments, the mutation is, or comprises, the insertion of one or more cysteines and/or one or more prolines into the amino acid sequence of SEQ ID NO: 22. In some embodiments, the mutation comprises an insertion of a trimer peptide of cysteine, proline, threonine (CPT) into the transmembrane domain. Alternative or additional mutations are contemplated (see, e.g., Tables 1 and 2)

In some embodiments, the present disclosure provides for a fusion protein comprising a transmembrane domain from or derived from a CD80 polypeptide or a portion or a variant thereof. In some embodiments, the transmembrane domain from or d CD80 polypeptide or portion or variant thereof comprises an amino acid sequence having at least 80%, at least 85%, at least 90%, at least 95%, or at least 99% or greater sequence identity to SEQ ID NO: 23.

In some embodiments, the present disclosure provides for a fusion protein comprising a transmembrane domain from or derived from a CD58 polypeptide or a portion or variant thereof. In some embodiments, the transmembrane domain from or derived from a CD58 polypeptide or portion or variant thereof comprises an amino acid sequence having at least 80%, at least 85%, at least 90%, at least 95%, at least 99% or greater sequence identity to SEQ ID NO: 24.

In some embodiments, the present disclosure provides for a fusion protein comprising a transmembrane domain from or derived from a SIRPα polypeptide or a portion or a variant thereof. In some embodiments, the transmembrane domain from or derived from a SIRPα polypeptide or portion or variant thereof comprises an amino acid sequence having at least 80%, at least 85%, at least 90%, at least 95%, or at least 99% or greater sequence identity to SEQ ID NO: 25.

In some embodiments, the present disclosure provides for a fusion protein comprising a transmembrane domain from or derived from a CD40L polypeptide or a portion or variant thereof. In some embodiments, the transmembrane domain from or derived from a CD40L polypeptide or portion or variant thereof comprises an amino acid sequence having at least 80%, at least 85%, at least 90%, at least 95%, or at least 99% or greater sequence identity to SEQ ID NO: 26.

The present disclosure provides, in part, fusion proteins comprising a transmembrane domain of a TGFβR2 polypeptide or a portion or variant thereof that is capable of binding a TGFβ polypeptide. In some embodiments, a TGFβR2 polypeptide or portion or variant thereof comprises an amino acid sequence having at least 80%, at least 85%, at least 90%, at least 95%, at least 99%, or greater sequence identity to SEQ ID NO: 21.

c. Extracellular Domains

In some embodiments, the present disclosure provides for a fusion protein comprising an extracellular domain from or derived from a Cluster of Differentiation 80 (CD80) polypeptide, or a portion or variant thereof, that is capable of binding a CD28 or CTLA-4 polypeptide. In some embodiments, the extracellular domain from or derived from a CD80 polypeptide or portion or variant thereof comprises an amino acid sequence having at least 80%, at least 85%, at least 90%, at least 95%, or at least 99% or greater sequence identity to SEQ ID NO: 16.

In some embodiments, an extracellular domain from or derived from a CD80 polypeptide or portion or variant thereof comprises one or more residues corresponding to Leu25, Asn63, Arg29, Asp60, Lys86, Gln-33, Tyr31, Met38, Val39, Met47, Ile49, Trp50, Tyr53, Ile67, Phe108, Pro111, Ile113, Gln157, Asp158, Glu162, or Leu163 relative to SEQ ID NO: 16 when optimally aligned

In some embodiments, the present disclosure provides for a fusion protein comprising an extracellular domain from or derived from a CD58 polypeptide or a portion or variant thereof that is capable of binding a Cluster of Differentiation 2 (CD2) polypeptide. In some embodiments, the extracellular domain from or derived from a CD58 polypeptide or portion or variant thereof comprises an amino acid sequence having at least 80%, at least 85%, at least 90%, at least 95%, or at least 99% or greater sequence identity to SEQ ID NO: 17.

In some embodiments, an extracellular domain from or derived from a CD58 polypeptide or portion or variant thereof comprises one or more residues corresponding to Glu25, Lys29, Lys32, Asp33, Lys34, Glu37, Glu39, Glu42, Arg44, Ser47, Glu78, or Asp84 relative to SEQ ID NO: 17.

In some embodiments, the present disclosure provides for a fusion protein comprising an extracellular domain from or derived from a SIRPα polypeptide or a portion or variant thereof that is capable of binding a Cluster of Differentiation 47 (CD47) polypeptide. In some embodiments, the extracellular domain from or derived from a SIRPα polypeptide or portion or variant thereof comprises an amino acid sequence having at least 80%, at least 85%, at least 90%, at least 95%, or at least 99% or greater sequence identity to SEQ ID NO: 18.

In some embodiments, an extracellular domain from or derived from a SIRPα polypeptide or a portion or variant thereof comprises one or more residues corresponding to L30, G34, Q52, S66, T67, R69, K93, G97, S98, K53, E54, K96, D100, S29, 136, Q37, 131, V33, P35, 136, K68, or F74 relative to SEQ ID NO: 18.

The present disclosure provides, in part, fusion proteins comprising an extracellular domain from or derived from a CD40L polypeptide or a portion or variant thereof that is capable of binding a CD40 polypeptide. In some embodiments, the extracellular domain from or derived from a CD40L polypeptide or portion or variant thereof comprises an amino acid sequence having at least 80%, at least 85%, at least 90%, at least 95%, or at least 99% or greater sequence identity to SEQ ID NO: 19.

In some embodiments, an extracellular domain from or derived from a CD40L receptor or a portion or variant thereof comprises one or more residues corresponding to A130, P217, V247, F253, 1190, E129, K143, G144, Y145, Y146, C178, C218, Q220, S245, Q246, S248, G250, T251, G252, G199, R200, R203, Q232, K133, E142, H249, S132, T134, R207, 1127, S128, S185, Q186, A187, F201, H249, Y170, H224, Q121, H125, T147, Y172, Q174, L195, R203, L205, L206, R207, A208, A209, N210, T211, A215, G219, Q221, S222, L225, G226, G227, V228, F229, E230, T251, G252, L259, or L261 relative to SEQ ID NO: 19.

The present disclosure provides, in part, fusion proteins comprising an extracellular domain of a CD2 polypeptide or a portion or variant thereof that is capable of binding a CD58 polypeptide. In some embodiments, a CD2 polypeptide or portion or variant thereof comprises an amino acid sequence having at least 80%, at least 85%, at least 90%, at least 95%, at least 99%, or greater sequence identity to SEQ ID NO: 20.

In some embodiments, the present disclosure provides for a fusion protein comprising a transmembrane domain of a CD2 polypeptide or a portion or variant thereof.

In some embodiments, an extracellular domain of a CD2 or a portion or variant thereof comprises one or more residues corresponding to Lys43, Tyr86, Asn92, Glu95, Asn92, Asp32, Gly90, Arg48, Lys51, Asp31, Lys89, Lys34, Lys41 relative to SEQ ID NO: 20.

The present disclosure provides, in part, fusion proteins comprising an extracellular domain of a TGFβR2 polypeptide or a portion or variant thereof that is capable of binding a TGFβ polypeptide. In some embodiments, a TGFβR2 polypeptide or portion or variant thereof comprises an amino acid sequence having at least 80%, at least 85%, at least 90%, at least 95%, at least 99%, or greater sequence identity to SEQ ID NO: 13.

In some embodiments, the present disclosure provides for a fusion protein comprising a transmembrane domain of a TGFβR2 polypeptide or a portion or variant thereof.

In some embodiments, an extracellular domain of an TGFβR2 receptor, or a portion or variant thereof comprises one or more residues corresponding to Glu119, Asp32, Glu75, Tyr85, Ser49-Cys54, Leu27, Phe30, Ile50, Thr51, or Ile53 relative to SEQ ID NO: 13.

### Polynucleotides

In some embodiments, the present disclosure provides for a polynucleotide encoding a fusion protein or polypeptide disclosed herein. In some embodiments, the polynucleotides of the present disclosure encode a T-cell receptor. In some embodiments, the polynucleotides of the present disclosure encode an IL7R fusion protein or polypeptide.

In certain embodiments, a polynucleotide encoding a binding protein or polypeptide of this disclosure, e.g., a TCR, can be codon optimized to enhance expression in a particular host cell, such, for example, as a cell of the immune system, a hematopoietic stem cell, a T cell, a primary T cell, a T cell line, a NK cell, or a natural killer T cell (Scholten et al., Clin. Immunol. 119:135, 2006).

### Vectors

Vectors containing a nucleotide sequence encoding a fusion protein or polypeptide of the present disclosure are also provided. In some embodiments, the vectors comprise a nucleotide sequence encoding a fusion protein or polypeptide of the present disclosure. In some embodiments, the vector further includes a promoter operably linked to the nucleotide sequence encoding a fusion protein or polypeptide. In a particular embodiment, the promoter is a murine stem cell virus (MSCV) promoter. In some embodiments, any suitable promoter for use in a given host cell may be used. For example, when the host is an animal cell, an SR.alpha. promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used.

The vectors used to express a fusion protein or polypeptide as described herein may be any suitable expression vector known and used in the art. In some embodiments, the vector is a prokaryotic or eukaryotic vector. In some embodiments, the vector is an expression vector, such as a eukaryotic (e.g., mammalian) expression vector. In another embodiment, the vector is a plasmid (prokaryotic or bacterial) or a nanoplasmid vector. In another embodiment, the vector is a viral vector, such as a lentiviral vector. In some embodiments, the vector is an RNA polynucleotide suitable for translation in a cell. In some embodiments, the vector is a lipid nanoparticle. In some embodiments, the vector is a non-viral vector comprising an expression cassette.

Also provided is a fusion protein or polypeptide, as described herein, produced by transfecting a host cell with a vector containing a polynucleotide encoding the fusion protein or polypeptide. Also provided in some embodiments is a fusion protein or polypeptide, as described herein, produced by transfecting a host cell with a vector encoding the fusion protein or polypeptide under conditions sufficient to allow for expression of the fusion protein or polypeptide. Collections of plasmids (vectors) are also contemplated. In certain embodiments, the collection of plasmids includes plasmids encoding a fusion protein or polypeptide as described herein.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest.

### Host Cells

Host cells can be a T cell, such as a naïve T cell, a central memory T cell, a stem cell memory T cell, an effector memory T cell. In some embodiments, the T cell is a CD4+ T cell, a CD8+ T cell, a CD4− CD8− double negative T cell, or a γδ T cell, a natural killer cell, a natural killer T cell, a dendritic cell, or any combination thereof. In some embodiments, the host cell comprises a chromosomal gene knockout, mutation, or indel in a TRAC and/or TRBC gene locus, an MHC gene locus, or a combination thereof.

In some embodiments, the T cell comprises a polynucleotide encoding an exogenous TCR. Such a cell (“TCR-T cell”) may recognize a particular tumor associated antigen (TAA) and initiate or direct an immune response to the tumor cell expressing the TAA.

In some embodiments of the present disclosure, the host cell comprises a chimeric antigen receptor (CAR). The extracellular domain of the CAR comprises an antigen binding domain of an antibody or a functional fragment or derivative thereof (e.g., an scFv). In some embodiments, the transmembrane domain localizes the CAR to the cell membrane and/or stabilizes its structure; such transmembrane domains include those from CD28. In some embodiments, the intracellular signaling domain of the CAR comprises one or more functional signaling domains derived from at least one stimulatory molecule. In some embodiments, the stimulatory molecule may be a stimulatory receptor molecule, such as, for example, a stimulatory receptor molecule of an immune cell. In some embodiments, the stimulatory molecule may be the zeta chain associated with the T cell receptor complex. A non-exhaustive list of stimulatory molecules includes Fc gamma RIg (FCER1G), Fc gamma RIIa (FCGR2A), Fc Epsilon RIb (FCER1B), CD3 gamma, CD3 delta, CD3 epsilon, CD79a, CD79b, DAP10, and DAP1. In some embodiments, the intracellular signaling domain of the CAR further comprises one or more functional signaling domains derived from at least one costimulatory molecule. In some embodiments, the costimulatory molecule may comprise 4-1BB (e.g., CD137), CD27, CD28 CD3β, CD40, PD-1, CD2, CD7, CD258, NKG2C, B7-H3, a ligand that binds to CD83, ICAM-1, LFA-1 (CD11A/CD18), or ICOS. In some embodiments, the CAR comprises an optional leader sequence at its amino-terminus (N-terminus). In some embodiments, the CAR further comprises a signal peptide sequence at the N-terminus of the extracellular antigen recognition domain, wherein the signal peptide sequence is optionally cleaved from the antigen recognition domain (e.g., a scFv) during cellular processing and localization of the CAR to the cellular membrane.

In another aspect, compositions and unit doses are provided herein that comprise a modified host cell of the present disclosure and a pharmaceutically acceptable carrier, diluent, or excipient.

In certain embodiments, a host cell composition or unit dose comprises (i) a composition comprising at least about 30%, at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8+ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of peripheral blood mononuclear cells (PBMCs).

In some embodiments, a host cell composition or unit dose comprises (i) a composition comprising at least about 50% modified CD4⁺ T cells, combined with (ii) a composition comprising at least about 50% modified CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10 ratio, wherein the host cell composition or unit dose contains a reduced amount or substantially no naïve T cells. In further embodiments, a host cell composition or unit dose comprises (i) a composition comprising at least about 60% modified CD4⁺ T cells, combined with (ii) a composition comprising at least about 60% modified CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10 ratio, wherein the unit dose contains a reduced amount or substantially no naïve T cells. In still further embodiments, a host cell composition or unit dose comprises (i) a composition comprising at least about 70% engineered CD4+ T cells, combined with (ii) a composition comprising at least about 70% engineered CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10 ratio, wherein the unit dose contains a reduced amount or substantially no naïve T cells. In some embodiments, a host cell composition or unit dose comprises (i) a composition comprising at least about 80% modified CD4⁺ T cells, combined with (ii) a composition comprising at least about 80% modified CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10 ratio, wherein the host cell composition or unit dose contains a reduced amount or substantially no naïve T cells. In some embodiments, a host cell composition or unit dose comprises (i) a composition comprising at least about 85% modified CD4⁺ T cells, combined with (ii) a composition comprising at least about 85% modified CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10 ratio, wherein the host cell composition or unit dose contains a reduced amount or substantially no naïve T cells. In some embodiments, a host cell composition or unit dose comprises (i) a composition comprising at least about 90% modified CD4⁺ T cells, combined with (ii) a composition comprising at least about 90% modified CD8⁺ T cells, in about a 10:1, 9:1, 8:1, 7:1, 6:1, 5:1, 4:1, 3:1, 2:1, 1:1, 0.5:1, 0.1:1, 1:0.1, 1:0.5, 1:2, 1:3, 1:4, 1:5, 1:6, 1:7, 1:8, 1:9, or 1:10 ratio, wherein the host cell composition or unit dose contains a reduced amount or substantially no naïve T cells.

In some embodiments, the composition comprises a CD4+ cell population comprising (i) at least about 30%, at least about 40%, at least about 50%, at least about 60%, at least about 70%, at least about 80%, at least about 85%, at least about 90%, or at least about 95% modified CD4+ T cells. In some embodiments, the composition further comprises a CD8+ cell population comprising (ii) at least about 30%, at least about 40%, at least about 50%, at least about 60%, at least about 70%, at least about 80%, at least about 85%, at least about 90%, or at least about 95% modified CD8+ T cells.

In some embodiments, a host cell composition or unit dose comprises about a 1:1 ratio, about a 1:2 ratio, about a 1:3 ratio, about a 1:4 ratio, about a 1:5 ratio, about a 1:6 ratio, about a 1:7 ratio, about a 1:8 ratio, about a 1:9 ratio, about a 1:10 ratio, about a 2:1 ratio, about a 3:1 ratio, about a 4:1 ratio, about a 5:1 ratio, about a 6:1 ratio, about a 7:1 ratio, about an 8:1 ratio, about a 9:1 ratio, about a 10:1 ratio, about a 3:2 ratio, or about a 2:3 ratio of CD4+ to CD8+ T cells (for example, of CD4+ T cells modified to comprise or express a binding protein disclosed herein to CD8+ T cells modified to comprise or express a binding protein disclosed herein).

In some embodiments, a host cell composition or unit dose comprises ratio of CD4+ to CD8+ T cells that is at least 1:1, at least 1:2, at least 1:3, at least 1:4, at least 1:5, at least 1:6, at least 1:7, at least 1:8, at least 1:9, at least 1:10, at least 2:1, at least 3:1, at least 4:1, at least 5:1, at least 6:1, at least 7:1, at least 8:1, at least 9:1, at least 10:1, at least 3:2, or at least 2:3.

In some embodiments, a host cell composition or unit dose comprises ratio of CD4+ to CD8+ T cells that is at most 1:1, at most 1:2, at most 1:3, at most 1:4, at most 1:5, at most 1:6, at most 1:7, at most 1:8, at most 1:9, at most 1:10, at most 2:1, at most 3:1, at most 4:1, at most 5:1, at most 6:1, at most 7:1, at most 8:1, at most 9:1, at most 10:1, at most 3:2, or at most 2:3.

In some embodiments, a host cell composition or unit dose comprises ratio of CD4+ to CD8+ T cells that is between about 1:10 and 10:1, 1:10 and 8:1, 1:10 and 7:1, 1:10 and 6:1, 1:10 and 5:1, 1:10 and 4:1, 1:10 and 3:1, 1:10 and 2:1, 1:10 and 1:1, 1:10 and 1:2, 1:10 and 1:3, 1:10 and 1:4, 1:10 and 1:5, 1:10 and 1:7, 1:5 and 10:1, 1:5 and 8:1, 1:5 and 7:1, 1:5 and 6:1, 1:5 and 5:1, 1:5 and 4:1, 1:5 and 3:1, 1:5 and 2:1, 1:5 and 1:1, 1:5 and 1:2, 1:5 and 1:3, 1:5 and 1:4, 1:3 and 10:1, 1:3 and 8:1, 1:3 and 7:1, 1:3 and 6:1, 1:3 and 5:1, 1:3 and 4:1, 1:3 and 3:1, 1:3 and 2:1, 1:3 and 1:1, 1:3 and 1:2, 1:2 and 10:1, 1:2 and 8:1, 1:2 and 7:1, 1:2 and 6:1, 1:2 and 5:1, 1:2 and 4:1, 1:2 and 3:1, 1:2 and 2:1, 1:2 and 1:1, 1:1 and 10:1, 1:1 and 8:1, 1:1 and 7:1, 1:1 and 6:1, 1:1 and 5:1, 1:1 and 4:1, 1:1 and 3:1, 1:1 and 2:1, 2:1 and 10:1, 2:1 and 8:1, 2:1 and 7:1, 2:1 and 6:1, 2:1 and 5:1, 2:1 and 4:1, 2:1 and 3:1, 3:1 and 10:1, 3:1 and 8:1, 3:1 and 7:1, 3:1 and 6:1, 3:1 and 5:1, 3:1 and 4:1, 5:1 and 10:1, 5:1 and 8:1, 5:1 and 7:1, or 5:1 and 6:1.

CD4+ T cells in a composition, host cell composition, or unit dose can be CD4+ T cells that are modified or engineered to express a CD8 co-receptor disclosed herein, for example, using a vector or polynucleotide disclosed herein.

It will be appreciated that a host cell composition or unit dose of the present disclosure may comprise any host cell as described herein, or any combination of host cells. In certain embodiments, for example, a host cell composition or unit dose comprises modified CD8+ T cells, modified CD4+ T cells, or both, wherein these T cells are modified to encode a binding protein specific for a Ras peptide:HLA-A*11:01 complex. In addition or alternatively, a host cell composition or unit dose of the present disclosure can comprise any host cell or combination of host cells as described herein, and can further comprise a modified cell (e.g., immune cell, such as a T cell) expressing a binding protein specific for a different antigen (e.g., a different Ras antigen, or an antigen from a different protein or target, such as, for example, BCMA, CD3, CEACAM6, c-Met, EGFR, EGFRvIII, ErbB2, ErbB3, ErbB4, EphA2, IGF1R, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, TNFR1, TNFR2, PD1, PD-L1, PD-L2, HVEM, MAGE-A (e.g., including MAGE-A1, MAGE-A3, and MAGE-A4), mesothelin, NY-ESO-1, PSMA, RANK, ROR1, TNFRSF4, CD40, CD137, TWEAK-R, HLA, tumor- or pathogen-associated peptide bound to HLA, hTERT peptide bound to HLA, tyrosinase peptide bound to HLA, WT-1 peptide bound to HLA, LTβR, LIFRβ, LRP5, MUC1, OSMRβ, TCRα, TCRβ, CD19, CD20, CD22, CD25, CD28, CD30, CD33, CD52, CD56, CD79a, CD79b, CD80, CD81, CD86, CD123, CD171, CD276, B7H4, TLR7, TLR9, PTCH1, WT-1, HA¹-H, Robol, α-fetoprotein (AFP), Frizzled, OX40, PRAME, and SSX-2. or the like). In some embodiments, the binding protein binds to a peptide (e.g., the different antigens presented above) complexed with an HLA protein, e.g., an HLA-A, -B, -C, E, -G, -H, -J, -K, or -L. For example, a unit dose can comprise modified CD8⁺ T cells expressing a binding protein that specifically binds to a Ras-HLA complex and modified CD4⁺ T cells (and/or modified CD8⁺ T cells) expressing a binding protein (e.g., a CAR) that specifically binds to a PSMA antigen. It will also be appreciated that any of the host cells disclosed herein may be administered in a combination therapy.

In any of the embodiments described herein, a host cell composition or unit dose comprises equal, or approximately equal numbers of engineered CD45RA⁻ CD3⁺ CD8⁺ and modified CD45RA⁻ CD3⁺ CD4⁺ T_{M} cells.

In any of the embodiments described herein, a host cell composition or unit dose comprises one or more populations of cells (e.g., CD4+ or CD8+ cells) that have undergone CD62L positive selection, for example, to improve in vivo persistence.

Host cells can be genetically engineered to comprise or express a binding protein ex vivo, in vitro, or in vivo.

### Pharmaceutical Compositions

Also contemplated are pharmaceutical compositions (i.e., compositions) that comprise a composition (fusion protein, polynucleotide, vector, host cell, host cell composition, unit dose, and/or immunogenic polypeptide) as disclosed herein and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity).

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

### Methods of Enhancing Cell Function

In one aspect, methods are provided for enhancing a cellular function a cell, the method comprising modifying a cell to express a fusion protein disclosed herein. In some embodiments, the cell is an immune cell such as a T cell (e.g., a CD4+ T cell, a CD8+ T cell, a CD4− CD8-double-negative T cell, a γδ T cell, or a TCR-T cell), a CAR-T cell, a natural killer cell, or a dendritic cell. In some embodiments, the immune cell is modified to express an exogenous TCR (i.e., a TCR-T cell) or a chimeric antigen receptor (i.e., a CAR-T cell). In some embodiments, the immune cell is an effector immune cell. In some embodiments, any effector cell may be modified to express fusion proteins of the present disclosure, with beneficial results.

In some embodiments, the enhanced cellular function is proliferation. In other words, cells modified to express a fusion protein described herein will have increased proliferation relative to unmodified cells. In some embodiments, the cells modified to express a fusion protein described herein will have increased persistence relative to unmodified cells.

In some embodiments, the enhanced cellular function is enhanced or improved cell-killing activity of an immune cell, wherein the modified cell has improved cell-killing activity relative to an unmodified immune cell.

In some embodiments of the methods, modifying the immune cell can involve delivering a polynucleotide encoding the fusion protein to the immune cell. Methods of delivering a polynucleotide to a cell are well known to those skilled in the art. For example, a polynucleotide encoding a fusion protein of the present disclosure can be delivered by vectors (e.g., viral or non-viral vectors), or by naked DNA, DNA complexes, lipid nanoparticles, or a combination of the aforementioned compositions.

### Methods of Modulating an Immune Reaction

In one aspect, methods are provided for modulating an immune reaction, the method comprising modifying an immune cell to express a fusion protein disclosed herein and contacting the immune cell with an antigen having binding specificity to the fusion protein. In some embodiments, the contacting involves contacting the cell in a specific context or environment, such as in a tumor microenvironment (TME). In some embodiments, the method involves administering the modified immune cell to a subject in need thereof, thereby modulating an immune reaction in the subject.

### Methods of Treatment

In some aspects, the present disclosure provides for methods for treating a disease or condition, the methods comprising administering to a subject in need thereof an effective amount of a host cell, composition, or unit dose of the present disclosure.

In some embodiments, the disease or condition being treated is a cancer. As used herein, “cancer” may refer to any accelerated proliferation of cells, including solid tumors, ascites tumors, blood or lymph or other hematological malignancies; connective tissue malignancies; metastatic disease; minimal residual disease following transplantation of organs or stem cells; multi-drug resistant cancers, primary or secondary malignancies, angiogenesis related to malignancy, or other forms of cancer.

In some embodiments, a cancer treatable according to the presently disclosed methods and uses comprises a carcinoma, a sarcoma, a glioma, a lymphoma, a leukemia, a myeloma, or any combination thereof. In some embodiments, cancer comprises a cancer of the head or neck, melanoma, pancreatic cancer including but not limited to pancreatic ductal adenocarcinoma (PDAC), cholangiocarcinoma, hepatocellular cancer, breast cancer including but not limited to triple-negative breast cancer (TNBC), gastric cancer, lung cancer including but not limited to small-cell lung cancer and non-small-cell lung cancer, prostate cancer, esophageal cancer, mesothelioma, colorectal cancer, glioblastoma, or any combination thereof. In certain embodiments, the cancer comprises a solid tumor. In some embodiments, the solid tumor is a sarcoma or a carcinoma.

In some embodiments, the disease to be treated is an infection, such as a bacterial, viral, or fungal infection.

In some embodiments, the host cell is an allogeneic cell, a syngeneic cell, or an autologous cell. In some embodiments, the host cell will further express or encode an antigen-binding protein. Subjects that can be treated by the present disclosure are, in general, human and other primate subjects, such as monkeys and apes for veterinary medicine purposes. In some embodiments, the subject may be a human subject. Cells according to the present disclosure may be administered in a manner appropriate to the disease, condition, or disorder to be treated as determined by persons skilled in the medical art.

In some embodiments, a cell comprising a fusion protein disclosed herein is administered intravenously, intraperitoneally, intratumorally, into the bone marrow, into a lymph node, or into the cerebrospinal fluid. An appropriate dose, suitable duration, and frequency of administration of the compositions can be determined upon consideration of one or more factors, including but not limited to, a patient's health status, size (e.g., weight, mass, or body area), type and severity of the disease, condition, or disorder, the particular form of the active ingredient; and the method of administration.

In some embodiments, methods of the present disclosure comprise administering a host cell expressing a fusion protein disclosed herein. In some embodiments, the host cell will further express or encode an antigen-binding protein (e.g., a TCR or CAR). The number or amount of cells in a composition is at least one cell (for example, one fusion protein-modified CD8+ T cell subpopulation; one fusion protein-modified CD4+ T cell subpopulation) or is greater than 10² cells, for example, up to 10⁶, up to 10⁷, up to 10⁸ cells, up to 10⁹ cells, or more than 10¹⁰ cells, such as about 10¹¹ cells or more. In certain embodiments, the cells are administered in a range from about 10⁵ to about 10¹¹ cells/m², in a range of about 10⁵ or about 10⁶ to about 10⁹ or about 10¹⁰ cells/m². Cell populations comprising cells expressing (or comprising a polynucleotide encoding) a fusion protein provided herein are contemplated. For example, some embodiments provide cell populations comprising cells modified to express a fusion protein provided herein are at least 30%, 35%, 40%, 45%, 50%, 55%, 60%, 65%, 70%, 75%, 80%, 85%, 90%, 95% or more of the total cell population. In some embodiments, cells expressing (or comprising a polynucleotide encoding) a fusion protein provided herein can have a volume of 1 liter or less, 500 ml or less, 250 ml or less, or 100 ml or less. In some embodiments, the density of the cells is greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁵, 10⁶, 10⁷, 10⁸, 10⁹, 10¹⁰, or 10¹¹ cells.

Also contemplated are pharmaceutical compositions that comprise a fusion protein provided herein or cells expressing or (comprising a polynucleotide encoding) a fusion protein as disclosed herein, and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the art. An appropriate dose and a suitable duration and frequency of administration of the compositions can be determined upon consideration of one or more factors, such as a patient's health status, size of the patient (e.g., weight, mass, or body area), the type and severity of the patient's condition, the type or level or activity of the fusion protein-expressing cells, the particular form of the active ingredient, and the method of administration. In general, an appropriate dose and treatment regimen comprise a composition provided herein in an amount sufficient to provide therapeutic or prophylactic benefit (such as described herein, including an improved clinical outcome, such as more frequent complete or partial remissions, or longer disease-free or overall survival, or a lessening of symptom severity). For prophylactic use, a dose may be sufficient to prevent, delay the onset of, or diminish the severity of a disease associated with a target (e.g., an antigen, such as a tumor-associated antigen). Prophylactic benefit of the immunogenic compositions administered according to the methods described herein can be determined by performing preclinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a host cell (which may be autologous, allogeneic or syngeneic) comprising a polynucleotide as described herein that is stably integrated into the chromosome of the cell. For example, such a cellular composition may be generated ex vivo using autologous, allogeneic or syngeneic immune system cells (e.g., T cells, antigen-presenting cells, natural killer cells) in order to administer a fusion protein-expressing T cell composition to a subject as an adoptive immunotherapy. In certain embodiments, the host cell comprises a hematopoietic progenitor cell or a human immune cell. In some embodiments, the cell comprises a CD4+ T cell, a CD8+ T cell, a CD4− CD8− double-negative T cell, a γδ T cell, a natural killer cell, a dendritic cell, or any combination thereof. In certain embodiments, the immune system cell comprises a naïve T cell, a central memory T cell, a stem cell memory T cell, an effector memory T cell, or any combination thereof

As used herein, administration of a composition refers to delivering the same to a subject, regardless of the route or mode of delivery. Administration may be affected continuously or intermittently, and parenterally. Administration may be for treating a subject already confirmed as having a recognized condition, disease or disease state, or for treating a subject susceptible to or at risk of developing such a condition, disease, or disease state. Co-administration with an adjunctive therapy may include simultaneous or sequential delivery of multiple agents in any order and on any dosing schedule (e.g., fusion protein-expressing recombinant (e.g., engineered) host cells with one or more cytokines; immunosuppressive therapy such as calcineurin inhibitors, corticosteroids, microtubule inhibitors, low dose of a mycophenolic acid prodrug, or any combination thereof).

In some embodiments, a plurality of doses of a host cell as described herein is administered to the subject, which may be administered at intervals between administrations of about two to about four weeks or more. In certain embodiments, the plurality of unit doses are administered at intervals between administrations of about two, three, four, five, six, seven, eight, or more weeks.

An effective amount of a pharmaceutical composition (e.g., host cell, fusion protein, unit dose, or composition) refers to an amount sufficient, at dosages and for periods of time needed, to achieve the predetermined clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

Methods disclosed herein may further include administering one or more additional agents to treat the disease or disorder in a combination therapy. For example, in certain embodiments, a combination therapy comprises administering a fusion protein (or an engineered host cell expressing the same) with (concurrently, simultaneously, or sequentially) an immune checkpoint inhibitor. In some embodiments, a combination therapy comprises administering a host cell expressing a fusion protein of the present disclosure with an agonist of a stimulatory immune checkpoint agent. In some embodiments, a combination therapy comprises administering a host cell expressing a fusion protein of the present disclosure with a secondary therapy, such as chemotherapeutic agent, a radiation therapy, a surgery, an antibody, or any combination thereof.

### Kits

In one aspect, the present disclosure provides kits for modifying a cell to express a fusion protein disclosed herein. In some embodiments, the kit comprises reagents useful in the introduction of polynucleotides encoding the fusion protein.

The disclosure also provides kits for the treatment or prevention of cancer. In some embodiments, the kit includes a therapeutic composition containing an immune cell that expresses a fusion protein provided herein. In some embodiments, the kit includes the immune cell expressing the fusion protein in unit dosage form in a sterile container. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired a pharmaceutical composition of the invention is provided together with instructions for administering the pharmaceutical composition to a subject having or at risk of developing or reoccurrence of cancer. The instructions will generally include information about the use of the composition for the treatment or prevention of cancer. In other embodiments, the instructions include at least one of the following: description of the pharmaceutical composition (i.e., a cellular composition); dosage schedule and administration for treatment or prevention of cancer or symptoms thereof, precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

TABLE 1
Sequences of Genes and Components Described Herein

| SEQ | | |
|---|---|---|
| ID | | |
| NO: | SOURCE | SEQUENCE |
| 1 | TGFbR2- | MGRGLLRGLWPLHIVLWTRIASTIPPHVQKSVNNDMIVTD |
| | EC TGFbR2- | NNGAVKFPQLCKFCDVRFSTCDNQKSCMSNCSITSICEKPQ |
| | TM_IL7Ra-IC(R2- | EVCVAVWRKNDENITLETVCHDPKLPYHDFILEDAASPKCI |
| | R2TM-7R) | MKEKKKPGETFFMCSCSSDECNDNIIFSEEYNTSNPDLLLVI |
| | | FQVTGISLLPPLGVAISVIIIFYCYKKRIKPIVWPSLPDHKKTLEH |
| | | LCKKPRKNLNVSFNPESFLDCQIHRVDDIQARDEVEGFLQDTFP |
| | | QQLEESEKQRLGGDVQSPNCPSEDVVITPESFGRDSSLTCLAGN |
| | | VSACDAPILSSSRSLDCRESGKNGPHVYQDLLLSLGTTNSTLPP |
| | | PFSLQSGILTLNPVAQGQPILTSLGSNQEEAYVTMSSFYQNQ |
| 2 | TGFbR2-EC_IL7Ra- | MGRGLLRGLWPLHIVLWTRIASTIPPHVQKSVNNDMIVTD |
| | TM_IL7Ra-IC(R2- | NNGAVKFPQLCKFCDVRFSTCDNQKSCMSNCSITSICEKPQ |
| | 7RTM-7R) | EVCVAVWRKNDENITLETVCHDPKLPYHDFILEDAASPKCI |
| | | MKEKKKPGETFFMCSCSSDECNDNIIFSEEYNTSNPDLLLVI |
| | | FQPILLTISILSFFSVALLVILACVLWKKRIKPIVWPSLPDHKKTL |
| | | EHLCKKPRKNLNVSFNPESFLDCQIHRVDDIQARDEVEGFLQD |
| | | TFPQQLEESEKQRLGGDVQSPNCPSEDVVITPESFGRDSSLTCL |
| | | AGNVSACDAPILSSSRSLDCRESGKNGPHVYQDLLLSLGTTNST |
| | | LPPPFSLQSGILTLNPVAQGQPILTSLGSNQEEAYVTMSSFYQN |
| | | Q |
| 3 | CD80-EC_CD80- | MGHTRRQGTSPSKCPYLNFFQLLVLAGLSHFCSGVIHVTKE |
| | TM_IL7Ra-IC(80- | VKEVATLSCGHNVSVEELAQTRIYWQKEKKMVLTMMSGD |
| | 80TM-7R) | MNIWPEYKNRTIFDITNNLSIVILALRPSDEGTYECVVLKYE |
| | | KDAFKREHLAEVTLSVKADFPTPSISDFEIPTSNIRRIICSTSG |
| | | GFPEPHLSWLENGEELNAINTTVSQDPETELYAVSSKLDEN |
| | | MTTNHSFMCLIKYGHLRVNQTFNWNTTKQEHFPDNLLPSW |
| | | AITLISVNGIFVICCLTYCKKRIKPIVWPSLPDHKKTLEHLCKKP |
| | | RKNLNVSFNPESFLDCQIHRVDDIQARDEVEGFLQDTFPQQLEE |
| | | SEKQRLGGDVQSPNCPSEDVVITPESFGRDSSLTCLAGNVSACD |
| | | APILSSSRSLDCRESGKNGPHVYQDLLLSLGTTNSTLPPPFSLQS |
| | | GILTLNPVAQGQPILTSLGSNQEEAYVTMSSFYQNQ |
| 4 | CD80-EC_IL7Ra- | MGHTRRQGTSPSKCPYLNFFQLLVLAGLSHFCSGVIHVTKE |
| | TM_IL7Ra-IC(80- | VKEVATLSCGHNVSVEELAQTRIYWQKEKKMVLTMMSGD |
| | 7RTM-7R) | MNIWPEYKNRTIFDITNNLSIVILALRPSDEGTYECVVLKYE |
| | | KDAFKREHLAEVTLSVKADFPTPSISDFEIPTSNIRRIICSTSG |
| | | GFPEPHLSWLENGEELNAINTTVSQDPETELYAVSSKLDEN |
| | | MTTNHSFMCLIKYGHLRVNQTFNWNTTKQEHFPDNPILLTI |
| | | SILSFFSVALLVILACVLWKKRIKPIVWPSLPDHKKTLEHLCKK |
| | | PRKNLNVSFNPESFLDCQIHRVDDIQARDEVEGFLQDTFPQQLE |
| | | ESEKQRLGGDVQSPNCPSEDVVITPESFGRDSSLTCLAGNVSAC |
| | | DAPILSSSRSLDCRESGKNGPHVYQDLLLSLGTTNSTLPPPFSLQ |
| | | SGILTLNPVAQGQPILTSLGSNQEEAYVTMSSFYQNQ |
| 5 | CD58-EC_CD58- | MVAGSDAGRALGVLSVVCLLHCFGFISCFSQQIYGVVYGN |
| | TM_IL7Ra-IC(58- | VTFHVPSNVPLKEVLWKKQKDKVAELENSEFRAFSSFKNR |
| | 58TM-7R) | VYLDTVSGSLTIYNLTSSDEDEYEMESPNITDTMKFFLYVLE |
| | | SLPSPTLTCALTNGSIEVQCMIPEHYNSHRGLIMYSWDCPM |
| | | EQCKRNSTSIYFKMENDLPQKIQCTLSNPLFNTTSSIILTTCI |
| | | PSSGHSRHRYALIPIPLAVITTCIVLYMNGILKCKKRIKPIVWPS |
| | | LPDHKKTLEHLCKKPRKNLNVSFNPESFLDCQIHRVDDIQARD |
| | | EVEGFLQDTFPQQLEESEKQRLGGDVQSPNCPSEDVVITPESFG |
| | | RDSSLTCLAGNVSACDAPILSSSRSLDCRESGKNGPHVYQDLLL |
| | | SLGTTNSTLPPPFSLQSGILTLNPVAQGQPILTSLGSNQEEAYVT |
| | | MSSFYQNQ |
| 6 | CD58-EC_IL7Ra- | MVAGSDAGRALGVLSVVCLLHCFGFISCFSQQIYGVVYGN |
| | TM_IL7Ra-IC(58- | VTFHVPSNVPLKEVLWKKQKDKVAELENSEFRAFSSFKNR |
| | 7RTM-7R) | VYLDTVSGSLTIYNLTSSDEDEYEMESPNITDTMKFFLYVLE |
| | | SLPSPTLTCALTNGSIEVQCMIPEHYNSHRGLIMYSWDCPM |
| | | EQCKRNSTSIYFKMENDLPQKIQCTLSNPLFNTTSSIILTTCI |
| | | PSSGHSRHRPILLTISILSFFSVALLVILACVLWKKRIKPIVWPSL |
| | | PDHKKTLEHLCKKPRKNLNVSFNPESFLDCQIHRVDDIQARDE |
| | | VEGFLQDTFPQQLEESEKQRLGGDVQSPNCPSEDVVITPESFGR |
| | | DSSLTCLAGNVSACDAPILSSSRSLDCRESGKNGPHVYQDLLLS |
| | | LGTTNSTLPPPFSLQSGILTLNPVAQGQPILTSLGSNQEEAYVTM |
| | | SSFYQNQ |
| 7 | SIRPa-EC_SIRPa- | MEPAGPAPGRLGPLLCLLLAASCAWSGVAGEEELQVIQPD |
| | TM_IL7Ra- | KSVLVAAGETATLRCTATSLIPVGPIQWFRGAGPGRELIYN |
| | IC(SIRP-SIRPTM- | QKEGHFPRVTTVSDLTKRNNMDFSIRIGNITPADAGTYYCV |
| | 7R) | KFRKGSPDDVEFKSGAGTELSVRAKPSAPVVSGPAARATPQ |
| | | HTVSFTCESHGFSPRDITLKWFKNGNELSDFQTNVDPVGES |
| | | VSYSIHSTAKVVLTREDVHSQVICEVAHVTLQGDPLRGTAN |
| | | LSETIRVPPTLEVTQQPVRAENQVNVTCQVRKFYPQRLQLT |
| | | WLENGNVSRTETASTVTENKDGTYNWMSWLLVNVSAHRD |
| | | DVKLTCQVEHDGQPAVSKSHDLKVSAHPKEQGSNTAAENT |
| | | GSNERNIYIVVGVVCTLLVALLMAALYLVKKRIKPIVWPSLPD |
| | | HKKTLEHLCKKPRKNLNVSFNPESFLDCQIHRVDDIQARDEVE |
| | | GFLQDTFPQQLEESEKQRLGGDVQSPNCPSEDVVITPESFGRDS |
| | | SLTCLAGNVSACDAPILSSSRSLDCRESGKNGPHVYQDLLLSLG |
| | | TTNSTLPPPFSLQSGILTLNPVAQGQPILTSLGSNQEEAYVTMSS |
| | | FYQNQ |
| 8 | SIRPa-EC IL7Ra- | MEPAGPAPGRLGPLLCLLLAASCAWSGVAGEEELQVIQPD |
| | TM_IL7Ra- | KSVLVAAGETATLRCTATSLIPVGPIQWFRGAGPGRELIYN |
| | IC(SIRP-7RTM-7R) | QKEGHFPRVTTVSDLTKRNNMDFSIRIGNITPADAGTYYCV |
| | | KFRKGSPDDVEFKSGAGTELSVRAKPSAPVVSGPAARATPQ |
| | | HTVSFTCESHGFSPRDITLKWFKNGNELSDFQTNVDPVGES |
| | | VSYSIHSTAKVVLTREDVHSQVICEVAHVTLQGDPLRGTAN |
| | | LSETIRVPPTLEVTQQPVRAENQVNVTCQVRKFYPQRLQLT |
| | | WLENGNVSRTETASTVTENKDGTYNWMSWLLVNVSAHRD |
| | | DVKLTCQVEHDGQPAVSKSHDLKVSAHPKEQGSNTAAENT |
| | | GSNERNIYPILLTISILSFFSVALLVILACVLWKKRIKPIVWPSLP |
| | | DHKKTLEHLCKKPRKNLNVSFNPESFLDCQIHRVDDIQARDEV |
| | | EGFLQDTFPQQLEESEKQRLGGDVQSPNCPSEDVVITPESFGRD |
| | | SSLTCLAGNVSACDAPILSSSRSLDCRESGKNGPHVYQDLLLSL |
| | | GTTNSTLPPPFSLQSGILTLNPVAQGQPILTSLGSNQEEAYVTMS |
| | | SFYQNQ |
| 9 | Constitutively | MLVRRGARAGPRMPRGWTALCLLSLLPSGFMSLDNNGTA |
| | active | TPELPTQGTFSNVSTNVSYQETTTPSTLGSTSLHPVSQHGNE |
| | IL-7 receptor | ATTNITETTVKFTSTSVITSVYGNTNSSVQSQTSVISTVFTTP |
| | | ANVSTPETTLKPSLSPGNVSDLSTTSTSLATSPTKPYTSSSPIL |
| | | SDIKAEIKCSGIREVKLTQGICLEQNKTSSCAEFKKDRGEGL |
| | | ARVLCGEEQADADAGAQVCSLLLAQSEVRPQCLLLVLANR |
| | | TEISSKLQLMKKHQSDLKKLGILDFTEQDVASHQSYSQKTP |
| | | ILLTCPTISILSFFSVALLVILACVLWKKRIKPIVWPSLPDHKKTL |
| | | EHLCKKPRKNLNVSFNPESFLDCQIHRVDDIQARDEVEGFLQD |
| | | TFPQQLEESEKQRLGGDVQSPNCPSEDVVITPESFGRDSSLTCL |
| | | AGNVSACDAPILSSSRSLDCRESGKNGPHVYQDLLLSLGTTNST |
| | | LPPPFSLQSGILTLNPVAQGQPILTSLGSNQEEAYVTMSSFYQN |
| | | Q |
| 10 | IL7Ra-IC CD40L- | MKKRIKPIVWPSLPDHKKTLEHLCKKPRKNLNVSFNPESFLDC |
| | TM CD40L-EC | QIHRVDDIQARDEVEGFLQDTFPQQLEESEKQRLGGDVQSPNC |
| | (7R-CD40LTM- | PSEDVVITPESFGRDSSLTCLAGNVSACDAPILSSSRSLDCRESG |
| | CD40L) | KNGPHVYQDLLLSLGTTNSTLPPPFSLQSGILTLNPVAQGQPILT |
| | | SLGSNQEEAYVTMSSFYQNQIFMYLLTVFLITQMIGSALFAVYL |
| | | HRRLDKIEDERNLHEDFVFMKTIQRCNTGERSLSLLNCEEI |
| | | KSQFEGFVKDIMLNKEETKKENSFEMQKGDQNPQIAAHVI |
| | | SEASSKTTSVLQWAEKGYYTMSNNLVTLENGKQLTVKRQ |
| | | GLYYIYAQVTFCSNREASSQAPFIASLCLKSPGRFERILLRA |
| | | ANTHSSAKPCGQQSIHLGGVFELQPGASVFVNVTDPSQVSH |
| | | GTGFTSFGLLKL |
| 11 | CD40L-EC | MALPVTALLLPLALLLHAARPHRRLDKIEDERNLHEDFVF |
| | (flip)_IL7Ra- | MKTIQRCNTGERSLSLLNCEEIKSQFEGFVKDIMLNKEETK |
| | TM_IL7Ra- | KENSFEMQKGDQNPQIAAHVISEASSKTTSVLQWAEKGYY |
| | IC(CD40L-7RTM- | TMSNNLVTLENGKQLTVKRQGLYYIYAQVTFCSNREASSQ |
| | 7R) | APFIASLCLKSPGRFERILLRAANTHSSAKPCGQQSIHLGGV |
| | | FELQPGASVFVNVTDPSQVSHGTGFTSFGLLKLPILLTISILSF |
| | | FSVALLVILACVLWKKRIKPIVWPSLPDHKKTLEHLCKKPRKN |
| | | LNVSFNPESFLDCQIHRVDDIQARDEVEGFLQDTFPQQLEESEK |
| | | QRLGGDVQSPNCPSEDVVITPESFGRDSSLTCLAGNVSACDAPI |
| | | LSSSRSLDCRESGKNGPHVYQDLLLSLGTTNSTLPPPFSLQSGIL |
| | | TLNPVAQGQPILTSLGSNQEEAYVTMSSFYQNQ |
| 12 | CD2-IL7RA | MSFPCKFVASFLLIFNVSSKGAVSKEITNALETWGALGQDIN |
| | receptor | LDIPSFQMSDDIDDIKWEKTSDKKKIAQFRKEKETFKEKDT |
| | | YKLFKNGTLKIKHLKTDDQDIYKVSIYDTKGKNVLEKIFDL |
| | | KIQERVSKPKISWTCINTTLTCEVMNGTDPELNLYQDGKHL |
| | | KLSQRVITHKWTTSLSAKFKCTAGNKVSKESSVEPVSCPEK |
| | | GLDPILLTCPTISILSFFSVALLVILACVLWKKRIKPIVWP |
| | | SLPDHKKTLEHLCKKPRKNLNVSFNPESFLDCQIHRVDDIQ |
| | | ARDEVEGFLQDTFPQQLEESEKQRLGGDVQSPNCPSEDVVI |
| | | TPESFGRDSSLTCLAGNVSACDAPILSSSRSLDCRESGKNG |
| | | PHVYQDLLLSLGTTNSTLPPPFSLQSGILTLNPVAQGQPIL |
| | | TSLGSNQEEAYVTMSSFYQNQ |
| 13 | TGFBR2 | MGRGLLRGLWPLHIVLWTRIASTIPPHVQKSVNNDMIVTDNNG |
| | Extracellular domain | AVKFPQLCKFCDVRFSTCDNQKSCMSNCSITSICEKPQEVCVA |
| | (TGFbR2-EC) | VWRKNDENITLETVCHDPKLPYHDFILEDAASPKCIMKEKKKP |
| | | GETFFMCSCSSDECNDNIIFSEEYNTSNPDLLLVIFQ |
| 15 | IL7RA intracellular | KKRIKPIVWPSLPDHKKTLEHLCKKPRKNLNVSFNPESFLDCQI |
| | domain | HRVDDIQARDEVEGFLQDTFPQQLEESEKQRLGGDVQSPNCPSE |
| | | DVVITPESFGRDSSLTCLAGNVSACDAPILSSSRSLDCRESGKN |
| | | GPHVYQDLLLSLGTTNSTLPPPFSLQSGILTLNPVAQGQPILTS |
| | | LGSNQEEAYVTMSSFYQNQ |
| 16 | CD80 extracellular | MGHTRRQGTSPSKCPYLNFFQLLVLAGLSHFCSGVIHVTKEVK |
| | domain | EVATLSCGHNVSVEELAQTRIYWQKEKKMVLTMMSGDMNIWPE |
| | | YKNRTIFDITNNLSIVILALRPSDEGTYECVVLKYEKDAFKRE |
| | | HLAEVTLSVKADFPTPSISDFEIPTSNIRRIICSTSGGFPEPH |
| | | LSWLENGEELNAINTTVSQDPETELYAVSSKLDFNMTTNHSFM |
| | | CLIKYGHLRVNQTFNWNTTKQEHFPDN |
| 17 | CD58 extracellular | MVAGSDAGRALGVLSVVCLLHCFGFISCFSQQIYGVVYGNVTF |
| | domain | HVPSNVPLKEVLWKKQKDKVAELENSEFRAFSSFKNRVYLDTV |
| | | SGSLTIYNLTSSDEDEYEMESPNITDTMKFFLYVLESLPSPTL |
| | | TCALTNGSIEVQCMIPEHYNSHRGLIMYSWDCPMEQCKRNSTS |
| | | IYFKMENDLPQKIQCTLSNPLFNTTSSIILTTCIPSSGHSRHR |
| 18 | SIRPa extracellular | MEPAGPAPGRLGPLLCLLLAASCAWSGVAGEEELQVIQPDKSV |
| | domain | LVAAGETATLRCTATSLIPVGPIQWFRGAGPGRELIYNQKEGHF |
| | | PRVTTVSDLTKRNNMDFSIRIGNITPADAGTYYCVKFRKGSPD |
| | | DVEFKSGAGTELSVRAKPSAPVVSGPAARATPQHTVSFTCESH |
| | | GFSPRDITLKWFKNGNELSDFQTNVDPVGESVSYSIHSTAKVV |
| | | LTREDVHSQVICEVAHVTLQGDPLRGTANLSETIRVPPTLEVTQ |
| | | QPVRAENQVNVTCQVRKFYPQRLQLTWLENGNVSRTETASTV |
| | | TENKDGTYNWMSWLLVNVSAHRDDVKLTCQVEHDGQPAVS |
| | | KSHDLKVSAHPKEQGSNTAAENTGSNERNIY |
| 19 | CD40L extracellular | HRRLDKIEDERNLHEDFVFMKTIQRCNTGERSLSLLNCEEIKSQ |
| | domain | FEGFVKDIMLNKEETKKENSFEMQKGDQNPQIAAHVISEASSK |
| | | TTSVLQWAEKGYYTMSNNLVTLENGKQLTVKRQGLYYIYAQ |
| | | VTFCSNREASSQAPFIASLCLKSPGRFERILLRAANTHSSAKPCG |
| | | QQSIHLGGVFELQPGASVFVNVTDPSQVSHGTGFTSFGLLKL |
| 20 | CD2 extracellular | MSFPCKFVASFLLIFNVSSKGAVSKEITNALETWGALGQDINLD |
| | domain | IPSFQMSDDIDDIKWEKTSDKKKIAQFRKEKETFKEKDTYKLFK |
| | | NGTLKIKHLKTDDQDIYKVSIYDTKGKNVLEKIFDLKIQERVSK |
| | | PKISWTCINTTLTCEVMNGTDPELNLYQDGKHLKLSQRVITHK |
| | | WTTSLSAKFKCTAGNKVSKESSVEPVSCPEKGLD |
| 21 | TGFBR2 | VTGISLLPPLGVAISVIIIFYCY |
| | transmembrane | |
| | domain (TGFbR2- | |
| | TM) | |
| 22 | IL7RA | PILLTISILSFFSVALLVILACVLW |
| | transmembrane | |
| | domain | |
| 23 | CD80 TM | LLPSWAITLISVNGIFVICCL |
| 24 | CD58 | YALIPIPLA VITTCIVLYMNGILKC |
| | transmembrane | |
| | domain | |
| 25 | SIRPa | IVVGVVCTLLVALLMAALYLV |
| | transmembrane | |
| | domain | |
| 26 | CD40L | IFMYLLTVFLITQMIGSALFAVYL |
| | transmembrane | |
| 27 | CD8 spacer | MALPVTALLLPLALLLHAAR |
| 28 | IL7RA | PILLTCPTISILSFFSVALLVILACVLW |
| | transmembrane | |
| | mutant CPT insert | |
| 29 | IL7RA TM mutant | ILLPPCLTISILS |
| | 243InsPPCL (SEQ | |
| | ID NO: 160) | |
| 30 | IL7RA TM mutant | ILLTISKCHLS |
| | 246 InsKCH | |
| 31 | IL7RA TM mutant | IFSCGPLTISILS |
| | 241 InsFSCGP (SEQ | |
| | ID NO: 161) | |
| 32 | IL7RA TM mutant | ILLTCHLISILS |
| | 244 InsCHL | |
| 33 | IL7RA TM mutant | ILLTPPVCSVTISILS |
| | 244 InsPPVCSVT | |
| | (SEQ ID NO: 162) | |
| 34 | IL7RA TM mutant | ILLFCRKDTISILS |
| | 243 Ins FCRKD | |
| | (SEQ ID NO: 163) | |
| 35 | IL7RA TM mutant | ILLRCTISILS |
| | 243 Ins RC | |
| 36 | IL7RA TM mutant | ILPCPLISILS |
| | 243 Ins PCPL (SEQ | |
| | ID NO: 164) | |
| 37 | IL7RA TM mutant | ILRCPSTISILS |
| | 243 Ins RCPS (SEQ | |
| | ID NO: 165) | |
| 38 | IL7RA TM mutant | ILLTHRGCILS |
| | 245 Ins HRGC | |
| | (SEQ ID NO: 166) | |
| 39 | IL7RA TM mutant | ILPLCSAISILS |
| | 243 Ins PLCSA | |
| | (SEQ ID NO: 167) | |
| 40 | IL7RA TM mutant | ILPIYRCVLISILS |
| | 243 Ins PIYRCVL | |
| | (SEQ ID NO: 168) | |
| 41 | IL7RA TM mutant | ILLECTISILS |
| | 242 Ins FEC | |
| 42 | IL7RA TM mutant | IFTCPSISILS |
| | 242 Ins FTCPS | |
| | (SEQ ID NO: 169) | |
| 43 | IL7RA TM mutant | ILCPSPTISILS |
| | 243 Ins CPSP (SEQ | |
| | ID NO: 170) | |
| 44 | IL7RA TM mutant | ILLTSHQPCILS |
| | 245 Ins SHQPC | |
| | (SEQ ID NO: 171) | |
| 45 | IL7RA TM mutant | ILLTISILSCSTISILSFFSVA |
| | 250 Ins CSTISILS | |
| | (SEQ ID NO: 172) | |
| 46 | IL7RA TM mutant | ILLTISICQSVA |
| | 248 Ins CQ | |
| 47 | IL7RA TM mutant | ICGIRETISILS |
| | 242 Ins CGIREI | |
| | (SEQ ID NO: 173) | |
| 48 | IL7RA TM mutant | ILLGCTISILS |
| | 243 Ins RC | |
| 49 | IL7RA TM mutant | ILLSCTISILS |
| | 244 Ins GC | |
| 50 | IL7RA TM mutant | ILLGCNISILS |
| | 243 Ins GCI | |
| 51 | IL7RA TM mutant | ILLTMRPCGSILS |
| | 245 Ins RPCG (SEQ | |
| | ID NO: 174) | |
| 52 | IL7RA TM mutant | ILLTKKCTNSILS |
| | 244 Ins KKCTN | |
| | (SEQ ID NO: 175) | |
| 53 | IL7RA TM mutant | SFFSGALLVIL |
| | V253G | |
| 54 | IL7RA TM mutant | SFFSVEKVLVIL |
| | Ins EKV | |
| 55 | IL7RA TM mutant | SFFSVEKALVIL |
| | Ins EKA | |
| 56 | IL7RA TM mutant | SFFSVEAVLVIL |
| | Ins EAV | |
| 57 | IL7RA TM mutant | ILLTMPEQDCPITILS |
| | to be combined with | |
| | S246T | |
| 58 | IL7RA | NNSSGASWCLTISILS |
| | juxtamembrane and | |
| | TM mutant 237 Ins | |
| | ASWC (SEQ ID | |
| | NO: 176) | |
| 59 | IL7RA | EMDPICPPISILS |
| | juxtamembrane and | |
| | TM mutant 242 Ins | |
| | CPP | |
| 60 | IL7RA | NLSCTKLTL |
| | juxtamembrane | |
| | mutant S185C | |

TABLE 2
Sequences of Genes and Components Described Herein

| SEQ | | |
|---|---|---|
| ID | | |
| NO: | SOURCE | SEQUENCE |
| 61 | IL7RA TM mutant | PILLPPCLTISILSFFSVALLVILACVLW |
| | 243InsPPCL (SEQ | |
| | ID NO: 160) | |
| 62 | IL7RA | NLSCTKLTL |
| | juxtamembrane | |
| | mutant S185C | |
| 63 | IL7RA TM mutant | PILLTISKCHLSFFSVALLVILACVLW |
| | 246 InsKCH | |
| 64 | IL7RA TM mutant | PIFSCGPLTISILSFFSVALLVILACVLW |
| | 241 InsFSCGP (SEQ | |
| | ID NO: 161) | |
| 65 | IL7RA TM mutant | PILLTCHLISILSFFSVALLVILACVLW |
| | 244 InsCHL | |
| 66 | IL7RA TM mutant | PILLTPPVCSVTISILSFFSVALLVILACVLW |
| | 244 InsPPVCSVT | |
| | (SEQ ID NO: 162) | |
| 67 | IL7RA TM mutant | PILLFCRKDTISILSFFSVALLVILACVLW |
| | 243 Ins FCRKD | |
| | (SEQ ID NO: 163) | |
| 68 | IL7RA TM mutant | PILLRCTISILSFFSVALLVILACVLW |
| | 243 Ins RC | |
| 69 | IL7RA TM mutant | PILPCPLISILSFFSVALLVILACVLW |
| | 243 Ins PCPL (SEQ | |
| | ID NO: 164) | |
| 70 | IL7RA TM mutant | PILLTMPEQDCPITILSFFSVALLVILACVLW |
| | to be combined with | |
| | S246T | |
| 71 | IL7RA | NNSSGASWCLTISILSFFSVALLVILACVLW |
| | juxtamembrane and | |
| | TM mutant 237 Ins | |
| | ASWC (SEQ ID | |
| | NO: 176) | |
| 72 | IL7RA | EMDPICPPISILSFFSVALLVILACVLW |
| | juxtamembrane and | |
| | TM mutant 242 Ins | |
| | CPP | |
| 73 | IL7RA TM mutant | PILRCPSTISILSFFSVALLVILACVLW |
| | 243 Ins RCPS (SEQ | |
| | ID NO: 165) | |
| 74 | IL7RA TM mutant | PILLTHRGCILSFFSVALLVILACVLW |
| | 245 Ins HRGC (SEQ | |
| | ID NO: 166) | |
| 75 | IL7RA TM mutant | PILPLCSAISILSFFSVALLVILACVLW |
| | 243 Ins PLCSA | |
| | (SEQ ID NO: 167) | |
| 76 | IL7RA TM mutant | PILPIYRCVLISILSFFSVALLVILACVLW |
| | 243 Ins PIYRCVL | |
| | (SEQ ID NO: 168) | |
| 77 | IL7RA TM mutant | PILLECTISILSFFSVALLVILACVLW |
| | 242 Ins FEC | |
| 78 | IL7RA TM mutant | PIFTCPSISILSFFSVALLVILACVLW |
| | 242 Ins FTCPS | |
| | (SEQ ID NO: 169) | |
| 79 | IL7RA TM mutant | PILCPSPTISILSFFSVALLVILACVLW |
| | 243 Ins CPSP | |
| 80 | IL7RA TM mutant | PILLTSHQPCILSFFSVALLVILACVLW |
| | 245 Ins SHQPC | |
| | (SEQ ID NO: 171) | |
| 81 | IL7RA TM mutant | PILLTISILSCSTISILSFFSVALLVILACVLW |
| | 250 Ins CSTISILS | |
| | (SEQ ID NO: 172) | |
| 82 | IL7RA TM mutant | PILLTISICQSVALLVILACVLW |
| | 248 Ins CQ | |
| 83 | IL7RA TM mutant | PICGIRETISILSFFSVALLVILACVLW |
| | 242 Ins CGIREI | |
| | (SEQ ID NO: 173) | |
| 84 | IL7RA TM mutant | PILLGCTISILSFFSVALLVILACVLW |
| | 243 Ins RC | |
| 85 | IL7RA TM mutant | PILLSCTISILSFFSVALLVILACVLW |
| | 244 Ins GC | |
| 86 | IL7RA | EMDPCRPHLTISILSFFSVALLVILACVLW |
| | juxtamembrane and | |
| | TM mutant 241 Ins | |
| | CRPH (SEQ ID NO: | |
| | 177) | |
| 87 | IL7RA TM mutant | PILLGCNISILSFFSVALLVILACVLW |
| | 243 Ins GCI | |
| 88 | IL7RA TM mutant | PILLTMRPCGSILSFFSVALLVILACVLW |
| | 245 Ins RPCG (SEQ | |
| | ID NO: 174) | |
| 89 | IL7RA TM mutant | PILLTKKCTNSILSFFSVALLVILACVLW |
| | 244 Ins KKCTN | |
| | (SEQ ID NO: 175) | |
| 90 | IL7RA TM mutant | PILLTISILSFFSGALLVILACVLW |
| | V253G | |
| 91 | IL7RA TM mutant | PILLTISILSFFSVEKVLVILACVLW |
| | Ins EKV | |
| 92 | IL7RA TM mutant | PILLTISILSFFSVEKALVILACVLW |
| | Ins EKA | |
| 93 | IL7RA TM mutant | PILLTISILSFFSVEAVLVILACVLW |
| | Ins EAV | |
| 94 | IL7RA | SGEMDPTCLTISILSFFSVALLVILACVLW |
| | juxtamembrane and | |
| | TM mutant IL241- | |
| | 242TC | |
| 95 | IL7RA | SGEMDPITLYCKTLLTISILSFFSVALLVILACVLW |
| | juxtamembrane and | |
| | TM mutant | |
| | I241 > ITLYCKT | |
| | (SEQ ID NO: 178) | |
| 96 | IL7RA TM mutant | PILLGCTISILSFFSVALLVILACVLW |
| | GCinsL243 | |
| 97 | IL7RA TM mutant | PISPCITISILSFFSVALLVILACVLW |
| | LL242-243>SPCI | |
| | (SEQ ID NO: 179) | |
| 98 | IL7RA TM mutant | PILLTISILSFFSGFSVALLVILACVLW |
| | V253>GFSV (SEQ | |
| | ID NO: 180) | |
| 99 | IL7RA TM mutant | PIDTRVYNSICLTISILSFFSVALLVILACVLW |
| | L242>DTRVYNSIC | |
| | (SEQ ID NO: 181) | |
| 100 | IL7RA TM mutant | PILLTISILSFFSVSLILIVPCACELALLVILACVLW |
| | SLILIVPCACEL | |
| | (SEQ ID NO: | |
| | 182)insA254 | |
| 101 | IL7RA TM mutant | PILLSRCLTISILSFFSVALLVILACVLW |
| | insLSRC (SEQ ID | |
| | NO: 183) (DND-41) | |
| 102 | IL7RA TM mutant | PIWAALLNCETISILSFFSVALLVILACVLW |
| | delLLinsWAALLN | |
| | CE (SEQ ID NO: | |
| | 184) (TLE39) | |
| 103 | IL7RA TM mutant | PILLTNDCSSILSFFSVALLVILACVLW |
| | dellinsNDCS (SEQ | |
| | ID NO: 185) | |
| | (TLE41) | |
| 104 | IL7RA TM mutant | PILLTISILSPLGEALLVILACVLW |
| | delFFSV (SEQ ID | |
| | NO: 186)insPLGE | |
| | (SEQ ID NO: 187) | |
| 105 | IL7RA TM mutant | PILNPCLTISILSFFSVALLVILACVLW |
| | P1 | |
| 106 | IL7RA TM mutant | PILLTCPTISILSFFSVALLVILACVLW |
| | P2 | |
| 107 | IL7RA | EMDPSANCGAISILSFFSVALLVILACVLW |
| | juxtamembrane and | |
| | TM mutant P3 | |
| 108 | IL7RA TM mutant | PILLVSCPTISILSFFSVALLVILACVLW |
| | P4 | |
| 109 | IL7RA TM mutant | PILLIISIQWLSFFSVALLVILACVLW |
| | P5 | |
| 110 | IL7RA | EMDQSPSCLTISILSFFSVALLVILACVLW |
| | juxtamembrane and | |
| | TM mutant P6 | |
| 111 | IL7RA | EMDPCLEGLTISILSFFSVALLVILACVLW |
| | juxtamembrane and | |
| | TM mutant P7 | |
| 112 | IL7RA TM mutant | PILLTISILSFFWNLLVILACVLW |
| | P8 | |
| 113 | IL7RA | EMDRFCPHISILSFFSVALLVILACVLW |
| | juxtamembrane and | |
| | TM mutant P9 | |
| 114 | IL7RA | EMDLKCILSFFSVALLVILACVLW |
| | juxtamembrane and | |
| | TM mutant P10 | |
| 115 | IL7RA TM mutant | PIFHPFNCGPISILSFFSVALLVILACVLW |
| | P11 | |
| 116 | IL7RA TM mutant | PILLMCPTISILSFFSVALLVILACVLW |
| | P12 | |
| 117 | IL7RA TM mutant | PILLTISILSFFSGPSLALLVILACVLW |
| | P13 | |
| 118 | IL7RA TM mutant | PILRLECVTISILSFFSVALLVILACVLW |
| | P14 | |
| 119 | IL7RA TM mutant | PIPQGGCILSFFSVALLVILACVLW |
| | P15 | |
| 120 | IL7RA | EMDIQSCILSFFSVALLVILACVLW |
| | juxtamembrane and | |
| | TM mutant P16 | |
| 121 | IL7RA TM mutant | PIFPHQHCTISILSFFSVALLVILACVLW |
| | P17 | |
| 122 | IL7RA TM mutant | PITLYCKTLLTISILS |
| | P18 | |
| 123 | IL7RA TM mutant | ISPCITISILS |
| | P19 | |

In the Table above, EC denotes an extracellular domain, TM denotes a transmembrane domain, and IC denotes an intracellular domain. For SEQ ID NOs. 1-12, extracellular domains are bolded, transmembrane domains are underlined and intracellular domains are neither bolded nor underlined. Where a CPT insert is present in a transmembrane domain, the sequence is both underlined and italicized. For SEQ ID NOs. 29-121, wild-type sequences are bolded and underlined, inserted sequences are bolded alone, and sequences that are neither bolded nor underlined denote juxtamembrane sequences.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Design and Testing of T Cells Expressing a Constitutively Active IL-7 Receptor Subunit

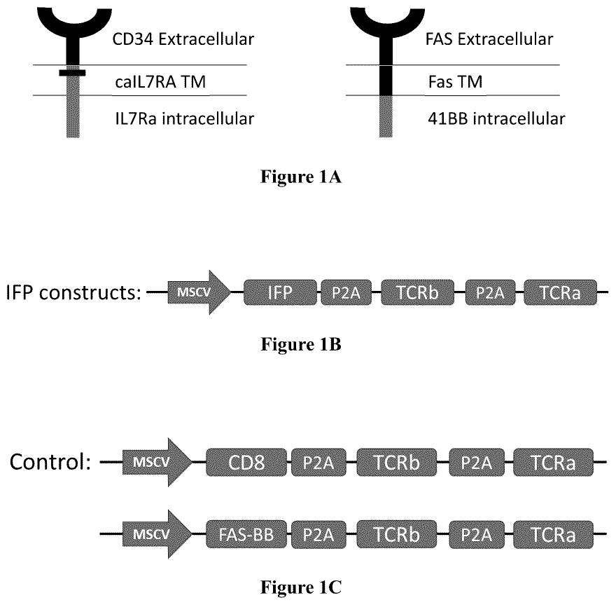

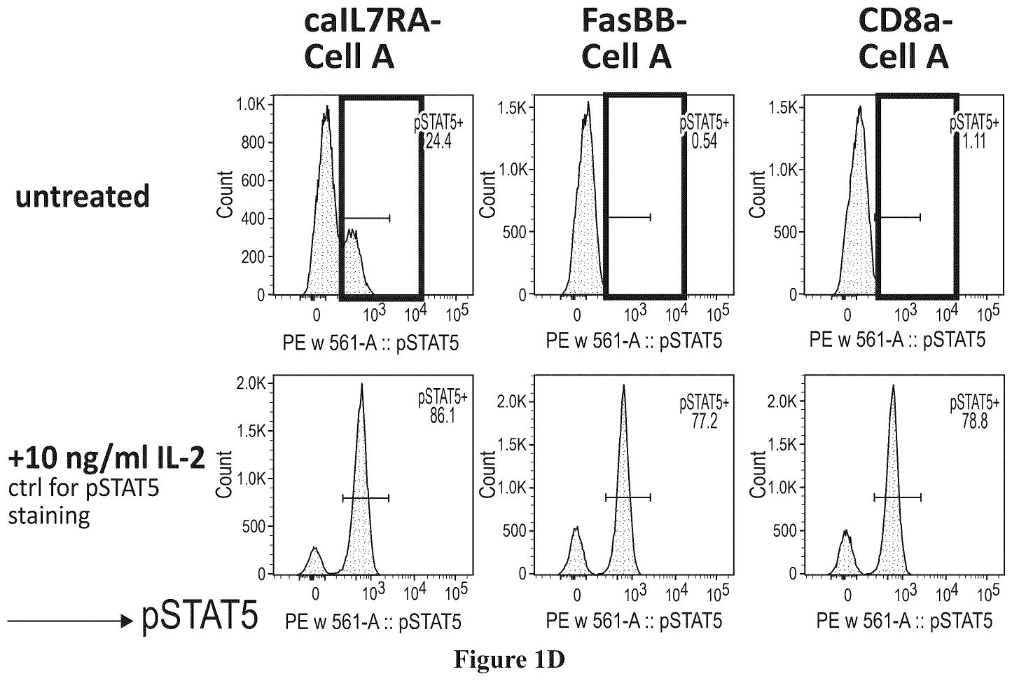

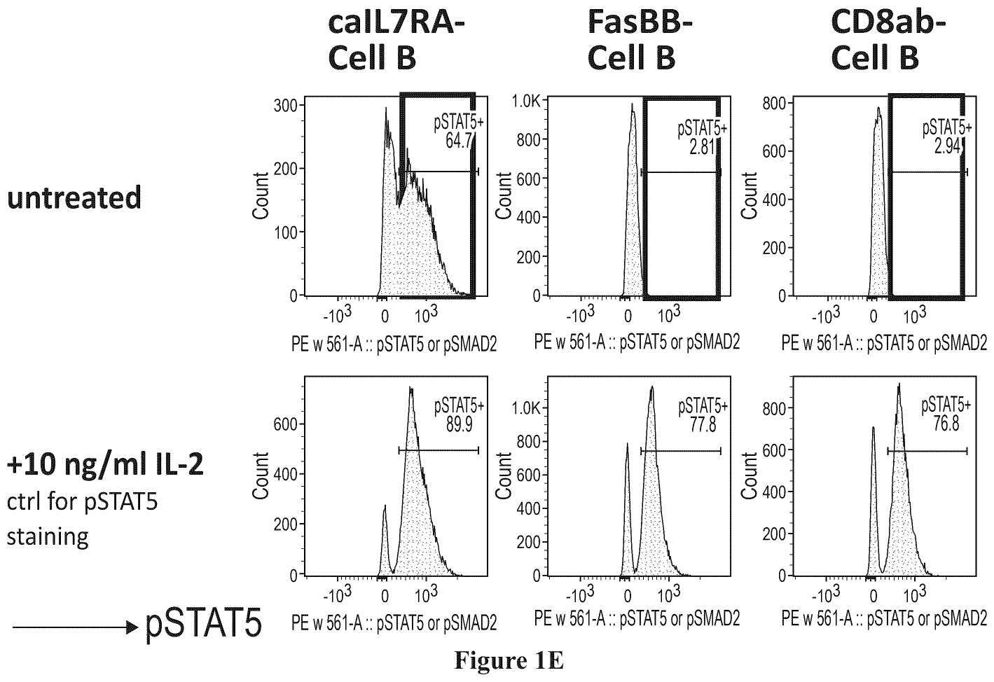

Fusion proteins were designed (FIGS. 1A-1C) and tested in anti-KRAS G12V TCR-expressing T cells (FIGS. 1D, 1E, and 2-5). In particular, a fusion protein including a CD34 extracellular domain, and a constitutively activated IL7RA receptor domain was designed and tested. The constitutively activated IL7RA receptor domain includes an IL7RA intracellular domain and an IL7RA transmembrane domain having an insertion of a trimer peptide of cysteine, proline, threonine (CPT).

pSTAT5 Signaling

Cells were cultured without IL-2, IL-15 or IL-7 for 24-72 hours before analysis. For pSTAT5 staining control, CD34-caILRA-TCR_{KRASG12V}-transduced primary CD8+ T cells were treated with 10 ng/ml IL-2 for 15-30 min, followed by pSTAT5 staining (FIGS. 1D and 1E). CD34− a CD34 extracellular domain, and a constitutively activated IL7RA receptor domain-TCRKRASG12V transduced primary CD8+ T cells showed constitutive pSTAT5 signaling. Interestingly, STAT5 staining was observed in T cells expressing the fusion protein comprising the CD34 extracellular domain, and constitutively activated IL7RA receptor domain even in the absence of ligand binding (FIGS. 1D and 1E).

### T Cell Killing

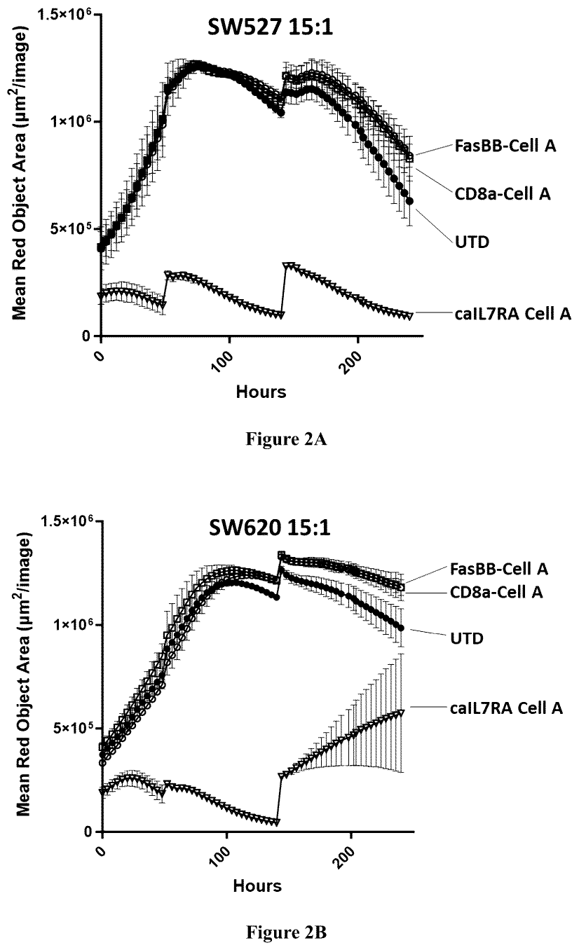

Tumor cells (e.g., from SW527 and SW620 tumor cell lines) expressing a red fluorescent protein were cultured alone or with anti-KRAS G12V TCR-transduced T cells for 240 hours at a 15:1 effector to target ratio (FIGS. 2A-2B). Total red object area was measured as metric of tumor cell growth/viability throughout the study as indicated. Additional tumor cells were added at 52 and 144 hours. anti-KRAS G12V TCR-T cells expressing the fusion protein containing a CD34 extracellular domain, and constitutively activated IL7RA receptor domain showed enhanced tumor cell killing.

TABLE 3
Transduced Cells

| Constructs | % Transduced (% ECD+) |
|---|---|
| CD34-IL7Rα | 27% |
| Fas-BB | 38% |
| Untreated (UTD) | N/A |

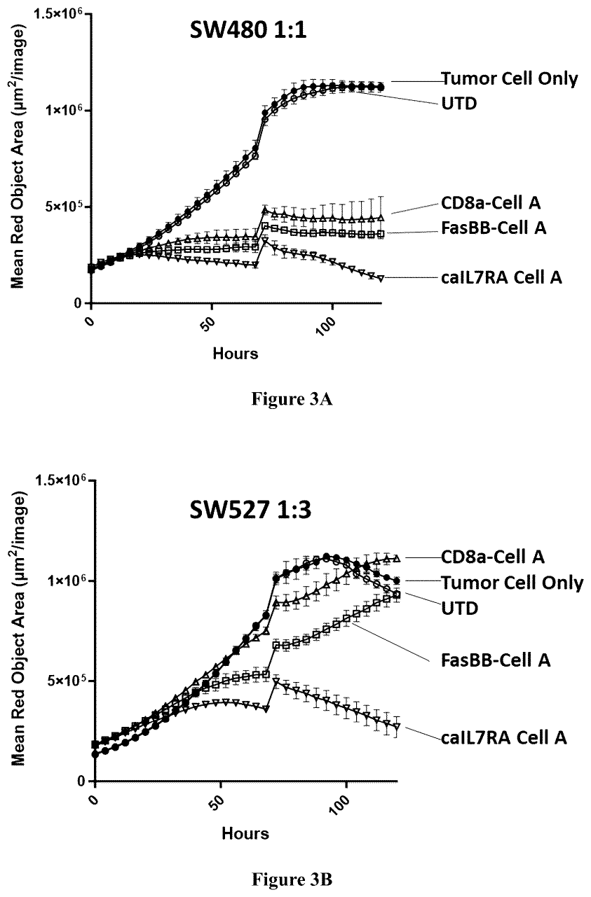

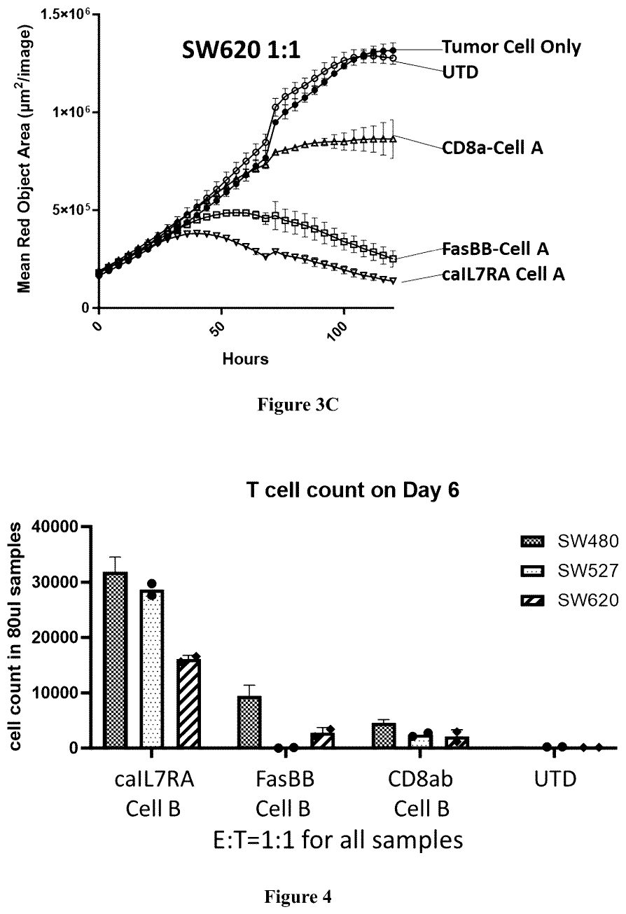

Tumor cells (e.g., from SW480, SW527 and SW620 tumor cell lines) expressing a red fluorescent protein were cultured alone or with anti-KRAS G12V TCR-transduced T cells for 120 hours at a 1:1 or 1:3 effector to target ratio (FIGS. 3A-3C). Total red object area was measured as metric of tumor cell growth/viability throughout the study as indicated. Additional tumor cells were added at 72 hours. anti-KRAS G12V TCR-T cells expressing the fusion protein containing a CD34 extracellular domain, and constitutively activated IL7RA receptor domain showed enhanced tumor cell killing.

After killing and repeated stimulation (1×), T cell count was determined by flow cytometry on Day 6 (FIG. 4). On Day 6, plates were taken out from Incucyte, transferred into equal volume (e.g., about 80 μL) samples to a new 96-well U bottom plate, and CD8+ T cells were counted using flow cytometry. Transduction of a fusion protein including a constitutively activated IL7RA receptor domain increased anti-KRAS G12V TCR-T cell proliferation upon tumor cell stimulation (FIG. 4).

### Cytokine Independent Growth Assay

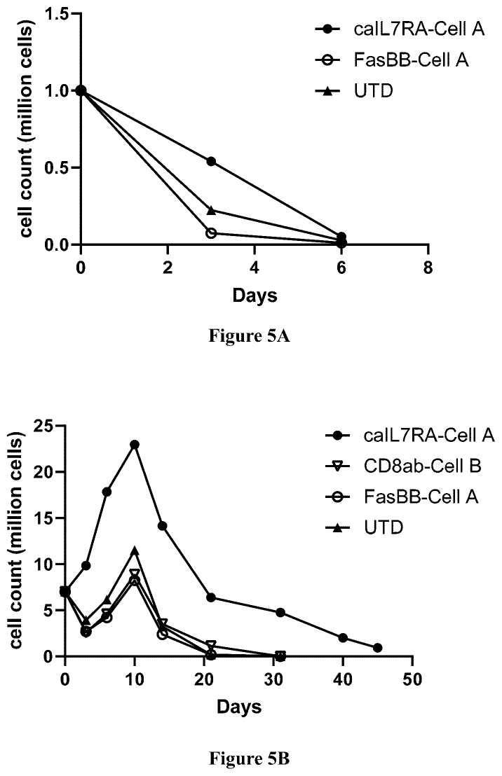

CD8 positive T cells expressing TCR_{KRASG12V} and also expressing a fusion protein containing a CD34 extracellular domain, and constitutively activated IL7RA receptor domain and controls were activated and expanded in medium with IL-2/IL-7/IL-15 for 7-10 days. On D0, 1-7 million T cells were transferred to a new 24 well bioreactor, G-Rex™, to start cytokine independent growth assays in medium without cytokine and half medium (without cytokine) was replenished every 2-4 days. Cell proliferation was monitored by counting cells every 2-4 days (FIG. 5A). In one experiment, cytokine-containing medium was used from day 3-day 10 after cytokine-independent growth assay was started and growth of all anti-KRAS G12V TCR-T samples was observed (FIG. 5B). Subsequently, medium without cytokine was used on Day 10 and continued to measure cell proliferation without cytokine after day 10. T cells expressing TCR_{KRASG12V} and expressing the fusion protein containing a CD34 extracellular domain, and constitutively activated IL7RA receptor domain did not proliferate in conditions without cytokine.

### Example 2: Design and Testing of IL7RA Fusion TCR-T Cells

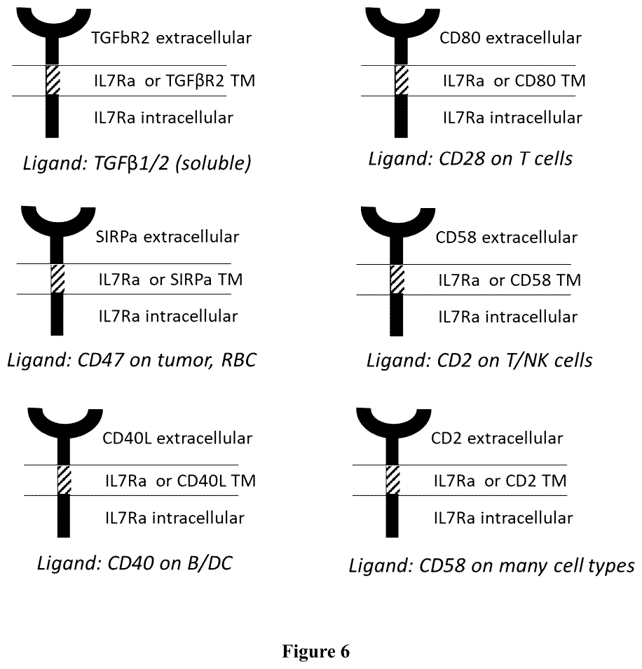

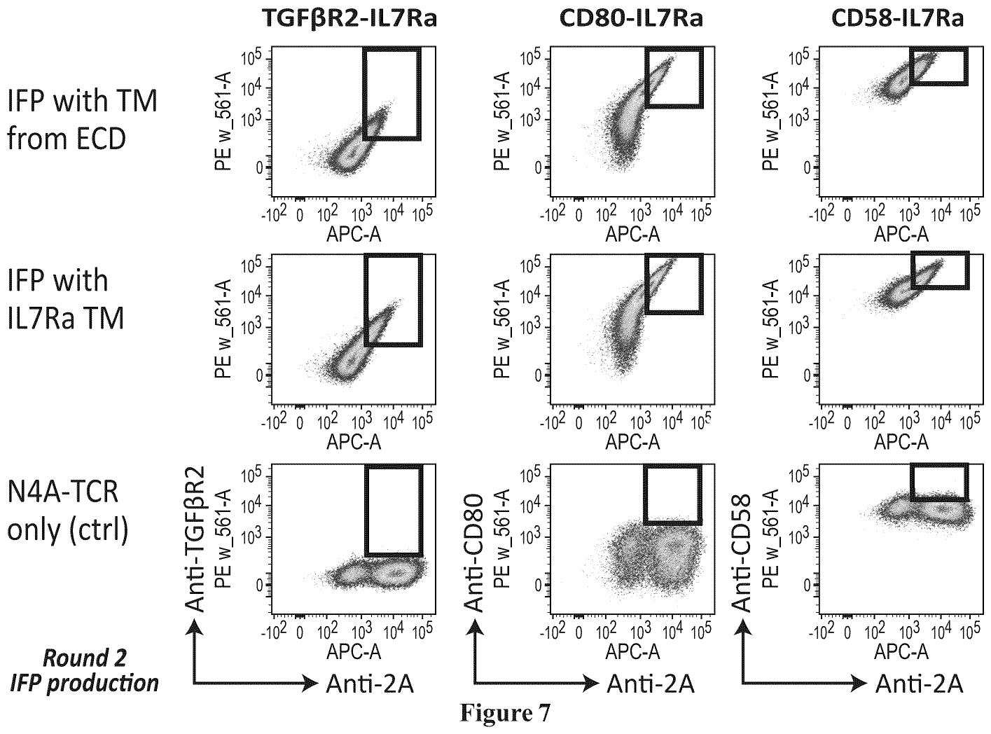

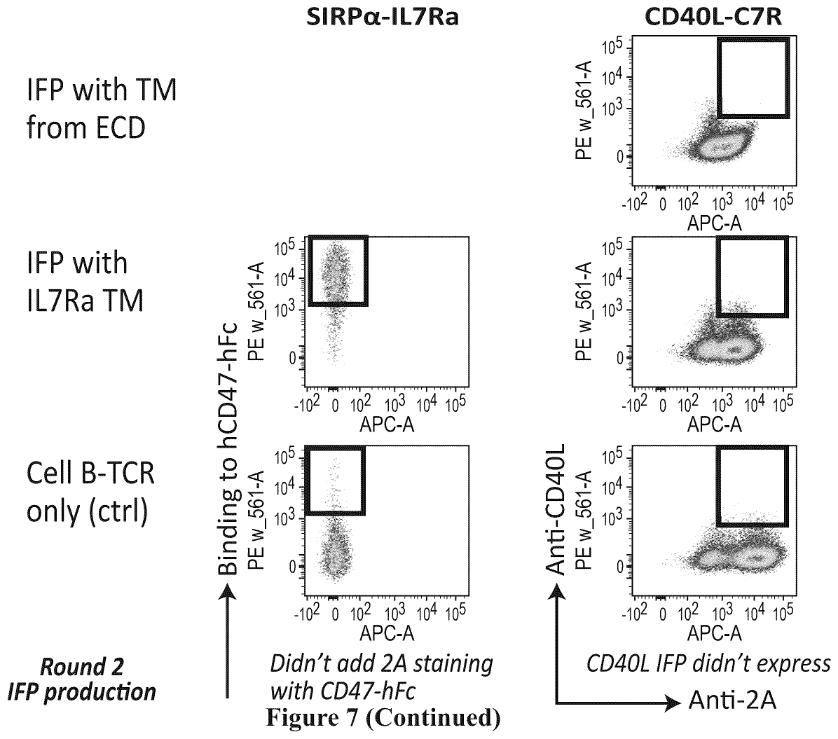

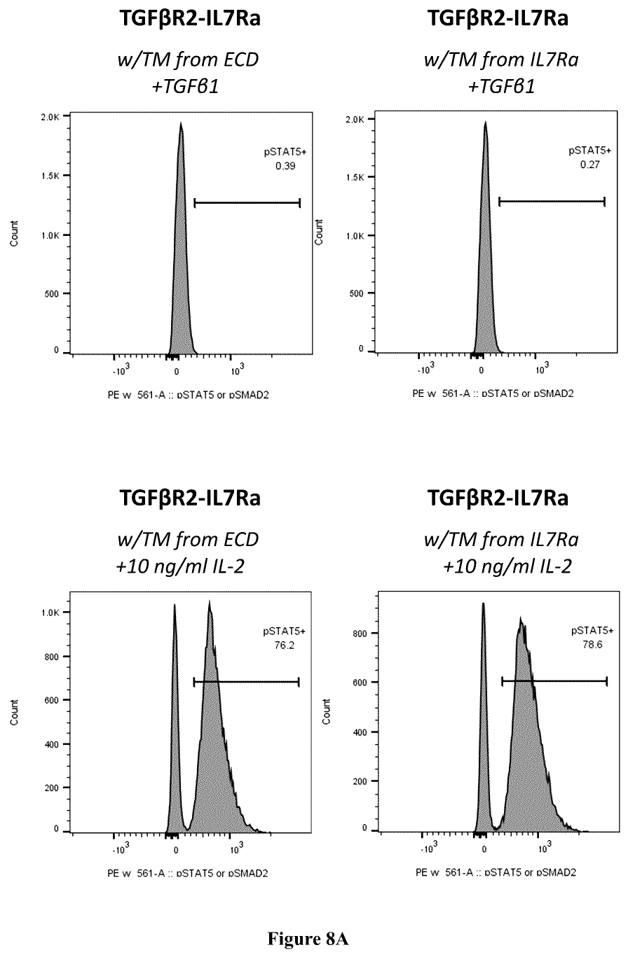

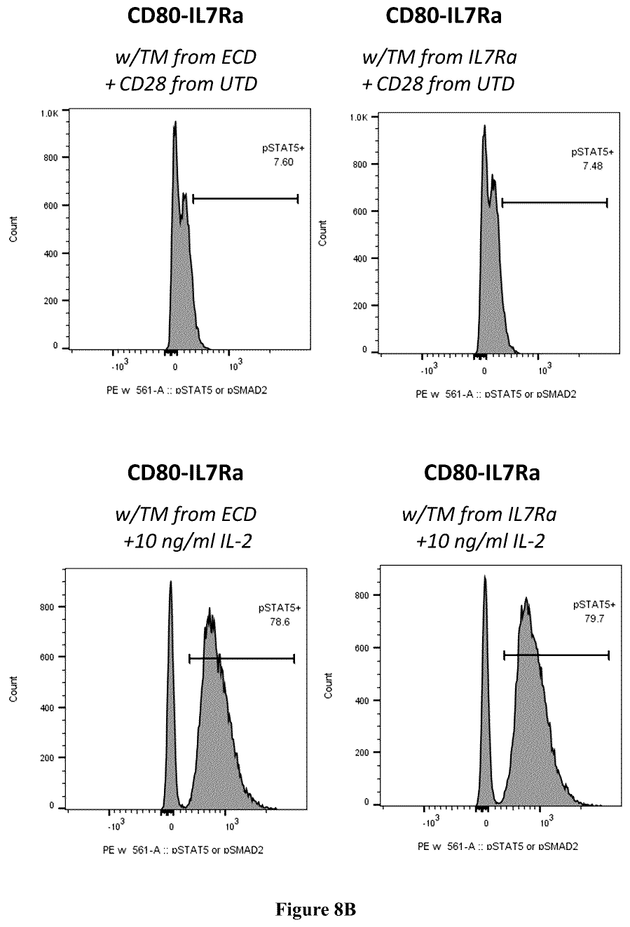

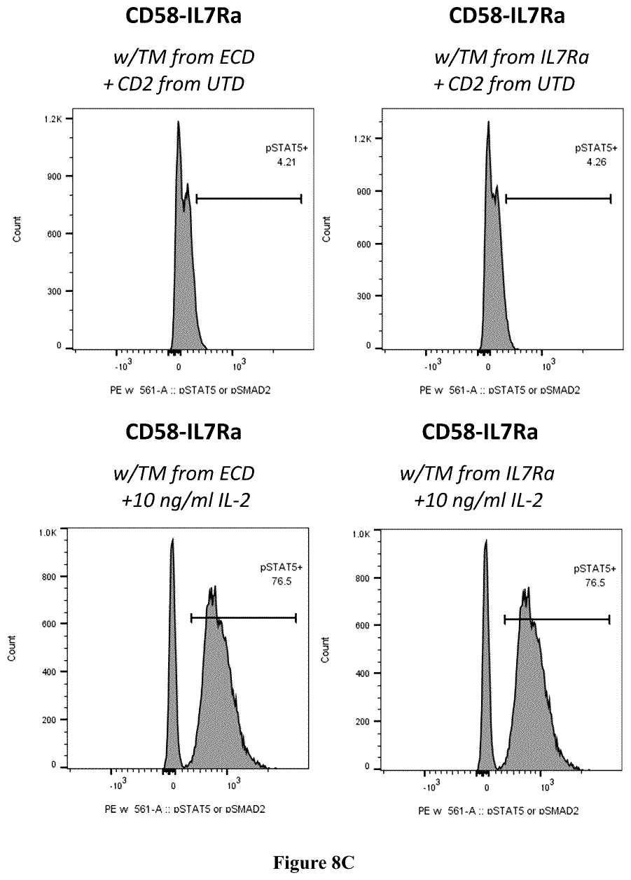

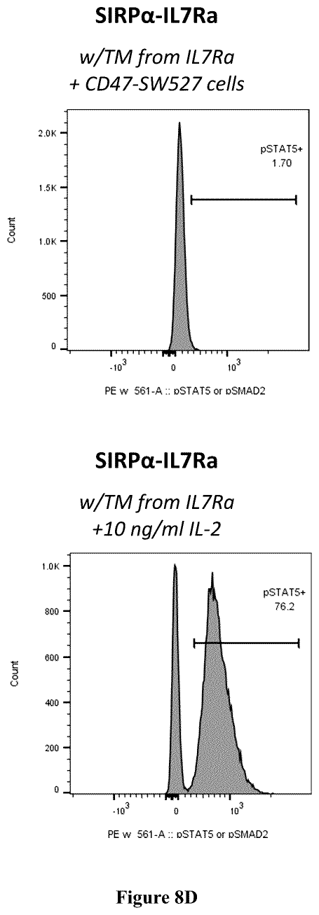

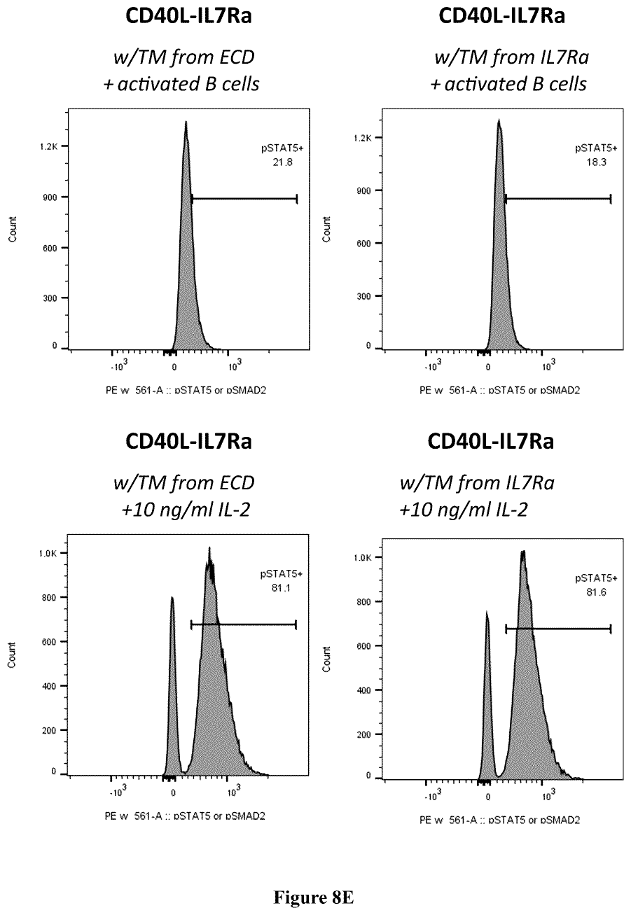

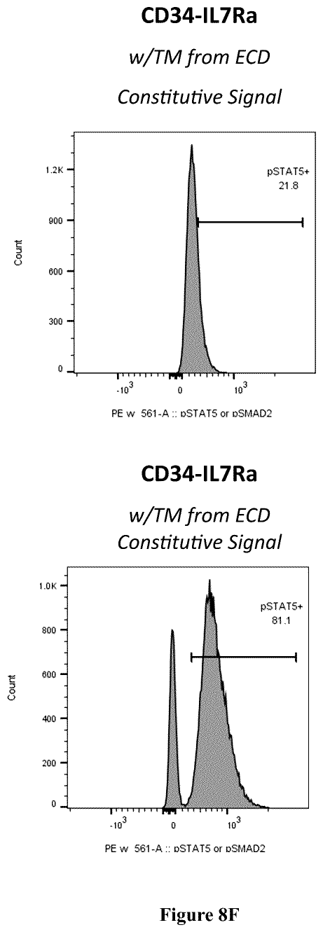

Various conditionally activated IL7RA fusion proteins were designed (FIG. 6) and tested in TCR-T cells (FIGS. 7 and 8A-8F).

### Expression

Conditional IL7RA fusion proteins comprising TGFβR2, CD80, CD58, and CD40L extracellular domains, activated upon binding of their cognate ligands, were detected by anti-TGFβR2, anti-CD80, anti-CD58, and anti-CD40L antibodies, respectively (FIG. 7). Conditional IL7RA comprising an SIRPα extracellular domain was detected through its binding to a recombinant human CD47-human Fc (hFc) fusion protein, followed by anti-Fc staining. Antibodies against a self-cleaving peptide 2A (2A) staining demonstrated transduction of the T cells by the lentivirus. Representative flow cytometric analysis confirmed expression of the conditional IL7RA fusion proteins in CD8+ T cells transduced with a polynucleotide encoding an anti-KRAS G12V TCR and the fusion proteins.

pSTAT5 Signaling

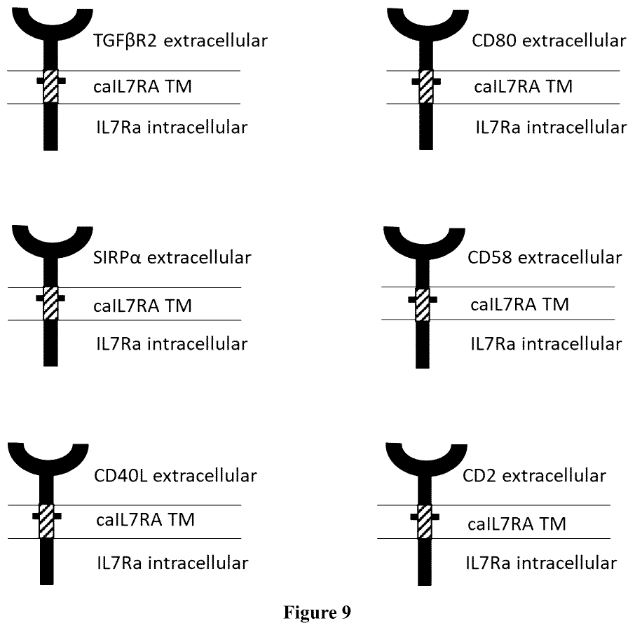

CD8+ T cells transduced with a polynucleotide encoding an anti-KRAS G12V TCR and a conditional IL7RA fusion protein described in FIG. 6 or a constitutively activated CD34-IL7RA fusion described in FIG. 9 were cultured without IL-2, IL-15 or IL-7 for 24-72 hours before analysis. For pSTAT5 staining control, the CD8+ T cells were treated with 10 ng/ml IL-2 for 15-30 min, followed by pSTAT5 staining (FIGS. 8A-8F). anti-KRAS G12V T cells transduced with polynucleotides encoding conditional IL7RA fusion proteins did not show constitutive STAT5 signaling.

### Example 3: Design and Testing of caIL7RA-TCR-T Cells

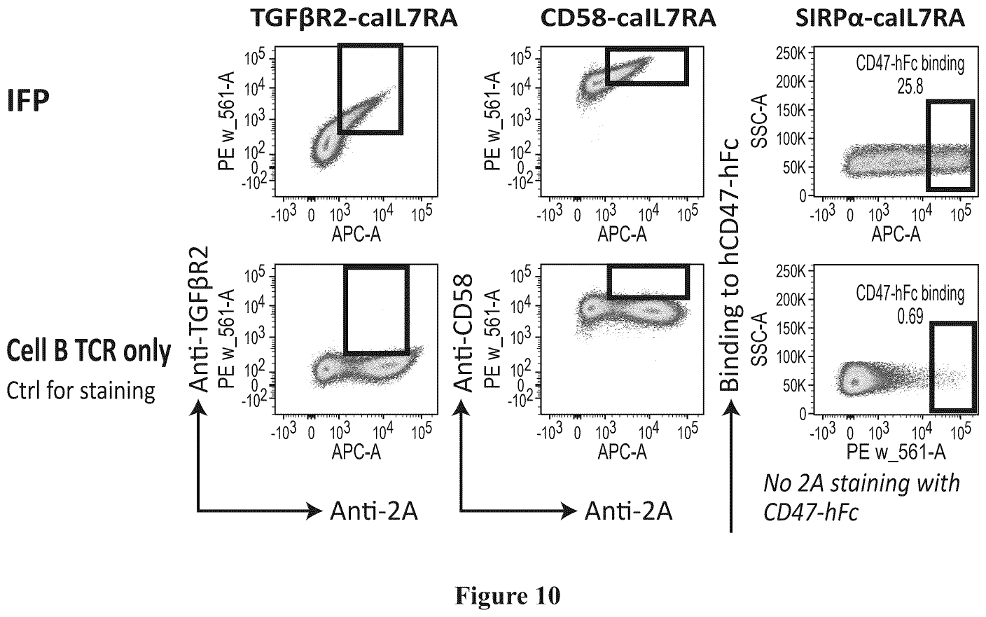

Various constitutively activated IL7RA (caIL7RA) fusion proteins were designed (FIG. 9) and tested in anti-KRAS G12V TCR-T cells (FIGS. 10-13).

### Expression

Primary CD8+ T cells were assessed by flow cytometry to detect expression of constitutively activated IL7RA fusion proteins comprising TGFβR2, CD58, or CD40L extracellular domains using anti-TGFβR2, anti-CD58, and anti-CD40L antibodies, respectively (FIG. 10). Expression of SIRPα was detected through binding of its ligand, a recombinant CD47-human Fc fusion protein, followed by anti-Fc staining. Antibodies against a self-cleaving peptide 2A (Anti-2A) staining demonstrated transduction of the anti-KRAS G12V T cells by the lentivirus. Flow cytometric analysis confirmed expression of each constitutively activated IL7RA fusion protein with the exception of CD40L-IL7R in the transduced primary CD8+ T cells.

pSTAT5 Signaling

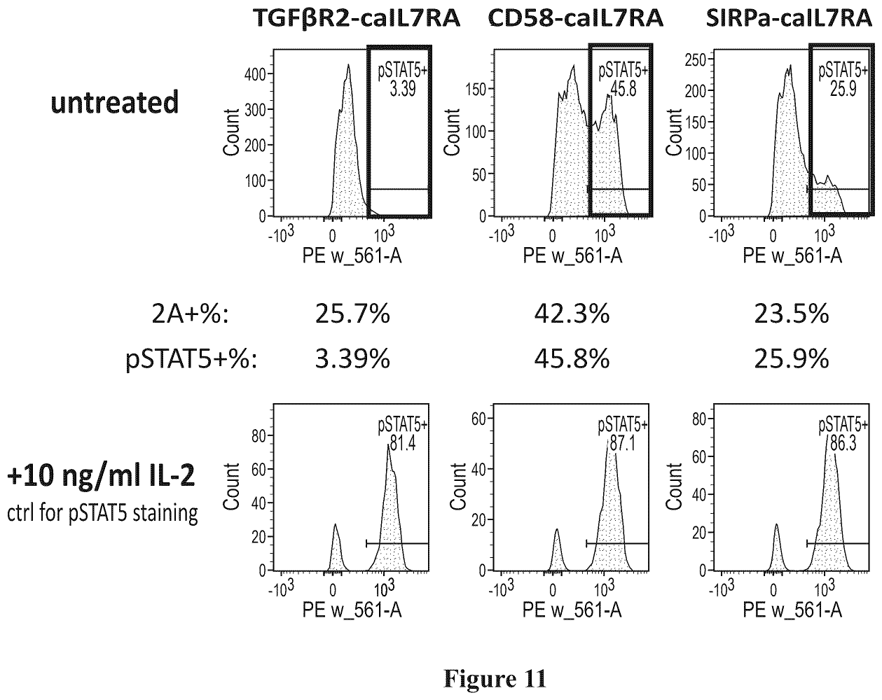

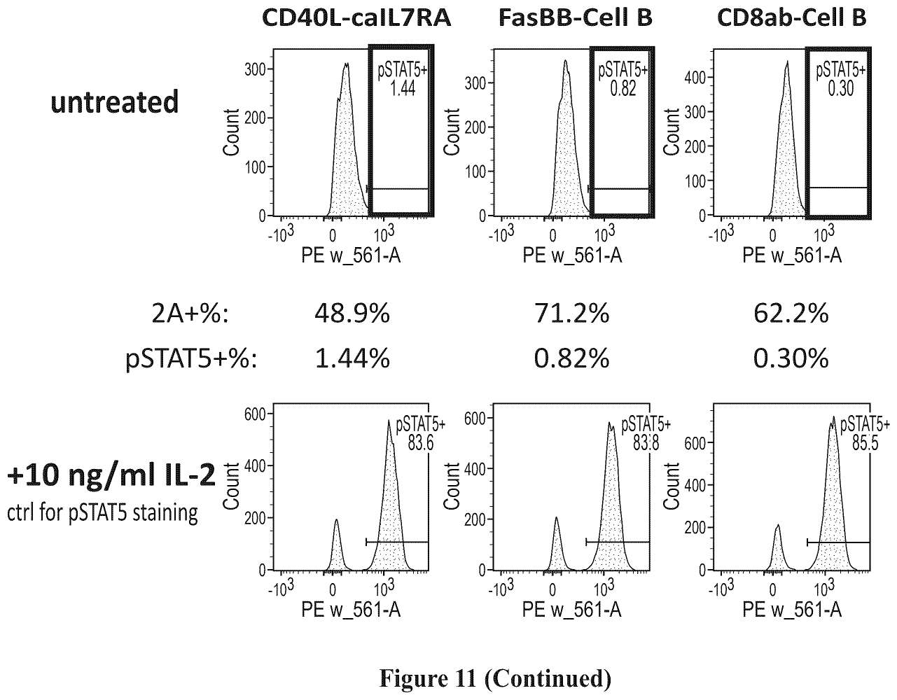

CD8+ T cells transduced with a polynucleotide encoding a constitutively activated IL7RA fusion protein and an anti-KRAS G12V TCR (see FIG. 9) were cultured without IL-2, IL-15 or IL-7 for 24-72 hours before being assessed for pSTAT expression by flow cytometry. For pSTAT5 staining control, a second sample of these cells were treated with 10 ng/ml IL-2 for 15-30 min, followed by pSTAT5 staining (FIG. 11). The experiment demonstrated that primary CD8+ T cells transduced with a polynucleotide encoding a constitutively activated CD58− or SIRPα-IL7RA fusion protein show constitutive pSTAT5 signaling FIG. 11.

### T Cell Killing

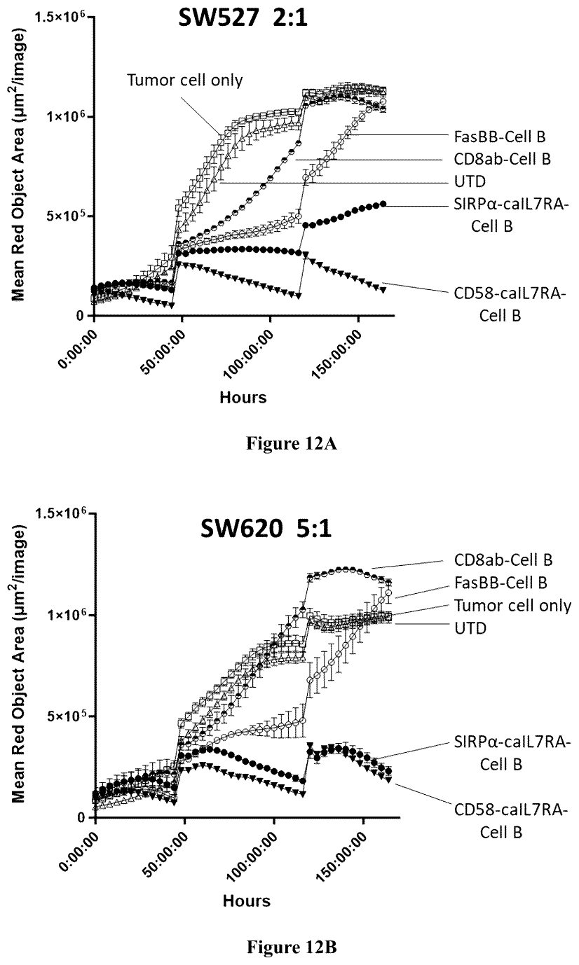

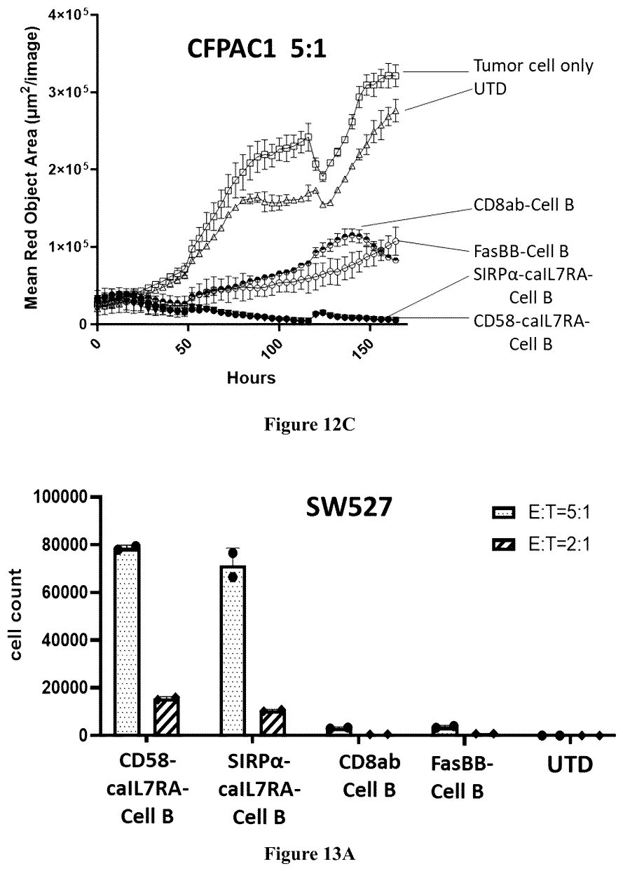

Tumor cells (e.g., from SW527, SW620, and CFPAC1 tumor cell lines) expressing a red fluorescent protein were cultured alone or with TCR− and constitutively activated IL7RA fusion protein-transduced anti-KRAS G12V T cells for 164 hours at a 2:1 or 5:1 effector to target ratio (FIGS. 12A-12C). Total red object area was measured as metric of tumor cell growth/viability throughout the study as indicated. Additional tumor cells were added at 48 and 120 hours. Primary CD8⁺ T cells transduced with a polynucleotide encoding constitutively activated IL7RA fusion proteins and an anti-KRAS G12V TCR showed enhanced tumor cell killing.

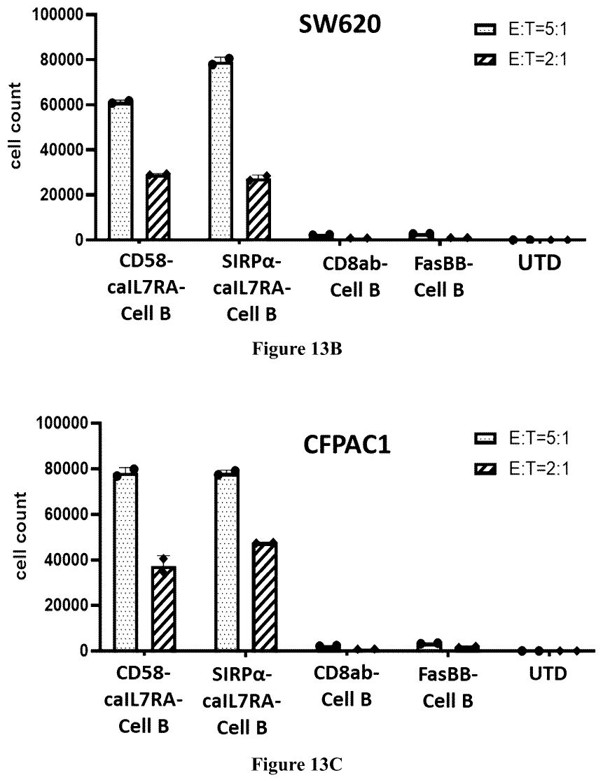

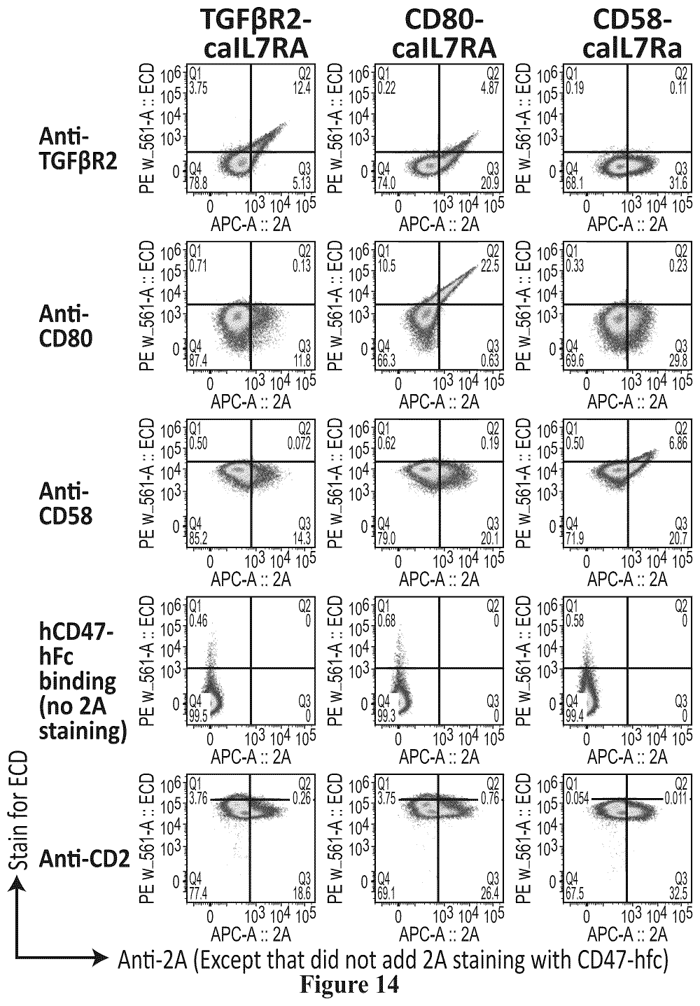

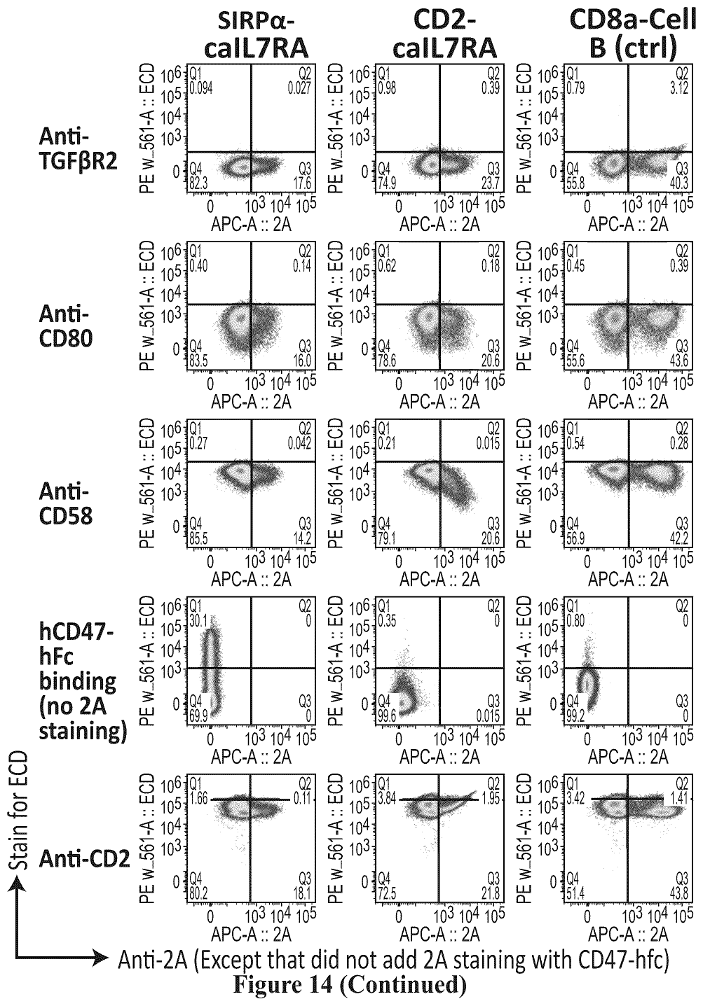

During the tumor cell killing assay with repeated stimulation (2×) with tumor cells, T cell count was determined by flow cytometry on Day 7 (FIGS. 13A-13C). On Day 7, plates were taken out from incucyte, transferred into equal volume (e.g., about 80 μL) samples to a new 96-well U bottom plate, and CD8+ T cells were counted using flow cytometry. Primary CD8⁺ T cells transduced with a polynucleotide encoding constitutively activated IL7RA fusion proteins and an anti-KRAS G12V TCR showed enhanced TCR-T cell proliferation upon tumor cell stimulation.

### Example 4: Design and Testing of caIL7RA-TCR-T Cells

### FIG. Expression

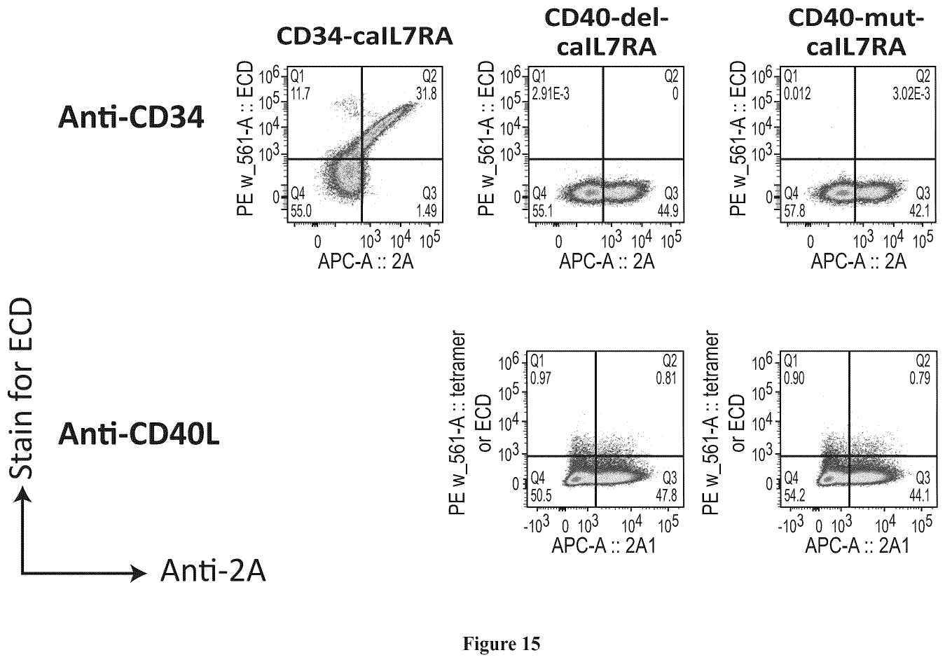

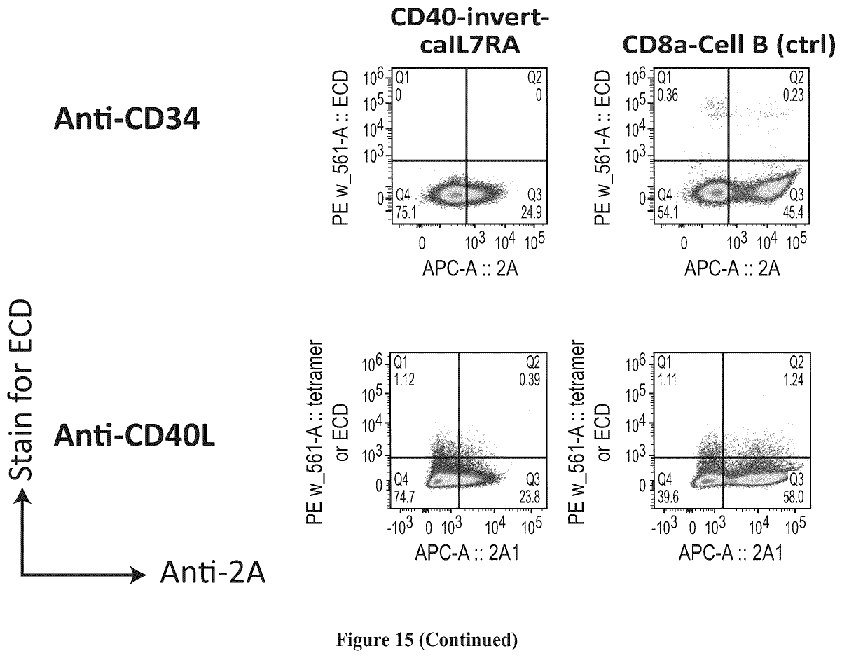

T cells transduced with a polynucleotide encoding an anti-KRAS G12V TCR and an IL7RA fusion protein comprising TGFbR2, CD80, CD58, CD2, or CD40L extracellular domain (see FIG. 9) were detected by anti-TGFbRII, CD80, CD58, CD2, CD40L antibodies, respectively (FIG. 14). Expression of CD40L-IL7RA fusion proteins having different variant IL7RA domains (e.g., mutated, inverted, or constitutively active (caIL7RA) were also assessed (FIG. 15). The T cells comprising an IL7R fusion protein having an SIRPα extracellular domain was detected through binding of its ligand, recombinant human CD47 protein with Fc fusion, followed by anti-Fc staining. T cells expressing a CD34-IL7R fusion protein were used as a control and were detected by anti-CD34 antibody. Anti-2A staining demonstrated transduction of the T cells by the lentivirus. Flow cytometric analysis confirmed expression of—IL7R fusion protein with neither a mutated nor an inverted IL7R domain in transduced primary CD8-T cells (FIGS. 14 and 15).

pSTAT5 Signaling

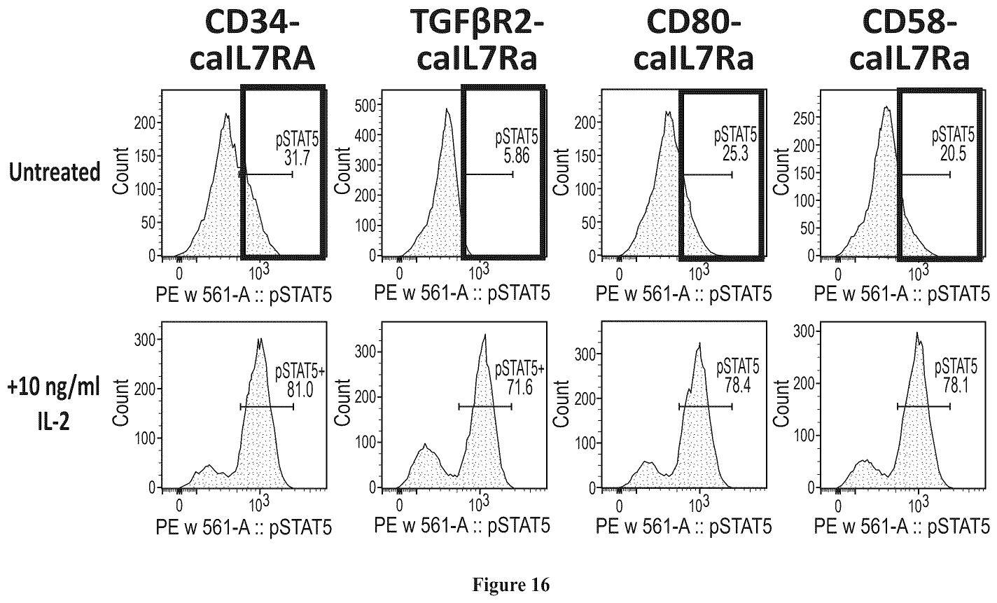

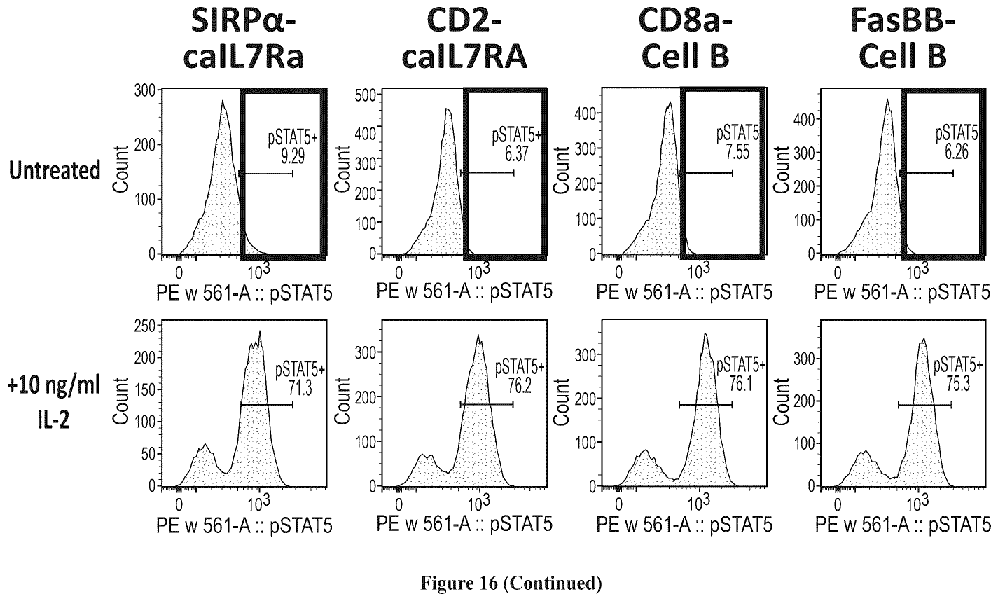

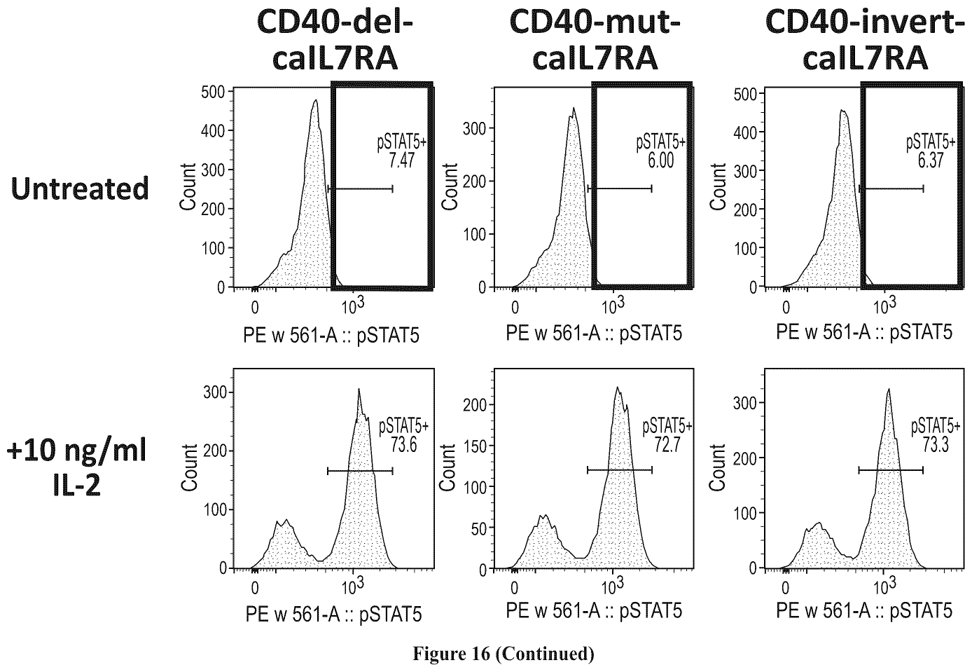

Primary CD8-T cells transduced as described above were cultured without IL-2, IL-15 or IL-7 for 24-72 hours before analysis. For pSTAT5 staining control, the transduced primary CD8-T cells were treated with 10 ng/ml IL-2 for 15-30 min, followed by pSTAT5 staining (FIG. 16). The experiment demonstrated that constitutively activated IL7RA (i.e., CD34-caIL7RA, CD58-caIL7RA, and CD80-caIL7RA)-TCR transduced primary CD8+ T cells showed constitutive pSTAT5 signaling.

### T Cell Activation

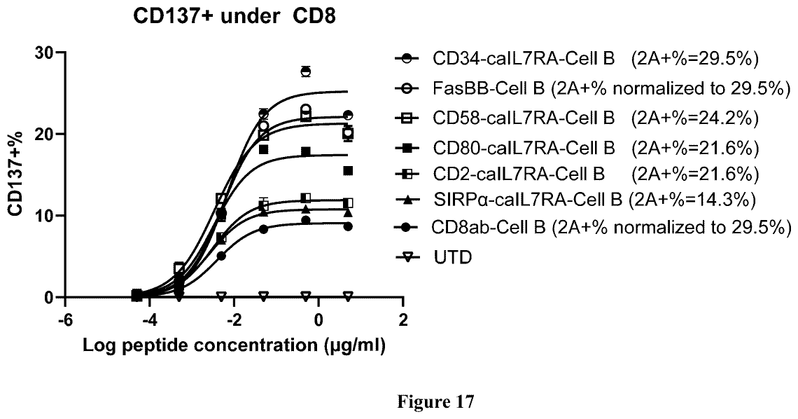

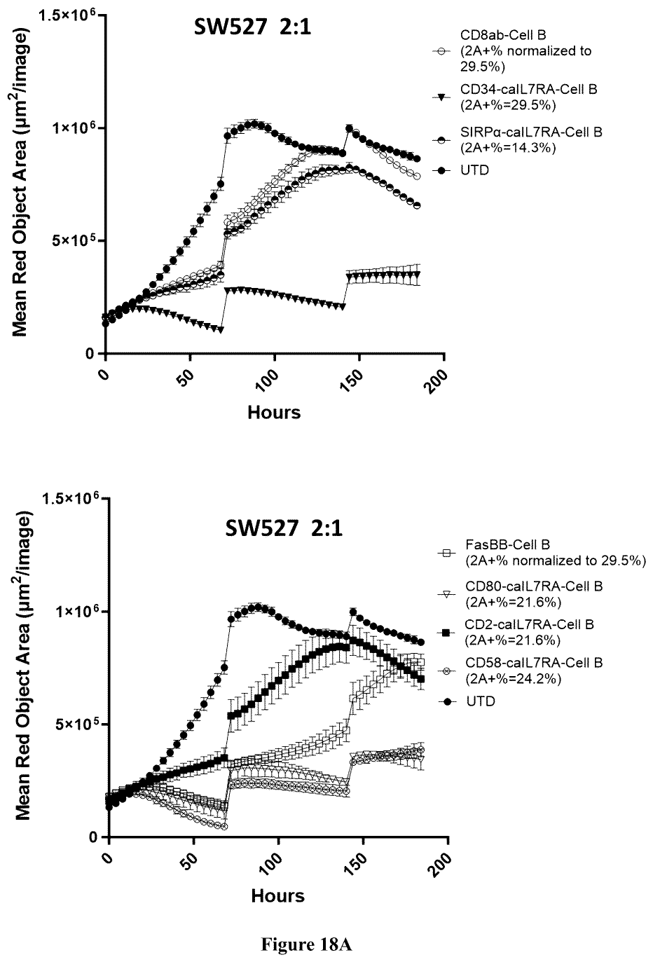

CD8+ T cells were cultured without IL-2, IL-7, and IL-15 for 1 day and then activated with index peptide at indicated concentration and expression of CD137 was quantified by flow cytometry. CD137 expression was utilized as a marker for identifying specifically activated CD8+ T cells. Primary CD8+ T cells transduced as described above showed upregulation of CD137 upon peptide-specific activation (FIG. 17).

### T Cell Killing

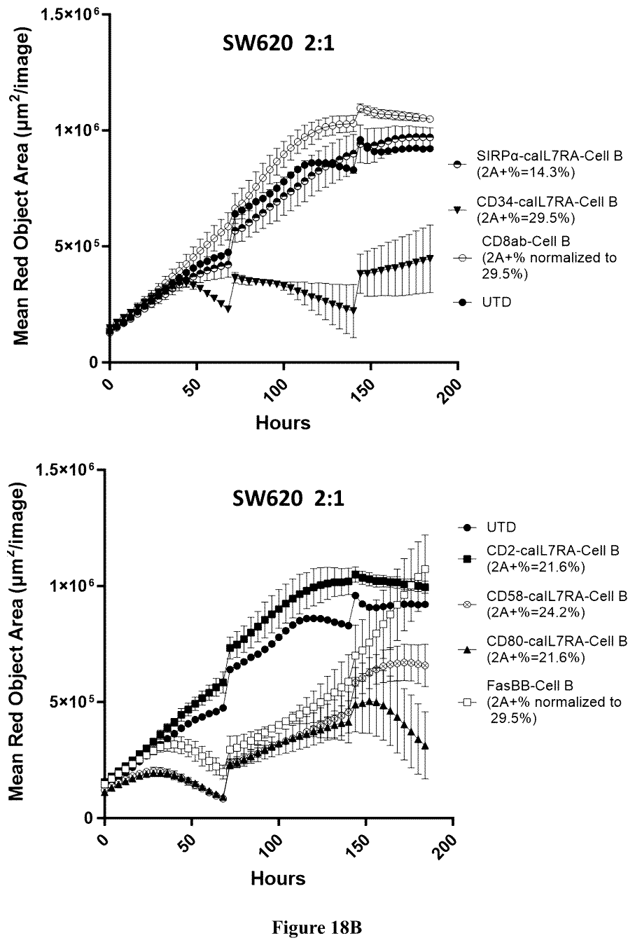

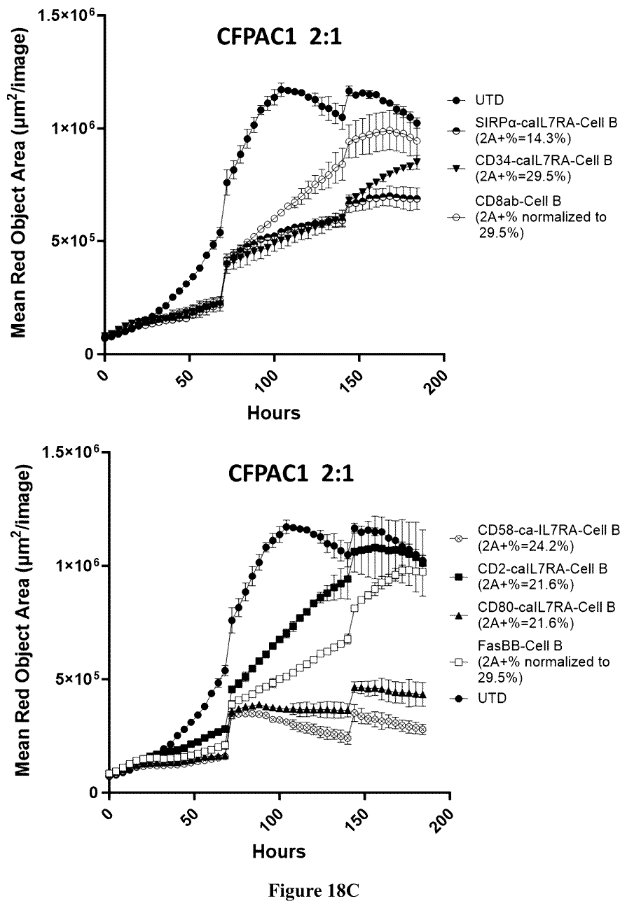

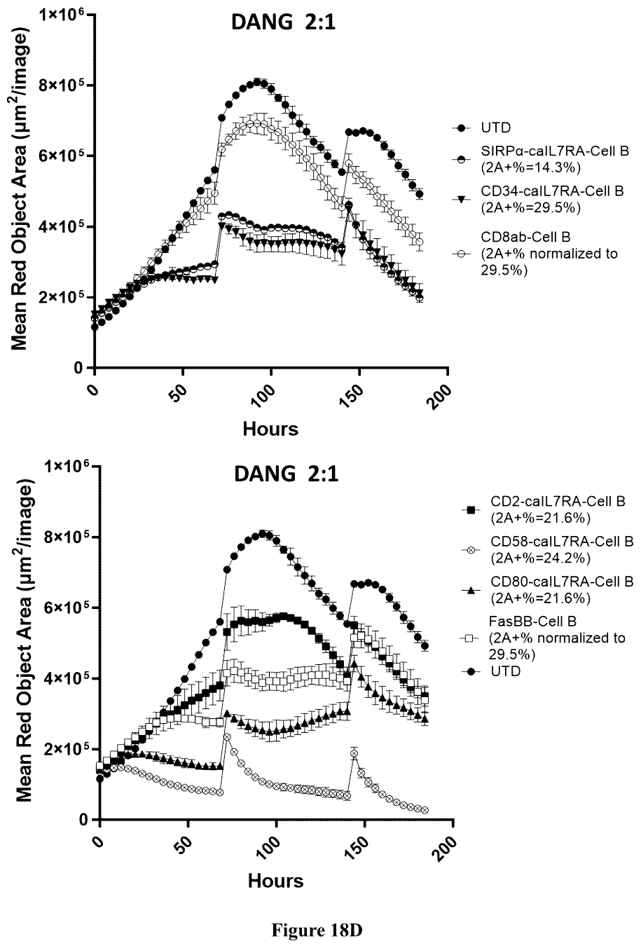

Tumor cells (e.g., from SW527, SW620, CFPAC1 and DAN-G tumor cell lines) expressing a red fluorescent protein were cultured alone or with anti-KRAS G12V TCR-transduced T cells for 184 hours at a 2:1 effector to target ratio (FIGS. 18A-18D). Total red object area was measured as metric of tumor cell growth/viability throughout the study as indicated. Additional tumor cells were added at 72 hours and 144 hours. Some transduced primary CD8+ T cells showed enhanced tumor cell killing.

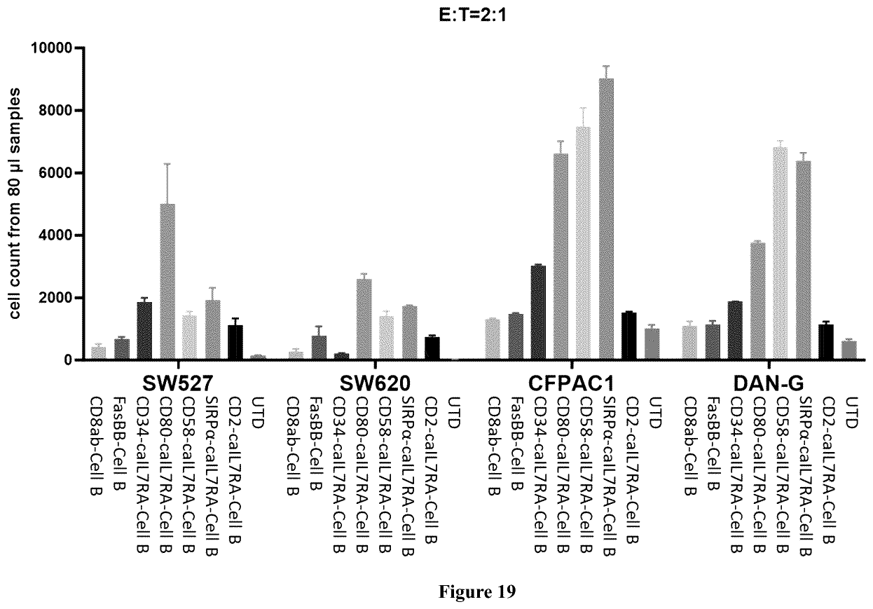

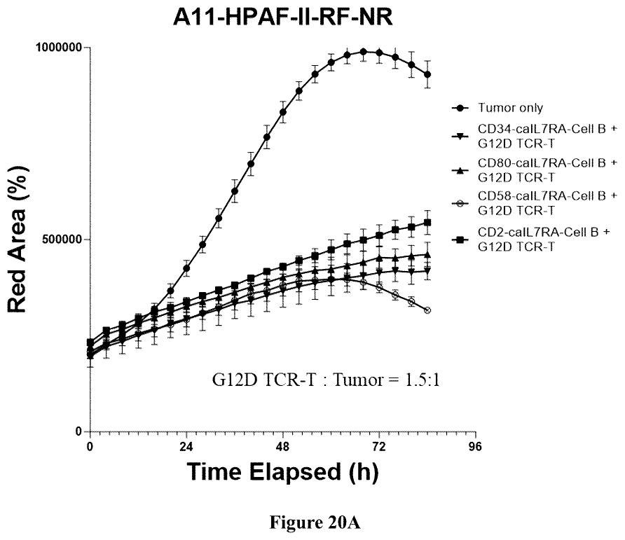

During killing and repeated stimulation (2×), T cell count was determined by flow cytometry on Day 8 (FIG. 19). On Day 8, plates were taken out from incucyte, transferred into equal volume (e.g., about 80 μL) samples to a new 96-well U bottom plate, and CD8+ T cells were counted using flow cytometry. Transduced primary CD8+ T cells showed enhanced anti-KRAS G12V TCR-T cell proliferation upon tumor cell stimulation.

### Bystander T Cell Activation

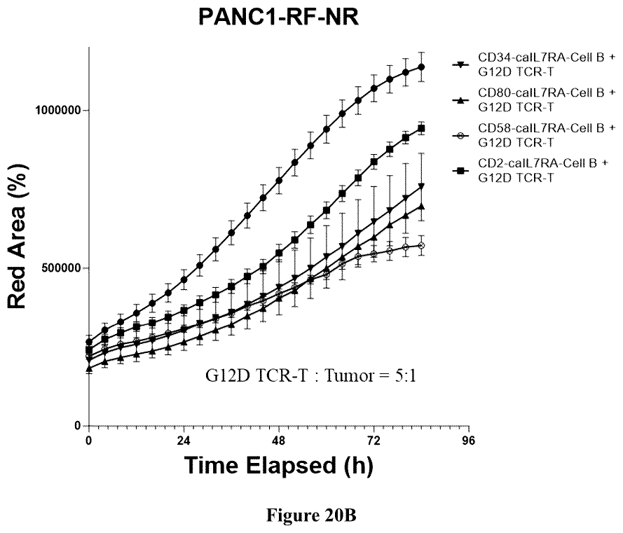

Kras G12D-mutant tumor cell lines (HPAF-II and PANC-1) expressing a red fluorescent protein were cultured alone or with TCR-transduced T cells. TCR-transduced T cells were comprised of Kras G12D TCR-T and Kras G12C TCR-T at a ratio of 1:1. Kras G12V TCR-T expressed the indicated constitutively activated IL7RA fusion protein. Kras G12D TCR-T to tumor cell ratio was 1.5:1 or 5:1. Constitutively activated CD58-IL7RA potentiate bystander T cell activation (FIGS. 20A and 20B).

### Cytokine Independent Growth Assay

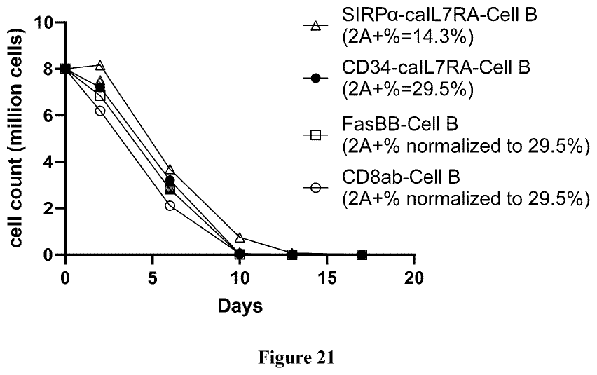

Constitutively activated IL7RA-TCR_{KRASG12V}-primary CD8+ T cells were activated and expanded in medium with IL-2, IL-7, IL-15 for 13 days. On D0, 8 million T cells were transferred to a new 24-well production platform (e.g., G-Rex™) to start cytokine independent growth assays. T cells then transferred into medium without cytokine and Half medium (without cytokine) were replenished every 2-4 days (FIG. 21). Cell proliferation was monitored by counting cells every 3-4 days. Primary CD8+ T cells transduced with polynucleotides encoding an anti-KRAS G12V TCR and a constitutively activated IL7RA fusion protein did not proliferate in conditions without cytokine.

### Example 5: Design and Testing of caIL7RA-TCR-T Cells

### Expression

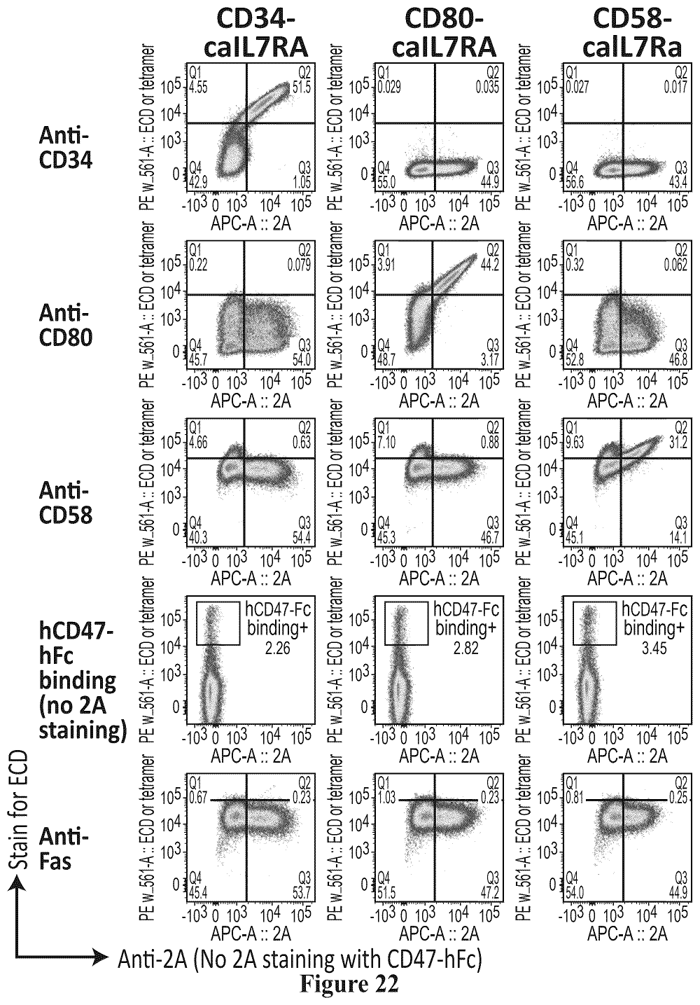

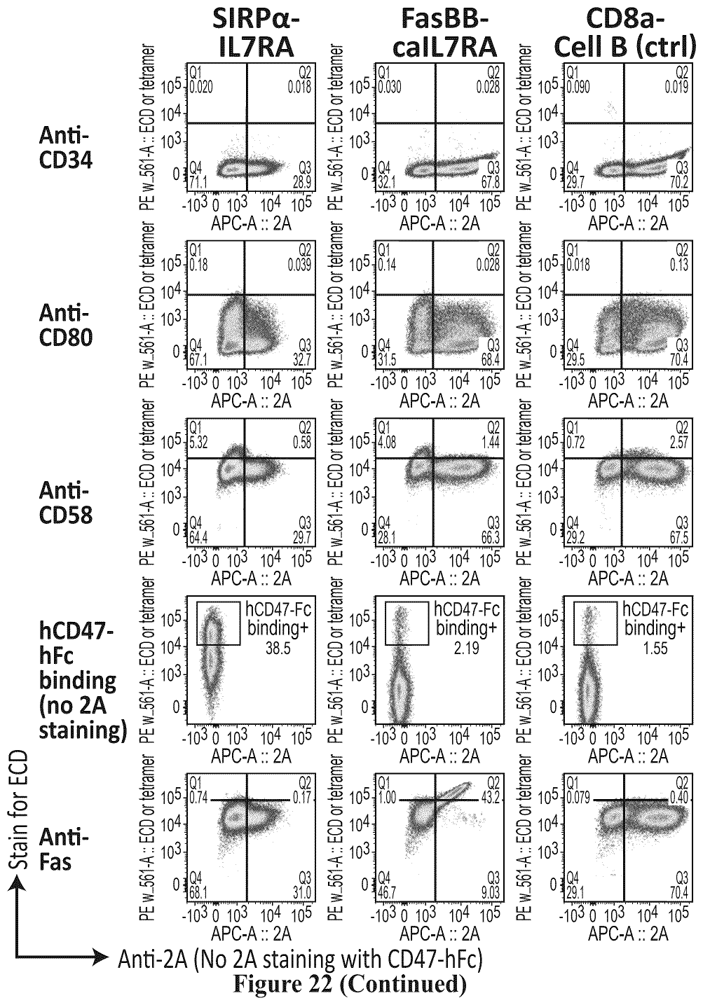

Constitutively activated IL7RA-fusion proteins comprising CD80 and CD58 extracellular domains and expressed in T cells expressing anti-KRAS G12V TCRs were detected by anti-CD80, and CD58 antibodies, respectively (FIG. 22). IL7R fusion proteins comprising an SIRPα extracellular domain were detected through binding of recombinant human CD47 protein with Fc fusion, followed by anti-Fc staining. CD34-IL7RA fusion protein was used as control, and the extracellular domain was detected by anti-CD34 antibody. Anti-2A staining demonstrated transduction of the T cells by the lentivirus. Flow cytometric analysis confirmed expression of—the exogenous TCR and the IL7RA fusion protein in transduced mixture of primary CD4 and CD8 T cells.

pSTAT5 Signaling

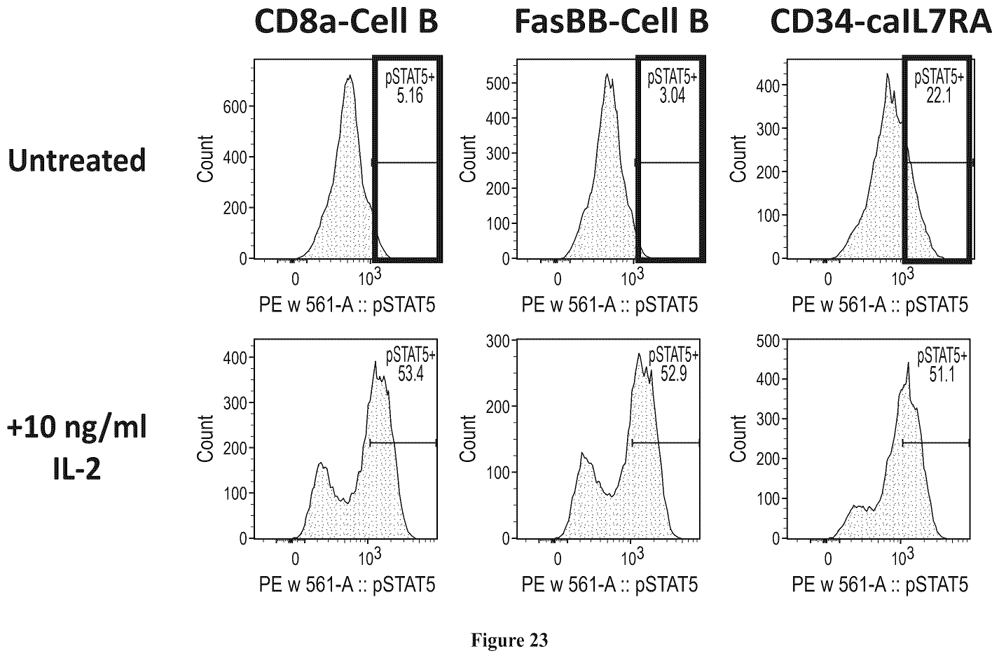

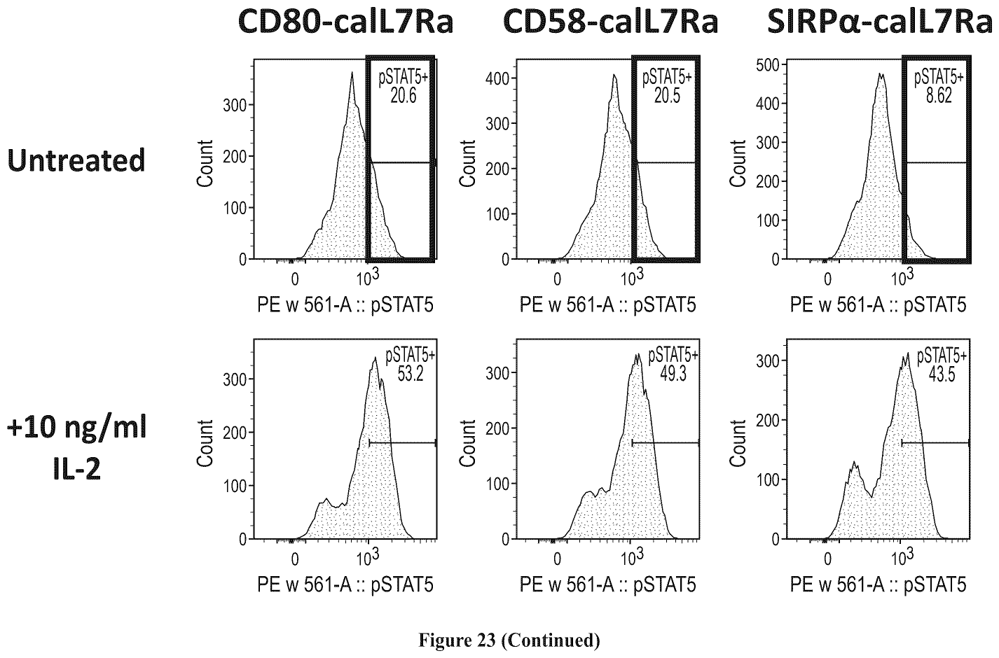

Primary CD4+ and CD8+ T cells transduced with polynucleotides encoding an anti-KRAS G12V TCR and an IL7RA fusion protein were cultured without IL-2, IL-15 or IL-7 for 24-72 hours before analysis. For pSTAT5 staining control, a transduced mixture of primary CD4 and CD8 T cells was treated with 10 ng/ml IL-2 for 15-30 min, followed by pSTAT5 staining (FIG. 23). The transduced mixture of primary CD4+ and CD8+ T cells induced pSTAT5 signaling without ligand.

T cell Activation

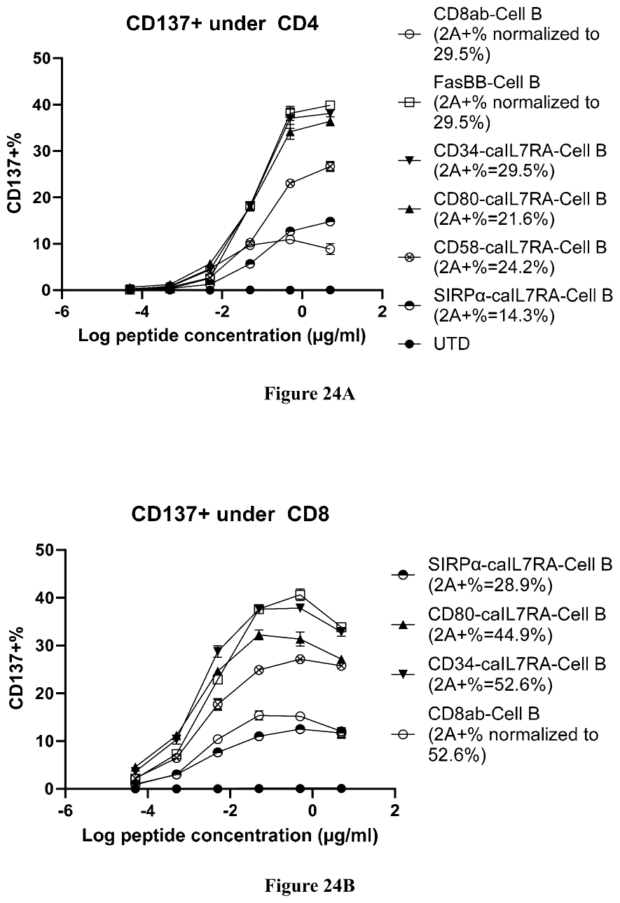

A mixture of CD4+ and CD8+ T cells were cultured without IL-2, IL-7, and IL-15 for 1 day and then activated with index peptide at indicated concentration and expression of CD137 was quantified by flow cytometry. CD137 expression was utilized as a marker for identifying specifically activated mixture of CD4+ and CD8+ T cells. A transduced mixture of primary CD4+ and CD8+ T cells show upregulation of CD137 upon peptide-specific activation (FIGS. 24A and 24B). It was noted that some receptors showed higher increases in CD137 upregulation versus others.

### T Cell Killing

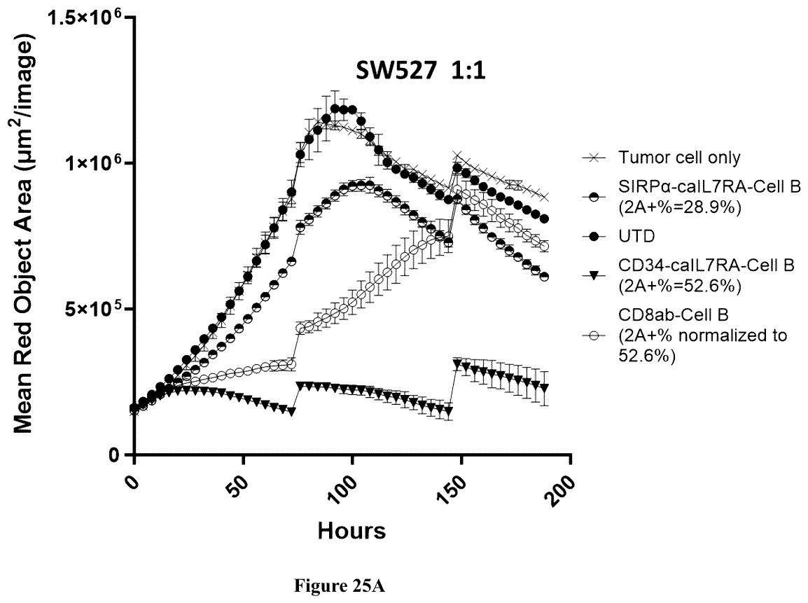

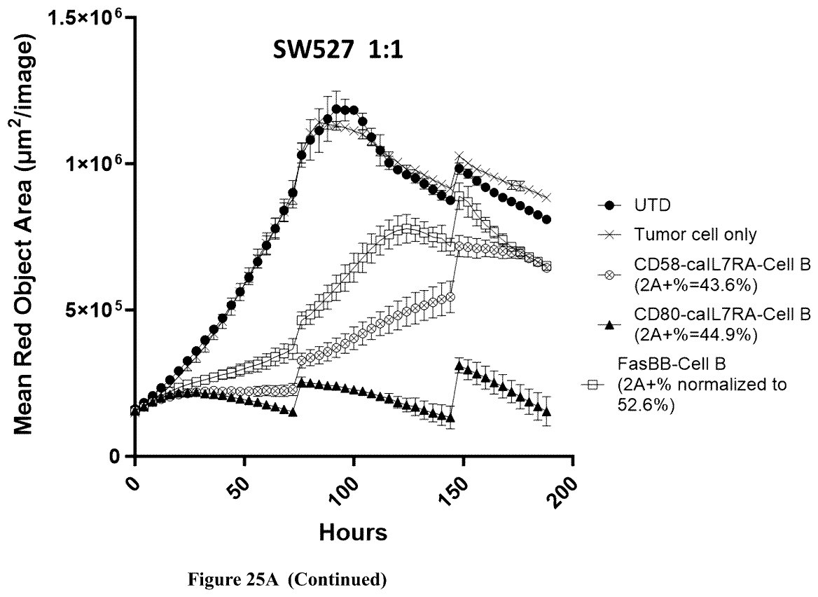

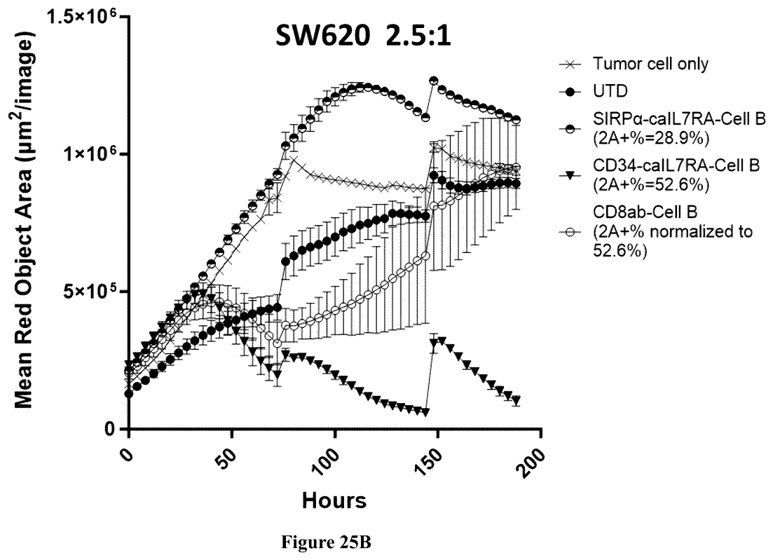

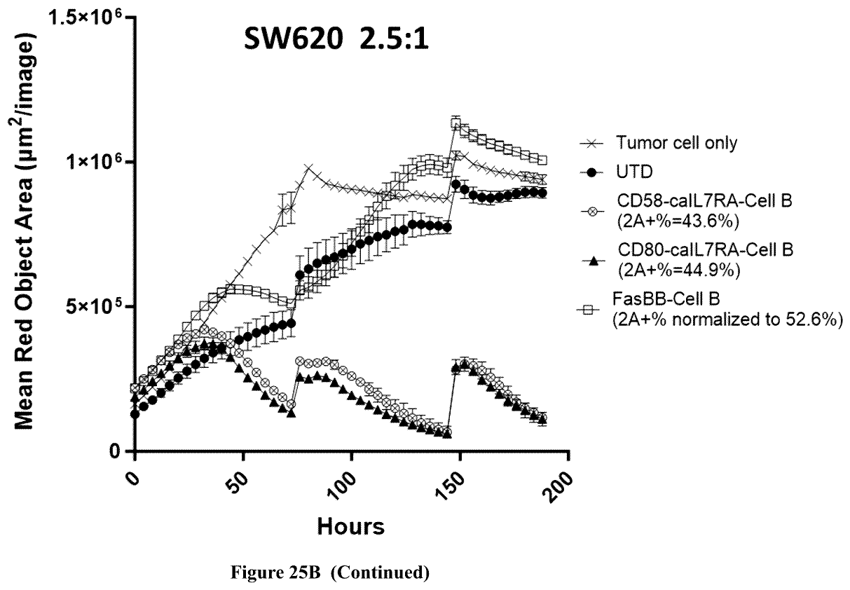

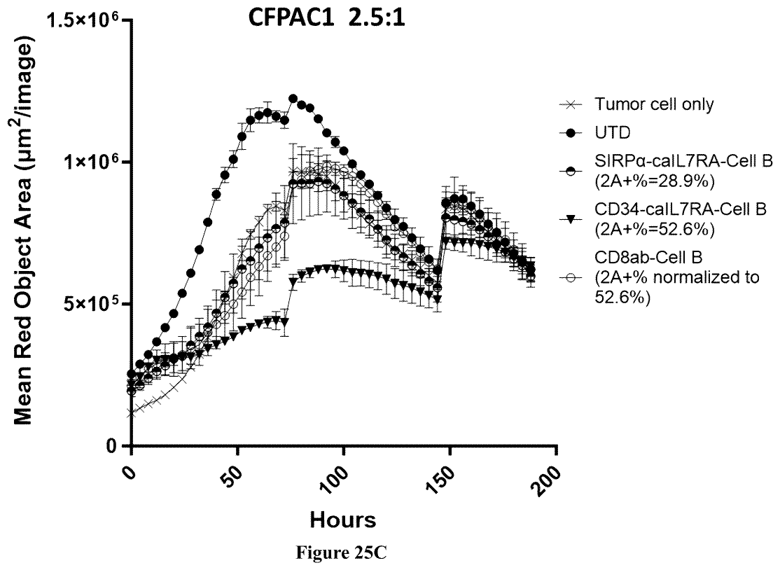

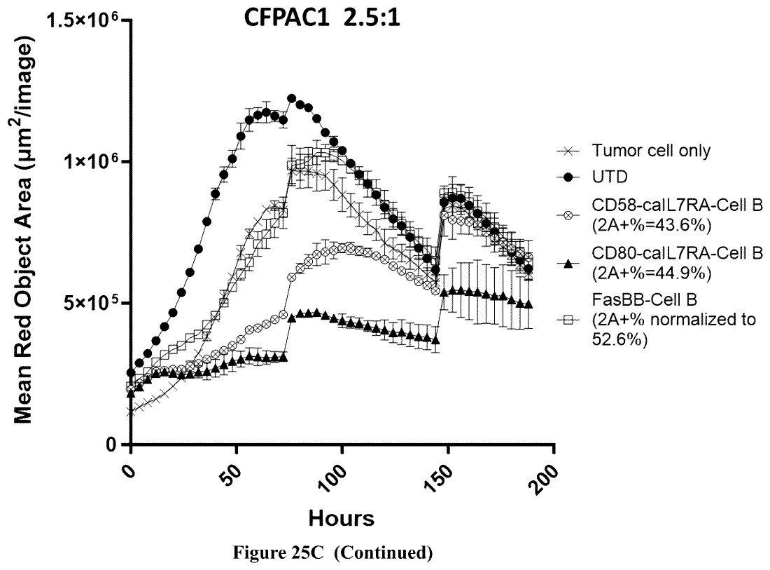

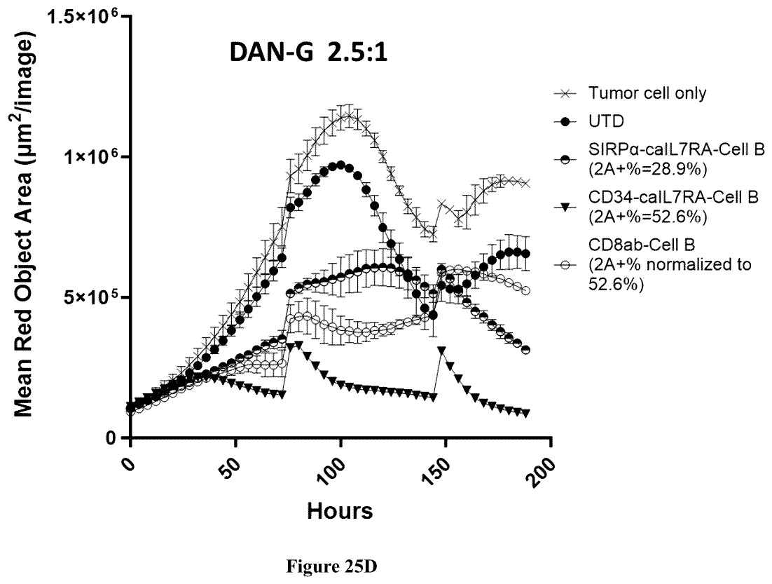

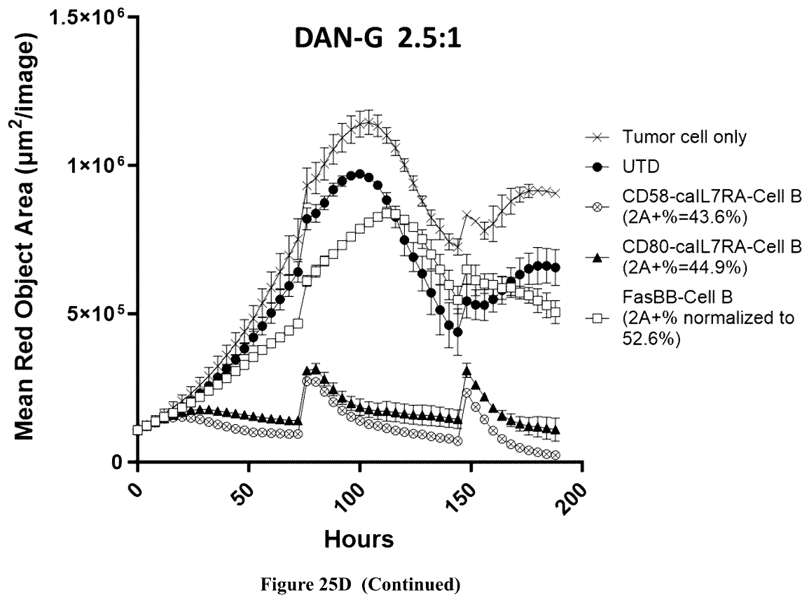

Tumor cells (e.g., from SW527, SW620, CFPAC1 and DAN-G tumor cell lines) expressing a red fluorescent protein were cultured alone or with TCR-transduced T cells for 188 hours at a 1:1 or 2.5:1 effector to target ratio (FIGS. 25A-25D). Total red object area was measured as metric of tumor cell growth/viability throughout the study as indicated. Additional tumor cells were added at 76 hours and 144 hours. A transduced mixture of primary CD4+ and CD8+ T cells showed enhanced tumor cell killing.

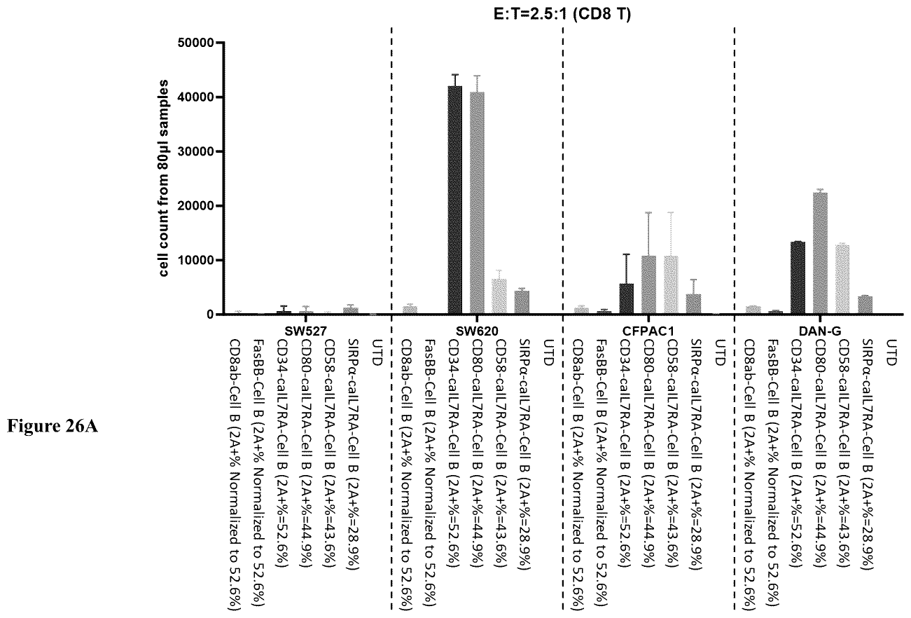

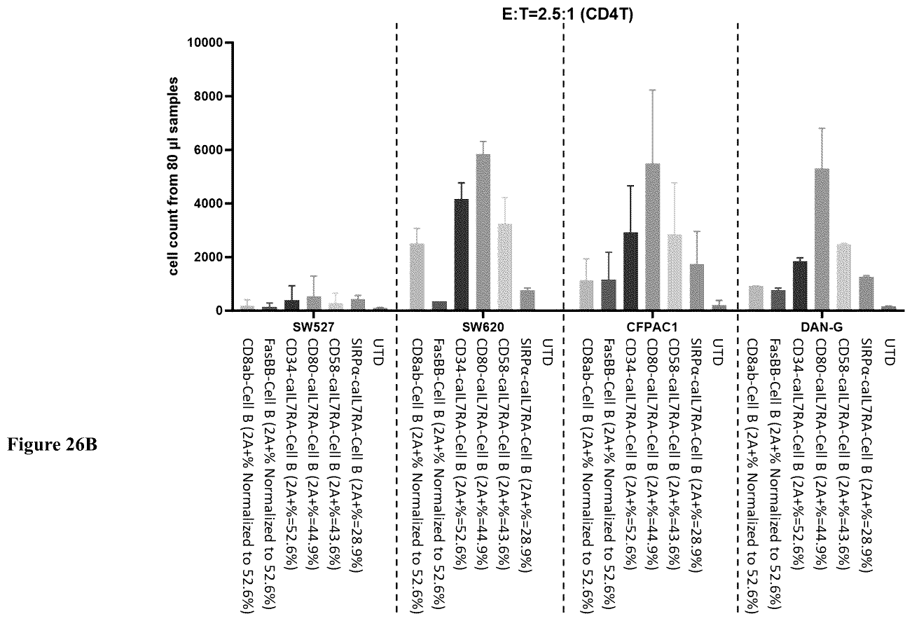

After killing and repeated stimulation (2×), T cell count was determined by flow cytometry on Day 8. On Day 8, plates were removed from the cell analysis system (Incucyte®), transferred into equal volume (e.g., about 80 μL) samples to a new 96-well U bottom plate, and a mixture of CD4+ and CD8+ T cells were counted using flow cytometry. The transduced primary CD4+ T cells and CD8+ T cells showed enhanced TCR-T cell proliferation upon tumor cell stimulation (FIGS. 26A and 26B).

### Cytokine Independent Growth Assay

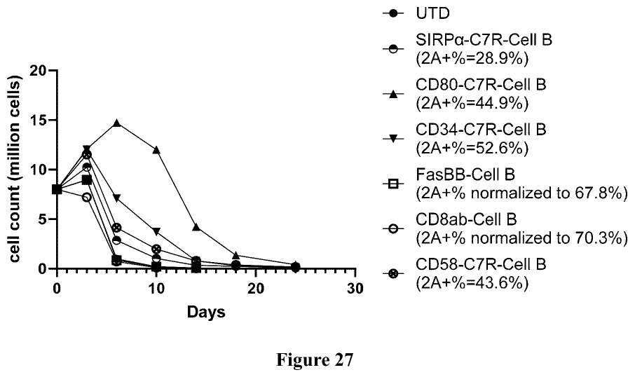

A transduced primary mixture of CD4 and CD8 T cells were activated and expanded in medium with IL-2, IL-7, and IL*15 for 10 days. On D0, 8 million T cells were transferred to a new 24-well G-Rex to start cytokine independent growth assays. T cells were then transferred into medium without cytokine and Half medium (without cytokine) were replenished every 2-4 days (FIG. 27). Cell proliferation was monitored by counting cells every 3-4 days. the transduced primary mixture of CD4+ and CD8+ T cells did not proliferate in conditions without cytokine.

### Example 6: Testing of Mutated IL7R Transmembrane Domain-TCR-T Cells

pSTAT5 Signaling

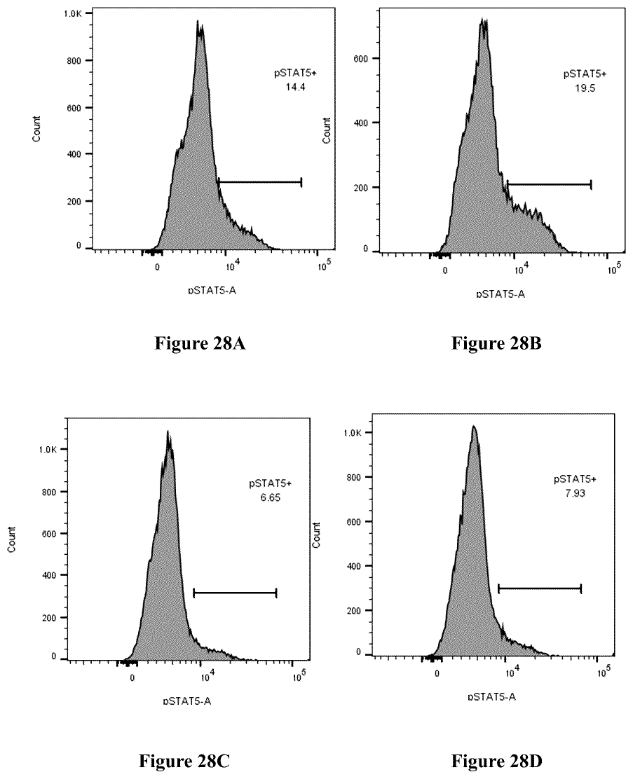

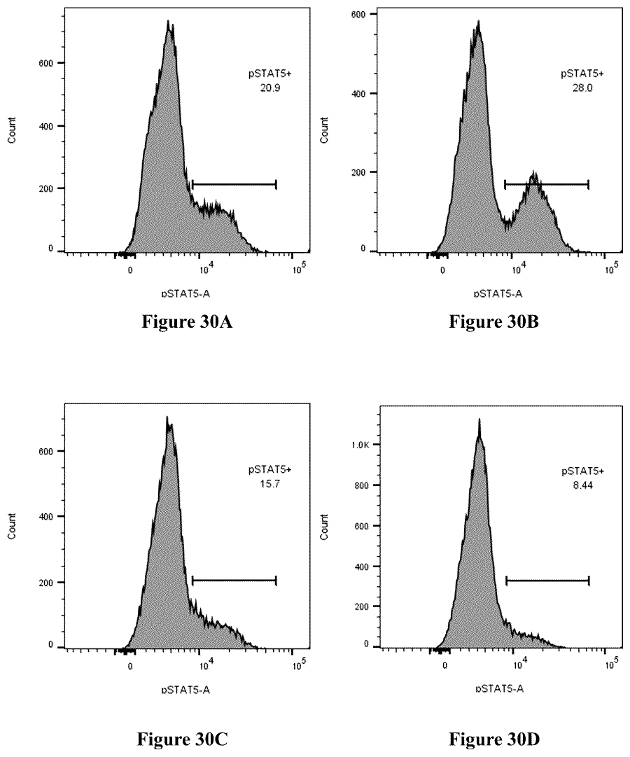

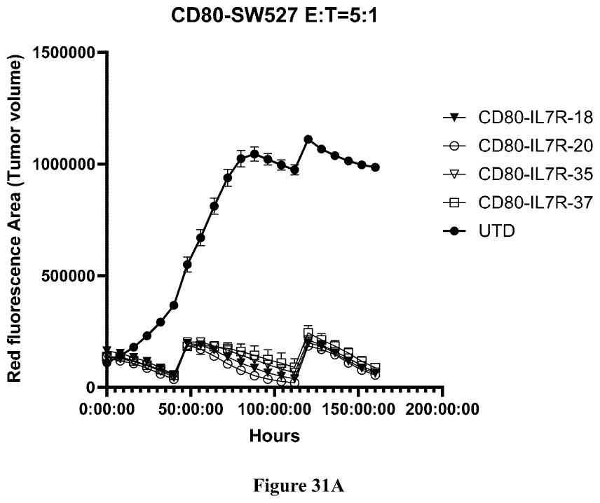

Primary CD4+ and CD8+ T cells transduced with polynucleotides encoding an anti-KRAS G12V TCR and an IL7RA fusion protein comprising a mutated IL7RA transmembrane domain of SEQ ID NOs: 42, 44, 122, or 123, and CD58 or CD80 extracellular domains. The transduced cells were cultured without IL-2, IL-15 or IL-7 for 24-72 hours before analysis. For pSTAT5 staining control, a transduced mixture of primary CD4 and CD8 T cells for each fusion protein was treated with 10 ng/ml IL-2 for 15-30 min, followed by pSTAT5 staining. The transduced mixture of primary CD4+ and CD8+ T cells with induced pSTAT5 signaling without ligand. FIGS. 28A-28D show the results of pSTAT5 staining for cells transduced with fusion proteins having a CD58 extracellular domain and mutated IL7RA transmembrane domains of: (1) SEQ ID NO: 42 (FIG. 28A); (2) SEQ ID NO: 44 (FIG. 28B); (3) SEQ ID NO: 122 (FIG. 28C); or SEQ ID NO: 123 (FIG. 28D). FIGS. 30A-30D show the results of pSTAT5 staining for cells transduced with fusion proteins having a CD80 extracellular domain and mutated IL7RA transmembrane domains of: (1) SEQ ID NO: 42 (FIG. 30A); (2) SEQ ID NO: 44 (FIG. 30B); (3) SEQ ID NO: 122 (FIG. 30C); or SEQ ID NO: 123 (FIG. 30D).

### T Cell Killing

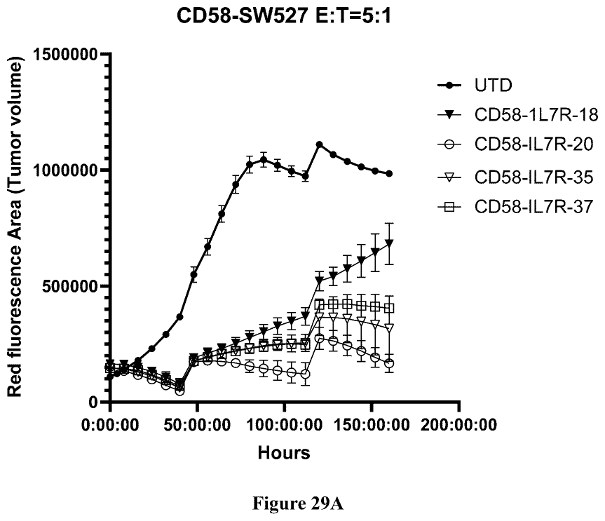

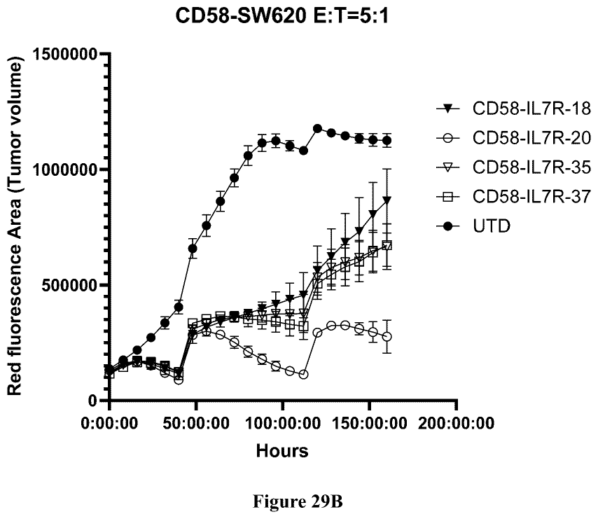

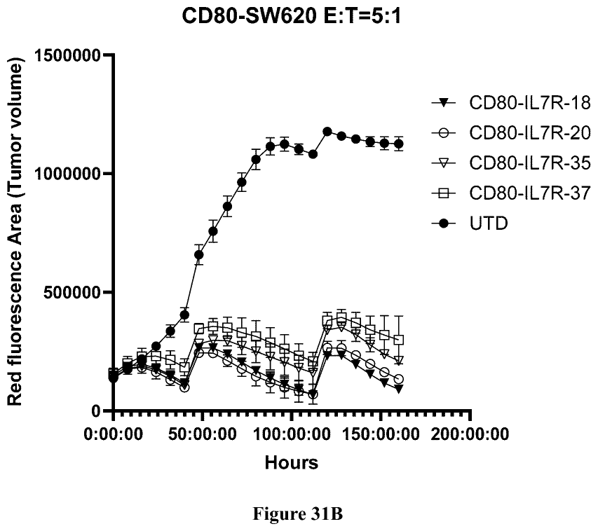

Tumor cells (e.g., from SW527, SW620 tumor cell lines) expressing a red fluorescent protein were cultured alone or with the TCR-transduced T cells described above for 188 hours at a 5:1 effector to target ratio (FIGS. 29A-29B and 31A-31B). Total red object area was measured as metric of tumor cell growth/viability throughout the study as indicated. Additional tumor cells were added at 76 hours and 144 hours. A transduced mixture of primary CD4+ and CD8+ T cells showed enhanced tumor cell killing.

CD58-IL7R-18 corresponds to the fusion protein of FIG. 28A; CD58-IL7R-20 corresponds to the fusion protein of FIG. 28B; CD58-IL7R-35 corresponds to the fusion protein of FIG. 28C; CD58-IL7R-37 corresponds to the fusion protein of FIG. 28D; CD80-IL7R-18 corresponds to the fusion protein of FIG. 30A; CD80-IL7R-20 corresponds to the fusion protein of FIG. 30B; CD80-IL7R-35 corresponds to the fusion protein of FIG. 30C; CD80-IL7R-37 corresponds to the fusion protein of FIG. 30D.

TABLE 4
Exemplary anti-KRAS G12V TCR Amino Acid Sequences.

| Name | Sequence |
|---|---|
| A11 KRAS | GAGVSQSPRYKVAKRGQDVALRCDPISGHVSLFWYQQ |
| TCR_220_21 | ALGQGPEFLTYFQNEAQLDKSGLPSDRFFAERPEGSV |
| Vbeta | STLKIQRTQQEDSAVYLCASSSEGLAGGPTAGELFFG |
| | EGSRLTVL (SEQ ID NO: 188) |
| A11 KRAS | GEDVEQSLFLSVREGDSSVINCTYTDSSSTYLYWYKQE |
| TCR_220_21 | PGAGLQLLTYIFSNMDMKQDQRLTVLLNKKDKHLSLRI |
| Valpha | ADTQTGDSAIYFCAEPIIGGNTPLVFGKGTRLSVIAN |
| | (SEQ ID NO: 189) |
| A11 KRAS | QKSPQPLTRRATMGTRLLCWVVLGFLGTDHTGAGVSQ |
| TCR_220_1 | SPRYKVAKRGQDVALRCDPISGHVSLFWYQQALGQGP |
| TCRbeta-P2A- | EFLTYFQNEAQLDKSGLPSDRFFAERPEGSVSTLKIQ |
| TCRalpha | RTQQEDSAVYLCASSSEGLAGGPTAGELFFGEGSRLT |
| fragment | VLEDLKNVFPPEVAVFEPSEAEISHTQKATLVCLATG |
| | FYPDHVELSWWVNGKEVHSGVCTDPQPLKEQPALNDS |
| | RYCLSSRLRVSATFWQNPRNHFRCQVQFYGLSENDEW |
| | TQDRAKPVTQIVSAEAWGRADCGFTSESYQQGVLSAT |
| | ILYEILLGKATLYAVLVSALVLMAMVKRKDSRGGSGA |
| | TNFSLLKQAGDVEENPGPMKTFAGFSFLFLWLQLDCM |
| | SRGEDVEQSLFLSVREGDSSVINCTYTDSSSTYLYWY |
| | KQEPGAGLQLLTYIFSNMDMKQDQRLTVLLNKKDKHL |
| | SLRIADTQTGDSAIYFCAEPIIGGNTPLVFGKGTRLS |
| | VIANIQNPDPAVYQLRDSKSSD (SEQ ID NO: 14) |

In the Table above, Vbeta denotes the beta chain variable region, V alpha denotes the alpha chain variable region, and TCRbeta-P2A-TCR-alpha fragment denotes a polypeptide including TCR alpha and TCR beta chains separated by a P2A self-cleaving peptide.

TABLE 5
Exemplary anti-KRAS G12V TCR Polynucleotide Sequence

| Name | Sequence |
|---|---|
| A11 KRAS | ctcaataaaa gagcccacaa cccctcactc ggcgcgccac catgggcaca agacttctct 60 |
| TCR_220_21 | gctgggtggt gcttggattt ctgggcacag atcatacagg agctggagtt agccagtctc 120 |
| TCRbeta-P2A- | ctaggtacaa agtggccaag agaggacagg atgtggctct gagatgtgac cctattagcg 180 |
| TCRalpha | gacatgtgag cctgttttgg taccagcaag ctctgggaca aggacccgag tttctgacct 240 |
| fragment | acttccagaa tgaagcccag ctggataaat ctggactgcc tagcgaccgg ttcttcgccg 300 |
| | aaagacctga aggatctgtt agcaccctga agattcagag aacacagcag gaggactctg 360 |
| | ccgtgtacct gtgtgcctct tcttctgaag gactggctgg aggacctaca gctggagaac 420 |
| | tgttttttgg agagggctct aggctgacag ttttggagga cctgaagaac gtgttccccc 480 |
| | cagaggtggc cgtgttcgag cctagcgagg ccgagatcag ccacacccag aaagccaccc 540 |
| | tcgtgtgcct ggccaccggc ttttaccccg accacgtgga actgtcttgg tgggtcaacg 600 |
| | gcaaagaggt gcacagcggc gtctgcaccg acccccagcc cctgaaagag cagcccgccc 660 |
| | tgaacgacag ccggtactgt ctgagcagca gactgagagt gtccgccacc ttctggcaga 720 |
| | acccccggaa ccacttcaga tgccaggtgc agttctacgg cctgagcgag aacgacgagt 780 |
| | ggacccagga ccgggccaag cccgtgaccc agatcgtgtc tgctgaggcc tggggcagag 840 |
| | ccgattgcgg cttcaccagc gagagctacc agcagggcgt gctgagcgcc accatcctgt 900 |
| | acgagatcct gctgggcaag gccaccctgt acgccgtgct ggtgtccgcc ctggtgctga 960 |
| | tggccatggt caagcggaag gacagccggg gcggttccgg agccacgaac ttctctctgt 1020 |
| | taaagcaagc aggagacgtg gaagaaaacc ccggtcccat gaagaccttt gccggattct 1080 |
| | ccttcctgtt cctgtggctg cagctggatt gtatgagcag aggcgaagat gtggaacaga 1140 |
| | gcctgtttct gagcgtgaga gagggagata gcagcgtgat caattgcacc tacaccgatt 1200 |
| | ctagcagcac ctacctgtac tggtacaagc aggaacctgg agccggatta caactgctga 1260 |
| | cctacatctt cagcaacatg gacatgaagc aggaccagag actgaccgtg ctgctgaaca 1320 |
| | agaaggacaa gcacctgagc ctgagaattg ccgatacaca gacaggagat agcgccatct 1380 |
| | acttctgtgc cgagcctatc attggcggca atacacctct ggtgtttgga aagggcacaa 1440 |
| | ggctgtctgt gattgccaac atccagaatc ccgaccctgc tgtgtaccag ctgcgggaca 1500 |
| | gcaagagcag cgac 1514 |
| | (SEQ ID NO: 144) |

The polynucleotide sequence in the Table above corresponds to the TCRbeta-P2A-TCR-alpha fragment polypeptide of Table 4.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A fusion protein comprising:
(a) an extracellular domain comprising one of the following:
(i) an extracellular domain of a Cluster of Differentiation 80 (CD80) polypeptide;
(ii) an extracellular domain of a Cluster of Differentiation 58 (CD58) polypeptide;
(iii) an extracellular domain of a Signal Regulatory Protein Alpha (SIRPα) polypeptide;
(iv) an extracellular domain of Cluster of Differentiation 40L (CD40L)polypeptide;
(v) an extracellular domain of a Cluster of Differentiation (CD2) receptor; or
(vi) an extracellular domain of Transforming Growth Factor beta receptor 2 (TGFβR2)
(b) an intracellular domain of an interleukin receptor polypeptide that is capable of interleukin signaling; and
(c) a transmembrane domain disposed between said extracellular domain and said intracellular domain.

2. The fusion protein of claim 1, wherein the fusion protein, when expressed by a host cell and contacted with a cognate ligand of the extracellular domain initiates interleukin signaling in said host cell or initiates interleukin signaling in said host cell in the absence of a ligand.

3. The fusion protein of claim 1, wherein the transmembrane domain comprises a transmembrane domain of one of the following polypeptides: TGRβR2, interleukin-7 receptor alpha subunit, CD80, CD58, Signal Regulatory Protein Alpha (SIRPα), CD40 Ligand (CD40L), or CD2; and/or the intracellular domain of the interleukin receptor polypeptide comprises an IL7R alpha subunit intracellular domain; and/or the extracellular component comprises the extracellular domain of said CD40L or a CD2 receptor.

4. A fusion protein comprising:
(a) an extracellular domain comprising an extracellular domain of a transforming growth factor beta receptor II (TGFBR2) polypeptide; or a transforming growth factor beta receptor I (TGFβR1) polypeptide, wherein said extracellular domain is capable of binding a TGFβ1, TGFβ2, or TGFβ3 polypeptide;
(b) an intracellular domain comprising an intracellular domain of an IL7RA polypeptide capable of IL7R alpha signaling; and
(c) a transmembrane domain disposed between the extracellular domain and the intracellular domain, wherein said transmembrane domain comprises a mutation that confers constitutive IL-7 signaling on the IL7R alpha intracellular domain.

5. The fusion protein of claim 4, wherein the fusion protein, when expressed by a host cell and bound to a TGFβ1, TGFβ2, or TGFβ3 polypeptide induces IL-7 signaling in the host cell.

6. The fusion protein of claim 4, wherein said transmembrane domain comprises a transmembrane domain of a TGFβR2, an IL7RA, a CD80, a CD58, a SIRPα, a CD40L, or a CD2 polypeptide, or a variant thereof.

7. A fusion protein comprising:
(a) an extracellular domain comprising a CD58 or CD80 extracellular domain;
(b) an IL7RA transmembrane domain comprising an amino acid sequence having at least 85% amino acid sequence identity to any one of amino acid sequences IFTCPSISILS (SEQ ID NO: 42), ILLTSHQPCILS (SEQ ID NO: 44), PITLYCKTLLTISILS (SEQ ID NO: 122), or ISPCITISILS (SEQ ID NO: 123); and
(c) an intracellular domain comprising an intracellular domain of an IL7RA polypeptide capable of IL7R alpha signaling.

8. A nucleic acid molecule encoding the fusion protein of claim 1 or a vector comprising the nucleic acid molecule encoding the fusion protein of claim 1.

9. A host cell comprising:
(a) the fusion protein of claim 1; and
(b) a nucleic acid molecule encoding a transgenic T cell receptor (TCR) receptor or chimeric antigen receptor (CAR), or an antigen binding fragment thereof.

10. The host cell of claim 9, wherein said TCR or said antigen binding fragment thereof specifically binds an HLA:peptide complex comprising a G12-mutant KRAS polypeptide.

11. The host cell of claim 9, wherein the host cell is a T cell.

12. The host cell of claim 9, wherein the host cell comprises one or more genetic alterations that reduce expression of a TRBC polypeptide, a T cell receptor polypeptide, and/or an MHC polypeptide.

13. A CD4+ or CD8+ T cell derived from a subject having a neoplasia, the T-cell comprising:
(a) a fusion protein of claim 1;
(b) a nucleic acid molecule encoding a transgenic T cell receptor (TCR) receptor or chimeric antigen receptor (CAR), or an antigen binding fragment thereof, wherein said TCR or said antigen binding fragment thereof specifically binds an HLA:peptide complex comprising a G12-mutant KRAS polypeptide or fragment thereof;
(c) one or more genetic alterations that reduce expression of a TRAC polypeptide, TRBC polypeptide, a T cell receptor polypeptide, and/or an MHC polypeptide

14. The cell of claim 13, wherein said HLA:peptide complex comprises an HLA-A11 polypeptide.

15. A CD4+ or CD8+ T cell derived from a subject having a cancer, the T-cell comprising:
(a) a fusion protein comprising a CD34, CD58, or CD80 extracellular domain, an IL7 receptor alpha transmembrane domain comprising the following amino acid sequence: PILLTCPTISILSFFSVALLVILACVLW, and an IL7 receptor alpha intracellular domain capable of IL7 receptor alpha signaling in the absence of ligand binding to the extracellular domain;
(b) a nucleic acid molecule encoding a transgenic T cell receptor (TCR) receptor or chimeric antigen receptor (CAR), or an antigen binding fragment thereof, wherein said TCR or said antigen binding fragment thereof specifically binds an HLA:peptide complex comprising a G12-mutant KRAS polypeptide or fragment thereof; and
(c) one or more genetic alterations that reduce expression of a TRAC polypeptide, TRBC polypeptide, a T cell receptor polypeptide, and/or an MHC polypeptide.

16. A pharmaceutical composition comprising an effective amount of a nucleic acid molecule encoding the fusion protein of claim 1.

17. A pharmaceutical composition comprising an effective amount of the cell of claim 9.

18. A method of activating a bystander T cell in a subject having a cancer, the method comprising administering to said subject an effective amount of the cell of claim 9 or a pharmaceutical composition comprising said cell.

19. The method of claim 18, wherein the cancer is selected from the group consisting of a cancer of the head or neck, melanoma, pancreatic cancer, cholangiocarcinoma, hepatocellular cancer, breast cancer, gastric cancer, lung cancer, prostate cancer, esophageal cancer, mesothelioma, colorectal cancer, and glioblastoma.

20. A kit for treating cancer in a subject, the kit comprising the pharmaceutical composition of claim 16, and directions for treating the subject.
